(19) United States
(12) Patent Application Publication
(10) Pub. No.: US 2023/0357838 A1
(43) Pub. Date: Nov. 9, 2023

(54) Double-Stranded DNA Deaminases and Uses Thereof

(71) Applicant: New England Biolabs, Inc., Ipswich, MA (US)

(72) Inventors: Zhiyi Sun, Gloucester, MA (US); Sean R. Johnson, Beverly, MA (US); Bo Yan, Ipswich, MA (US); Lixin Chen, Beverly, MA (US); G. Brett Robb, Somerville, MA (US); Thomas C. Evans, JR., Topsfield, MA (US); Romualdas Vaisvila, Ipswich, MA (US)

(73) Assignee: New England Biolabs, Inc., Ipswich, MA (US)

(21) Appl. No.: 18/323,143

(22) Filed: May 24, 2023

Related U.S. Application Data
(63) Continuation-in-part of application No. 18/058,115, filed on Nov. 22, 2022.
(60) Provisional application No. 63/264,513, filed on Nov. 24, 2021.

(51) Int. Cl. C12Q 1/6869; C12N 9/02; C12N 9/10; C12N 9/78

(52) U.S. Cl. CPC C12Q 1/6869; C12N 9/0071; C12Y 114/11; C12N 9/1051; C12Y 204/01027; C12N 9/78; C12Y 305/04

(57) ABSTRACT

Provided herein, among other things, is a method for deaminating a double-stranded nucleic acid. In some embodiments, the method may comprise contacting a double-stranded DNA substrate that comprises cytosines and a double-stranded DNA deaminase having an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163 and/or 164 to produce a deamination product that comprises deaminated cytosines. Enzymes and kits for performing the method are also provided.

### CROSS-REFERENCING

This application is a continuation-in-part of U.S. application Ser. No. 18/058,115, filed on Nov. 22, 2022, which claims priority to provisional application Ser. No. 63/264,513, filed on Nov. 24, 2021, which applications are incorporated by reference herein in their entirety.

### SEQUENCE LISTING

A Sequence Listing is provided herewith as a Sequence Listing XML, “NEB-451-CIP.xml” created on May 24, 2023, and having a size of 350 KB. The contents of the Sequence Listing XML are incorporated by reference herein in their entirety.

### BACKGROUND

In many organisms, cytosine in the genome can be covalently modified to, for example, 5-methylcytosine (5mC) or 5-hydroxymethylcytosine (5hmC). These epigenetic changes are believed to play a role in a wide variety of phenomena, including gene expression. Global or regional changes of DNA methylation are among the earliest events known to occur in cancer. The identification of methylation profiles in humans is a key step in studying disease processes and is increasingly used for diagnostic purposes.

Current methods for identifying modified cytosine include a deamination step in which cytosines are converted to uracils, leaving the modified cytosines undeaminated. Uracils in these deaminated DNA molecules are copied into thymines during amplification and, after sequencing the amplification products, each of the modified cytosines in the starting sequences can be readily identified as a “C” in the sequenced amplification product, whereas each of the cytosines appear as a “T” in the sequenced amplification product.

DNA may be deaminated chemically (using, e.g., bisulfite; see Frommer et al PNAS 1992 89: 1827-1831) or enzymatically using a DNA deaminase (e.g., APOBEC3A, see, e.g., Sun et al, Genome Res. 2021 31: 291-300 and Vaisvila et al Genome Res. 2021 31: 1280-1289). However, both of these approaches require a single-stranded substrate. As such, current workflows for analyzing modified cytosines typically involve a denaturation step. It would be desirable to eliminate the denaturation step from current workflows. Use of DNA deaminases having particular specificities, such as a bias for deaminating cytosines in a particular sequence context (e.g., the “CpG” context, the most common context for mammalian cytosine methylation) and/or selectivity for deaminating or not deaminating particular modifications, may further simplify such workflows as well as enable other genome analysis and engineering tools.

### SUMMARY

The present disclosure relates, in some embodiments, to deaminases having one or more desirable properties including, for example, cytosine deaminases that are active on DNA substrates.

These enzymes may deaminate cytosines in a double-stranded DNA substrate (e.g., without denaturing the DNA). Double-stranded DNA deaminases may deaminate cytosines in single-stranded DNA, in addition to deaminating cytosines in double-stranded DNA. Cytosines adjacent to guanines (“CG”) may be deaminated by disclosed deaminases as well as, not as well as, or better than cytosines in other sequence contexts (“CH”, H=A, C, T). Double-stranded DNA deaminase compositions may comprise a deaminase and, optionally, a buffer, one or more enzymes that alter the deamination susceptibility of one or more modified cytosines (e.g., a TET methylcytosine dioxygenase and/or a DNA beta-glucosyltransferase).

The present disclosure relates, in some embodiments, to methods for deaminating double-stranded DNA substrates. For example, deaminating a double-stranded DNA may comprise contacting the double-stranded DNA substrate and a double-stranded DNA deaminase to deaminate cytosines in the double-stranded substrate, for example, without denaturing the substrate or otherwise using any agents that unwind or otherwise separate the strands of the substrate (e.g., a gyrase or a helicase), to produce deamination products. In some embodiments, a double-stranded DNA deaminase may be used to deaminate cytosines in a single-stranded substrate, which may be preceded by separating the strands of the substrate. In some embodiments, methods may include sequencing at least one strand of the product of a deamination reaction (which is a deaminated double-stranded DNA molecule referred to herein as a “deamination product”) to produce sequence reads. A method may include amplifying a deamination product to produce an amplification product and then sequencing the amplification product to produce sequence reads. Disclosed cytosine deaminases may deaminate cytosines without deaminating modified cytosines (e.g., 5mC, 5hmC, 5fC, 5caC, 5ghmC, N4mC) also present in a DNA substrate or may both deaminate cytosines and deaminate one or more modified cytosines in a substrate. Accordingly, the positions of modified cytosines (e.g., 5mC or 5hmC) in a double-stranded DNA substrate can be identified by analysis of sequence reads. Some of the double-stranded DNA deaminases do not deaminate N4mC, but can deaminate other modified cytosines, others do not deaminate 5mC, and 5hmC, others do not deaminate 5hmC but can deaminate 5mC, others do not deaminate 5ghmC but can deaminate 5mC and/or 5hmC, and others that do not deaminate 5fC and 5caC but can deaminate 5mC and 5hmC (see, for example, Table 3). As such, the positions of one or more modified cytosines may be determined in a double-stranded substrate by contacting the substrate with a deaminase having a selected specificity and, optionally, pre-treating the substrate with one or more enzymes that alter the deamination susceptibility of one or more modified cytosines. For example, a method may include pre-treating the double-stranded DNA substrate with: (a) a TET methylcytosine dioxygenase and DNA beta-glucosyltransferase or (b) a TET methylcytosine dioxygenase but not DNA beta-glucosyltransferase. These enzymes modify 5mC and/or 5hmC in double-stranded nucleic acids to make those residues resistant to certain double-stranded DNA deaminases. In some embodiments, a method may include contacting a double-stranded DNA deaminase with a double-stranded nucleic acid not contacted (previously or concurrently) with a TET methylcytosine dioxygenase or a DNA beta-glucosyltransferase, for example, where the double-stranded DNA deaminase does not deaminate 5mC and/or 5hmC. In some embodiments, methods may include base editing and other genome engineering approaches.ln some embodiments, the double-stranded DNA substrate may comprise at least one N4mC or pyrrolo-dC. N4mC is found in prokaryotes and archaea. As such, in some embodiments, a double-stranded DNA substrate may be prokaryotic or archaeal. In some embodiments, a double-stranded DNA substrate may be made by ligating a hairpin adapter to a double-stranded fragment of DNA to produce a ligation product, enzymatically generating a free 3′ end in a double-stranded region of the hairpin adapter in the ligation product, and extending the free 3′ end in a dCTP-free reaction mix that comprises a strand-displacing or nick-translating polymerase, dGTP, dATP, dTTP and modified dCTP. In this method, the modified dCTP is incorporated into the new strand, to produce a double-stranded nucleic acid that has modified Cs. Enzymes and kits for performing the methods are also provided including, for example, a double-stranded DNA deaminase and a reaction buffer.

### BRIEF DESCRIPTION OF FIGS.

The file of this patent contains at least one drawing executed in color. Copies of this patent with color drawing(s) will be provided by the Patent and Trademark Office upon request and payment of the necessary fee.

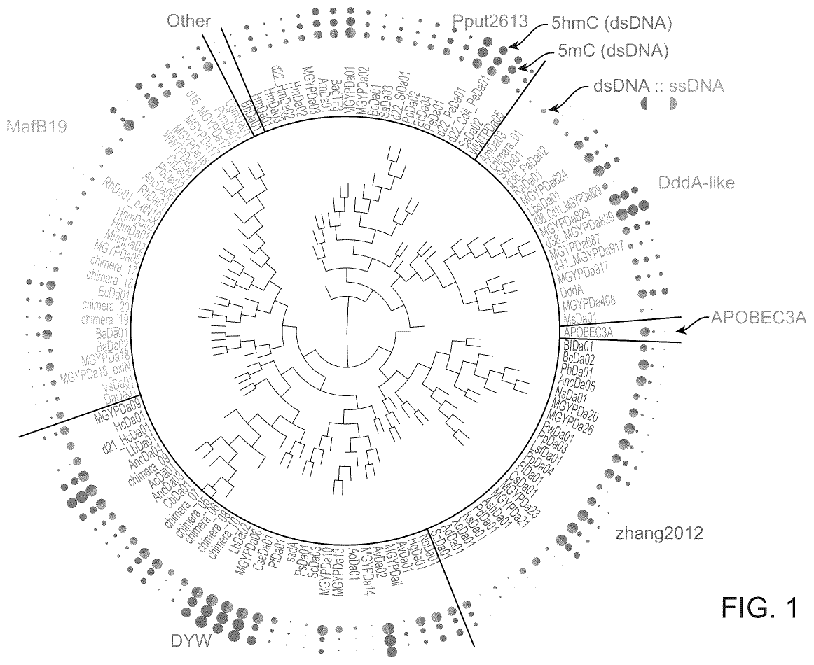

FIG. 1 shows the topology of a maximum likelihood phylogenetic tree of cytosine deaminases surrounded by illustrative activity data arranged in concentric rings, with each phylogenetic tree terminus, enzyme name, and set of activity results aligned along a radial axis. The enzymatic activity results for various substrates shown in these rings were measured by an in vitro screening assay with an

Illumina short-read sequencing-based detection method (Example 3). Total area of the circles corresponds to total activity and the relative sizes of colored sectors show relative activity on the indicated substrates. The inner-most ring shows relative deamination activity on unmodified cytosines in double-stranded DNA (blue sectors) compared to single-stranded DNA (red sectors). The middle ring shows activity on 5-methylated cytosine in double-stranded DNA. The outermost ring shows activity on 5-hydroxymethylated cytosine in double-stranded DNA. Enzyme names are colored according to their phylogenetic family.

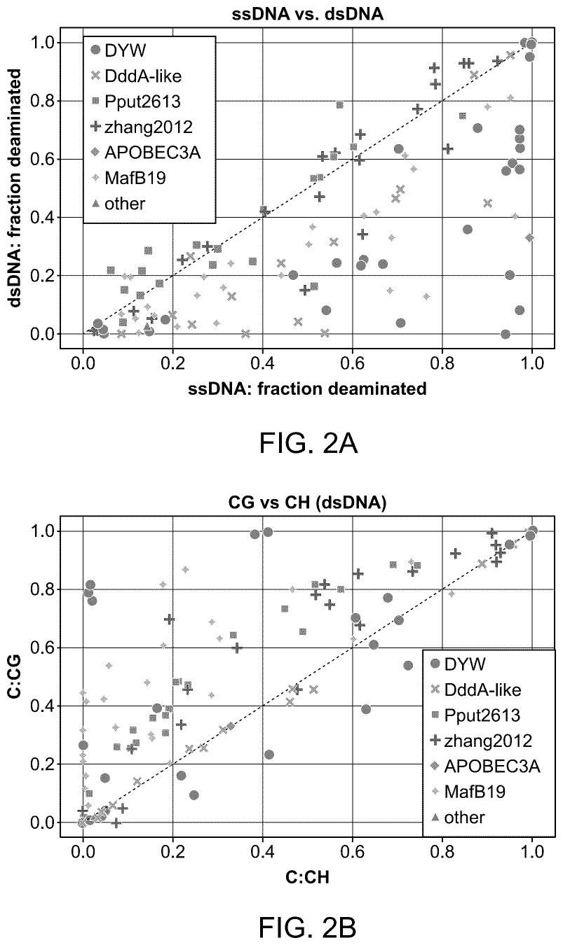

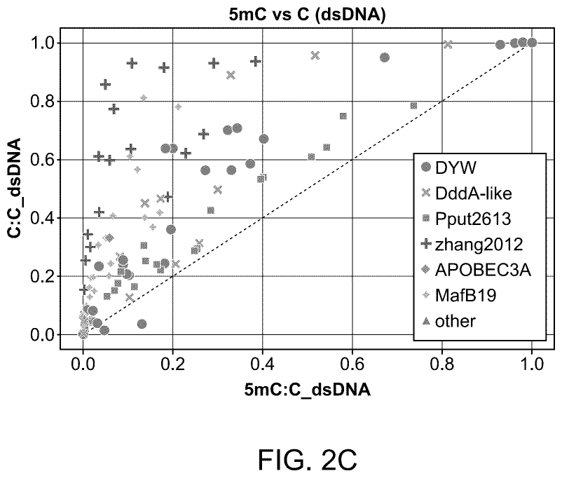

FIGS. 2A-C show enzymatic activity for cytosine deaminases assayed in accordance with the screening method of Example 3. Activities are expressed as deaminated fraction of total cytosines in the sample. FIG. 2A shows activity results for example deaminases on double stranded DNA vs. single stranded DNA. FIG. 2B shows activity results for example deaminases on unmodified cytosine in the CG context vs the CH (combination of CA, CC, and CT) context. FIG. 2C shows activity results for example deaminases on cytosine vs. 5-methylcytosine in all sequence contexts.

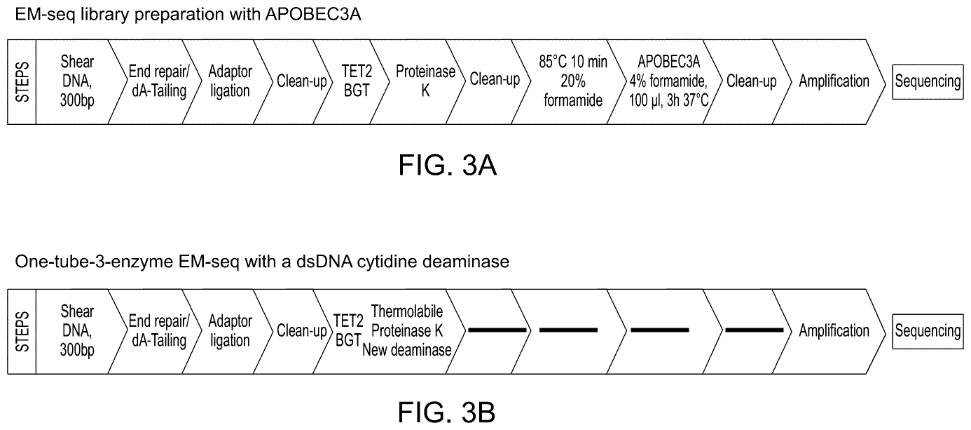

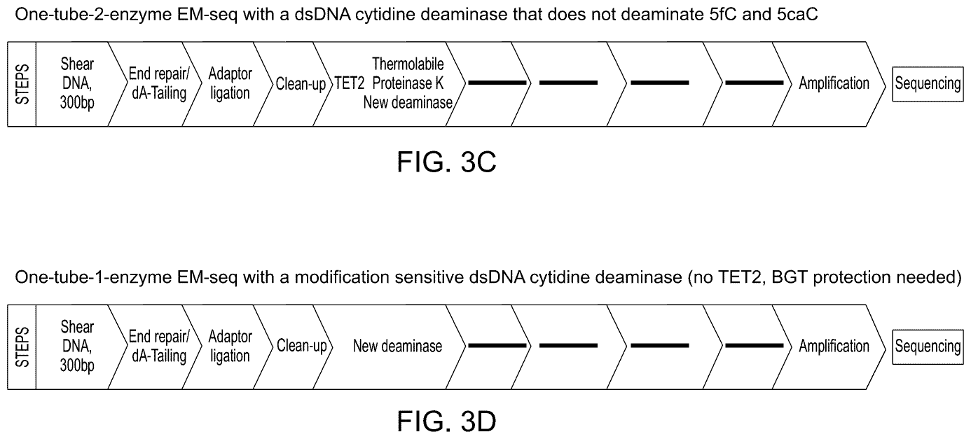

FIGS. 3A-3D shows example workflows for identifying the positions of modified cytosines in a DNA. FIG. 3A shows an example workflow of APOBEC3A deamination of ssDNA while FIGS. 3B, 3C, and 3D show example workflows in which APOBEC3Ais substituted by a cytosine deaminase that deaminates dsDNA. FIG. 3B shows an example single pot workflow in which use of a dsDNA deaminase that is active on ssDNA and dsDNA eliminates a DNA denaturation step. As shown, a DNA deaminase can be added to a reaction mix following reactions with TET and BGT without intermediate clean up and denaturing steps thereby enhancing detection of target methylated sites on genomic DNA and methylome mapping. FIG. 3C shows an example workflow in which the substrate is contacted with a deaminase that does not deaminate 5fC or 5caC without requiring or including pre-treatment with BGT. FIG. 3D shows an example methylome analysis workflow in which the substrate is contacted with a single enzyme—a dsDNA deaminase.

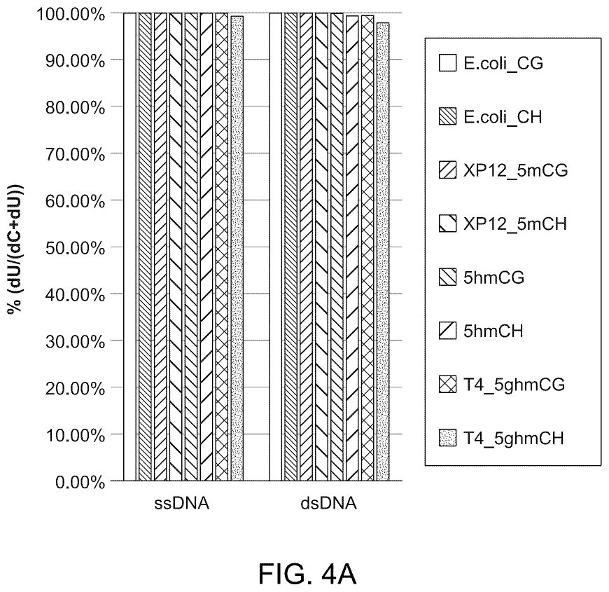

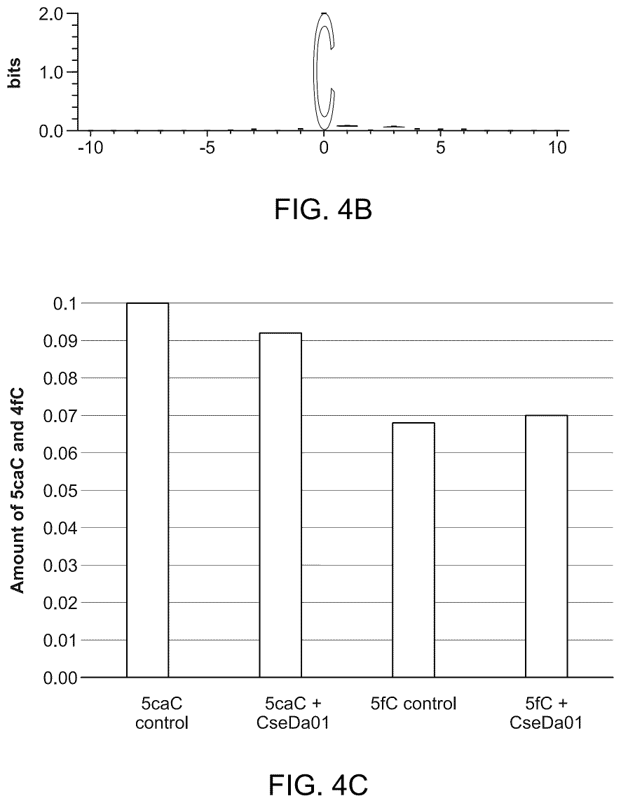

FIGS. 4A-4C show example results of a workflow to detect 5mC and 5hmC that, like FIG. 3C, does not require or include a BGT glycosyltransferase pretreatment and the dsDNA deaminase used, CseDa01, does not deaminate 5caC and 5fC. FIG. 4A shows that CseDa01 DNA deaminase efficiently deaminates cytosine C, 5mC, 5hmC and 5ghmC in both single-stranded and double-stranded substrates. FIG. 4B shows that CseDa01 DNA deaminase exhibits no sequence bias and the deamination efficiencies were greater than 95% for both the CpG and CpH contexts in E. coli genome for both ssDNA and dsDNA substrates. FIG. 4C shows that CseDa01 DNA deaminase does not deaminate 5caC and 5fC and may be useful to detect 5mC and 5hmC without a BGT glucosylation step.

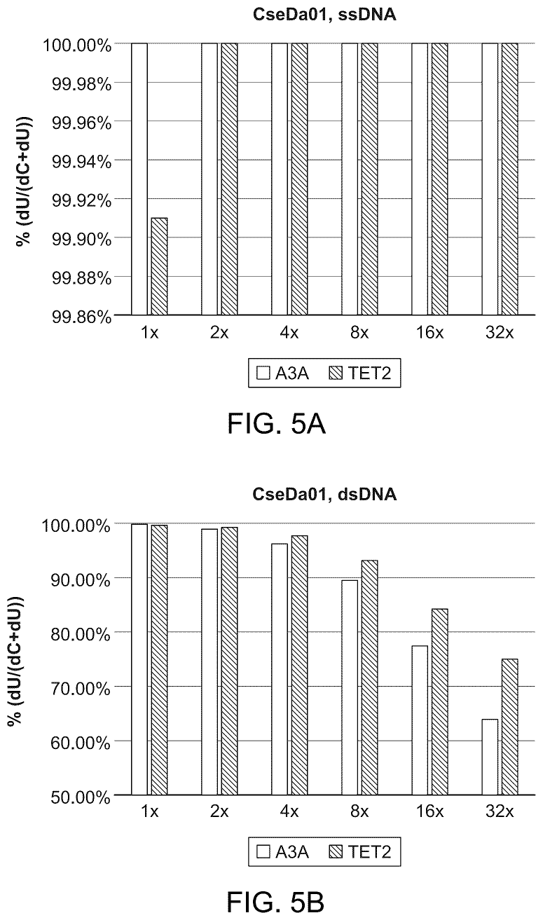

FIGS. 5A-5B show example results of using CseDa01 and TET2 to perform single tube oxidation of 5mC. The X-axis labels show serial dilutions of the deaminase, with 1x being the most concentrated enzyme, and 32x being a dilution by a factor of 32 compared to 1x. FIG. 5A shows results illustrating efficient deamination of a single-stranded substrate. FIG. 5B shows results illustrating efficient deamination of a double-stranded substrate.

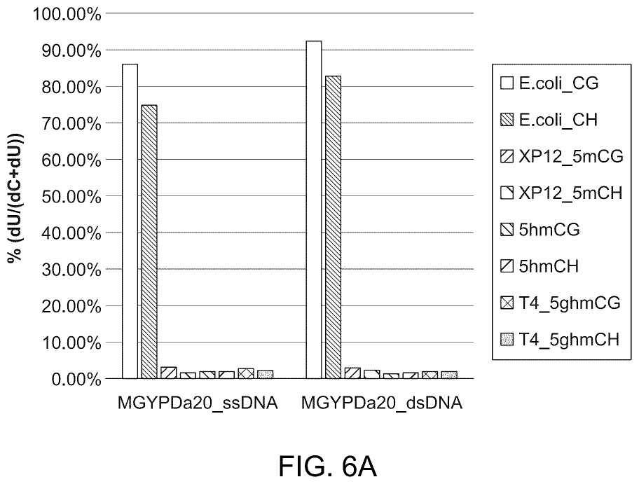

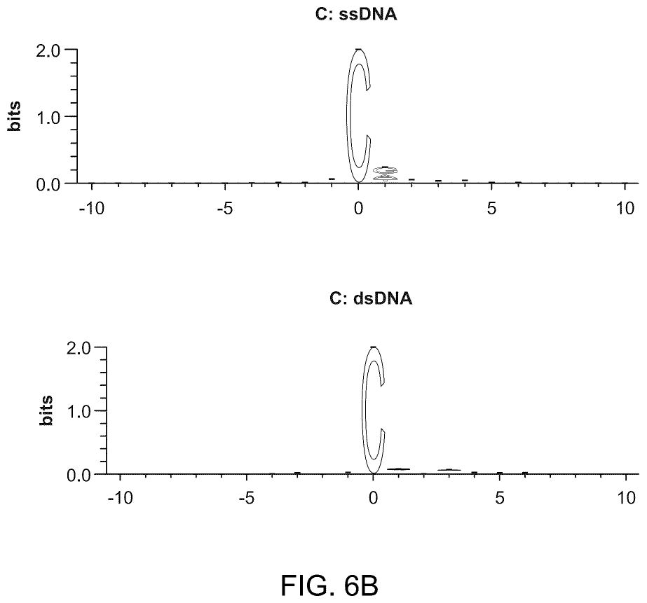

FIGS. 6A-6B show example results of using MGYPDa20, a modification-sensitive deaminase to efficiently deaminate cytosines to uracil. However, it does not deaminate 5-methylcytosine and 5-hydroxymethylcytosine in dsDNA and ssDNA. This deaminase may be used to detect 5mC and 5hmC without the protection of these modified bases. FIG. 6A shows that MGYPDa20 DNA deaminase efficiently deaminates cytosine C but not 5mC, 5hmC or 5ghmC. FIG. 6B shows that MGYPDa20 DNA deaminase exhibits no sequence bias. The sequence logos were generated using the cytosine sites that have >=90% deamination efficiency in the E. coli genome.

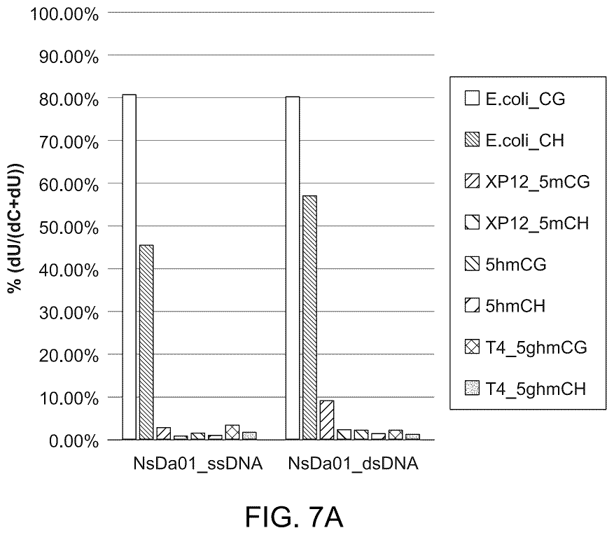

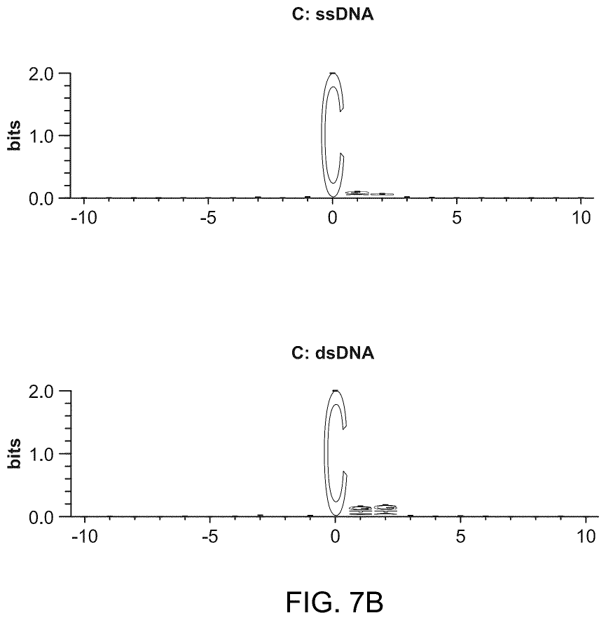

FIGS. 7A-7B show example results of using another modification-sensitive dsDNA deaminase, NsDa01, which may be used to detect 5mC and 5hmC without the protection of modified bases. FIG. 7A shows that NsDa01 DNA deaminase efficiently deaminates cytosine C but not 5mC, 5hmC or 5ghmC.

FIG. 7B shows that NsDa01 DNA deaminase exhibits no sequence bias. The sequence logos were generated using the cytosine sites that have >=90% deamination efficiency in the E. coli genome.

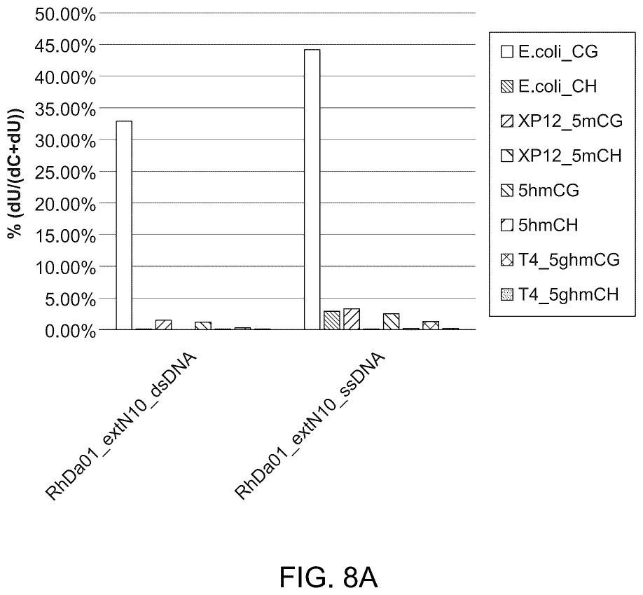

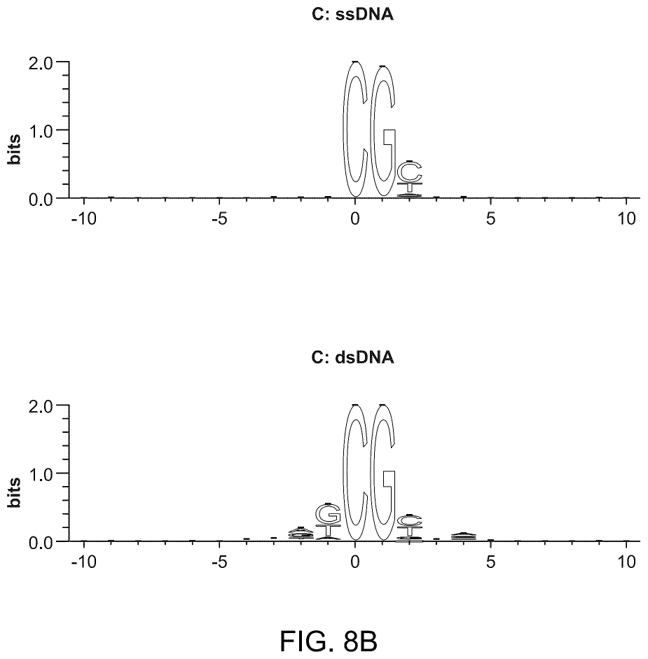

FIGS. 8A-8B show example results of using a CpG-specific modification-sensitive dsDNA deaminase, RhDa01, which may be used to detect 5mC and 5hmC in the CpG context with or without the protection of modified bases. FIG. 8A shows that RhDa01 DNA deaminase efficiently deaminates cytosine C in CpG context but not 5mC, 5hmC or 5ghmC. FIG. 8B shows that RhDa01 DNA deaminase exhibits CpG sequence specificity. The sequence logos were generated using the cytosine sites that have >=90% deamination efficiency in the E. coli genome.

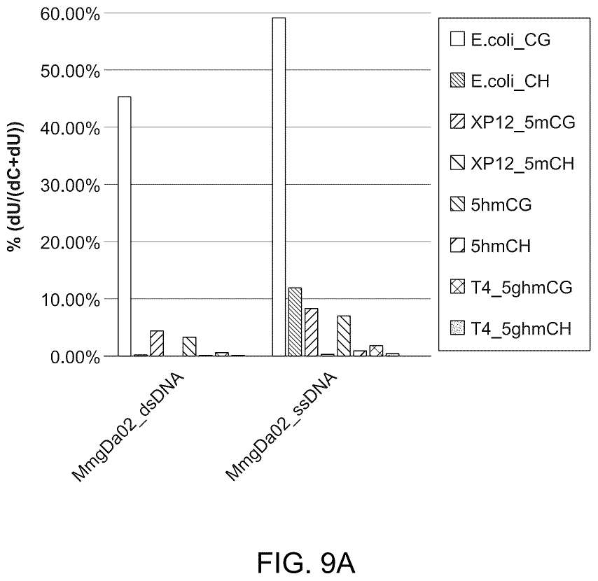

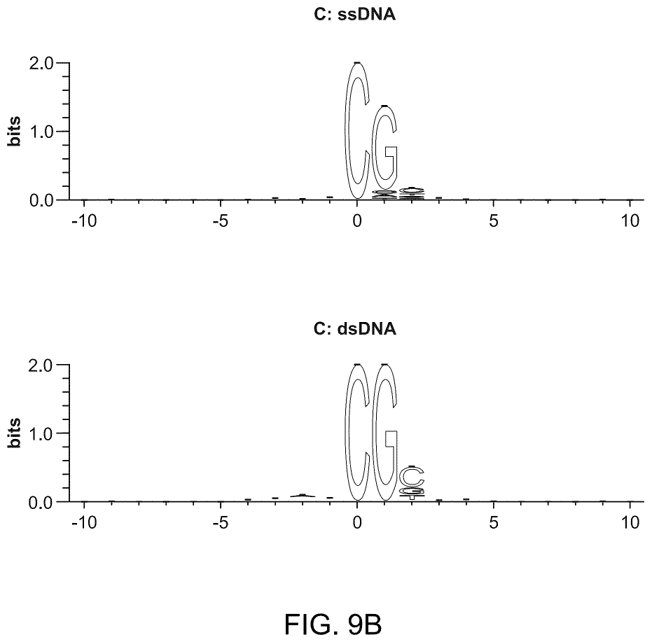

FIGS. 9A-B shows example results of using a CpG-specific modification-sensitive dsDNA deaminase, MmgDa02, which may be used to detect 5mC and 5hmC in the CpG context with or without the protection of modified bases. FIG. 9A shows that MmgDa02 DNA deaminase efficiently deaminates cytosine C in CpG context but not 5mC, 5hmC or 5ghmC. FIG. 9B shows that MmgDa02 DNA deaminase exhibits a CpG sequence specificity. The sequence logos were generated using the cytosine sites that have >=90% deamination efficiency in the E. coli genome.

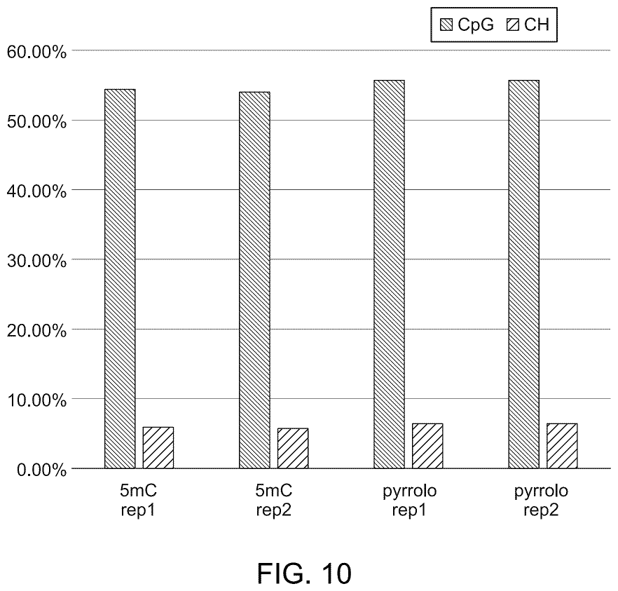

FIG. 10 shows example results of using a one-tube-one-enzyme EM-seq method to map 5mC in human using a modification-sensitive dsDNA deaminase, MGYPDa20. It shows that 5mC and 5hmC in the human GM12878 genome may be correctly detected using a modification-sensitive DNA deaminase MGYPDa20. Two types of adapters were used in these experiments,—all Cs were replaced by 5mC or Pyrrolo-dC. In both cases the overall methylation level in the human GM12878 genome was identified correctly.

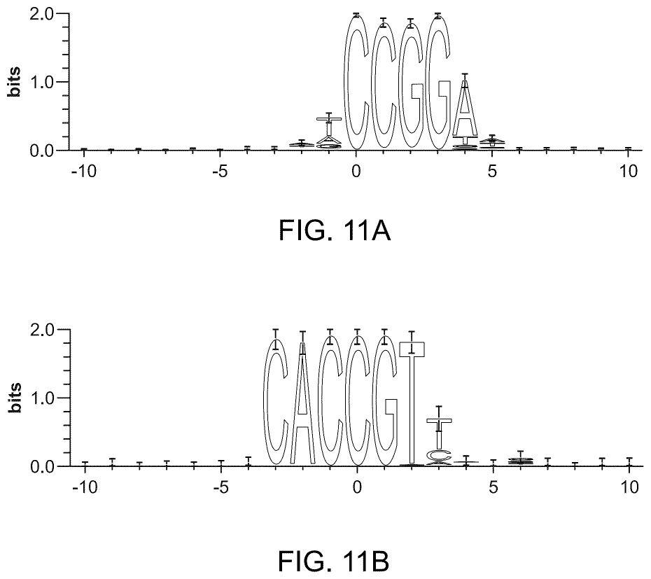

FIG. 11A-11B shows example results of using sequence logos of not deaminated sites by the CseDa01 deaminase from the N4mC-containing substrates of different genomes with different methyltransferase sequence specificities, namely Paenibacillus species JDR-2 (CCGG target sequence) and Salmonella enterica FDAARGOS_312 (CACCGT target sequence). Eukaryotic deaminase family of APOBEC3A deaminates N4mC, but bacterial deaminases do not, therefore, the newly characterized bacterial deaminases may be used to detect N4mC modifications. FIG. 11A shows that the detected N4mC motif matches the expected CCGG methyltransferase motif in Paenibacillus species JDR-2.

FIG. 11B shows that the detected N4mC motif matches CACCGT from Salmonella enterica FDAARGOS_312.

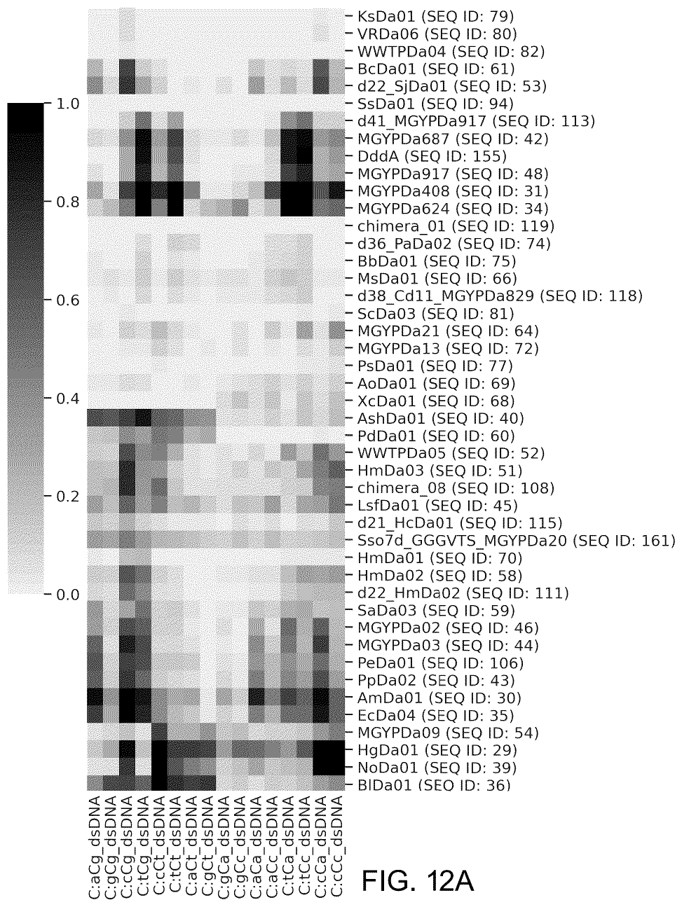

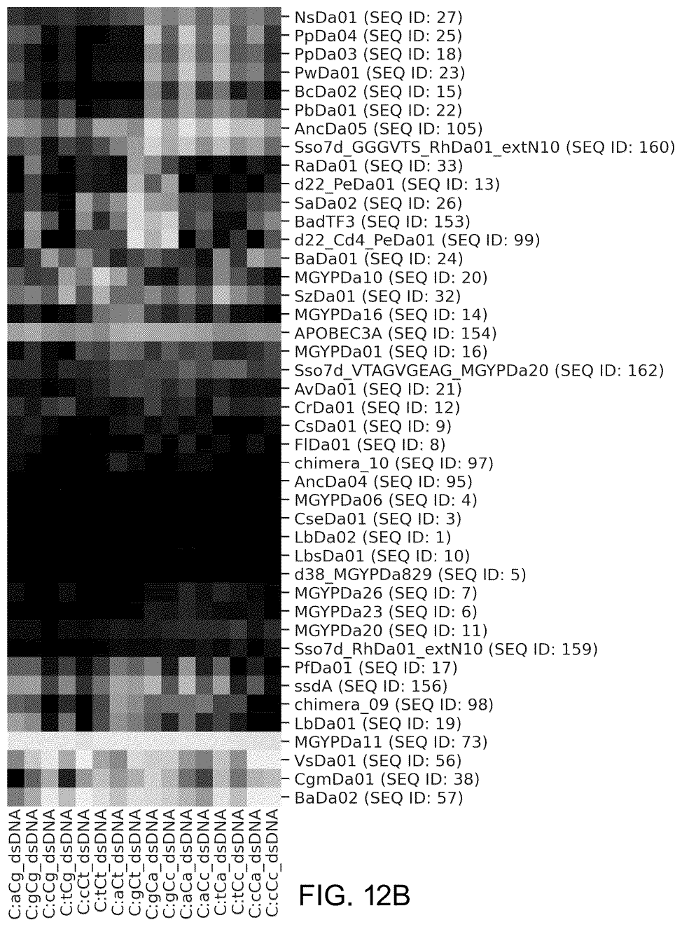

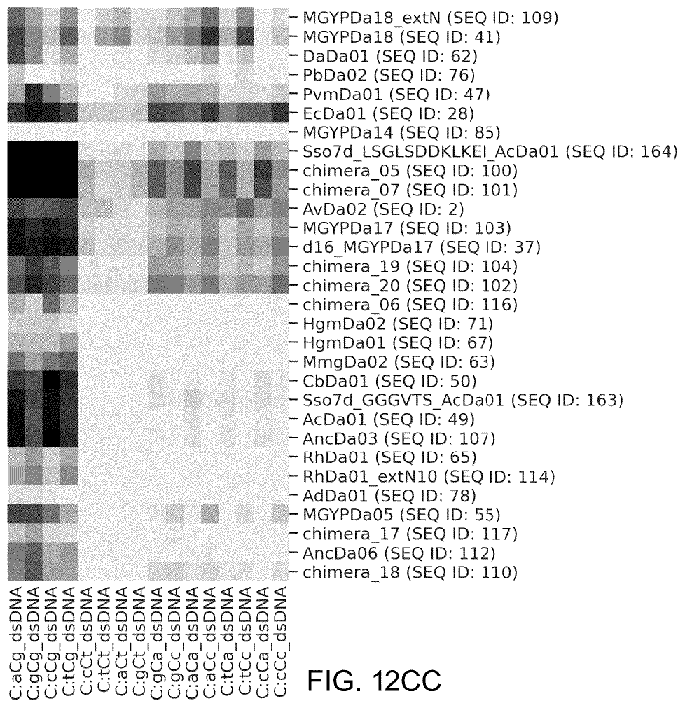

FIG. 12A-124C shows deamination efficiency on nCn contexts of unmodified dsDNA. Rows and columns are sorted based on average linkage clustering of cosine distances. Darker spots indicate higher activity on the three base context specified by the column, as indicated by the scale depicted on

FIG. 12A. FIG. 12B is continued from FIG. 12A; FIG. 12C is continued from FIG. 12B.

### DETAILED DESCRIPTION

The present disclosure provides double-stranded DNA deaminases, variants, ancestors, fusions, compositions, systems, apparatus, methods, and workflows for deaminating double-stranded DNA (in duplex form, without denaturation). Applications of these deaminases include, for example, EM-seq, methyl-SNP-seq, and N4mC detection, among others.

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor,

Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like. As used herein and in the appended claims, the singular forms “a”, “an”, and “the” include plural referents unless the context clearly dictates otherwise. For example, the term “a protein” refers to one or more proteins, i.e., a single protein and multiple proteins. Optional elements may be expressly excluded where exclusive terminology is used, such as “solely,” “only”, in connection with the recitation of the optional elements or when a negative limitation is specified.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “deaminase substrate” refers to a polynucleotide (e.g., a DNA) molecule that optionally may be exclusively double-stranded, partially double-stranded and partially single-stranded, or exclusively single-stranded. A deaminase substrate may comprise one or more cytosines, one or more modified cytosines, one or more adenines, one or more modified adenines, or combinations thereof. A DNA substrate may comprise one or more adapters. As described in Example 10, such adapters may contain modified nucleotides that are not deaminated during the deamination step. Adapters that do not contain modified nucleotides may be used, so long as base pairing is sufficient to allow the adapters to attach to cognate binding partners as required for a particular method. Additionally, adapters containing modified nucleotides are not required, for example, when the adapters are attached after the deamination step.

In the context of the present disclosure, “double-stranded DNA deaminase” refers to a hydrolyase that deaminates cytosines in double-stranded DNA to uracils and/or deaminates adenines in double-stranded DNA to hypoxanthines. A double-stranded DNA deaminase may deaminate cytosines and/or adenines in double-stranded DNA as well as or better than it deaminates cytosines and/or adenines, respectively, in single-stranded DNA. For example, a double-stranded DNA deaminase may deaminate cytosines double-stranded DNA, but not deaminate cytosines in single-stranded DNA. A double-stranded DNA deaminase may be modification sensitive. For example, a double-stranded DNA deaminase may deaminate an unmodified cytosine or adenine in double-stranded DNA, but not deaminate one or more corresponding modified cytosines or adenines.

In the context of the present disclosure, “duplex” and “double stranded” refer to any conformation of a polynucleotide in which two polynucleotide strands (e.g., separate molecules or spatially separated portions of a single molecule) are arranged anti parallel to one another in a helix with complementary bases of each strand paired with one another (e.g., in Watson-Crick base pairs). Paired bases may be stacked relative to one another to permit pi electrons of the bases to be shared.

Duplex stability, in part, may be related to the ratio of complementary bases to mismatches (if any) in the two strands, ratio of pairs with three hydrogen bonds (e.g., G:C) to pairs with two hydrogen bonds (e.g., A:T, A:U) in the duplex, and the length of the strands with higher ratios and longer strands generally associated with higher stability. Duplex stability, in part, may be related to ambient conditions including, for example, temperature, pH, salinity, and/or the presence, concentration and identity of any buffer(s), denaturant(s) (e.g., formamide), crowding agent(s) (e.g., PEG), detergent(s) (e.g., SDS), surfactant(s), polysaccharide(s) (e.g., dextran sulfate), chelator(s) (e.g., EDTA), and nucleic acid(s) (e.g., salmon sperm DNA). A duplex polynucleotide may comprise one or more unpaired bases including, for example, a mismatched base, a hairpin loop, a single-stranded (5′ and/or 3′) end.

Duplex polynucleotides (e.g., double-stranded DNA deaminase substrates) may have any desired length. For example, a duplex polynucleotide may have a length of 50 nucleotides, 10-200 nucleotides, 80-400 nucleotides, 50-500 nucleotides, 500 nucleotides, 1 kb, 2 kb, 5 kb or 10 kb.

Duplex polynucleotides may have any desired number of mismatched or unpaired nucleotides, for example, 1 per 100 nucleotides, 2 per 100 nucleotides, 3 per 100 nucleotides, 5 per 100 nucleotides, or 10 per 100 nucleotides.

In the context of the present disclosure, “fusion protein” refers to a protein composed of two or more polypeptide components that are un-joined in their native state. Fusion proteins may be a combination of two, three or four or more different proteins. For example, a fusion protein may comprise two naturally occurring polypeptides that are not joined in their respective native states. A fusion protein may comprise two polypeptides, one of which is naturally occurring and the other of which is non-naturally occurring. The term polypeptide is not intended to be limited to a fusion of two heterologous amino acid sequences. A fusion protein may have one or more heterologous domains added to the N-terminus, C-terminus, and or the middle portion of the protein. If two parts of a fusion protein are “heterologous”, they are not part of the same protein in its natural state. Examples of fusion proteins include proteins comprising a double-stranded DNA deaminase fused to a protein such as albumin, another enzyme (e.g., an endonuclease), an antibody, a binding domain suitable for immobilization such as maltose binding domain (MBP), a histidine tag (“His-tag”), a chitin binding domain, an alpha mating factor or a SNAP-Tag® (New England Biolabs, Ipswich, MA (see for example U.S. Pat. Nos. 7,939,284 and 7,888,090)), a DNA-binding domain (e.g., the DNA binding domain of a transcription factor, a non-specific DNA-binding domain (e.g., Sso7d), or a specific DNA binding domain (e.g., BD09; see, for example, U.S. Pat. No. 9,963,687), or a methyl binding domain (MBD), with the deaminase optionally positioned closer to the N-terminus or closer to the C-terminus than the other component(s). A binding peptide may be used to improve solubility or yield of the deaminase during the production of the protein reagent. Other examples of fusion proteins include fusions of a deaminase and a heterologous targeting sequence, a linker, an epitope tag, a detectable fusion partner, such as a fluorescent protein, (3-galactosidase, luciferase and/or functionally similar peptides. Components of a fusion protein may be joined by one or more peptide bonds, disulfide linkages, and/or other covalent bonds.

In the context of the present disclosure, “modified cytosine” refers to any covalent modification of cytosine including naturally occurring and non-naturally occurring modifications. Modified cytosines include, for example, 1-methylcytosine (1mC), 2-O-methylcytosine (m2C), 3-ethylcytosine (e3C), 3,^{N}4 -ethylenocytosine (SC), 3-methylcytosine (3mC), 4-methylcytosine (4mC), 5-carboxylcytosine (5CaC), 5-formylcytosine (5fC), 5-hydroxymethylcytosine (5hmC), 5-methylcytosine (5mC), ^{N}4 -methylcytosine (N4mC), and pyrrolo-cytosine (pyrrolo-C). 5-carboxylcytosine (5caC) is the final oxidized derivative of 5-methylcytosine (5mC). 5mC is oxidized to 5-hydroxymethylcytosine (5hmC) which is then oxidized to 5-formylcytosine (5fC) then 5caC. Additional examples of modified nucleotides may be found at https://dnamod.hoffmanlab.org.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′- end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature; (b) having components in concentrations not found in nature; (c) omitting one or components otherwise found in naturally occurring compositions; (d) having a form not found in nature, e.g., dried, freeze dried, crystalline, aqueous; and (e) having one or more additional components beyond those found in nature (e.g., buffering agents, a detergent, a dye, a solvent or a preservative).

With reference to an amino acid, “position” refers to the place such amino acid occupies in the primary sequence of a peptide or polypeptide numbered from its amino terminus to its carboxy terminus. A position in one primary sequence may correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence may correspond to a position within a functionally equivalent motif or structural motif that can be identified within one or more other sequence(s) in a database by alignment of the motifs. Analogously, with reference to a nucleotide, “position” refers to the place such nucleotide occupies in the nucleotide sequence of an oligonucleotide or polynucleotide numbered from its 5′ end to its 3′ end.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

Double-stranded DNA Deaminases The present disclosure relates to naturally occurring and non-naturally occurring double-stranded DNA deaminases. A non-naturally occurring double-stranded DNA deaminase may relate to, but differ from, a naturally occurring protein. Naturally-occurring proteins often include a deaminase as a single domain of a larger, multi-domain structure with the deaminase domain positioned at the most C-terminal end. Non-naturally occurring double-stranded DNA deaminases may constitute truncated versions of a naturally-occurring protein, in which cases, the non-naturally occurring double-stranded

DNA deaminases may have a high degree of identity to a portion of a naturally-occurring sequence, but lack, for example, structural and/or functional domains or sub-units of the corresponding naturally-occurring proteins. A non-naturally occurring double-stranded DNA deaminase may have any number of insertions, deletions, or substitutions relative to a naturally occurring enzyme. For example, a non-naturally occurring double-stranded DNA deaminase may have less than 100% identity, less than 99% identity, less than 98% identity, less than 90% identity, less than 85% identity, less than 80% identity, less than 70% identity, less than 60% identity, less than 50% identity, less than 40% identity, less than 30% identity, or less than 20% identity to a naturally occurring enzyme. Non-naturally occurring double-stranded DNA deaminases may include expression and/or purification tags. Non-naturally occurring double-stranded DNA deaminase disclosed herein may have an amino acid sequence that is at least 80% identical (e.g., at least 90% identical, at least 95% identical or at least 98% identical or at least 99% identical to) the C-terminal deaminase domain of a naturally-occurring protein, wherein the double-stranded DNA deaminase possesses a double-stranded DNA deaminase activity and does not comprise the N-terminus of the corresponding naturally-occurring protein (if any). In some embodiments, a non-naturally occurring double-stranded DNA deaminase lacks at least 10, at least 20, at least 50 or at least 100 of the N-terminal amino acids of the corresponding naturally-occurring protein. In some embodiments, a double-stranded DNA deaminase is no more than 300 amino acids in length, e.g., no more than 200 amino acids in length or no more than 150 amino acids in length.

According to some embodiments, a double-stranded DNA deaminase may comprise an amino acid sequence having at least 80%, at least 85%, at least 88% identical, at least 90%, at least 92%, at least 93%, at least 95%, at least 96%, at least 97%, at least 98% or at least 99% identity to any of SEQ ID NOS: 1-152. In some embodiments, a double-stranded DNA deaminase may be encoded by a nucleic acid sequence that, when transcribed, translated, and/or processed, results in an amino acid sequence having at least 80%, at least 85%, at least 90%, at least 93%, at least 96%, at least 97%, at least 98% or at least 99% identity to any of SEQ ID NOS: 1-152. A double-stranded DNA deaminase may have an amino acid sequence at least 90% (e.g., at least 95%, at least 98%, at least 99%) identical to any of SEQ ID NOS:
2, 3, 4, 5, 6, 7, 8, 9, 10, 11, 12, 14, 15, 16, 19, 24, 26, 27, 28, 33, 40, 49, 50, 63, 95, 96, 97, and/or 99. In some embodiments, a double-stranded DNA deaminase may have an amino acid sequence at least 90% (e.g., at least 95%, at least 98%, at least 99%) identical to any of SEQ ID NOS: 21, 40, 47, 49, 50, 55, 58, 59, 62, 63, 65, 67, 70, 71, 76, 106, 107, 110, 112, 114, 117, 163, and 164. In some embodiments, a non-naturally occurring double-stranded DNA deaminase lacks the N-terminus of its corresponding naturally-occurring protein, for example, at least 10, at least 20, at least 50 or at least 100 of the N-terminal amino acids. Variants can be designed using sequence alignments and structural information. In some embodiments, a double-stranded DNA deaminase may contain a fragment of a wild type protein, where the fragment contains a deaminase domain, but lacks other domains of the wild type protein that may be C-terminal and/or N-terminal to the deaminase domain. Examples of non-naturally-occurring double-stranded DNA deaminases include SEQ ID NOS: 2, 3, 4, 5, 6, 7, 8, 9, 10, 11, 12, 14, 15, 16, 19, 24, 26, 27, 28, 33, 40, 49, 50, 63, 95, 96, 97, and/or 99. Other examples of non-naturally-occurring double-stranded DNA deaminases include SEQ ID NOS: 21, 21, 40, 47, 49, 50, 55, 58, 59, 62, 63, 65, 67, 70, 71, 76, 106, 107, 110, 112, 114, 117, 163, and 164.

In some embodiments, a double-stranded DNA deaminase may be a fusion protein. For example, a double-stranded DNA deaminase may have a purification tag (e.g., a His tag or the like) at either end. In some embodiments, a double-stranded DNA deaminase may be fused to a DNA binding protein (e.g., the DNA binding domain of a transcription factor) or the protein component of a nucleic acid-guided endonuclease (e.g., a catalytically dead Cas9 (dCas9) or a Cas9 nickase (nCas9) or TALEN (transcription activator-like effector nucleases)) so that the fusion protein can affect site-specific C to T substitutions in a genome. Example methods of “base editing” are described in, for example, Komor et al (Nature 533: 420-424), among other publications.

A double-stranded DNA deaminase optionally may deaminate cytosine, but not adenine (a “dsDNA cytosine deaminase”), deaminate adenine, but not cytosine (a “dsDNA adenine deaminase”), or deaminase both adenine and cytosine (appreciating that one may be a better substrate than the other under otherwise equivalent conditions). A double-stranded DNA deaminase may be modification sensitive. For example, a double-stranded DNA deaminase may deaminate cytosine, but not deaminate one or more modified cytosines in double stranded DNA. For example, a double-stranded DNA deaminase may deaminate cytosine, but not deaminate 5mC or N4mC or it may deaminate C and 5mC, but not 5hmC, 5ghmC or N4mC.

### Double-stranded DNA Deaminase Compositions

The present disclosure provides double-stranded DNA deaminase compositions including, for example, reaction mixtures. According to some embodiments, deaminase compositions may comprise (a) a double-stranded DNA deaminase and (b) a double-stranded DNA. A deaminase composition may comprise, for example, a deaminase variant (e.g., having an amino acid sequence at least 80% identical to one or more of SEQ ID NOS:1-152). A double-stranded DNA deaminase composition may be free of one or more other catalytic activities. For example, a double-stranded DNA deaminase composition may be free of nucleases that cleave dsDNA, free of nucleases that cleave ssDNA, free of polymerase activity, free of DNA modification activity, and/or free of protease activity, in each case, under desired test conditions (e.g., conditions of time, temperature, pH, salinity, model substrate and/or others), for example, conditions intended to replicate conditions of a specific use of the double-stranded DNA deaminase composition or intended to represent conditions for a range of uses.

In some embodiments, double-stranded DNA deaminases and compositions comprising one or more double-stranded DNA deaminase may have any desirable form including, for example, a liquid, a gel, a film, a powder, a cake, and/or any dried or lyophilized form. A double-stranded DNA deaminase composition may comprise a double-stranded DNA deaminase and a support or matrix, for example, a film, gel, fabric, or bead comprising, for example, a magnetic material, agarose, polystyrene, polyacrylamide, and/or chitin.

In some embodiments, a reaction mix may comprise: a double-stranded DNA substrate that comprises cytosines and a double-stranded DNA deaminase. A double-stranded DNA substrate may comprise cytosines and at least one modified cytosine, e.g., a 5fC, 5CaC, 5mC, 5hmC, N4mC or pyrrolo-C. A double-stranded DNA substrate may be eukaryotic DNA (e.g., plant or animal) or bacterial. In some embodiments, the double-stranded DNA substrate may be mammalian, e.g., from a human. In some embodiments, the double-stranded DNA substrate may be human cfDNA. The reaction mix may additionally comprise one or more of a TET methylcytosine dioxygenase (e.g., TET2) and a DNA beta-glucosyltransferase, as described herein and/or a ligase, a polymerase, a proteinase K, and/or a thermolabile proteinase K. A reaction mix may be free of unwinding agents (e.g., gyrases, topoisomerases, single-stranded DNA binding proteins, or helicases) and/or free of denaturants.

### Double-stranded DNA Deaminase Methods

The present disclosure provides methods for identifying the type and/or position of modified nucleotides in, for example, DNA using a deaminase. In some embodiments, a method may comprise providing a double-stranded DNA substrate of any desired length. For example, a double-stranded DNA substrate may have a length of 50 nucleotides, 10-200 nucleotides, 80-400 nucleotides, 50-500 nucleotides, 500 nucleotides, 1 kb, 2 kb, 5 kb or 10 kb. A double-stranded DNA substrate, in some embodiments, may be a fragment of genomic DNA, organelle DNA, cDNA, or other DNAs of interest and can be or arise from any desired source (e.g., human, non-human mammal, plants, insects, microbial, viral, or synthetic DNA). A DNA substrate may be prepared, in some embodiments by extracting (e.g., genomic DNA) from a biological sample and, optionally, fragmenting it. In some embodiments, fragmenting DNA may comprise mechanically fragmenting the DNA (e.g., by sonication, nebulization, or shearing) or enzymatically fragmenting the DNA (e.g., using a double stranded DNA “dsDNA” fragmentation mix). Examples of enzymes for fragmentation include NEBNext® Fragmentase®, Ultrashear, and FS systems (New England Biolabs, Ipswich MA)), among others. In some embodiments, DNA for deamination may already be fragmented (e.g., as is the case for FFPE samples and circulating cell-free DNA (cfDNA)).

According to some embodiment, a method may include polishing DNA ends (e.g., the ends of fragmented DNA). For example, DNA ends may be contacted with (a) a proofreading polymerase to excise 3′ overhanging nucleotides, if any, (b) a proofreading and/or non-proofreading polymerase to fill in 5′ overhangs, if any, and/or (c) a polynucleotide kinase (PNK) to phosphorylate unphosphorylated 5′ ends, if any. In some embodiments, a method may comprise contacting DNA ends (e.g., blunt ends) with a non-proofreading polymerase to add an untemplated A-tail (e.g., a single base overhang comprising adenine) to the 3′ end. Methods may include, according to some embodiments, ligating one or more adapters to DNA ends. Adapters may comprise one or more sample tags, unique molecular identifiers (UMIs), modified nucleotides, primer sequences (e.g., for sequencing). In some embodiments, adapters may comprise cytosines (or adenines) that are not substrates for the deaminase to be used. If desired, polishing products and/or ligation products may be cleaned up, for example, to separate polishing products or ligation products, as applicable, from enzymes, unreacted nucleotides and/or adapters.

In some embodiments, a method may comprise contacting (a) a deaminase substrate and (b) a glucosyltransferase (e.g., T4-BGT) and/or Ten-eleven translocation (TET) dioxygenase to produce a modified deaminase substrate. BGT may glucosylate 5hmC to form 5ghmC. TET may oxidize 5mC to 5caC. If subsequently treated with sodium bisulfite or Apolipoprotein B mRNA editing enzyme subunit 3A (APOBEC3A), all Cs except 5ghmC in the modified deaminase substrate would be deaminated. Deaminases disclosed herein may obviate the need to denature the DNA prior to deamination (e.g., as with APOBEC3A) and may provide methylation sensitivities.

A method may comprise contacting a double-stranded DNA substrate that comprises cytosines and a double-stranded DNA deaminase to produce a deamination product that comprises deaminated cytosines. A double-stranded DNA substrate may further comprise one or more modified cytosines, e.g., one or more modified cytosines selected from 5fC, 5CaC, 5mC, 5hmC, N4mC and pyrrolo-C, 4mC, EC, 3mC, e3C, m2C, and 1mC. A double-stranded DNA deaminase substrate does not need to be denatured before or during deamination. As such, methods can be practiced in the absence of a denaturation step. In some embodiments, deamination methods may comprise contacting a double-stranded DNA substrate comprising cytosines and a double-stranded DNA deaminase to produce a reaction mix to produce a deamination product comprising deaminated cytosines.

Deamination methods may further comprise amplifying the deamination product to produce an amplification product, thereby copying any deaminated Cs in the original strand to Ts in the amplification product. Deamination methods may further comprise ligating an asymmetric (or “Y”) adapter, e.g., an Illumina P5/P7 adapter, onto the deamination product and amplifying the deaminated product using primers complementary to sequences in the adapter. In some embodiments, a method may comprise sequencing a deamination product, or amplifying a deamination product to produce amplification products and sequencing the amplification products, in each case, to produce sequence reads. Deamination products and/or amplification products may be sequenced using any suitable system including Illumina's reversible terminator method (see, e.g., Shendure et al, Science 2005 309: 1728). In some embodiments, a deaminated product may be sequenced directly, without amplification, for example, by nanopore or PacBio sequencing. A sequencing step may result in at least 10,000, at least 100,000, at least 500,000, at least 1M, at least 10M, at least 100M, at least 1B or at least 106 sequence reads per reaction. In some cases, the reads may be paired-end reads. A method may comprise analyzing sequence reads to identify a modified cytosine in the double-stranded DNA substrate, where a modified cytosine can be identified as a “C” because it is deaminase-resistant.

Double-stranded DNA deaminases that are “blocked” by or do not deaminate modified cytosines (e.g., 5mC, 5hmC, 5ghmC, N4mC) may be used in a variety of “EM-seq”-like workflows for the analysis of modified cytosines (e.g., see FIG. 3D). Double-stranded DNA deaminases that deaminate modified cytosines may also be used in a variety of “EM-seq” like workflows for the analysis of modified cytosines (e.g, see FIGS. 3B and 3C). Current implementations of EM-seq employ a deaminase that has a preference for single-stranded substrates. As such, the current EM-seq workflow has a denaturation step (see, e.g., FIG. 3A, Sun et al Genome Res. 2021 31: 291-300 and Vaisvila et al Genome Res. 2021 31: 1280-1289). In the present workflow, the denaturation step can be eliminated, thereby making EM-seq workflow faster and more efficient. Use of a double-stranded DNA deaminase that has CpG bias may make methylation sequencing analysis more efficient by reducing the number of cytosines in the double-stranded DNA sample that are deaminated. For example, a double-stranded DNA substrate may contain cytosines in both CpG and CpH contexts, as well as modified cytosines in a CpG context. The sequences obtained from the top and bottom strands of such a deaminated substrate will contain positions that do not base pair. Take as an example a double-stranded DNA substrate 21 base pairs long, having 2 pairs of symmetric modified cytosines in a CpG context, 1 pair of symmetric unmodified cytosine in a CpG context, and 4 unmodified cytosines not in a CpG context on the top strand and 3 unmodified cytosines not in a CpG context on the bottom strand (C T G T 5mC G G A C 5mC G C A G TCT AC G A (SEQ ID NO:169). After deamination using bisulfite or a non-selective deaminase, 42% of the positions are no longer base-pairing because the unmodified Cs in each strand will read as Ts (5 from one strand, 4 from the other). Thus, the top and bottom strands are about 25% different from the original DNA sequence. To reduce this variation to provide more accurate sequence of the genetic basis and allow identification of modified cytosines by comparing unconverted sequencing reads to a standard reference sequence that is not C to T converted or to a sequence that is assembled directly from the sequencing reads, a double-stranded deaminase selective for unmodified cytosines in CpG context (with CpG bias) may be used as described in Example 8, Table 2, Application 6 and Example 16.

Workflows for example deamination methods are shown in FIGS. 3B-3D. The steps of such workflows may be performed in any logically possible order, e.g., the double-stranded DNA substrate may be subjected to deamination prior to steps such as end repair/dA-tailing and/or adaptor ligation. As illustrated in FIG. 3B, a double-stranded DNA substrate may be prepared by pre-treating a double-stranded DNA with a TET methylcytosine dioxygenase (e.g., TET2) and DNA beta-glucosyltransferase to convert the 5mC and 5hmC in the starting DNA to forms resistant to double-stranded DNA deaminases, e.g., the MGYPDa829, MGYPDa06, CrDa01, AvDa02, CsDa01, LbsDa01, FIDa01, MGYPDa26, MGYPDa23, chimera_10 and AncDa04. Double-stranded DNA deaminases useful in the illustrated workflow may have an amino acid sequence that is at least 80% identical to the amino acid sequence of any of MGYPDa829 (SEQ ID NO:96), MGYPDa06 (SEQ ID NO: 4), CrDa01 (SEQ ID NO: 12), AvDa02 (SEQ ID NO: 21), CsDa01 (SEQ ID NO: 9), LbsDa01 (SEQ ID NO: 10), FIDa01 (SEQ ID NO: 8), MGYPDa26 (SEQ ID NO: 7), MGYPDa23 (SEQ ID NO: 6), chimera_10 (SEQ ID NO: 97) and AncDa04 (SEQ ID NO: 95) double-stranded DNA deaminases. For methods in which a double-stranded DNA deaminase that has sequence bias for the CpG context is used, a double-stranded DNA deaminase useful in the illustrated workflow may have an amino acid sequence that is at least 80% identical to the amino acid sequence of any of PvmDa01 (SEQ ID NO:47), AcDa01 (SEQ ID NO:49), CbDa01 (SEQ ID NO:50), MGYPDa05 (SEQ ID NO:55), HmDa02 (SEQ ID NO:58), SaDa03 (SEQ ID NO:59), HmDa01 (SEQ ID NO:70), PbDa02 (SEQ ID NO:76), PeDa01 (SEQ ID NO:106), AncDa03 (SEQ ID NO:107), Sso7d_GGGVTS_AcDa01 (SEQ ID NO:163), and Sso7d_LSGLSDDKLKEI_AcDa01(SEQ ID NO:164) double-stranded DNA deaminases.

As illustrated, the double-stranded DNA deaminase can be added to the reaction without any clean-up, denaturation or addition of unwinding agents.

As illustrated in FIG. 3C, a double-stranded DNA substrate may be prepared by pre-treating a double-stranded DNA with a TET methylcytosine dioxygenase (e.g., TET2) but not DNA beta-glucosyltransferase to convert 5mC in the starting DNA to a form resistant to double-stranded DNA deaminases, e.g., the CseDa01 and LbDa02. Double-stranded DNA deaminases useful in the illustrated workflow may have an amino acid sequence that is at least 90% identical to the amino acid sequence of any of CseDa01 (SEQ ID NO: 3) and LbDa02 (SEQ ID NO: 1) double-stranded DNA deaminases. In this embodiment, the double-stranded DNA deaminase can be added to the reaction without any clean-up, denaturation or addition of unwinding agents.

As illustrated in FIG. 3D, a double-stranded nucleic acid may not be contacted with a TET methylcytosine dioxygenase nor a DNA beta-glucosyltransferase (nor any other enzyme that converts a modified cytosine to a form resistant to a selected double-stranded DNA deaminase) at any point in the workflow. For example, a selected double-stranded DNA deaminase may be blocked by 5-hydroxymethylcytosine and 5-methylcytosine. Double-stranded DNA deaminases useful in the illustrated workflow may have an amino acid sequence that is at least 90% identical to the amino acid sequence of any of MGYPDa20 (SEQ ID NO: 11), NsDa01 (SEQ ID NO: 27), and AshDa01 (SEQ ID NO: 40) double-stranded DNA deaminases. For methods in which a double-stranded DNA deaminase with bias for CpG context is used, a double-stranded DNA deaminase useful in an illustrated workflow may have an amino acid sequence that is at least 80% identical to the amino acid sequence of any of AshDa01 (SEQ ID NO:40), DaDa01 (SEQ ID NO:62), MmgDa02 (SEQ ID NO:63), RhDa01 (SEQ ID NO:65), HgmDa01 (SEQ ID NO:67), HgmDa02 (SEQ ID NO:71), chimera_18 (SEQ ID NO:110), AncDa06 (SEQ ID NO:112), RhDa01_extN10 (SEQ ID NO:114), and Chimera_17 (SEQ ID NO:117) double-stranded DNA deaminases.

In some embodiments, a double-stranded DNA substrate may comprise at least one N4mC (N4-methyl-cytosine) which is a cytosine modification that is resistant to some double-stranded DNA deaminases. Double-stranded DNA deaminases useful for detecting N4mC may have an amino acid sequence that is at least 90% identical to the amino acid sequence of any of SEQ ID NOS:1-28. For example, double-stranded DNA deaminases useful for detecting N4mC may have an amino acid sequence that is at least 90% identical to the amino acid sequence of any of CseDa01 (SEQ ID NO:3) and LbDa01 (SEQ ID NO:19) double-stranded DNA deaminases. In these embodiments, the double-stranded DNA substrate may be or comprise prokaryotic or archaeal DNA.

In some embodiments, the double-stranded DNA deaminase may be used in a “methyl-SNP-seq” workflow (see, e.g., Yan et al, Genome Res. 2022; gr.277080.122). For example, a method may comprise; (a) ligating a hairpin adapter to a double-stranded fragment of DNA to produce a ligation product, (b) enzymatically generating a free 3′ end in a double-stranded region of the hairpin adapter in the ligation product; and (c) extending the free 3′ end in a dCTP-free reaction mix that comprises a strand-displacing or nick-translating polymerase, dGTP, dATP, dTTP and modified dCTP to produce the double-stranded DNA substrate, as described in U.S. Provisional Application Ser. No. 63/399,970, filed on Aug. 22, 2022, which application is incorporated by reference herein. Examples of modified dCTPs include 5mdCTP, pyrrolo-dCTP, and N4mdCTP among other modified dCTPs that can be incorporated by a polymerase. Deaminases may have an amino acid sequence that is at least 90% identical to the amino acid sequence of any of MGYPDa20 (SEQ ID NO: 11), NsDa01 (SEQ ID NO: 27), AshDa01 (SEQ ID NO: 40).

Other current workflows for sequencing genomic DNA employ multi-copy sequencing strategies to simultaneously obtain both genetic bases (e.g., G, A T, C) and epigenetic bases (e.g., methylated bases), for example to better distinguish between genetic C-to-T mutations and epigenetically modified cytosine. These workflows (e.g., Yan et al, ibid, and Fullgrabe, et al., Nat Biotechnol (2023). https://doi.org/10.1038/s41587-022-01652-0) involve copying strands of genomic DNA fragments to generate two copies of the strand on a single DNA strand. The multi-copy strands are then deaminated, followed by sequencing. This results in obtaining two sequence reads of the same strand of the genomic DNA fragment (and if desired four reads because both strands of the genomic DNA fragment may be subjected to this process); bioinformatics tools are used to discern whether Cs arose from modified C or from a mutation in the genomic DNA, as well as to identify errors arising from sequencing or amplification steps. Such workflows involve linking together the two strands of the genomic DNA, e.g., using a hairpin; breaking that linkage to synthesize the copy, thereby creating the multi-copy strand. Then, in either order, deaminating the multi-copy strand, and adding sequencing primers to the multi-copy strands to obtain reads of the original and copied sequences. The sequences are determined using rules based on the selected deamination process. A double-stranded DNA deaminase described herein may be used to reduce the complexity of such workflows (see, e.g, Example 10). A double-stranded DNA deaminase described herein may also be used to sequence genetic and epigenetic bases using a standard sequencing workflow by adding a deamination step, without the need for making a multi-copy strand (e.g., see FIG. 3D). Not using a multi-copy strand simplifies data analysis because standard base calling, sequence analysis, and methylation calling may be used rather than custom bioinformatics tools for resolving sequences obtained using the published dual-copy processes referenced above. The sequencing methods described herein may also allow identification of modified cytosines by using a standard reference sequence that is not C to T converted, or using a sequence that is assembled directly from the sequencing reads generated from the same library. Whereas published methods for genetic and epigenetic sequencing using multi-copy strands requires at least nanogram amounts of sample, the sequencing methods described herein may be carried out using input DNA quantities of about 50 nanograms or less, including about 20 nanograms or less, 10 nanograms or less, 5 nanograms or less, 2 nanograms or less; 1 nanogram or less, 100 picograms or less, 50 picograms or less, 20 picograms or less, 10 picograms or less, 5 picograms or less. For example, as described in Example 17, 10 picograms was the input DNA quantity.

According to some embodiments, a double-stranded DNA deaminase composition may comprise a double-stranded DNA deaminase and, optionally, any of (including one or more of) a buffering agent (e.g., a storage buffer, a reaction buffer), an excipient, a salt (e.g., NaCl, MgClₗ2, CaCₗ2), a protein (e.g., albumin, an enzyme), a stabilizer, a detergent (for example, ionic, non-ionic, and/or zwitterionic detergents (e.g., octoxinol, polysorbate 20)), a polynucleotide, a cell (e.g., intact, digested, or any cell-free extract), a biological fluid or secretion (e.g., mucus, pus), an aptamer, a crowding agent, a sugar (e.g., a mono, di, tri, tetra, or higher saccharide), a starch, cellulose, a glass-forming agent (e.g., for lyophilization), a lipid, an oil, aqueous media, a support (e.g., a bead) and/or (non-naturally occurring) combinations thereof. Combinations may include for example, two or more of the listed components (e.g., a salt and a buffer) or a plurality of a single listed component (e.g., two different salts or two different sugars). Examples of proteins that may be included in a double-stranded DNA deaminase composition include one or more enzymes that alter the deamination susceptibility of one or more modified cytosines (e.g., a TET methylcytosine dioxygenase and/or a DNA beta-glucosyltransferase).

### Double-Stranded DNA Deaminase Kits

The present disclosure relates, in some embodiments, to a deaminase kit comprising a double-stranded DNA deaminase. A kit may comprise any of the components described herein. A double-stranded DNA deaminase composition or kit may include, for example, double-stranded DNA deaminase and, optionally, a storage buffer (e.g., comprising a buffering agent and comprising or lacking glycerol), and/or a reaction buffer. A reaction buffer for a deaminase composition or a deaminase kit may be in concentrated form, and the buffer may include one or more additives (e.g., glycerol), one or more salts (e.g. KCl), one or more reducing agents, EDTA, one or more detergents, one or more non-ionic surfactants, one or more ionic (e.g. anionic or zwitterionic) surfactants, and/or crowding agents. A kit comprising dNTPs may include one, two, three of all four of dATP, dTTP, dGTP and dCTP. A kit may further comprise one or more modified nucleotides.

One or more components of a kit may be included in one container for a single step reaction, or one or more components may be contained in one container, but separated from other components for sequential use or parallel use. For example, a kit may comprise two components in a single tube (e.g., a deaminase and a storage buffer) and all other components in separate, individual tubes, in each case, with the contents provided in any desired form (e.g., liquid, dried, lyophilized). One tube in a kit may contain a mastermix, for example, for receiving and amplifying a DNA (e.g., a deaminated DNA). For example, a double-stranded DNA deaminase may be deposited in the cap of a tube while components for transcribing a template nucleic acid are deposited in the body of the tube. As desired, for example, upon completion of the deamination reaction, the tube may be tapped, shaken, turned, spun, or otherwise moved to contact the deposited double-stranded DNA deaminase with the deamination reaction mixture. A kit may include a double-stranded DNA deaminase and the reaction buffer in a single tube or in different tubes and, if included in a single tube, the double-stranded DNA deaminase and the buffer may be present in the same or separate locations in the tube. For example, a kit may comprise a double-stranded DNA deaminase, as described above, and a reaction buffer (e.g., a 5× or 10× buffer). The contents of a kit may be formulated for use in a desired method or process. In some embodiments, the kit may further comprise (a) a TET methylcytosine dioxygenase (e.g., TET2) and a DNA beta-glucosyltransferase or (b) a TET methylcytosine dioxygenase and no DNA beta-glucosyltransferase. In some embodiments, a kit does not contain either a TET methylcytosine dioxygenase or DNA beta-glucosyltransferase. In some embodiments, a kit further comprises a modified dCTP selected from 5hmdCTP, 5fdCTP, 5cadCTP, 5mdCTP, pyrrolo-dCTP and N4mdCTP and/or a strand-displacing or nick translating polymerase. In some embodiments, a kit may additionally comprise a ligase, a polymerase, a proteinase K, and/or a thermolabile proteinase K. A double-stranded DNA deaminase may be lyophilized or in a buffered storage solution that contains glycerol.

As would be apparent to those having the benefit of the present disclosure, a double-stranded DNA deaminase may be used in a variety of genome analysis methods, particularly methods whose goal is to identify the position and/or identity of one or more modified cytosines and/or determine the methylation status of a cytosine. In other embodiments, a double-stranded DNA deaminase can be a component of a fusion protein for based editing, i.e., generating site-specific C to T substitutions in a genome.

### Embodiments

The present disclosure further relates to embodiments disclosed in U.S. Provisional Application No. 63/264,513 including all of the following:

Embodiment 1. A polypeptide comprising at least 90% sequence identity with any of SEQ ID NOs: 1-8, not including 100% identity to SEQ ID NO: 3.

Embodiment 2. The polypeptide according to embodiment 1, comprising at least 90% sequence identity with any of SEQ ID NOs: 1-3 not including 100% identity to SEQ ID NO: 3.

Embodiment 3. The polypeptide according to embodiment 1, comprising at least 90% sequence identity with any of SEQ ID NOs: 1 or 2.

Embodiment 4. The polypeptide according to any of embodiments 1-3, capable of deaminating cytosine in double stranded DNA (dsDNA) with no sequence bias.

Embodiment 5. The polypeptide according to any of embodiments 1-3, capable of deaminating cytosine in single stranded DNA (ssDNA) with no sequence bias.

Embodiment 6. The polypeptide of any of embodiments 1-5, comprising a fusion protein.

Embodiment 7. The polypeptide of any of embodiments 1-6, wherein the polypeptide is lyophilized.

Embodiment 8. The polypeptide of any of embodiments 1-7, wherein the polypeptide is immobilized on a substrate.

Embodiment 9. The polypeptide of any of embodiments 1-8, wherein the polypeptide is combined with one or more reagents in a mixture wherein one or more reagents in the mixture comprises a second polypeptide.

Embodiment 10. The polypeptide of embodiment 9, wherein the second polypeptide is selected from the group consisting of a ligase, a polymerase, a methylcytosine (mC) dioxygenase, DNA glucosyltransferase, a Proteinase K, and a Thermolabile Proteinase K.

Embodiment 11. The polypeptide of any of embodiments 9-10, wherein the one or more reagents in the mixture further comprises a reversible inhibitor of the deaminase.

Embodiment 12. The polypeptide of any of embodiments 1-11, wherein the mixture further comprises DNA.

Embodiment 13. A method for methylome analysis comprising
(a) combining a reaction mixture containing genomic DNA with a double stranded DNA (dsDNA) deaminase having no sequence bias;
(b) deaminating at least 50% of the cytosine in the genomic DNA to uracil, without a denaturing step to convert dsDNA into single stranded (ssDNA).

Embodiment 14. The method according to embodiment 13, wherein prior to (a) adding to the reaction mixture, a methylcytosine (mC) dioxygenase to the genomic DNA for converting mC to hydroxymethylcytosine (hmC).

Embodiment 15. The method according to any of embodiments 13-14, wherein prior to (a) adding a hydroxymethylcytosine (hmC) modifying reagent to the reaction mixture.

Embodiment 16. The method according to any of embodiments 13-15, wherein (b) further comprises inactivating the DNA deaminase with a Proteinase K or Thermolabile Proteinase K.

Embodiment 17. The method according to any of embodiments 13-16, wherein (b) further comprises amplifying the DNA containing the converted cytosines.

Embodiment 18. The method according to any of embodiments 13-17, further comprising sequencing the amplified DNA.

Embodiment 19. The method according to any of embodiments 13-18, further comprising determining the location of methylcytosine (mC) in genomic DNA.

Embodiment 20. A kit comprising a deaminase capable of deaminating cytosine in double stranded DNA (dsDNA) and optionally single stranded DNA (ssDNA) with no sequence bias.

Embodiment 21. The kit according to embodiment 20, further comprising a methyl dioxygenase in a separate container from the dixoygenase.

Embodiment 22. The kit according to embodiment 20 or 21, further comprising a hydroxymethylcytosine (hmC) modifying enzyme in the same container with the dioxygenase or in a different container.

Embodiment 23. A method for deaminating a double-stranded nucleic acid, the method comprising:

contacting:
a double-stranded DNA substrate that comprises cytosines; and
a double-stranded DNA deaminase having an amino acid sequence that is at least 80% identical to any of SEQ ID NOS: 2, 3, 4, 5, 6, 7, 8, 9, 10, 11, 12, 14, 15, 16, 19, 24, 26, 27, 28, 33, 40, 49, 50, 63, 95, 96, 97, and 99;
   to produce a deamination product that comprises deaminated cytosines.

Embodiment 24. The method according to Embodiment 23, wherein the double-stranded DNA substrate further comprises a modified cytosine.

Embodiment 25. The method according to Embodiment 24, wherein the modified cytosine is a 5fC, 5CaC, 5mC, 5hmC, N4mC, 5ghmC, or pyrrolo-C.

Embodiment 26. The method according to Embodiment 23, wherein the method further comprises:
sequencing the deamination product, or amplifying the deamination product to produce amplification products and sequencing the amplification products, in each case, to produce sequence reads.

Embodiment 27. The method according to Embodiment 26, wherein the method further comprises:
analyzing the sequence reads to identify a modified cytosine in the double-stranded DNA substrate.

Embodiment 28. The method according to Embodiment 23, wherein the double-stranded DNA substrate is eukaryotic or bacterial DNA.

Embodiment 29. The method according to Embodiment 23, wherein the double-stranded DNA substrate is human cfDNA.

Embodiment 30. The method according to Embodiment 23, wherein the double-stranded DNA deaminase has an amino acid sequence that is at least 90% identical to any of SEQ ID NOS: 2, 3, 4, 5, 6, 7, 8, 9, 10, 11, 12, 14, 15, 16, 19, 24, 26, 27, 28, 33, 40, 49, 50, 63, 95, 96, 97, and 99.

Embodiment 31. The method according to Embodiment 23, wherein the double-stranded DNA substrate is pre-treated with a TET methylcytosine dioxygenase and DNA beta-glucosyltransferase.

Embodiment 32. The method according to Embodiment 31, wherein the double-stranded DNA deaminase has an amino acid sequence that is at least 90% identical to any of the SEQ ID NOS for MGYPDa829 (SEQ ID NO: 96), MGYPDa06 (SEQ ID NO: 4), CrDa01 (SEQ ID NO: 12), AvDa02 (SEQ ID NO: 2), CsDa01 (SEQ ID NO: 9), LbsDa01 (SEQ ID NO: 10), FIDa01 (SEQ ID NO: 8), MGYPDa26 (SEQ ID NO: 7), MGYPDa23 (SEQ ID NO: 6), chimera_10 (SEQ ID NO: 97) and AncDa04 (SEQ ID NO: 95).

Embodiment 33. The method according to Embodiment 23, wherein the double-stranded DNA substrate is pre-treated with a TET methylcytosine dioxygenase but not DNA beta-glucosyltransferase.

Embodiment 34. The method according to Embodiment 33, wherein the double-stranded DNA deaminase has an amino acid sequence that is at least 90% identical to any of the SEQ ID NOS for CseDa01 (SEQ ID NO: 3) and LbDa02 (SEQ ID NO: 1).

Embodiment 35. The method according to Embodiment 23, wherein the double-stranded DNA substrate is not pre-treated with either a TET methylcytosine dioxygenase or DNA beta-glucosyltransferase.

Embodiment 36. The method according to Embodiment 23, wherein the double-stranded DNA substrate comprises at least one N4mC.

Embodiment 37. The method according to Embodiment 36, wherein the double-stranded DNA substrate is bacterial DNA.

Embodiment 38. The method according to Embodiment 36, wherein the double-stranded DNA deaminase has an amino acid sequence that is at least 90% identical to any of the SEQ ID NOS for MGYPDa20 (SEQ ID NO: 11), NsDa01 (SEQ ID NO: 27), and AshDa01 (SEQ ID NO: 40).

Embodiment 39. The method according to Embodiment 23, further comprising:
(a) ligating a hairpin adapter to a double-stranded fragment of DNA to produce a ligation product;
(b) enzymatically generating a free 3′ end in a double-stranded region of the hairpin adapter in the ligation product; and
(c) extending the free 3′ end in a dCTP-free reaction mix that comprises a strand-displacing or
   nick-translating polymerase, dGTP, dATP, dTTP and modified dCTP. to produce the double-stranded DNA substrate.

Embodiment 40. The method according to Embodiment 39, wherein the modified dCTP is 5mdCTP, pyrrolo-dCTP, 5hmdCTP or N4-mdCTP.

Embodiment 41. The method according to Embodiment 39, wherein the double-stranded DNA deaminase has an amino acid sequence that is at least 90% identical to any of the SEQ ID NOS for MGYPDa20 (SEQ ID NO: 11), NsDa01 (SEQ ID NO: 27), AshDa01 (SEQ ID NO:40).

Embodiment 42. An enzyme comprising an amino acid sequence that is at least 80% identical to the C-terminal deaminase domain of a naturally-occurring protein, wherein the enzyme:
(a) has a double-stranded DNA deaminase activity; and
(b) does not comprise the N-terminus of the naturally-occurring protein.

Embodiment 43. The enzyme according to Embodiment 42, wherein the enzyme is no more than 300 amino acids in length.

Embodiment 44. The enzyme according to Embodiment 42, wherein the enzyme is at least 80% identical to any of SEQ ID NOS: 2, 3, 4, 5, 6, 7, 8, 9, 10, 11, 12, 14, 15, 16, 19, 24, 26, 27, 28, 33, 40, 49, 50, 63, 95, 96, 97, and 99.

Embodiment 45. The enzyme according to Embodiment 42, wherein the enzyme is fused with a catalytically dead Cas9 (dCas9) or a nicking Cas9 (nCas9) or Transcription activator-like effector nucleases (TALEN).

Embodiment 46. A kit comprising:
(a) an enzyme of Embodiment 42; and
(b) a reaction buffer.

Embodiment 47. The kit according to Embodiment 46, wherein the kit further comprises:
a TET methylcytosine dioxygenase and a DNA beta-glucosyltransferase; or
a TET methylcytosine dioxygenase and no DNA beta-glucosyltransferase

Embodiment 48. The kit according to Embodiment 46, wherein the kit is free of TET methylcytosine dioxygenase and DNA beta-glucosyltransferase.

Embodiment 49. The kit according to Embodiment 46, wherein the kit further comprises a modified dCTP selected from 5mdCTP, pyrrolo-dCTP, 5hmdCTP and N4-mdCTP.

Embodiment 50. A reaction mix comprising:
(a) a double-stranded DNA substrate that comprises cytosines; and
(b) a double-stranded DNA deaminase having an amino acid sequence that is at least 80% identical to any of SEQ ID NOS: 2, 3, 4, 5, 6, 7, 8, 9, 10, 11, 12, 14, 15, 16, 19, 24, 26, 27, 28, 33, 40, 49, 50, 63, 95, 96, 97, and 99.

Embodiment 51. The reaction mix according to Embodiment 50, wherein the double-stranded DNA substrate comprises cytosines and at least one modified cytosine.

Embodiment 52. The reaction mix according to Embodiment 50, wherein the modified cytosine is a 5fC, 5caC, 5mC, 5hmC, N4mC or pyrrolo-C.

Embodiment 53. The reaction mix according to Embodiment 50, wherein the double-stranded DNA substrate comprises eukaryotic or bacterial DNA.

Embodiment 54. The reaction mix according to Embodiment 50, wherein the double-stranded DNA substrate is human cfDNA.

Embodiment 55. The reaction mix according to Embodiment 50, wherein the deaminase has an amino acid sequence that is at least 90% identical to any of SEQ ID NOS: 2, 3, 4, 5, 6, 7, 8, 9, 10, 11, 12, 14, 15, 16, 19, 24, 26, 27, 28, 33, 40, 49, 50, 63, 95, 96, 97, and 99.

Embodiment 56. A method for sequencing, comprising: contacting a single-stranded DNA substrate comprising a genomic DNA fragment with a double-stranded DNA deaminase to produce a deamination product; sequencing the deamination product, or amplifying the deamination product to produce amplification products and sequencing the amplification products, in each case, to produce sequence reads, wherein the double-stranded DNA deaminase is an enzyme of Embodiment 42.

Embodiment 57. A method for sequencing, comprising:
contacting a double-stranded DNA substrate comprising a genomic DNA fragment with a double-stranded DNA deaminase to produce a deamination product; sequencing the deamination product, or amplifying the deamination product to produce amplification products and sequencing the amplification products, in each case, to produce sequence reads.

Embodiment 58. The method of Embodiment 57, wherein the double-stranded DNA deaminase has sequence bias for cytosine in a CpG context.

Embodiment 59. The method of Embodiment 58, wherein the double-stranded DNA deaminase is modification sensitive.

Embodiment 60. The method of Embodiment 59, wherein the double-stranded DNA deaminase does not deaminate one or more of 5fC, 5CaC, 5mC, 5hmC, N4mC, or 5ghmC.

Embodiment 61. The method of Embodiment 58, wherein the double-stranded DNA deaminase is not modification sensitive.

Embodiment 62. The method of Embodiment 58, wherein the double-stranded DNA substrate or the genomic fragment is not pre-treated with either a TET methylcytosine dioxygenase or DNA beta-glucosyltransferase.

Embodiment 63. The method of Embodiment 58, wherein the double-stranded DNA substrate or the genomic DNA fragment is pre-treated with a TET methylcytosine dioxygenase, and optionally is pre-treated with a DNA beta-glucosyltransferase.

Embodiment 64. The method of Embodiment 59, wherein the double-stranded DNA deaminase has an amino acid sequence that is at least 80% identical to any of SEQ ID NOS: 40, 62, 63, 65, 67, 71, 110, 112, 114, and 117.

Embodiment 65. The method of Embodiment 61, wherein the double-stranded DNA deaminase has an amino acid sequence that is at least 80% identical to any of SEQ ID NOS: 47, 49, 50, 55, 58, 59, 70, 76, 106, 107, 163 and 164.

Embodiment 66. The method of Embodiment 57, wherein the double-stranded DNA substrate further comprises a genomic fragment linked to an adapter.

Embodiment 67. The method of Embodiment 66, wherein the adapter comprises a primer.

Embodiment 68. The method of Embodiment 57, wherein the strands of the double-stranded DNA substrate are not linked together by an adapter.

Embodiment 69. The method of Embodiment 57, wherein the deamination product is double-stranded.

Embodiment 70. The method of Embodiment 57, wherein the double-stranded DNA substrate is not a multi-copy strand.

Embodiment 71. The method of Embodiment 57, further comprising analyzing the sequence reads to identify a modified cytosine in the double-stranded DNA substrate.

Embodiment 72. The method of Embodiment 71, wherein a reference sequence is not used for the analyzing.

Embodiment 73. The method of Embodiment 71, wherein the modified cytosine is one or more of 5fC, 5CaC, 5mC, 5hmC, N4mC, or 5ghmC.

Embodiment 74. The method of Embodiment 71, wherein the modified cytosine is 5hmC. and the double-stranded DNA deaminase has an amino acid sequence that is at least 80% identical to any of SEQ ID NOS: 4, 5, 10, 13, 16, 96, 99 and 106.

Embodiment 75. A method for deaminating a nucleic acid, the method comprising: contacting:
a DNA substrate that comprises cytosines; and
a double-stranded DNA deaminase having an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163, and 164.
to produce a deamination product that comprises deaminated cytosines.

Embodiment 76. The method of Embodiment 75, wherein the DNA substrate further comprises a modified cytosine.

Embodiment 77. The method of Embodiment 76, wherein the modified cytosine is a 5fC, 5CaC, 5mC, 5hmC, N4mC, 5ghmC, or pyrrolo-C.

Embodiment 78. An enzyme comprising an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163, and 164.

Embodiment 79. The enzyme of Embodiment 78, wherein the enzyme is fused with a DNA binding domain.

Embodiment 80. The enzyme of Embodiment 78, wherein the DNA binding domain is selected from a Cas9 domain, a Cas12 domain, a transcription activator-like effector nuclease (TALEN domain), a zinc finger (ZF) domain, a transcription activator-like effector (TALE) domain, an Sso7d domain, and a methyl binding domain (MBD) domain.

Embodiment 81. The enzyme of Embodiment 78, wherein the enzyme is no more than 300 amino acids in length.

Embodiment 82. A method for sequencing, comprising:
contacting a single-stranded DNA substrate comprising a genomic DNA fragment with a double-stranded DNA deaminase to produce a deamination product;
sequencing the deamination product, or amplifying the deamination product to produce amplification products and sequencing the amplification products, in each case, to produce sequence reads,
wherein the double-stranded DNA deaminase is an enzyme of Embodiment 22.

Embodiment 83. A kit comprising:
(a) an enzyme of Embodiment 78; and
(b) a reaction buffer.

Embodiment 84. The kit of Embodiment 83, wherein the kit further comprises:
a TET methylcytosine dioxygenase and a DNA beta-glucosyltransferase; or
a TET methylcytosine dioxygenase and no DNA beta-glucosyltransferase

Embodiment 85. The kit of Embodiment 83, wherein the kit is free of TET methylcytosine dioxygenase and DNA beta-glucosyltransferase.

Embodiment 86. A reaction mix comprising:
(a) a DNA substrate that comprises cytosines; and
(b) a double-stranded DNA deaminase having an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163 and 164.

Embodiment 87. The reaction mix of Embodiment 86, wherein the DNA substrate comprises cytosines and at least one modified cytosine.

Embodiment 88. The reaction mix of Embodiment 87, wherein the modified cytosine is a 5fC, 5caC, 5mC, 5hmC, N4mC or pyrrolo-C.

Embodiment 89. A method for base editing comprising:
contacting a fusion protein with a target sequence to produce an edited target sequence comprising at least one deaminated cytosine or deaminated modified cytosine, wherein the fusion protein comprises a dsDNA deaminase fused to a DNA binding domain.

Embodiment 90. The method of Embodiment 89, wherein the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain.

Embodiment 91. The method of Embodiment 90, wherein the fusion protein further comprises a guide RNA complementary to at least a portion of the targeted sequence.

Embodiment 92. The method of Embodiment 89 wherein the fusion protein comprises an enzyme at is at least 80% identical to any of SEQ ID NOS:1-152.

### Examples

### Example 1: Expression of DNA deaminases In vitro

Candidate DNA deaminase genes first were codon-optimized and then flanking sequences were added to each end, specifically, sequences containing T7 promoter at 5′ end and T7 terminator at 3′ end. These sequences were ordered as liner gBlocks from Integrated DNA Technologies (Coralville, IA, USA). Template DNA for in vitro protein synthesis was generated with Phusion® Hot Start Flex DNA Polymerase using gBlocks as template and flanking primers. The PCR products were purified using

Monarch PCR and DNA Cleanup kit (New England Biolabs, Inc., Ipswich, MA, USA). DNA concentration was quantified using a NanoDrop spectrophotometer (Thermo Fisher Scientific, Inc., Waltham, MA, USA). 100-400 ng PCR fragments were used as template DNA to synthesize analytic amounts of DNA deaminases using PURExpress In Vitro Protein Synthesis kit (New England Biolabs, Inc., Ipswich, MA, USA) following manufacturer's recommendations.

### Example 2: Deamination Assay on Single and Double Stranded Substrates

To test the activity of in vitro expressed DNA deaminases, a 2 ul aliquot of PURExpress sample was mixed with 300 ng of ΦX174 Virion DNA (ssDNA substrate) or ΦX174 RF I DNA (dsDNA substrate) in buffer containing 50 mM Bis-Tris pH 6.0, 0.1% Triton X-100 and incubated for 1 h at 37° C. The deaminated ΦX174 DNA was purified using Monarch PCR and DNA Cleanup kit (New England Biolabs, Inc., Ipswich, MA, USA). DNA concentration was quantified using a NanoDrop spectrophotometer (Thermo Fisher Scientific, Inc., Waltham, MA, USA). 150 ng of deaminated DNAs were digested to nucleosides with the Nucleoside Digestion Mix (New England Biolabs, Inc., Ipswich, MA, USA) following manufacturer's recommendations. LC-MS/MS analysis was performed by injecting digested DNAs on an Agilent 1290 Infinity II UHPLC equipped with a G7117A diode array detector and a 6495C triple quadrupole mass detector operating in the positive electrospray ionization mode (+ESI). UHPLC was carried out on a Waters XSelect HSS T3 XP column (2.1×100 mm, 2.5 um) with a gradient mobile phase consisting of methanol and 10 mM aqueous ammonium acetate (pH 4.5). MS data acquisition was performed in the dynamic multiple reaction monitoring (DMRM) mode. Each nucleoside was identified in the extracted chromatogram associated with its specific MS/MS transition: dC [M+H^{]}+at m/z 228.1112.1; dU [M+H]⁺ at m/z 229.1113.1; dmC [m+Fi]⁺ at m/z 242.1126.1; and dT [m+Fi]⁺ at m/z 243.1127.1. External calibration curves with known amounts of the nucleosides were used to calculate their ratios within the samples analyzed.

### Example 3: NGS Deamination Assay

50 ng of E. coli C2566 genomic DNA was combined with control modified DNA's:

| | | |
|---|---|---|
| | | DNA amount |
| DNA | Modification | (ng) |

| | | |
|---|---|---|
| E. coli C2566 | C | 46.8 |
| Lambda phage, dcm- | C | 1 |

| | | | |
|---|---|---|---|
| XP12 phage | 5 | mC | 1 |
| 1783 bp PCR fragment | 5 | hmC | 0.1 |
| amplified with 5 hmdCTP | | | |
| T4 phage, AGT- | 5 | ghmC | 1 |
| pRSSM1.PleII | N4 | mC | 0.1 |

### DNA Prep

Then the DNA was transferred to a Covaris microTUBE (Covaris, Woburn, MA, USA) and sheared to 300 bp using the Covaris S2 instrument. The 50 μL of sheared material was transferred to a PCR strip tube to begin library construction. NEBNext DNA Ultra II Reagents (New England Biolabs, Ipswich, MA,

USA) were used according to the manufacturer's instructions for end repair, A-tailing, and adaptor ligation using an Illumina-compatible adapter. The ligated samples were mixed with 110 μL of resuspended NEBNext Sample Purification Beads and cleaned up according to the manufacturer's instructions. The library was eluted in 17 μL of water.

### Deamination

The DNA was then deaminated in 50 mM Bis-Tris pH 6.0, 0.1% Triton X-100, using 1μI of dsDNA deaminase synthesized as described above with an incubation time of 1 hour at 37° C. After deamination reaction, 1μI of Thermolabile Proteinase K (New England Biolabs, Ipswich, MA) was added and incubated additional 30 min at 37° C. 5p.M of NEBNext Unique Dual Index Primers and 25 μL NEBNext Q5U Master Mix (New England Biolabs, Ipswich, MA, USA) were added to the DNA and PCR amplified. The PCR reaction samples were mixed with 50 ul of resuspended NEBNext Sample Purification Beads and cleaned up according to the manufacturer's instructions. The library was eluted in 15 ul of water. The libraries were analyzed and quantified by High sensitivity DNA analysis using a chip inserted into an Agilent Bioanalyzer 2100. The whole-genome libraries were sequenced using the Illumina NextSeq platform. Pair-end sequencing of 150 cycles (2×75 bp) was performed for all the sequencing runs. Base calling and demultiplexing were carried out with the standard Illumina pipeline. Results of CseDa01 are shown in FIGS. 4A and 4B.

### Example 4: 1-Tube-3-Enzyme EM-Seq (dsDNA Deaminase MGYPDa829+TET2+BGT)

50 ng of NA12878 genomic DNA was combined with 0.1 ng of CpG methylated pUC19 and 1 ng of unmethylated lambda control DNA and made up to 50 ul with 5 mM Tris pH=8.0. DNA was prepared according to Example 3 and the library was eluted in 29 ul of water. DNA was oxidized in a 50 ul reaction volume containing 50 mM Tris HCl pH 8.0, 1 mM DTT, 5 mM Sodium-L-Ascorbate, 20 mM a-KG, 2 mM ATP, 50 mM Ammonium Iron (II) sulfate hexahydrate, 0.04 mM UDG-glucose (NEB, Ipswich, MA), 16 μg mTET2, 10 U T4-BGT (NEB, Ipswich, MA). The reaction was initiated by adding Fe (II) solution to a final reaction concentration of 40 μM and then incubated for 1h at 37° C. The DNA was then deaminated, using 1 ul of MGYPDa829 dsDNA deaminase with an incubation time of 3 hour at 37° C. After deamination reaction, 1 ul of Thermolabile Proteinase K (P81115, New England Biolabs, Ipswich, MA) was added and incubated additional 30 min at 37° C. and 15 min at 60° C. At the end of the incubation, DNA was purified using 70 ul of resuspended NEBNext Sample Purification Beads according to the manufacturer's protocol. The sample was eluted in 16 ul water and 15 ul was transferred to a new tube. 1 μM of NEBNext Unique Dual Index Primers and 25 ul NEBNext Q5U Master Mix (M0597, New England Biolabs, Ipswich, MA) were added to the DNA and PCR amplified. The libraries were analyzed and quantified with an Agilent Bioanalyzer 2100 DNA analyzer. The whole-genome libraries were sequenced, and analyzed as described below.

Raw reads were first trimmed by the Trim Galore software to remove adapter sequences and low-quality bases from the 3′ end. Unpaired reads due to adapter/quality trimming were also removed during this process. The trimmed read sequences were C to T converted and were then mapped to a composite reference sequence including the human genome (GRCh38) and the complete sequences of lambda and pUC19 controls using the Bismark program with default Bowtie2 setting (Langmead and Salzberg 2012). The aligned reads were then subjected to two post-processing QC steps: 1, alignment pairs that shared the same alignment start positions (5′ ends) were regarded as PCR duplicates and were discarded; 2, reads that aligned to the human genome and contained excessive cytosines in non-CpG context (e.g., more than 3 in 75 bp) were removed because they are likely resulted from conversion errors. The numbers of T's (converted not methylated) and C's (unconverted modified) of each covered cytosine position were then calculated from the remaining good quality alignments using Bismark methylation extractor, and the methylation level was calculated as # of C/(# of C+# of T). FIG. 3C illustrates this workflow.

### Example 5: CseDa01 DNA Deaminase does not Deaminate 5caC and 5fC

1500 ng of oligonucleotides

(ACACCCATCACATTTACAC(5caC)GGGAAAGAGTTGAATGTAGAGTTGG; SEQ ID NO: 157) or ACACCCATCACATTTACAC(5fC)GGGAAAGAGTTGAATGTAGAGTTGG; SEQ ID NO:158 with one modified cytosine (5caC or 5fC) were treated with CseDa01 DNA deaminase for 4 h in buffer containing 50 mM Bis-Tris pH 6.0, 0.1% Triton X-100 and incubated for 1 h at 37° C. The deaminated oligonucleotides were purified using Monarch PCR and DNA Cleanup kit (New England Biolabs, Inc., Ipswich, MA, USA). DNA concentration was quantified using a NanoDrop spectrophotometer (Thermo Fisher Scientific, Inc., Waltham, MA, USA). 1500 ng of deaminated DNAs were digested to nucleosides with the Nucleoside

Digestion Mix (New England Biolabs, Inc., Ipswich, MA, USA) following manufacturer's recommendations. UHPLC-MS analysis was performed using an Agilent 1290 Infinity II UHPLC equipped with G7117A Diode Array Detector and 6135 XT MS Detector, on a Waters XSelect HSS T3 XP column (2.1×100 mm, 2.5 μm) with the gradient mobile phase consisting of methanol and 10 mM ammonium acetate buffer (pH 4.5). The identity of each peak was confirmed by MS. The relative abundance of each nucleoside was determined by the integration of each peak at 260 nm or their respective UV absorption maxima. Results are shown in FIG. 4C.

### Example 6: 1-Tube-2-Enzyme EM-Seq Using the dsDNA Deaminase CseDa01+TET2

50 ng of NA12878 genomic DNA was combined with 0.1 ng of CpG methylated pUC19 and 1 ng of unmethylated lambda control DNA and made up to 50 μL with 5 mM Tris pH=8.0. DNA was prepared according to Example 3 and the library was eluted in 29 μL of water. DNA was oxidized in a 50 μL reaction volume containing 50 mM Tris HCl pH 8.0, 1 mM DTT, 5 mM Sodium-L-Ascorbate, 20 mM a-KG, 2 mM ATP, 50 mM Ammonium Iron (II) sulfate hexahydrate, and 16 μg mTET2. The reaction was initiated by adding Fe (II) solution to a final reaction concentration of 40 μM and then incubated for 1 h at 37° C. The DNA was then deaminated, using 1 μl of CseDa01 dsDNA deaminase with an incubation time of 3 hour at 37° C. After deamination reaction, 1 μl of Thermolabile Proteinase K (P81115, New England Biolabs, Ipswich, MA) was added and incubated additional 30 min at 37° C. and 15 min at 60° C. At the end of the incubation, DNA was purified using 70 μL of resuspended NEBNext Sample Purification Beads according to the manufacturer's protocol. The sample was eluted in 16 μL water and 15 μL was transferred to a new tube. 1 μM of NEBNext Unique Dual Index Primers and 25 μL NEBNext Q5U Master Mix (M0597, New England Biolabs, Ipswich, MA) were added to the DNA and PCR amplified. The libraries were analyzed and quantified with an Agilent Bioanalyzer 2100 DNA analyzer. The whole-genome libraries were sequenced, and analyzed as described below. Raw reads were first trimmed by the Trim Galore software to remove adapter sequences and low-quality bases from the 3′ end. Unpaired reads due to adapter/quality trimming were also removed during this process. The trimmed read sequences were C to T converted and were then mapped to a composite reference sequence including the human genome

(GRCh38) and the complete sequences of lambda and pUC19 controls using the Bismark program with default Bowtie2 setting (Langmead and Salzberg 2012). The aligned reads were then subjected to two post-processing QC steps: 1, alignment pairs that shared the same alignment start positions (5′ ends) were regarded as PCR duplicates and were discarded; 2, reads that aligned to the human genome and contained excessive cytosines in non-CpG context (e.g., more than 3 in 75 bp) were removed because they are likely resulted from conversion errors. The numbers of T's (converted not methylated) and C's (unconverted modified) of each covered cytosine position were then calculated from the remaining good quality alignments using Bismark methylation extractor, and the methylation level was calculated as # of C/(# of C+# of T). FIG. 3C illustrates this workflow.

### Example 7: DNA Deaminase CseDa01 Works Very Efficiently in the TET2 Buffer Allowing to Perform Single-Tube 5mC Oxidation and DNA Deamination Reactions

To test the activity of CseDa01 DNA deaminase in TET2 buffer a 2μI of PURExpress sample was mixed with 300 ng of ΦX174 Virion DNA (ssDNA substrate) or ΦX174 RF I DNA (dsDNA substrate) in buffer containing 50 mM Tris HCl pH 8.0, 1 mM DTT, 5 mM Sodium-L-Ascorbate, 20 mM a-KG, 2 mM ATP, 50 mM Ammonium Iron (II) sulfate hexahydrate, 0.04 mM, and incubated for 1 h at 37° C. The deaminated ΦX174 DNA was purified using Monarch PCR and DNA Cleanup kit (New England Biolabs, Inc., Ipswich, MA, USA). DNA concentration was quantified using a NanoDrop spectrophotometer (Thermo Fisher Scientific, Inc., Waltham, MA, USA). 150 ng of deaminated DNAs were digested to nucleosides with the Nucleoside Digestion Mix (New England Biolabs, Inc., Ipswich, MA, USA) following manufacturer's recommendations. LC-MS/MS analysis was performed by injecting digested DNAs on an Agilent 1290 Infinity II UHPLC equipped with a G7117A diode array detector and a 6495C triple quadrupole mass detector operating in the positive electrospray ionization mode (+ESI). UHPLC was carried out on a Waters XSelect HSS T3 XP column (2.1×100 mm, 2.5 μm) with a gradient mobile phase consisting of methanol and 10 mM aqueous ammonium acetate (pH 4.5). MS data acquisition was performed in the dynamic multiple reaction monitoring (DMRM) mode. Each nucleoside was identified in the extracted chromatogram associated with its specific MS/MS transition: dC [m+H]⁺ at m/z 228.1→112.1; dU [M+H]⁺ at m/z 229.1→113.1; d^{m}C [m+H]⁺ at m/z 242.1→126.1; and dT [m+H]⁺ at m/z 243.1→127.1. External calibration curves with known amounts of the nucleosides were used to calculate their ratios within the samples analyzed. Results are shown in FIGS. 4A, 4B, 4C, 5A, and 5B.

### Example 8: Modification-Sensitive Deaminases Efficiently Deaminate Cytosines to Uracil, However, do not Deaminate 5-Methylcytosine and 5-Hydroxymethylcytosine in dsDNA and ssDNA

50 ng of E. coli C2566 genomic DNA was combined with 2 ng unmethylated lambda, phage XP12 (all cytosines are 5-methylcytosines) and T4 phage DNA (all cytosines are 5-hydroxymethyl cytosines) control DNAs and made up to 50 μL with 10 mM Tris, pH 8.0. Then the DNA was prepared according to Example 3 with a sheared size of 240-290 bp and a library elution volume of 15 μL of water. The DNA was then deaminated in 50 mM Bis-Tris pH 6.0, 0.1% Triton X-100, using 1μI of a modification-sensitive dsDNA deaminase (e.g., MGYPDa20 or NsDa01) synthesized as described above with an incubation time of 1 hour at 37° C. After deamination reaction, 1μI of Thermolabile Proteinase K (P81115, New England Biolabs, Ipswich, MA) was added and incubated additional 30 min at 37° C. 1μM of NEBNext Unique Dual Index Primers and 25 μL NEBNext Q5U Master Mix (M0597, New England Biolabs, Ipswich, MA) were added to the DNA and PCR amplified. The PCR reaction samples were mixed with 50 μL of resuspended NEBNext Sample Purification Beads and cleaned up according to the manufacturer's instructions. The library was eluted in 15 μL of water. The libraries were analyzed and quantified by High sensitivity DNA analysis using a chip inserted into an Agilent Bioanalyzer 2100. The whole-genome libraries were sequenced using the Illumina NextSeq platform. Pair-end sequencing of 150 cycles (2×75 bp) was performed for all the sequencing runs. Base calling and demultiplexing were carried out with the standard Illumina pipeline. Raw reads were first trimmed by the Trim Galore to remove adapter sequences and low-quality bases from the 3′ end. Unpaired reads owing to adapter/quality trimming were also removed during this process. The trimmed read sequences were C-to-T converted and were then mapped to a composite reference sequence including the E. coli C2566 genome and the complete sequences of lambda, phage XP12, and T4 controls using the Bismark program with the default Bowtie 2 setting.

The first 5 bp at the 5′ end of R2 reads were removed to reduce end-repair errors and aligned read pairs that shared the same alignment start positions (5′ ends) were regarded as PCR duplicates and were discarded. Next deamination events (C->T) were called by comparing the remaining good alignment sequences to the reference sequences using Bismark methylation extractor program. The 20 bp flanking sequences (10 bp upstream and 10 bp downstream) of all the covered cytosines from the individual genomes were then extracted and the cytosines sites were divided into different groups based on their deamination rates (>=90%, >=50%, >=25% or <=10%). Flanking sequences of each cytosine group were used to make sequence logo using WebLogo 3 to infer deamination sequence preference. Results are shown in FIGS. 6A and 6B for MGYPDa20, FIGS. 7A and 7B for NsDa01, FIGS. 8A and 8B for RhDa01_extN10, and FIGS. 9A and 9B for MmgDa02.

### Example 9: Applying the 1-Tube-1-Enzyme EM-Seq Method to Map 5mC in Human Using a Modification-Sensitive dsDNA Deaminase MGYPDa20

50 ng of NA12878 genomic DNA was combined with 0.1 ng of CpG methylated pUC19 and 1 ng of unmethylated lambda control DNA and made up to 50 μL with 5 mM Tris pH=8.0. DNA was prepared according to Example 3 and the library was eluted in 17 μL of molecular grade water. The DNA was then deaminated in 50 mM Bis-Tris pH 6.0, 0.1% Triton X-100, using 1μI of MGYPDa20 dsDNA deaminase with an incubation time of 3 hours at 37° C. Other modification sensitive deaminases may be substituted (e.g., see Table 3). After deamination reaction, 1μI of Thermolabile Proteinase K (P81115, New England Biolabs, Ipswich, MA) was added and incubated additional 30 min at 37° C. 5 μM of NEBNext Unique Dual Index Primers, 20 μM deaminated DNA and 25 μL NEBNext Q5U Master Mix (M0597, New England Biolabs, Ipswich, MA) were combined and PCR amplified. The PCR reaction samples were mixed with 50 μl of resuspended NEBNext Sample Purification Beads and cleaned up according to the manufacturer's instructions. The library was eluted in 15 μL of water. The libraries were analyzed and quantified by High sensitivity DNA analysis using a chip inserted into an Agilent Bioanalyzer 2100. The whole-genome libraries were sequenced using the Illumina NextSeq platform and analyzed as described below. Raw reads were first trimmed by the Trim Galore software to remove adapter sequences and low-quality bases from the 3′ end. Unpaired reads due to adapter/quality trimming were also removed during this process. The trimmed read sequences were C to T converted and were then mapped to a composite reference sequence including the human genome (GRCh38) and the complete sequences of lambda and pUC19 controls using the Bismark program with default Bowtie2 setting (Langmead and Salzberg 2012). The aligned reads were then subjected to two post-processing QC steps: 1, alignment pairs that shared the same alignment start positions (5′ ends) were regarded as PCR duplicates and were discarded; 2, reads that aligned to the human genome and contained excessive cytosines in non-CpG context (e.g., more than 3 in 75 bp) were removed because they are likely resulted from conversion errors. The numbers of T's (converted not methylated) and C's (unconverted modified) of each covered cytosine position were then calculated from the remaining good quality alignments using Bismark methylation extractor, and the methylation level was calculated as # of C/(# of C+# of T). FIG. 3D illustrates this workflow. Results are shown in FIG. 10.

### Example 10: Preparation of Methyl-SNP-Seq Library Using MGYPDa20 DNA Deaminase

For whole human genome methyl-SNP-seq sequencing 4μg of NA12878 gDNA and 40 ng of unmethylated lambda DNA as spiked in to monitor the deamination efficiency were used. The genomic DNA was fragmented using 250 bp sonication protocol using a Covaris S2 sonicator. Two technical replicates were set up. The fragmented gDNA was end repaired and dA-tailed (NEB Ultra II E7546 module), then ligated to the custom hairpin adapter using NEB ligase master mix (NEB, M0367). The incomplete ligation product (fragment having only one or no adaptor ligated) was removed using two exonucleases (NEB exolll and NEB exoVIl). Two nick sites were created at the uracil positions in the hairpin adapters at both ends after being treated with UDG and EndoVIll. The nick sites were translated towards 3′ terminus by DNA polymerase I in the presence of dATP, dGTP, dTGP and 5-methyl-dCTP. The nick translation causes double stranded DNA break when DNA polymerase I encounters the other nick on the opposite strand. The resulting fragments have one end ligated to a hairpin adapter and blunt end on the other side. The blunt end was dA-tailed and ligated with methylated Illumina adapter. The ligated product was deaminated at 37° C. for 3 h with double stranded DNA deaminase MGYPDa20. The deaminated DNA product was amplified using NEBNext Q5U Master Mix (NEB, M0597). The resulting indexed library was used for Illumina sequencing. The human Methyl-SNP-seq libraries were sequenced using an Illumina Novaseq 6000 sequencer for 100 bp paired end reads.

### Example 11: Detection of N4mC Modified DNA with CseDa01 dsDNA Deaminase

50 ng of Paenibacillus species JDR-2 (CCGG target sequence) and Salmonella enterica FDAARGOS_312 (CACCGT target sequence) DNAs were combined with 0.1 ng of CpG methylated pUC19 and 1 ng of unmethylated lambda control DNA and made up to 50 μL with 5 mM Tris pH=8.0. DNA was prepared according to Example 3 with a sheared size of240-290 bp and an elution volume of 15 μL of water. The DNA was then deaminated in 50 mM Bis-Tris pH 6.0, 0.1% Triton X-100, using 1μI of CseDa01 dsDNA deaminase synthesized as described above with an incubation time of 1 hour at 37° C. After deamination reaction, 1 μl of Thermolabile Proteinase K (P8111S, New England Biolabs, Ipswich, MA) was added and incubated additional 30 min at 37° C. 1 μM of NEBNext Unique Dual Index Primers and 25 μl NEBNext Q5U Master Mix (M0597, New England Biolabs, Ipswich, MA) were added to the DNA and PCR amplified. The PCR reaction samples were mixed with 50 ul of resuspended NEBNext Sample Purification Beads and cleaned up according to the manufacturer's instructions. The library was eluted in 15 μl of water. The libraries were analyzed and quantified by High sensitivity DNA analysis using a chip inserted into an Agilent Bioanalyzer 2100. The whole-genome libraries were sequenced using the Illumina NextSeq platform. Pair-end sequencing of 150 cycles (2×75 bp) was performed for all the sequencing runs. Raw reads were first trimmed by the Trim Galore to remove adapter sequences and low-quality bases from the 3′ end. Unpaired reads owing to adapter/quality trimming were also removed during this process. The trimmed read sequences were C-to-T converted and were then mapped to the reference sequence and the complete sequences of lambda and pUC19 controls using the Bismark program with the default Bowtie 2 setting. The first 5 bp at the 5′ end of R2 reads were removed to reduce end-repair errors and aligned read pairs that shared the same alignment start positions (5′ ends) were regarded as PCR duplicates and were discarded. Next deamination events (C->T) were called by comparing the remaining good alignment sequences to the reference sequences using Bismark methylation extractor program. An N4mC modified site is called when it is largely un-deaminated (C->T conversion rate <=20%). The flanking 20 bp sequences of all the called N4mC sites were extracted and a sequence logo using WebLogo 3 was generated. Results are shown in FIGS. 11A and 11B.

### Example 12: Detection of N4mC and 5mC Modified DNA with CseDa01 dsDNA Deaminase and MGYPDa20 dsDNA Deaminase

50 ng of NEB1569 Thermus species M and NEB 394 Acinetobacter species H genomic DNAs was combined with 0.1 ng of CpG methylated pUC19 and 1 ng of unmethylated lambda control DNA and made up to 50 μl with 5 mM Tris pH=8.0. Then the DNA was prepared according to Example 3 with a sheared size of 240-290 bp and a library elution volume of 15 μl of water. The DNA was then deaminated in 50 mM Bis-Tris pH 6.0, 0.1% Triton X-100, using 1 μl of dsDNA deaminase synthesized as described above with an incubation time of 1 hour at 37° C. After deamination reaction, 1 μl of Thermolabile Proteinase K (P8111S, New England Biolabs, Ipswich, MA) was added and incubated additional 30 min at 37° C. 1 μM of NEBNext Unique Dual Index Primers and 25 μl NEBNext Q5U Master Mix (M0597, New England Biolabs, Ipswich, MA) were added to the DNA and PCR amplified. The PCR reaction samples were mixed with 50 μl of resuspended NEBNext Sample Purification Beads and cleaned up according to the manufacturer's instructions. The library was eluted in 15 μl of water. The libraries were analyzed and quantified by High sensitivity DNA analysis using a chip inserted into an Agilent Bioanalyzer 2100. The whole-genome libraries were sequenced using the Illumina NextSeq platform. Pair-end sequencing of 150 cycles (2×75 bp) was performed for all the sequencing runs. Base calling and demultiplexing were carried out with the standard Illumina pipeline. Raw reads were first trimmed by the Trim Galore to remove adapter sequences and low-quality bases from the 3′ end. Unpaired reads owing to adapter/quality trimming were also removed during this process. The trimmed read sequences were C-to-T converted and were then mapped to a composite reference sequence including the NEB1569 Thermus species M and NEB 394 Acinetobacter species H and the complete sequences of lambda and pUC19 controls using the Bismark program with the default Bowtie 2 setting. The first 5 bp at the 5′ end of R2 reads were removed to reduce end-repair errors and aligned read pairs that shared the same alignment start positions (5′ ends) were regarded as PCR duplicates and were discarded. Next deamination events (C->T) were called by comparing the remaining good alignment sequences to the reference sequences using Bismark methylation extractor program. The N4mC modification is called from the CseDa01 deaminase-treated library. An N4mC modified site is called when it is largely un-deaminated (C->T conversion rate <=20%). For 5mC modification detection, a differential methylation analysis was conducted between the MGYPDa20 deaminase-treated library (detect both N4mC and 5mC) and the CseDa01 deaminase-treated library (detect only N4mC) of the same sample to identify modified sites (i.e., 5mC) that are only detected in the MGYPDa20 library. The differentially methylated sites were called by a logistic regression method with SLIM corrected Q value <=0.01, and methylation difference >=80% using the Methylkit program. To identify methyltransferase recognition sequences, the 9 bp flanking sequences were extracted, including 4 bp upstream and 4 bp downstream of all the modified sites, and the unique 9 bp sequences were clustered using a hierarchical linkage method based on the difference between each pair of sequences. A sequence logo was generated using WebLogo 3 for each cluster representing a distinct methyltransferase recognition motif.

### Example 13: Candidate Selection

A list of HMMER3 (Eddy, S. R. Accelerated Profile HMM Searches. PLOS Comput. Biol. 7, e1002195 (2011)) cytosine deaminase sequence profiles was curated. 29 profiles came from the CDA clan (CL0109) from the Pfam (Mistry, J. et al. Pfam: The protein families database in 2021. Nucleic Acids Res. 49, D412—D419 (2021)) database (excluding the TM1506, Lpxl_C, FdhD-NarQ, and AICARFT_IMPCHas, which do not encode deaminases), 17 profiles were built from multiple sequence alignments (MSAs) of deaminase families defined by lyer et al. (Nucleic Acids Res. 39, 9473-9497, 2011), and one profile was built from a multiple sequence alignment found in Zhang et al. (Biol. Direct 7, 18, 2012).

Some candidate sequences were selected directly from the MSAs listed in lyer et al. (2011), and Zhang et al. (2012). Others were selected from hmmsearch hits of the profiles described above against six different databases: UniProt, Mgnify, IMG/VR, IMG/M, wastewater treatment plant metagenomes, and GenBank (respectively, The UniProt Consortium. UniProt: the universal protein knowledgebase in 2021. Nucleic Acids Res. 49, D480—D489 (2021); Mitchell, A. L. et al. MGnify: the microbiome analysis resource in 2020. Nucleic Acids Res. 48, D570—D578 (2020); Paez-Espino, D. et al. IMG/VR: a database of cultured and uncultured DNA Viruses and retroviruses. Nucleic Acids Res. 45, gkw1030 (2017); Chen, I.-M. A. et al. The IMG/M data management and analysis system v.6.0: new tools and advanced capabilities. Nucleic Acids Res. 49, D751—D763 (2021); Singleton, C. M. et al. Connecting structure to function with the recovery of over 1000 high-quality metagenome-assembled genomes from activated sludge using long-read sequencing. Nat. Commun. 12, 2009 (2021); and Da, B. et al. GenBank. Nucleic Acids Res. 41, (2013)).

Most of the deaminases tested were found as fusions to larger proteins, for example as parts of polymorphic toxin systems. To determine the boundaries of the deaminase domain, AlphaFold2 (Jumper, J. et al. Highly accurate protein structure prediction with AlphaFold. Nature 1-11 (2021) doi:10.1038/s41586-021-03819-2) structural predictions were generated and visualized. N-terminal truncation sites were generally selected at several amino acids before helix 1 of the deaminase domain.

For convenience, each screened sequence was given a short name. The names are arbitrary, but relate somehow to the database or species of origin for the sequence. Da=deaminase, MGYP=Mgnify protein, Hm=hot metagenome, VR=IMG/VR, WWTP=waste water treatment plant, chimera=chimeric sequence, Anc=ancestral sequence reconstruction. Other prefixes are mostly two or three letters drawn from the name of the source organism or the source environment of the metagenome data. Some sequences also have prefixes or suffixes of the form extN #, extC #, d #, Cd #, which indicate, respectively, N-terminal extensions, C-terminal extensions, N-terminal deletions, and C-terminal deletions of the indicated number of residues, compared to the candidate with the un-affixed name.

Amino acid sequence alignments were all calculated using MAFFT (v7.490) (Katoh, K. & Standley, D. M. MAFFT Multiple Sequence Alignment Software Version 7: Improvements in Performance and Usability. Mol. Biol. Evol. 30, 772-780 (2013)) using globalpair mode. Trees were generated using raxml-ng (v. 1.1)(Kozlov, A. M., Darriba, D., Flouri, T., Morel, B. & Stamatakis, A. RAxML-NG: a fast, scalable and user-friendly tool for maximum likelihood phylogenetic inference. Bioinformatics 35, 4453-4455 (2019)). Ancestral sequence reconstructions were built from phylogenetic trees using raxml-ng (v. 1.1)

### Example 14: Summary tables

Assay results for 29 deaminases are shown in Table 1 below, in which APOBEC3A (a single-stranded DNA deaminase) served as a negative control. The other 28 deaminases (double-stranded DNA deaminases) in the table all have significant activity on a double-stranded DNA substrate.

Double-stranded DNA deaminases disclosed herein may be used in many methods, processes, and workflows including, for example, the applications shown in Table 2 below. Deamination products may contain one or more modified cytosines, for example, where the substrate dsDNA included such modified cytosines and the operative deaminase does not or only poorly deaminases such modified cytosines. Each of the listed methods/applications may further comprise (a)(i) sequencing the deamination products and/or (ii) amplifying (e.g., by PCR) the deamination products to produce amplification products and sequencing the amplification products, in each of (a)(i) and (a)(ii), to produce sequence reads, and (b) optionally determining the kind and/or position of modified cytosines in the dsDNA substrate from the sequence reads.

Screening results for over 100 deaminases are shown in Table 3 below, in which APOBEC3A (a single-stranded DNA deaminase) served as a negative control. Many were observed to have double-stranded DNA deaminase activity under the conditions tested. Relatedness of the enzymes tested is illustrated in FIG. 1 and, in this light, deaminases that displayed limited or modest activity under the specific conditions tested may have higher activity under alternative or optimized conditions.

The names and SEQ ID NOS of certain double-stranded DNA deaminases disclosed herein are shown in Table 4 along with the corresponding names included in U.S. Provisional Application No. 63/264,513 filed Nov. 24, 2021.

### Example 15: Use of 1-Tube-3-Enzyme Protocol for Simultaneous Detection of DNA Methylation and Genetic Bases Using a CpG Specific dsDNA Deaminase and Modification Protection Enzymes

Combine 50 ng of NA12878 genomic DNA with 0.1 ng of CpG methylated pUC19 and 1 ng of unmethylated lambda control DNAs and make up to 50 μl with 5 mM Tris pH=8.0. Shear the DNA to about 300 bp using any method. For example, transfer DNA to a Covaris microtube (Covaris, Woburn, MA) and shear according to the manufacturer's protocol.

Add 50 ng of sheared DNA to a PCR strip tube to begin library construction. Use NEBNext DNA Ultra II Reagents (NEB, Ipswich, MA) according to the manufacturer's instructions for end repair, A-tailing, and adaptor ligation of the custom made Pyrollo-dC adaptor, where all dC's are replaced with Pyrollo-dC: ACACTCTTTCCCTACACGACGCTCTTCCGATCT (SEQ ID NO:165) and [Phos]GATCGGAAGAGCACACGTCTGAACTCCAGTCA (SEQ ID NO:166).. EM-seq adaptor (E7120S/L, NEB, Ipswich, MA) or any other desired adapter with or without replacement of dCs with modified dCs may also be used. Purify the adapter ligated DNA using 1× NEBNext Sample Purification Beads according to the manufacturer's instructions.

Combine the adapter ligated DNA with 5X TET2 reaction buffer and the mTET2 and T4-BGT enzymes in a 50 μl reaction volume. Initiate 5mC oxidation reaction by adding Fe (II) solution (Ammonium Iron (II) sulfate hexahydrate) to a final reaction concentration of 50 μM and then incubate for 1 h at 37° C. Add 1 μl of Thermolabile Proteinase K (P8111S, New England Biolabs, Ipswich, MA) and incubate additional 30 min at 37° C. and 10 min at 60° C.

Purify DNA using 1X NEBNext Sample Purification Beads according to the manufacturer's protocol and elute in 17 μl water. Add 2 μl of 10X deaminase buffer, 1 μl of CbDa01 and incubate for 3 h at 37° C. Incubation time may be shortened or extended depending on factors such as temperature, enzyme concentration, etc. The deamination reaction may be stopped in a variety of ways (e.g., enzymatically, separation step, etc.). For example, add 1 μl of Thermolabile Proteinase K (P8111S, New England Biolabs, Ipswich, MA) to each tube and incubate for an additional 30 min at 37° C. and 10 min at 60° C. Mix with 5 μl of H20, 15 μl of deamination reaction, 5 μl of NEBNext Unique Dual Index Primers and 25 μl NEBNext Q5U Master Mix (M0597, New England Biolabs, Ipswich, MA) and amplify using EM-seq protocol (6 PCR cycles). After PCR reaction, purify amplified DNA using 1X resuspended NEBNext Sample Purification Beads according to the manufacturer's protocol. Use a Bioanalyzer or TapeStation to determine the size distribution and concentration of the libraries. Data analysis may be conducted as described in Example 22.

### Example 16: Use of 1-Tube-1-Enzyme Protocol with a Methylation Sensitive CpG Specific dsDNA Deaminase for Simultaneous Detection of DNA Methylation and Genetic Bases

Combine 50 ng of NA12878 genomic DNA with 0.1 ng of CpG methylated pUC19 and 1 ng of unmethylated lambda control DNAs and make up to 50 μl with 5 mM Tris pH=8.0. Shear the DNA to about 300 bp using any method. For example, transfer DNA to a Covaris microtube (Covaris, Woburn, MA) and shear according to the manufacturer's protocol. Add 50 ng of sheared DNA to a PCR strip tube to begin library construction. Use NEBNext DNA Ultra II Reagents (NEB, Ipswich, MA) according to the manufacturer's instructions for end repair, A-tailing, and adaptor ligation of the custom made Pyrollo-dC adaptor, where all dC's are replaced with Pyrollo-dC: ACACTCTTTCCCTACACGACGCTCTTCCGATCT (SEQ ID NO:165) and [Phos]GATCGGAAGAGCACACGTCTGAACTCCAGTCA (SEQ ID NO:166). EM-seq adaptor (E7120S/L, NEB, Ipswich, MA) or any other desired adapter with or without replacement of dCs with modified dCs may also be used. Purify the adapter ligated DNA using 1× NEBNext Sample Purification Beads according to the manufacturer's instructions and elute in 17 μl of water.

Add 2 μl of 10X deaminase buffer and 1 μl of the RhDa01 deaminase and incubate for 3 h at 37° C. Incubation time may be shortened or extended depending on factors such as temperature, enzyme concentration, etc. After deamination reaction, add 1μI of Thermolabile Proteinase K (P81115, New England Biolabs, Ipswich, MA) to each tube and incubated additional 30 min at 37° C. and 10 min at 60° C. Note that the deamination reaction may be stopped in a variety of alternative ways (e.g., enzymatically, separation step, etc.). Mix with 5μI of H2O, 15 μL of deamination reaction, 5μI of NEBNext Unique Dual Index Primers and 25 μL NEBNext Q5U Master Mix (M0597, New England Biolabs, Ipswich, MA) and amplify using EM-seq protocol (6 PCR cycles). After PCR reaction, purify amplified DNA using 1X resuspended NEBNext Sample Purification Beads according to the manufacturer's protocol. Use a Bioanalyzer or TapeStation to determine the size distribution and concentration of the libraries. Data analysis may be conducted as described in Example 22.

### Example 17: Use of 1-Tube-1-Enzyme Protocol for Simultaneous Detection of DNA Methylation and Genetic Bases Using Low-Input or Single-Cell DNA

Combine 5˜10 pg of NA12878 genomic DNA or DNA from a single cell with 0.2% of CpG methylated pUC19 and 2% of unmethylated lambda control DNAs and make up to 50 μL with 5 mM Tris pH=8.0. Shear the DNA to about 300 bp using any method. For example, transfer DNA to a Covaris microtube (Covaris, Woburn, MA) and shear according to the manufacturer's protocol. Transfer the sheared DNA to a PCR strip tube to begin library construction. Use NEBNext DNA Ultra II Reagents (NEB, Ipswich, MA) according to the manufacturer's instructions but reduce the reaction volumes to half for end repair, A-tailing, and adaptor ligation of the custom made Pyrollo-dC: ACACTCTTTCCCTACACGACGCTCTTCCGATCT (SEQ ID NO:165) and [Phos]GATCGGAAGAGCACACGTCTGAACTCCAGTCA (SEQ ID NO:166). EM-seq adaptor (E7120S/L, NEB, Ipswich, MA) or any other desired adapter with or without replacement of dCs with modified dCs may also be used. Purify the adapter ligated DNA using 1× NEBNext Sample Purification Beads according to the manufacturer's instructions and elute in 16 μl of water.

Add 2 μL of 10X deaminase buffer, 1 μL of 10 ng carrier DNA (any not adapter ligated DNA could be used as carrier DNA), and 1 μL the RhDa01 enzyme. Mix and incubate for 3 h at 37° C. Incubation time could be shortened or extended depending on enzyme concentration. For deaminases acting on ssDNA, the DNA substrate could be denatured using heat or any chemical denaturing agent. The deamination reaction may be stopped in a variety of ways (e.g., enzymatically, separation step, etc.). For example, add 1 μL of Thermolabile Proteinase K (P81115, New England Biolabs, Ipswich, MA) to each tube and incubated additional 30 min at 37° C. and 10 min at 60° C.). Mix with 5 μL of H20, 15 pi of deamination reaction, 5 μL of NEBNext Unique Dual Index Primers and 25 μL NEBNext Q5U Master Mix (M0597, New England Biolabs, Ipswich, MA) and amplify using EM-seq protocol (1215 PCR cycles). After PCR reaction, purify amplified DNA twice using 1X resuspended NEBNext Sample Purification Beads according to the manufacturer's protocol. Use a Bioanalyzer or TapeStation to determine the size distribution and concentration of the libraries.

The library was sequenced on an Illumina NovaSeq machine and resulted in 154 million 2x100 bp paired-end reads. Data analysis of 5mC and genetic base detection was conducted using the method described in the Example 22. About 95% of the reads were mapped to the human reference genome using a standard DNA sequencing aligner Bowtie2. The final average sequencing coverage of the human genome after removing PCR duplicates and muti-mapped reads is about 6×. The base-resolution methylation results of this method agree well with of the published EM-seq method, with a high Pearson correlation value of 0.87 for the CpG sites that have a minimum 5× coverage. The CpG island methylation levels are also highly correlated between the two methods (Pearson correlation=0.95). This method also produced SNP calling results even at a low sequencing depth of 6× average coverage. Using chromosome 21 as an example and benchmarking against the variants obtained using the NA12878 whole-genome sequencing data set (WGS, performed by the JIMB NIST project), more than 90% of the SNPs detected by this method are identified in the reference dataset.

### Example 18: Simultaneous Detection of 5hmC and Genetic Bases Using CpG Specific dsDNA Deaminase and BGT

Combine 50 ng of NA12878 genomic DNA with 0.1 ng of CpG methylated pUC19 and 1 ng of unmethylated lambda control DNAs and make up to 50 μL with 5 mM Tris pH=8.0. Shear the DNA to about 300 bp using any method. For example, transfer DNA to a Covaris microtube (Covaris, Woburn, MA) and shear according to the manufacturer's protocol. Add 50 ng of sheared DNA to a PCR strip tube to begin library construction. Use NEBNext DNA Ultra II Reagents (NEB, Ipswich, MA) according to the manufacturer's instructions for end repair, A-tailing, and adaptor ligation of the custom made Pyrollo-dC adaptor, where all dC's are replaced with Pyrollo-dC: ACACTCTTTCCCTACACGACGCTCTTCCGATCT (SEQ ID NO:165) and [Phos]GATCGGAAGAGCACACGTCTGAACTCCAGTCA (SEQ ID NO:166). EM-seq adaptor (E7120S/L, NEB, Ipswich, MA) or any other desired adapter with or without replacement of dCs with modified dCs may also be used. Purify the adapter ligated DNA using 1× NEBNext Sample Purification Beads according to the manufacturer's instructions.

Combine the adapter ligated DNA with T4-BGT enzyme in T4-BGT buffer (NEB, Ipswich, MA) in a 50 μL reaction volume, and incubate for 1 h at 37° C. Reaction time may be adjusted according to substrate quantity. After deamination, stop the reaction (e.g., add 1 μL of Thermolabile Proteinase K (P8111S, New England Biolabs, Ipswich, MA) and incubate for an additional 30 min at 37° C. and 10 min at 60° C.).

Purify DNA using 1× NEBNext Sample Purification Beads according to the manufacturer's protocol and elute in 17 μL water. Add 2 μL of 10X deaminase buffer, 1 μL of CbDa01 (SEQ ID NO: 50), AcDa01 (SEQ ID NO: 49), Sso7d_GGGVTS_AcDa01 (SEQ ID NO: 163) or Sso7d_LSGLSDDKLKEI_AcDa01 (SEQ ID NO: 164), and incubate for a sufficient time for deamination, e.g., 3 h at 37° C. Incubation time may be shortened or extended depending on factors such as temperature, enzyme concentration, etc. For deaminases acting on ssDNA, the DNA substrate may be denatured using heat, enzymatic methods, or chemical methods. The deamination reaction may be stopped in a variety of ways (e.g., enzymatically, separation step, etc.). For example, add 1 μL of Thermolabile Proteinase K (P8111S, New England Biolabs, Ipswich, MA) to each tube and incubate for an additional 30 min at 37° C. and 10 min at 60° C. Mix with 5 μL of_{H}20, 15 μL of deamination reaction, 5 μL of NEBNext Unique Dual Index Primers and 25 μL NEBNext Q5U Master Mix (M0597, New England Biolabs, Ipswich, MA) and amplify using EM-seq protocol (6 PCR cycles). After PCR reaction, purify amplified DNA using 1X resuspended NEBNext Sample Purification Beads according to the manufacturer's protocol. Use a Bioanalyzer or TapeStation to determine the size distribution and concentration of the libraries. Data analysis may be conducted as described in Example 22.

### Example 19: Conducting Deamination Reactions Prior to Adapter Ligation when Using dsDNA Deaminase with CpG Bias for Sequencing

50 ng of NA12878 genomic DNA is combined with 0.1 ng of CpG methylated pUC19 and 1 ng of unmethylated lambda control DNA. Deamination is performed in a suitable buffer (e.g., 50 mM Bis-Tris pH 6.0, 0.1% Triton X-100), using 1 μL of dsDNA deaminase having bias for CpG (see, e.g., Table 3) with an incubation time, for example, of 3 hours at 37° C. Enzyme amount, temperature, and incubation time could be adjusted depending on deaminase activity. As is described herein, dsDNA deaminases are active on both ssDNA and dsDNA. For deaminases acting on ssDNA, the DNA substrate may be denatured using heat, enzymatic methods, or chemical methods. The deamination reaction may be stopped in a variety of ways (e.g., enzymatically, separation step, etc.). For example, add 1 μL of Thermolabile Proteinase K (P81115, New England Biolabs, Ipswich, MA) and incubate for an additional 30 min at 37° C. The DNA samples are mixed with purification beads and cleaned up according to the manufacturer's instructions. DNA is fragmented to about 300 bp. The 50 μL of sheared material is transferred to a PCR strip tube to begin library construction. NEBNext DNA Ultra II Reagents (NEB, Ipswich, MA) are used according to the manufacturer's instructions for end repair, A-tailing, and adaptor ligation. Any adapter sequence could be used. The ligated samples are mixed with 110 pi of resuspended NEBNext Sample Purification Beads and cleaned up according to the manufacturer's instructions. 5 μL of NEBNext Unique Dual Index Primers, 20 μL of deaminated DNA and 25 μL NEBNext Q5U Master Mix (M0597, New England Biolabs, Ipswich, MA) are combined and PCR amplified. The PCR reaction samples are mixed with 50 μL of resuspended NEBNext Sample Purification Beads and cleaned up according to the manufacturer's instructions. The library is eluted in 15 μL of water. The libraries are analyzed and quantified by High sensitivity DNA analysis using a chip inserted into an Agilent Bioanalyzer 2100. The whole-genome libraries are sequenced, e.g., using the Illumina NextSeq platform. Data analysis of 5mC and genetic base detection may be conducted as described in Example 22.

### Example 20: Simultaneous Detection of DNA Modifications and Genetic Bases of Long DNA Fragments Using a dsDNA Deaminase Having CpG Bias

For 5mC detection, 200 ng of human genomic DNA is oxidized by incubating with 16 μg of TET2 for 30 min at 37° C. followed 30-min incubation with BGT in the same buffer at 37° C. For 5hmC detection, genomic DNA is glucosylated by incubating with BGT enzyme for 2 h at 37° C. Modification protected genomic DNA is incubated for an additional 30 min with Proteinase K at 37° C. and subsequently purified using a genomic DNA purification kit. Purified DNA is deaminated with 2 μL of CbDa01 CpG dsDNA deaminase in 100 μL reaction volume for 3 hours. Incubation time may be shortened or extended depending on factors such as temperature, enzyme concentration, etc. For deaminases acting on ssDNA, the DNA substrate may be denatured using heat, enzymatic methods, or chemical methods. The deamination reaction may be stopped in a variety of ways (e.g., enzymatically, separation step, etc.). For example, add 1 μL of Thermolabile Proteinase K and incubate for an additional 30 min at 37° C. and 10 min at 60° C. Targeted genomic regions are amplified from the purified deaminated DNA using custom designed primers. After purification of PCR product, the long amplicons are used to prepare a PacBio SMRT sequencing (Pacific Biosciences) library following the “amplicon template preparation and sequencing” protocol, and the library is sequenced on a PacBio machine following manufacturer's instruction. Circular Consensus Sequences (CCS) are extracted from the raw data and converted into FASTQ file using SMRT Link program. 5mC and sequence analysis may be conducted as described in Example 22.

### Example 21: Detecting Epigenetic Modifications and Genetic Bases of Long DNA Fragments Using Nanopore Sequencing

For 5mC detection, 200 ng of human genomic DNA is oxidized by incubating with 16 μg of TET2 for 30 min at 37° C. followed 30-min incubation with BGT in the same buffer at 37° C. For 5hmC detection, genomic DNA is glucosylated by incubating with BGT enzyme for 2 h at 37° C. Modification protected genomic DNA was incubated for an additional 30 min with Proteinase K at 37° C. and subsequently purified using a genomic DNA purification kit. Purified DNA is deaminated with 2 μL of CbDa01 CpG deaminase in 100 μL reaction volume for 3 hours. Incubation time may be shortened or extended depending on factors such as temperature, enzyme concentration, etc. For deaminases acting on ssDNA, the DNA substrate could be denatured using heat or any chemical denaturing agent. The deamination reaction may be stopped in a variety of ways (e.g., enzymatically, separation step, etc.). For example, add 1 μL of Thermolabile Proteinase K and incubate additional 30 min at 37° C. and 10 min at 60° C. Targeted genomic regions are amplified from the purified deaminated DNA using custom designed primers. After purification of PCR product, the long amplicons are used to prepare a Nanopore sequencing library using the 1D Native barcoding genomic DNA kit, and the library is sequenced on a MinION flow cell. Raw reads are base called using the Guppy base caller. Analysis of 5mC and sequence may be conducted as described in Example 22.

### Example 22: Bioinformatics Analysis for Simultaneous Detection of DNA Methylation and Genetic Sequences

Raw sequencing reads are quality trimmed to remove adapter sequences and low-quality bases from the 3′ end. Unpaired reads due to adapter/quality trimming are also removed during this process. The trimmed read sequences are then mapped to a composite reference sequence including the human genome (GRCh38) and the complete sequences of lambda and pUC19 controls using a standard sequence alignment tool e.g., Bowtie2. Alignment pairs that shared the same alignment start positions (5′ ends) are regarded as PCR duplicates and are discarded. The alignments are used for SNP and other genetic variants detection (except for variant detection of Cs in CpG context) using a standard variant calling analysis pipeline, such as GATK.

For variant and methylation analysis of Cs in CpG context, C to T conversion events in CpG context are detected and summarized in a strand-specific manner using the samtools by the mpileup function. The G to A conversion events in the G positions that pair with the Cs of CpGs on the opposite strand are also detected and summarized using the samtools mpileup program. For each cytosine position in a CpG context, the C->T conversion rate and paired opposite strand G->A conversion rate is compared. If the two conversion rates are not statistically different, then all the C->T conversions are considered as a results of genetic variant, and no deamination on this cytosine position. If the C->T conversion rate is significantly larger than the G->A conversion rate, then the difference is considered as a result of deamination of unmodified cytosines. The methylation level of a cytosine in CpG context is calculated as #of methylated unconverted Cs/(#of methylated unconverted Cs+#of unmodified and converted Cs), whereas #of unmodified and converted Cs=number of C->T conversion events—number of G->A conversion events on the opposite strand.

### Example 23: 5hmC Detection Using a dsDNA Deaminase

This example describes detecting 5hmC using a dsDNA deaminase having activity on unmodified and methylated dsDNA and is blocked by 5ghmC (e.g., MGYPDa829, LbsDa01, MGYPDa01, PeDa01, MGYPDa06, or any of these deaminases fused to Sso7d, or a combination). 50 ng of NA12878 genomic DNA is combined with 0.1 ng of CpG methylated pUC19 and 1 ng of unmethylated lambda control DNA and made up to 50 μL with 5 mM Tris pH=8.0. DNA according to Example 3 and the library is eluted in 29 μL of water. The adapter ligated DNA is combined with T4-BGT enzyme in T4-BGT buffer (NEB, Ipswich, MA) in a 50 μL reaction volume, and incubated for 1 h at 37° C. Reaction time may be adjusted, for example, according to substrate quantity. 1 μL of Thermolabile Proteinase K (P81115, New England Biolabs, Ipswich, MA) is added and incubated additional 30 min at 37° C. and 10 min at 60° C. The DNA is purified using 1× NEBNext Sample Purification Beads according to the manufacturer's protocol and eluted in 17 μL water. The DNA is then deaminated, using, e.g., 1 μL of MGYPDa829 dsDNA deaminase and 2 μL 5× deamination buffer, incubation time of 3 hour at 37° C. After deamination reaction, the reaction is stopped, e.g., using 1 μL of Thermolabile Proteinase K (P81115, New England Biolabs, Ipswich, MA) and incubation for an additional 30 min at 37° C. and 15 min at 60° C. At the end of the incubation, DNA is purified using 70 μL of resuspended NEBNext Sample Purification Beads according to the manufacturer's protocol. The sample is eluted in 16 μL water and 15 μL is transferred to a new tube. 1 μM of NEBNext Unique Dual Index Primers and 25 μL NEBNext Q5U Master Mix (M0597, New England Biolabs, Ipswich, MA) are added to the DNA and PCR amplification is performed. The libraries are analyzed and quantified with an Agilent Bioanalyzer 2100 DNA analyzer.

Raw reads are first trimmed by the Trim Galore software to remove adapter sequences and low-quality bases from the 3′ end. Unpaired reads due to adapter/quality trimming are also removed during this process. The trimmed read sequences were C to T converted and were then mapped to a composite reference sequence including the human genome (GRCh38) and the complete sequences of lambda and pUC19 controls using the Bismark program with default Bowtie2 setting (Langmead and Salzberg 2012). The aligned reads were then subjected to two post-processing QC steps: 1, alignment pairs that shared the same alignment start positions (5′ ends) were regarded as PCR duplicates and were discarded; 2, reads that aligned to the human genome and contained excessive cytosines in non-CpG context (e.g., more than 3 in 75 bp) were removed because they are likely resulted from conversion errors. The numbers of T's (converted not methylated) and C's (unconverted modified) of each covered cytosine position were then calculated from the remaining good quality alignments using Bismark methylation extractor, and the 5hmC level was calculated as #of C/(#of C+#of T).

### Example 24: Combinations of Deaminases for Detecting Modifications in Specific Contexts

Multiple deaminases can be combined in the same mixture to achieve sequence specificities not accessible from a single deaminase. For example, a C proceeded by C or T can be selectively deaminated by a mixture of MGYPDa917 (SEQ ID NO: 48) and NoDa01 (SEQ ID NO: 39). C followed by a G or C can be selectively deaminated by a mixture of XcDa01 (SEQ ID NO: 68), MGYPDa21 (SEQ ID NO: 64), and AcDa01 (SEQ ID NO: 49). A C followed by a T or G can be selectively deaminated by a mixture of PdDa01 (SEQ ID NO: 60) and CbDa01 (SEQ ID NO: 50). Enzymes in all three of the described mixtures are blocked by the 5ghmC modification, and in combination with TET2 and BGT would be suitable for selectively mapping C modifications in their target contexts. Libraries can be constructed, and data analyzed as in Example 4. Three base context preferences for individual deaminases is described in FIG. 12A-12C; sensitivity to 5mC 5hmC and 5ghmC is described, for example, in Tables 1 and 3. Thus, a deaminase may be selected to suit the purpose of desired analysis.

### Example 25: Fusion Proteins of Double-Stranded DNA Deaminases with TALE for Base Editing

For base editing, a dsDNA deaminase (e.g., CseDa01 or other deaminase from Table 3) may be split into two (or more) inactive subdomains. The breakpoint of the deaminase domain is selected such that when brought together, the deaminase domain is competent for cytosine deamination of the cytidine (the “DD” refers to this deaminase domain below). Each subdomain of the DD is genetically fused to a TALE (Transcription activator-like effector) protein with the N- to C-terminal arrangement as follows: bpNLS-TALELeft-DDN-TERM-UGI; and bpNLS-TALERight-DDC-TERM-UGI, where DD is the deaminase domain, UGI is the uracil glycosidase inhibitor from Bacillus subtilis bacteriophage, bpNLS is a bi-partite nuclear localization signal, and DDN-TERM and DDC-TERM denote the N-terminal and C-terminal subdomains of the DD. The TALE protein pair are designed to target the WTAP (Wilms tumor 1 associated protein) gene locus. Codon sequences are optimized for mammalian expression. DNA constructs encoding the TALE-base editors are placed into mammalian expression plasmids where transcription is directed by the CMV immediate-early promoter enhancer. mRNA cleavage and polyadenylation are directed by the bovine growth hormone polyadenylation signal. The plasmids are co-electroporated into HEK293 cells using a Lonza nucleofector 4D. 48 hours post electroporation, genomic DNA is extracted from the cells and the WTAP locus is PCR amplified using primers spanning the site targeted by the TALE base editor pair. Amplified products are deep sequenced, and reads are analyzed using CRISPResso2. C to T mutations are measured in a quantification window measured centered between the two TALE binding sites.

### Example 26: Fusion Proteins of Double-Stranded DNA Deaminases with Cas9 Nickase for Base Editing

A deaminase domain (DD) of HcDa01 or other deaminase from Table 3 is genetically fused to a S. pyogenes Cas9 nickase variant D10A to encode a polypeptide with the N- to C-terminal arrangement as follows: bpNLS-DD-Cas9(D10A)-UGI-UGI-bpNLS, where DD is the deaminase domain, UGI is the uracil glycosidase inhibitor from Bacillus subtilis bacteriophage, and bpNLS is a bi-partite nuclear localization signal. Codon sequences are optimized for mammalian expression. The DNA construct encoding the genetic fusion is placed into a mammalian expression plasmid where transcription was directed by the cytomegalovirus (CMV) enhancer and the chicken beta-actin promoter. mRNA processing is directed by the chimeric intron (chicken/rabbit beta-globin) at the 5′-end of the transcript and cleavage and poly(A) tailing is directed from the rabbit beta-globin polyadenylation signal. Single guide RNAs targeting the WTAP locus are expressed from a separate DNA plasmid in which transcription of the sgRNA is directed from the U6 promoter. The sgRNA targets the sequence 5′-GGATTTAAGTGTAAATGTAC-3′ (SEQ ID NO:168). Plasmids are co-transfected into HEK293 cells. After 48 hours, genomic DNA is extracted and the WTAP locus is PCR amplified using primers spanning the site targeted by the transfected sgRNA and Cas9 base editor fusion. Amplified products are deep sequenced, and reads are analyzed using CRISPResso2. C to T mutations are measured in a quantification window measured −10 bp relative to the 3′-end of the hybridizing region of the sgRNA.

### Example 27: R-Loop Mapping

MapR may be performed, as described (Yan and Sarma, 2020 (DOI: https://doi.org/10.1002/cpmb.113, PM ID: 31943854); Yan et al., 2019 (DOI: https://doi.org/10.1016/j.celrep.2019.09.052), with the exception that RNase A may be omitted from the stop buffer. Following DNA extraction, the DNA sample is enzymatically deaminated with the ssDNA specific DNA deaminase activity of a deaminase set forth herein, e.g., HcDa01, followed by separation or removal from the reaction and/or inactivation by any means (e.g., heat, chemical, or specific or non-specific enzymatic degradation such as proteinase K digestion at 60° C. for 10 minutes). The DNA sample is purified by column purification and the eluted product used as a template for second-strand synthesis, e.g., using reagents from the NEBNext Ultra II Directional RNA Library Prep Kit (NEB E7760) following manufacturer's instructions. Libraries are PCR amplified with dual-index barcode primers for Illumina sequencing (NEB E7600) using Q5 DNA polymerase (NEB M0491) and purified. Uracil DNA glycosylase (NEB) are added to the PCR amplification mix to degrade dUTP-containing molecules and remove adaptor hairpins. Sequencing is performed on a NextSeq 500 instrument (Illumina) with 38×2 paired-end cycles.

### Example 28: Random Mutagenesis (C to T)

A dsDNA deaminase with low sequence context preference, such as CseDa01, LbDa02, BaDa01, MGYPDa01, MGYPDa20, MGYPDa06, CrDa01, AvDa01, or AvDa02, is added to a dsDNA substrate, such as a plasmid, genome, or amplicon, containing the mutagenesis target, to cause base mutations resulting from deamination of one or more bases in the dsDNA substrate. Alternatively, a dsDNA deaminase with stronger sequence preference may be used to bias the mutagenesis towards or away from specific parts of the target sequence (see, for example, context preferences set forth in FIG. 12A-12C). After incubation for a time depending on the desired level of mutagenesis, the deaminase is separated or removed from the reaction and/or inactivated by any means (e.g., heat, chemical, or specific or non-specific enzymatic degradation such as proteinase K digestion at 60° C. for 10 minutes). The mutated mutagenesis target is amplified by PCR with target-specific primers (e.g., using Q5U Hot Start High Fidelity DNA polymerase (NEB)). The amplicon is cloned into an expression vector, resulting in a library of mutants containing C to T and G to A mutations relative to the forward strand of the unmutated target sequence.

### Example 29: Deaminase Activity on 5fC and 5caC Modified DNA

To test activity of dsDNA deaminases on 5fC and 5caC modified DNA, a set of three oligonucleotide substrates (40 bp, listed below) containing four modified cytosines were prepared. The set of oligonucleotides included preferable deamination sites for eleven DNA deaminases described herein from five representative clades. In each reaction, the modified oligonucleotide (dcaC or dfC) was mixed with the control oligonucleotide (C only) in a ratio of 1:1 (800 ng+800 ng) to monitor deamination of cytosine to uracil. After incubation for 5 h at 37° C. in—reaction buffer containing 50 mM Bis-Tris pH 6.0, 0.1% Triton X-100 with different DNA deaminases, the oligonucleotide substrates were purified using Monarch PCR and DNA Cleanup kit, digested to nucleosides with the Nucleoside Digestion Mix (NEB, Ipswich MA) and the reaction products were quantified with LC-MS/MS. The results are shown in Table 5.

Oligonucleotides used to test deaminase activity on 5fC and 5caC were as follows:

″C″ in Table 5 (control)--
(SEQ ID NO: 167)
AAATTTAATTATAAAACGATCGACGACGAAATAATAAAAA

“5caC” in Table 5—each of the four Cs are substituted with 5caC

“5fC” in Table 5 —each of the four Cs are substituted with 5fC

### Example 30: Sequence Preferences of Exemplary dsDNA Deaminases

The NGS assay described in Example 3 was used to survey sequence preference among selected dsDNA deaminases. Deaminase recognition sequences sites can extend beyond the nCn context, with preferences for sequences of various lengths and compositions (FIG. 12A-12C).

TABLE 1

| Name | SEQ ID | C:C_dsDNA | C:C_ssDNA | C:CG_dsDNA | C:CH_dsDNA | 5mC:C_dsDNA | 5hmC:C_dsDNA |
|---|---|---|---|---|---|---|---|
| AcDa01 | 49 | 0.243 | 0.566 | 0.790 | 0.014 | 0.180 | 0.053 |
| AncDa04 | 95 | 0.992 | 0.998 | 0.985 | 0.995 | 0.929 | 0.733 |
| AshDa01 | 40 | 0.342 | 0.623 | 0.699 | 0.193 | 0.010 | 0.005 |
| AvDa02 | 2 | 0.998 | 1.000 | 1.000 | 0.998 | 0.979 | 0.763 |
| BaDa01 | 24 | 0.612 | 0.718 | 0.632 | 0.603 | 0.104 | 0.041 |
| BcDa02 | 15 | 0.772 | 0.746 | 0.863 | 0.734 | 0.069 | 0.028 |
| chimera_10 | 97 | 0.950 | 0.995 | 0.954 | 0.949 | 0.671 | 0.676 |
| CbDa01 | 50 | 0.240 | 0.668 | 0.760 | 0.022 | 0.089 | 0.059 |
| CrDa01 | 12 | 0.811 | 0.952 | 0.786 | 0.821 | 0.135 | 0.310 |
| CsDa01 | 9 | 0.913 | 0.783 | 0.896 | 0.920 | 0.180 | 0.084 |
| CseDa01 | 3 | 0.998 | 0.984 | 0.998 | 0.998 | 0.999 | 0.981 |
| d22_Cd4_PeDa01 | 99 | 0.641 | 0.602 | 0.800 | 0.575 | 0.541 | 0.506 |
| d38_MGYPDa829 | 5 | 0.993 | 0.993 | 0.994 | 0.993 | 0.812 | 0.770 |
| EcDa01 | 28 | 0.566 | 0.736 | 0.801 | 0.468 | 0.121 | 0.214 |
| FlDa01 | 8 | 0.928 | 0.848 | 0.926 | 0.929 | 0.290 | 0.059 |
| LbDa02 | 19 | 1.000 | 1.000 | 1.000 | 1.000 | 0.999 | 1.000 |
| LbsDa01 | 10 | 0.889 | 0.872 | 0.889 | 0.889 | 0.328 | 0.216 |
| MGYPDa01 | 16 | 0.748 | 0.845 | 0.885 | 0.691 | 0.578 | 0.330 |
| MGYPDa06 | 4 | 0.997 | 0.984 | 0.996 | 0.998 | 0.961 | 0.782 |
| MGYPDa16 | 14 | 0.780 | 0.901 | 0.894 | 0.732 | 0.211 | 0.339 |
| MGYPDa20 | 11 | 0.857 | 0.785 | 0.924 | 0.829 | 0.049 | 0.021 |
| MGYPDa23 | 6 | 0.935 | 0.923 | 0.994 | 0.911 | 0.383 | 0.275 |
| MGYPDa26 | 7 | 0.929 | 0.860 | 0.952 | 0.919 | 0.109 | 0.040 |
| MGYPDa829 | 96 | 0.956 | 0.952 | 0.954 | 0.957 | 0.517 | 0.326 |
| MmgDa02 | 63 | 0.133 | 0.256 | 0.446 | 0.002 | 0.017 | 0.011 |
| NsDa01 | 27 | 0.597 | 0.616 | 0.783 | 0.519 | 0.059 | 0.017 |
| RaDa01 | 33 | 0.465 | 0.697 | 0.460 | 0.467 | 0.173 | 0.136 |
| SaDa02 | 26 | 0.607 | 0.558 | 0.819 | 0.519 | 0.508 | 0.391 |
| APOBEC3A (control) | 154 | 0.331 | 0.995 | 0.333 | 0.330 | 0.058 | 0.006 |

Key:
C:C_dsDNA: fraction of unmodified cytosines deaminated in double-stranded DNA
C:C_ssDNA: fraction of unmodified cytosines deaminated in single-stranded DNA
C:CG_dsDNA: fraction of unmodified cytosines in CpG context, deaminated in double-stranded DNA
C:CH_dsDNA: fraction of unmodified cytosines followed by an adenine, cytosine, or thymine, deaminated in double-stranded DNA
5mC:C_dsDNA: fraction of cytosines with the 5-methyl modification, deaminated in double-stranded DNA.
5hmC:C_dsDNA: fraction of cytosines with the 5-hydroxymethyl modification, deaminated in double-stranded DNA.

TABLE 2

| | | Deaminase | Contacting (in one | Deaminase | |
|---|---|---|---|---|---|
| No | Applications | specificity | or more steps) | properties | Example deaminases |
| 1 | 1-tube3- | NCN | A TET, a BGT, a | High activity | MGYPDa829, MGYPDa06, |
| | enzyme EM- | | deaminase | on dsDNA, | CrDa01, AvDa02, CsDa01, |
| | seq (dsDNA | | substrate, and a dsDNA | blocked by | LbsDa01, FlDa01, |
| | deaminase + | | deaminase to produce | 5 ghmC | MGYPDa26, MGYPDa23, |
| | TET + BGT) | | deamination products | | chimera_10, AncDa04 |
| | | | comprising deaminated | | |
| | | | cytosines (uracils) | | |
| | | | and optionally 5 ghmC | | |
| 2 | 1-tube2- | NCN | A TET, a deaminase | High activity | CseDa01, LbDa02 |
| | enzyme EM- | | substrate, and a dsDNA | on dsDNA, | |
| | seq (dsDNA | | deaminase to produce | blocked by 5 fC | |
| | deaminase + | | deamination products | and 5 CaC | |
| | TET) | | comprising deaminated | | |
| | | | cytosines (uracils) | | |
| | | | and optionally 5 fC | | |
| | | | and/or 5 CaC | | |
| 3 | 1-tube1- | NCN | A deaminase | High activity | MGYPDa20, NsDa01, |
| | enzyme EM- | | substrate and a dsDNA | on dsDNA, | AshDa01 |
| | seq (dsDNA | | deaminase to produce | blocked by | |
| | modification | | deamination products | 5 mC and | |
| | sensitive | | comprising deaminated | 5 hmC | |
| | deaminase) | | cytosines (uracils) | | |
| | | | and optionally 5 mC | | |
| | | | and/or 5 hmC | | |
| 4 | 1-tube3- | NCG | A TET, a BGT, a | High activity | AncDa03, AcDa01, CbDa01, |
| | enzyme CpG- | | deaminase substrate, | on dsDNA in | RhDa01, MmgDa02, |
| | specific | | and a dsDNA | CpG context, | AncDa06, AshDa01 |
| | deaminase EM- | | deaminase to produce | blocked by | |
| | seq (CpG- | | deamination products | 5 ghmC | |
| | specific dsDNA | | comprising deaminated | | |
| | deaminase + | | cytosines (uracils) in | | |
| | TET + BGT) | | CpG context and | | |
| | | | optionally 5 ghmC | | |
| 5 | 1-tube2- | NCG | A TET, a deaminase | High activity | AncDa03, AcDa01, CbDa01, |
| | enzyme CpG | | substrate (e.g., a | on dsDNA in | RhDa01, MmgDa02, |
| | specific EM- | | dsDNA), and a dsDNA | CpG context, | AncDa06, AshDa01, |
| | seq (CpG- | | deaminase to produce | blocked by 5 fC | MgypDa05 |
| | specific dsDNA | | deamination products | and 5 CaC | |
| | deaminase + | | comprising deaminated | | |
| | TET) | | cytosines (uracils) in | | |
| | | | CpG context and | | |
| | | | optionally 5 fC | | |
| | | | and/or 5 CaC | | |
| 6 | 1-tube1- | NCG | A deaminase | High activity | RhDa01, MmgDa02,, |
| | enzyme CpG | | substrate (e.g., a | on dsDNA in | RhDa01_ext10 |
| | specific EM- | | dsDNA) and a dsDNA | CpG context, | |
| | seq (CpG- | | deaminase to produce | blocked by | |
| | specific and | | deamination products | 5 mC and | |
| | modification- | | comprising deaminated | 5 hmC | |
| | sensitive | | cytosines (uracils) in | | |
| | dsDNA | | CpG context and | | |
| | deaminase) | | optionally 5 mC | | |
| | | | and/or 5 hmC | | |
| 7 | N4 mC | NCN | A deaminase | High activity | CseDa01, LbDa02 |
| | detection | | substrate (e.g., a | on dsDNA of C | |
| | | | dsDNA) and a dsDNA | and 5 mC, | |
| | | | deaminase to produce | blocked by | |
| | | | deamination products | N4 mC | |
| | | | comprising deaminated | | |
| | | | cytosines (uracils) | | |
| | | | and optionally N4 mC | | |
| 8 | Detection of | NCN | (1) A deaminase | 1st enzyme: | Any enzyme from |
| | N4 mC and 5 mC | | substrate and a dsDNA | High activity | Application 7 (N4 mC |
| | (two enzymes, | | deaminase to produce | on dsDNA of C | detection) + |
| | two reactions) | | deamination products | and 5 mC, | Any enzyme from |
| | | | comprising deaminated | blocked by | Application 3 (One-enzyme |
| | | | cytosines (uracils) | N4 mC | EM-seq) |
| | | | and optionally N4 mC; | 2^{nd} enzyme: | |
| | | | and (2) a deaminase | High activity | |
| | | | substrate and a | on dsDNA of C, | |
| | | | (second) dsDNA | blocked by | |
| | | | deaminase to produce | 5 mC and | |
| | | | deamination products | N4 mC | |
| | | | comprising deaminated | | |
| | | | cytosines (uracils) | | |
| | | | and optionally N4 mC, | | |
| | | | 5 mC and/or 5 hmC | | |
| 9 | Simultaneous | NCN | A deaminase | dsDNA | MGYPDa829, Chimera_10, |
| | detection of | | substrate and a dsDNA | deaminase | MGYPDa23, LbsDa01, |
| | N4 mC and 5 mC | | deaminase to produce | with | FlDa01 |
| | (one enzyme | | deamination products | differential | |
| | on reaction) | | comprising deaminated | activity on C | |
| | | | cytosines (uracils) | and 5 mC, and | |
| | | | and optionally 5 mC | blocked by | |
| | | | and N4 mC | N4 mC | |
| 10 | Methyl-SNP- | NCN | A deaminase | High activity | Any enzyme from |
| | seq | | substrate and a dsDNA | on dsDNA, | Application 3 (One-enzyme |
| | | | deaminase to produce | blocked by | EM-seq) |
| | | | deamination products | 5 mC and | |
| | | | comprising deaminated | 5 hmC | |
| | | | cytosines (uracils) | | |
| | | | and optionally 5 mC | | |
| | | | and 5 hmC, wherein | | |
| | | | the dsDNA substrate | | |
| | | | is prepared by (1) | | |
| | | | ligating a hairpin | | |
| | | | adapter to a | | |
| | | | double-stranded | | |
| | | | fragment of DNA to | | |
| | | | produce a ligation | | |
| | | | product, (2) | | |
| | | | enzymatically | | |
| | | | generating a free 3′ | | |
| | | | end in a double- | | |
| | | | stranded region of | | |
| | | | the hairpin adapter | | |
| | | | in the ligation | | |
| | | | product; and (3) | | |
| | | | extending the free | | |
| | | | 3′ end in a dCTP- | | |
| | | | free reaction mix | | |
| | | | that comprises a | | |
| | | | strand-displacing or | | |
| | | | nick-translating | | |
| | | | polymerase, dGTP, | | |
| | | | dATP, dTTP and | | |
| | | | modified dCTP to | | |
| | | | produce the | | |
| | | | double-stranded | | |
| | | | DNA substrate | | |
| 11 | Base editing by | ACN, GCN | A fusion protein | High activity | MBO1351307, BaDa01, |
| | fusing dsDNA | CCN, TCN, | with a target | on dsDNA with | DddA, Chimera_17 |
| | cytosine | etc. | sequence to | various | (there are many more, |
| | deaminases | | produce an edited | specificities | almost any active enzyme) |
| | with the ZF or | | target sequence | | |
| | TALE-DNA | | comprising at least | | |
| | binding | | one deaminated | | |
| | modules | | cytosine or deaminated | | |
| | | | modified cytosine, | | |
| | | | wherein the fusion | | |
| | | | protein comprises a | | |
| | | | dsDNA deaminase fused | | |
| | | | to a ZF and/or TALE | | |
| | | | DNA binding module | | |
| 12 | Base editing by | ACN, GCN | A fusion protein | High activity | HcDa01, Chimera_01, |
| | fusing ssDNA | CCN, TCN, | with a target | on ssDNA and | SsDa01, MGYPDa13, |
| | cytosine | etc. | sequence to | no/low activity | d38_Cd11_MGYPDa829, |
| | deaminases with | | produce an edited | on dsDNA with | HgmDa02, etc. |
| | catalytically | | target sequence | various | |
| | inactivated | | comprising at least | specificities | |
| | Cas9 | | one deaminated | | |
| | | | cytosine or deaminated | | |
| | | | modified cytosine, | | |
| | | | wherein the fusion | | |
| | | | protein comprises a | | |
| | | | dsDNA deaminase fused | | |
| | | | to a catalytically | | |
| | | | inactivated type II-A | | |
| | | | Cas (e.g., Cas9) and | | |
| | | | optionally further | | |
| | | | comprising a guide | | |
| | | | RNA complementary to | | |
| | | | at least a portion of | | |
| | | | the targeted sequence | | |
| 13 | Heavily | NCN | A fusion protein | High activity | CseDa01, LbDa02 |
| | modified | | with a target | on modified C | |
| | jumbo phages | | sequence to | | |
| | base editing | | produce an edited | | |
| | | | target sequence | | |
| | | | comprising at least | | |
| | | | one deaminated | | |
| | | | cytosine or deaminated | | |
| | | | modified cytosine, | | |
| | | | wherein the fusion | | |
| | | | protein comprises a | | |
| | | | dsDNA deaminase | | |
| | | | fused to a ZF and/or | | |
| | | | TALE DNA binding | | |
| | | | module or the | | |
| | | | fusion protein | | |
| | | | comprises a dsDNA | | |
| | | | deaminase fused to | | |
| | | | a catalytically | | |
| | | | inactivated type II-A | | |
| | | | Cas (e.g., Cas9) and | | |
| | | | optionally further | | |
| | | | comprising a guide | | |
| | | | RNA complementary to | | |
| | | | at least a portion of | | |
| | | | the targeted sequence | | |
| 14 | Genome wide | NCN | A deaminase | activity on | HcDa01, Chimera_01, |
| | single- | | substrate (e.g., a | ssDNA only | SsDa01 |
| | stranded-DNA | | genomic DNA | | |
| | region | | substrate) and a | | |
| | detection (e.g., | | ssDNA deaminase, | | |
| | R-loop, stem- | | in non-denaturing | | |
| | loop structure) | | conditions | | |
| 15 | BisMapR | NCN | A dsDNA plus | activity on | Any enzyme from |
| | (strand-specific | | ssDNA substrate | ssDNA only | application 14 (Single- |
| | R-loop | | and a ssDNA | | stranded DNA mapping) |
| | detection | | deaminase, in non- | | |
| | method) | | denaturing | | |
| | | | conditions, to produce | | |
| | | | deamination products | | |
| | | | comprising deaminated | | |
| | | | cytosines (uracils) in | | |
| | | | ssDNA regions of | | |
| | | | the substrate | | |
| 16 | Screening for | NCN | A dsDNA substrate | A combination | CseDa01 + APOBEC3A |
| | novel cytosine | | or a dsDNA plus | of deaminases | |
| | modifications | | ssDNA substrate | that | |
| | | | and one or more | deaminate all | |
| | | | dsDNA or ssDNA | the known | |
| | | | deaminases to produce | cytosine | |
| | | | deamination products | modifications | |
| | | | comprising deaminated | in all sequence | |
| | | | cytosines (uracils) | context (e.g., | |
| | | | and optionally | CseDa01 + | |
| | | | modified cytosines | APOBEC3A) | |
| 17 | Mapping of | NCN | A dsDNA substrate | Activity on | CseDa01 |
| | chromatin | | from a eukaryotic | dsDNA | |
| | accessibility | | source and a dsDNA | | |
| | including Long- | | deaminase to produce | | |
| | range single- | | deamination products | | |
| | molecule | | comprising deaminated | | |
| | applications | | cytosines and/or | | |
| | | | deaminated | | |
| | | | modified cytosines | | |
| | | | in non-histone | | |
| | | | bound regions of | | |
| | | | the substrate, in | | |
| | | | conditions that | | |
| | | | preserve the | | |
| | | | histones and the | | |
| | | | natural DNA- | | |
| | | | histone contacts | | |
| 18 | Z-DNA mapping | NCN | A dsDNA plus | Activity on | CseDa01 |
| | | | ssDNA substrate | dsDNA | |
| | | | and a dsDNA | | |
| | | | deaminase, in non- | | |
| | | | denaturing | | |
| | | | conditions, to produce | | |
| | | | deamination products | | |
| | | | comprising deaminated | | |
| | | | cytosines and/or | | |
| | | | deaminated | | |
| | | | modified cytosines | | |
| | | | in non Z-form DNA | | |
| | | | regions of the | | |
| | | | substrate | | |
| 19 | Genome-wide | NCN | A dsDNA or a | Activity on | CseDa01, LbDa02, |
| | protein-DNA | | dsDNA plus a ssDNA | dsDNA | MGYPDa829, MGYPDa06, |
| | interaction site | | substrate and a | | CrDa01, AvDa02 |
| | mapping | | fusion protein | | |
| | | | comprising a dsDNA | | |
| | | | deaminase fused to | | |
| | | | any DNA binding | | |
| | | | protein to produce | | |
| | | | deamination products | | |
| | | | comprising deaminated | | |
| | | | cytosines and/or | | |
| | | | deaminated | | |
| | | | modified cytosines, | | |
| | | | in the bound | | |
| | | | regions of the DNA- | | |
| | | | binding protein. | | |
| 20 | Inactivation of | NCN | A deaminase | Activity on | Any enzyme from |
| | single stranded | | substrate (e.g., a | ssDNA only, or | application 14 (Single- |
| | DNA viruses | | ssDNA viral | high activity | stranded DNA mapping) |
| | (e.g., where a | | substrate) and one | on ssDNA and | |
| | plant variety | | or more ssDNA | low activity on | |
| | comprising a | | deaminases to produce | dsDNA | |
| | cytoplasmic | | deamination products | | |
| | ssDNA | | comprising deaminated | | |
| | deaminase is to | | cytosines and/or | | |
| | be engineered | | deaminated | | |
| | to have innate | | modified cytosines | | |
| | immunity) | | | | |
| 21 | Removing | NCN | (1) A deaminase | Activity on | Any enzyme from |
| | primers from | | substrate (e.g., a | ssDNA only, or | application 14 (Single- |
| | PCR reaction | | ssDNA substrate) and | high activity | stranded DNA mapping) |
| | | | one or more ssDNA | on ssDNA and | (combined with USER ®) |
| | | | deaminases to produce | low activity on | enzyme) |
| | | | deamination products | dsDNA | |
| | | | comprising deaminated | | |
| | | | cytosines and/or | | |
| | | | deaminated | | |
| | | | modified cytosines | | |
| | | | in ssDNA regions | | |
| | | | (2) Deamination | | |
| | | | products with a | | |
| | | | uracil DNA | | |
| | | | glycosylase and an | | |
| | | | endonuclease VIII | | |
| | | | (e.g., USER ® | | |
| | | | Enzyme, M5505, | | |
| | | | NEB, Inc.) | | |
| 22 | Random | NCN | A deaminase | Activity on | CseDa01, LbDa02, |
| | mutagenesis | | substrate (e.g., | dsDNA | MGYPDa829, MGYPDa06, |
| | (C −> T) | | dsDNA or a dsDNA plus | | CrDa01, AvDa02 (all the |
| | | | ssDNA) and a dsDNA | | non-specific dsDNA |
| | | | deaminase to produce | | deaminases) |
| | | | deamination products | | |
| | | | comprising deaminated | | |
| | | | cytosines and/or | | |
| | | | deaminated | | |
| | | | modified cytosines | | |
| | | | randomly | | |
| | | | distributed in the | | |
| | | | substrate | | |
| 23 | EasyScreen ™ & | NCN | (1) A deaminase | High activity | CseDa01 |
| | 3base ™ | | substrate (e.g., | on dsDNA | |
| | Technology | | genomic DNA with | | |
| | (Genetic | | a high GC content) | | |
| | Signatures) | | and a dsDNA | | |
| | | | deaminase to produce | | |
| | | | deamination products | | |
| | | | comprising deaminated | | |
| | | | cytosines (uracils) | | |
| | | | (2) the deamination | | |
| | | | products (or | | |
| | | | amplification | | |
| | | | products thereof) | | |
| | | | with a primer | | |
| | | | complementary to | | |
| | | | a target sequence | | |
| | | | comprising one or | | |
| | | | more of the | | |
| | | | deaminated cytosines | | |
| 24 | Making dsDNA | NCN | A dsDNA substrate | Activity on | CseDa01, LbDa02, |
| | deaminase | | and a dsDNA | dsDNA | MGYPDa829, MGYPDa06, |
| | converted | | deaminase to produce | | CrDa01, AvDa02 |
| | duplexes for | | deamination products | | (dsDNA deaminase creates |
| | the strand- | | comprising deaminated | | C > T transitions at unique |
| | specific | | cytosines and/or | | positions in each strand. |
| | detection and | | deaminated | | Amplification of the (+) and |
| | quantification | | modified cytosines | | (−) strands with primers that |
| | of rare | | that also include | | are amplicon and strand- |
| | mutations | | the positions of the | | specific allows for targeted |
| | | | rare mutations. | | amplification and addition |
| | | | | | of molecular barcodes; |
| | | | | | Mattox, Austin K., et al. |
| | | | | | “Bisulfite-converted |
| | | | | | duplexes for the strand- |
| | | | | | specific detection and |
| | | | | | quantification of rare |
| | | | | | mutations.” Proceedings of |
| | | | | | the National Academy of |
| | | | | | Sciences 114.18 (2017): |
| | | | | | 4733-4738.) |

TABLE 3

| SEQ | | C:C_ | C:C_ | C:CG_ | C:CH_ | 5mC:C_ | 5hmC:C_ | 5ghmC:C_ |
|---|---|---|---|---|---|---|---|---|
| ID | Name | dsDNA | ssDNA | dsDNA | dsDNA | dsDNA | dsDNA | dsDNA |
| 1 | LbDa02 | 1.00 | 1.00 | 1.00 | 1.00 | 1.00 | 1.00 | 0.37 |
| 2 | AvDa02 | 1.00 | 1.00 | 1.00 | 1.00 | 0.98 | 0.76 | 0.01 |
| 3 | CseDa01 | 1.00 | 0.98 | 1.00 | 1.00 | 1.00 | 0.98 | 0.97 |
| 4 | MGYPDa06 | 1.00 | 0.98 | 1.00 | 1.00 | 0.96 | 0.78 | |
| 5 | d38_MGYPDa829 | 0.99 | 0.99 | 0.99 | 0.99 | 0.81 | 0.77 | 0.09 |
| 6 | MGYPDa23 | 0.94 | 0.92 | 0.99 | 0.91 | 0.38 | 0.28 | 0.07 |
| 7 | MGYPDa26 | 0.93 | 0.86 | 0.95 | 0.92 | 0.11 | 0.04 | 0.03 |
| 8 | FlDa01 | 0.93 | 0.85 | 0.93 | 0.93 | 0.29 | 0.06 | 0.03 |
| 9 | CsDa01 | 0.91 | 0.78 | 0.90 | 0.92 | 0.18 | 0.08 | 0.02 |
| 10 | LbsDa01 | 0.89 | 0.87 | 0.89 | 0.89 | 0.33 | 0.22 | 0.04 |
| 11 | MGYPDa20 | 0.86 | 0.79 | 0.92 | 0.83 | 0.05 | 0.02 | 0.02 |
| 12 | CrDa01 | 0.81 | 0.95 | 0.79 | 0.82 | 0.14 | 0.31 | 0.11 |
| 13 | d22_PeDa01 | 0.79 | 0.57 | 0.88 | 0.74 | 0.74 | 0.64 | |
| 14 | MGYPDa16 | 0.78 | 0.90 | 0.89 | 0.73 | 0.21 | 0.34 | 0.06 |
| 15 | BcDa02 | 0.77 | 0.75 | 0.86 | 0.73 | 0.07 | 0.03 | 0.01 |
| 16 | MGYPDa01 | 0.75 | 0.85 | 0.89 | 0.69 | 0.58 | 0.33 | |
| 17 | PfDa01 | 0.70 | 0.97 | 0.69 | 0.70 | 0.32 | 0.64 | 0.03 |
| 18 | PpDa03 | 0.69 | 0.62 | 0.86 | 0.61 | 0.27 | 0.12 | 0.03 |
| 19 | LbDa01 | 0.67 | 0.97 | 0.54 | 0.73 | 0.40 | 0.37 | |
| 20 | MGYPDa10 | 0.64 | 0.97 | 0.61 | 0.65 | 0.18 | 0.18 | |
| 21 | AvDa01 | 0.64 | 0.70 | 0.70 | 0.61 | 0.20 | 0.13 | 0.02 |
| 22 | PbDa01 | 0.64 | 0.81 | 0.68 | 0.62 | 0.11 | 0.02 | 0.01 |
| 23 | PwDa01 | 0.62 | 0.56 | 0.82 | 0.54 | 0.23 | 0.09 | 0.03 |
| 24 | BaDa01 | 0.61 | 0.72 | 0.63 | 0.60 | 0.10 | 0.04 | 0.02 |
| 25 | PpDa04 | 0.61 | 0.54 | 0.75 | 0.55 | 0.04 | 0.02 | 0.02 |
| 26 | SaDa02 | 0.61 | 0.56 | 0.82 | 0.52 | 0.51 | 0.39 | |
| 27 | NsDa01 | 0.60 | 0.62 | 0.78 | 0.52 | 0.06 | 0.02 | 0.01 |
| 28 | EcDa01 | 0.57 | 0.74 | 0.80 | 0.47 | 0.12 | 0.21 | 0.03 |
| 29 | HgDa01 | 0.56 | 0.94 | 0.39 | 0.63 | 0.27 | 0.32 | |
| 30 | AmDa01 | 0.53 | 0.52 | 0.73 | 0.45 | 0.40 | 0.38 | 0.02 |
| 31 | MGYPDa408 | 0.50 | 0.71 | 0.46 | 0.51 | 0.30 | 0.32 | 0.04 |
| 32 | SzDa01 | 0.47 | 0.53 | 0.46 | 0.48 | 0.19 | 0.11 | 0.01 |
| 33 | RaDa01 | 0.47 | 0.70 | 0.46 | 0.47 | 0.17 | 0.14 | 0.04 |
| 34 | MGYPDa624 | 0.45 | 0.90 | 0.42 | 0.46 | 0.14 | 0.09 | 0.01 |
| 35 | EcDa04 | 0.43 | 0.40 | 0.65 | 0.34 | 0.28 | 0.19 | |
| 36 | BlDa01 | 0.42 | 0.41 | 0.60 | 0.34 | 0.04 | 0.01 | 0.01 |
| 37 | d16_MGYPDa17 | 0.42 | 0.65 | 0.87 | 0.23 | 0.17 | 0.16 | 0.02 |
| 38 | CgmDa01 | 0.40 | 0.96 | 0.63 | 0.31 | 0.14 | 0.10 | 0.02 |
| 39 | NoDa01 | 0.36 | 0.86 | 0.23 | 0.42 | 0.20 | 0.33 | 0.05 |
| 40 | AshDa01 | 0.34 | 0.62 | 0.70 | 0.19 | 0.01 | 0.01 | 0.00 |
| 41 | MGYPDa18 | 0.33 | 0.69 | 0.44 | 0.29 | 0.05 | 0.02 | 0.01 |
| 42 | MGYPDa687 | 0.32 | 0.56 | 0.32 | 0.31 | 0.26 | 0.19 | 0.01 |
| 43 | PpDa02 | 0.31 | 0.25 | 0.47 | 0.24 | 0.14 | 0.15 | |
| 44 | MGYPDa03 | 0.29 | 0.30 | 0.49 | 0.21 | 0.25 | 0.09 | |
| 45 | LsfDa01 | 0.26 | 0.22 | 0.34 | 0.22 | 0.01 | 0.01 | 0.00 |
| 46 | MGYPDa02 | 0.25 | 0.38 | 0.39 | 0.19 | 0.14 | 0.12 | |
| 47 | PvmDa01 | 0.24 | 0.33 | 0.48 | 0.14 | 0.06 | 0.05 | 0.01 |
| 48 | MGYPDa917 | 0.24 | 0.44 | 0.25 | 0.24 | 0.21 | 0.14 | 0.01 |
| 49 | AcDa01 | 0.24 | 0.57 | 0.79 | 0.01 | 0.18 | 0.05 | |
| 50 | CbDa01 | 0.24 | 0.67 | 0.76 | 0.02 | 0.09 | 0.06 | 0.00 |
| 51 | HmDa03 | 0.24 | 0.29 | 0.37 | 0.18 | 0.16 | 0.16 | 0.01 |
| 52 | WWTPDa05 | 0.22 | 0.06 | 0.31 | 0.19 | 0.17 | 0.10 | 0.01 |
| 53 | d22_SjDa01 | 0.22 | 0.13 | 0.36 | 0.16 | 0.08 | 0.11 | 0.01 |
| 54 | MGYPDa09 | 0.20 | 0.95 | 0.10 | 0.25 | 0.10 | 0.07 | |
| 55 | MGYPDa05 | 0.20 | 0.44 | 0.54 | 0.06 | 0.06 | 0.03 | |
| 56 | VsDa01 | 0.20 | 0.09 | 0.21 | 0.20 | 0.02 | 0.01 | 0.02 |
| 57 | BaDa02 | 0.20 | 0.11 | 0.29 | 0.16 | 0.02 | 0.01 | 0.00 |
| 58 | HmDa02 | 0.17 | 0.17 | 0.32 | 0.11 | 0.08 | 0.05 | 0.01 |
| 59 | SaDa03 | 0.16 | 0.52 | 0.28 | 0.12 | 0.11 | 0.03 | |
| 60 | PdDa01 | 0.15 | 0.50 | 0.25 | 0.11 | 0.00 | 0.00 | 0.00 |
| 61 | BcDa01 | 0.15 | 0.09 | 0.26 | 0.11 | 0.07 | 0.04 | |
| 62 | DaDa01 | 0.15 | 0.68 | 0.33 | 0.07 | 0.02 | 0.01 | 0.00 |
| 63 | MmgDa02 | 0.13 | 0.26 | 0.45 | 0.00 | 0.02 | 0.01 | 0.00 |
| 64 | MGYPDa21 | 0.08 | 0.12 | 0.05 | 0.09 | 0.01 | 0.00 | 0.00 |
| 65 | RhDa01 | 0.07 | 0.09 | 0.23 | 0.00 | 0.00 | 0.00 | |
| 66 | MsDa01 | 0.07 | 0.20 | 0.06 | 0.07 | 0.01 | 0.01 | 0.00 |
| 67 | HgmDa01 | 0.06 | 0.16 | 0.21 | 0.00 | 0.00 | 0.00 | 0.00 |
| 68 | XcDa01 | 0.05 | 0.16 | 0.00 | 0.08 | 0.02 | 0.01 | 0.00 |
| 69 | AoDa01 | 0.05 | 0.19 | 0.04 | 0.05 | 0.01 | 0.01 | 0.00 |
| 70 | HmDa01 | 0.04 | 0.09 | 0.10 | 0.02 | 0.02 | 0.02 | 0.00 |
| 71 | HgmDa02 | 0.04 | 0.30 | 0.12 | 0.01 | 0.00 | 0.00 | 0.00 |
| 72 | MGYPDa13 | 0.04 | 0.71 | 0.02 | 0.04 | 0.03 | 0.01 | |
| 73 | MGYPDa11 | 0.04 | 0.04 | 0.03 | 0.04 | 0.13 | 0.03 | 0.00 |
| 74 | d36_PaDa02 | 0.03 | 0.24 | 0.02 | 0.04 | 0.00 | 0.00 | |
| 75 | BbDa01 | 0.03 | 0.14 | 0.03 | 0.03 | 0.00 | 0.00 | 0.00 |
| 76 | PbDa02 | 0.03 | 0.21 | 0.06 | 0.01 | 0.00 | 0.00 | 0.00 |
| 77 | PsDa01 | 0.02 | 0.05 | 0.01 | 0.02 | 0.05 | 0.02 | 0.00 |
| 78 | AdDa01 | 0.01 | 0.03 | 0.04 | 0.00 | 0.01 | 0.00 | 0.00 |
| 79 | KsDa01 | 0.01 | 0.03 | 0.03 | 0.01 | 0.00 | 0.00 | 0.00 |
| 80 | VRDa06 | 0.01 | 0.01 | 0.02 | 0.01 | 0.00 | 0.00 | 0.00 |
| 81 | ScDa03 | 0.01 | 0.15 | 0.01 | 0.01 | 0.00 | 0.01 | 0.00 |
| 82 | WWTPDa04 | 0.00 | 0.13 | 0.01 | 0.00 | 0.00 | 0.00 | 0.00 |
| 83 | CaDa01 | 0.00 | 0.00 | 0.01 | 0.00 | 0.00 | 0.00 | 0.00 |
| 84 | SpDa01 | 0.00 | 0.00 | 0.00 | 0.00 | 0.00 | 0.00 | 0.00 |
| 85 | MGYPDa14 | 0.00 | 0.05 | 0.00 | 0.00 | 0.00 | 0.00 | 0.00 |
| 86 | AmDa03 | 0.00 | 0.09 | 0.00 | 0.00 | 0.00 | 0.00 | 0.00 |
| 87 | xp12da | 0.00 | 0.00 | 0.00 | 0.00 | 0.00 | 0.00 | |
| 88 | gp317 | 0.00 | 0.00 | 0.00 | 0.00 | 0.00 | 0.00 | |
| 89 | AbcDa01 | 0.00 | 0.00 | 0.00 | 0.00 | 0.00 | 0.00 | |
| 90 | WcDa01 | 0.00 | 0.00 | 0.00 | 0.00 | 0.00 | 0.00 | 0.00 |
| 91 | XinDa01 | 0.00 | 0.00 | 0.00 | 0.00 | 0.00 | 0.00 | 0.00 |
| 92 | XjaDa01 | 0.00 | 0.00 | 0.00 | 0.00 | 0.00 | 0.00 | 0.00 |
| 93 | HcDa01 | 0.00 | 0.94 | 0.00 | 0.00 | 0.00 | 0.00 | |
| 94 | SsDa01 | 0.00 | 0.54 | 0.00 | 0.00 | 0.01 | 0.00 | 0.00 |
| 95 | AncDa04 | 0.99 | 1.00 | 0.99 | 1.00 | 0.93 | 0.73 | |
| 96 | MGYPDa829 | 0.96 | 0.95 | 0.96 | 0.96 | 0.52 | 0.33 | |
| 97 | chimera 10 | 0.95 | 1.00 | 0.95 | 0.95 | 0.67 | 0.68 | |
| 98 | chimera_09 | 0.71 | 0.88 | 0.77 | 0.68 | 0.34 | 0.38 | 0.03 |
| 99 | d22_Cd4_PeDa01 | 0.64 | 0.60 | 0.80 | 0.58 | 0.54 | 0.51 | 0.03 |
| 100 | chimera_05 | 0.59 | 0.96 | 1.00 | 0.41 | 0.37 | 0.25 | |
| 101 | chimera_07 | 0.56 | 0.97 | 0.99 | 0.38 | 0.33 | 0.24 | |
| 102 | chimera 20 | 0.41 | 0.63 | 0.69 | 0.29 | 0.07 | 0.09 | 0.01 |
| 103 | MGYPDa17 | 0.37 | 0.51 | 0.82 | 0.18 | 0.16 | 0.13 | 0.03 |
| 104 | chimera_19 | 0.31 | 0.50 | 0.61 | 0.18 | 0.03 | 0.05 | 0.01 |
| 105 | AncDa05 | 0.30 | 0.28 | 0.46 | 0.23 | 0.02 | 0.01 | |
| 106 | PeDa01 | 0.29 | 0.15 | 0.48 | 0.21 | 0.25 | 0.10 | |
| 107 | AncDa03 | 0.25 | 0.63 | 0.82 | 0.02 | 0.09 | 0.05 | |
| 108 | chimera_08 | 0.23 | 0.62 | 0.39 | 0.17 | 0.03 | 0.07 | 0.00 |
| 109 | MGYPDa18_extN | 0.20 | 0.27 | 0.30 | 0.15 | 0.02 | 0.01 | 0.01 |
| 110 | chimera_18 | 0.16 | 0.31 | 0.43 | 0.05 | 0.02 | 0.01 | 0.00 |
| 111 | d22_HmDa02 | 0.13 | 0.13 | 0.26 | 0.08 | 0.05 | 0.03 | 0.00 |
| 112 | AncDa06 | 0.13 | 0.77 | 0.42 | 0.01 | 0.01 | 0.01 | |
| 113 | d41_MGYPDa917 | 0.13 | 0.33 | 0.14 | 0.12 | 0.10 | 0.04 | |
| 114 | RhDa01_extN10 | 0.10 | 0.15 | 0.32 | 0.00 | 0.01 | 0.01 | 0.00 |
| 115 | d21_HcDa01 | 0.08 | 0.97 | 0.16 | 0.05 | 0.01 | 0.04 | 0.00 |
| 116 | chimera_06 | 0.08 | 0.54 | 0.27 | 0.00 | 0.02 | 0.01 | |
| 117 | chimera_17 | 0.05 | 0.12 | 0.16 | 0.01 | 0.00 | 0.00 | 0.00 |
| 118 | d38_Cd11_MGY | 0.04 | 0.48 | 0.04 | 0.04 | 0.00 | 0.00 | |
| | PDa829 | | | | | | | |
| 119 | chimera_01 | 0.00 | 0.36 | 0.00 | 0.00 | 0.00 | 0.00 | |
| 153 | BadTF3 | 0.54 | 0.53 | 0.66 | 0.49 | 0.40 | 0.31 | 0.02 |
| 154 | APOBEC3A | 0.33 | 1.00 | 0.33 | 0.33 | 0.06 | 0.01 | 0.00 |
| 155 | DddA | 0.27 | 0.24 | 0.26 | 0.27 | 0.08 | 0.10 | 0.01 |
| 156 | ssdA | 0.20 | 0.47 | 0.16 | 0.22 | 0.10 | 0.06 | 0.01 |
| 159 | Sso7d RhDa01_ | 0.93 | | 0.97 | 0.92 | 0.05 | 0.02 | |
| | extN10 | | | | | | | |
| 160 | Sso7d_GGGVTS_ | 0.45 | | 0.69 | 0.34 | 0.03 | 0.01 | |
| | RhDa01_extN10 | | | | | | | |
| 161 | Sso7d_GGGVTS_ | 0.21 | 0.61 | 0.35 | 0.15 | 0.01 | 0.00 | |
| | MGYPDa20 | | | | | | | |
| 162 | Sso7d_ | 0.72 | 0.75 | 0.82 | 0.67 | 0.03 | 0.01 | |
| | VTAGVGEAG_ | | | | | | | |
| | MGYPDa20 | | | | | | | |
| 163 | Sso7d_GGGVTS_ | 0.26 | 0.61 | 0.79 | 0.03 | 0.16 | 0.15 | |
| | AcDa01 | | | | | | | |
| 164 | Sso7d_ | 0.41 | 0.83 | 0.99 | 0.17 | 0.32 | 0.40 | |
| | LSGLSDDKLKEI_ | | | | | | | |
| | AcDa01 | | | | | | | |

C:C_dsDNA: fraction of unmodified cytosines deaminated in double-stranded DNA
C:C_ssDNA: fraction of unmodified cytosines deaminated in single-stranded DNA
C:CG_dsDNA: fraction of unmodified cytosines in CpG context, deaminated in double-stranded DNA
C:CH_dsDNA: fraction of unmodified cytosines followed by an adenine, cytosine, or thymine, deaminated in double-stranded DNA
5mC:C_dsDNA: fraction of cytosines with the 5-methyl modification, deaminated in double-stranded DNA.
5hmC:C_dsDNA: fraction of cytosines with the 5-hydroxymethyl modification, deaminated in double-stranded DNA.
5ghmC:C_dsNDA fraction of cytosines with the 5ghmC modification, deaminated in double-stranded DNA (5hmC bases modified by glucosylation yielding 5ghmC)

TABLE 4

| SEQ ID | Current name | Provisional name |
|---|---|---|
| 5 | d38_MGYPDa829 | d38_MGYP001104162829 |
| 31 | MGYPDa408 | MGYP000983427408 |
| 34 | MGYPDa624 | MGYP001011623624 |
| 42 | MGYPDa687 | MGYP000859226687 |
| 48 | MGYPDa917 | MGYP000473187917 |
| 96 | MGYPDa829 | MGYP001104162829 |

TABLE 5

deamination efficiency

| SEQ ID | Name | C | 5 caC | 5 fC |
|---|---|---|---|---|
| 2 | AvDa02 | 0.95 | 0.00 | 0.08 |
| 12 | CrDa01 | 0.98 | 0.00 | 0.09 |
| 28 | EcDa01 | 0.60 | 0.00 | 0.01 |
| 10 | LbsDa01 | 0.92 | 0.10 | 0.14 |
| 16 | MGYPDa01 | 0.12 | 0.00 | 0.00 |
| 6 | MGYPDa23 | 0.97 | 0.02 | 0.23 |
| 5 | d38_MGYPDa829 | 0.88 | 0.10 | 0.12 |
| 11 | MGYPDa20 | 0.59 | 0.00 | 0.00 |
| 13 | d22_PeDa01 | 0.14 | 0.00 | 0.00 |
| 33 | RaDa01 | 0.98 | 0.47 | 0.66 |

What is claimed is:

1. A method for sequencing, comprising:
contacting a double-stranded DNA substrate comprising a genomic DNA fragment with a double-stranded DNA deaminase to produce a deamination product;
sequencing the deamination product, or amplifying the deamination product to produce amplification products and sequencing the amplification products, in each case, to produce sequence reads.

2. The method of claim 1, wherein the double-stranded DNA deaminase has sequence bias for cytosine in a CpG context.

3. The method of claim 2, wherein the double-stranded DNA deaminase is modification sensitive.

4. The method of claim 3, wherein the double-stranded DNA deaminase does not deaminate one or more of 5fC, 5CaC, 5mC, 5hmC, N4mC, or 5ghmC.

5. The method of claim 2, wherein the double-stranded DNA deaminase is not modification sensitive.

6. The method of claim 2, wherein the double-stranded DNA substrate or the genomic fragment is not pre-treated with either a TET methylcytosine dioxygenase or DNA beta-glucosyltransferase.

7. The method of claim 2, wherein the double-stranded DNA substrate or the genomic DNA fragment is pre-treated with a TET methylcytosine dioxygenase, and optionally is pre-treated with a DNA beta-glucosyltransferase.

8. The method of claim 3, wherein the double-stranded DNA deaminase has an amino acid sequence that is at least 80% identical to any of SEQ ID NOS: 40, 62, 63, 65, 67, 71, 110, 112, 114, and 117.

9. The method of claim 5, wherein the double-stranded DNA deaminase has an amino acid sequence that is at least 80% identical to any of SEQ ID NOS: 47, 49, 50, 55, 58, 59, 70, 76, 106, 107, 163 and 164.

10. The method of claim 1, wherein the double-stranded DNA substrate further comprises a genomic fragment linked to an adapter.

11. The method of claim 10, wherein the adapter comprises a primer.

12. The method of claim 1, wherein the strands of the double-stranded DNA substrate are not linked together by an adapter.

13. The method of claim 1, wherein the deamination product is double-stranded.

14. The method of claim 1, wherein the double-stranded DNA substrate is not a multi-copy strand.

15. The method of claim 1, further comprising analyzing the sequence reads to identify a modified cytosine in the double-stranded DNA substrate.

16. The method of claim 15, wherein a reference sequence is not used for the analyzing.

17. The method of claim 15, wherein the modified cytosine is one or more of 5fC, 5CaC, 5mC, 5hmC, N4mC, or 5ghmC.

18. The method of claim 17, wherein the modified cytosine is 5hmC and the double-stranded DNA deaminase has an amino acid sequence that is at least 80% identical to any of SEQ ID NOS: 4, 5, 10, 13, 16, 96, 99 and 106.

19. A method for deaminating a nucleic acid, the method comprising:
contacting:
a DNA substrate that comprises cytosines; and
a double-stranded DNA deaminase having an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163, and 164.
to produce a deamination product that comprises deaminated cytosines.

20. The method of claim 19, wherein the DNA substrate further comprises a modified cytosine.

21. The method of claim 21, wherein the modified cytosine is a 5fC, 5CaC, 5mC, 5hmC, N4mC, 5ghmC, or pyrrolo-C.

22. An enzyme comprising an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163, and 164.

23. The enzyme of claim 22, wherein the enzyme is fused with a DNA binding domain.

24. The enzyme of claim 23, wherein the DNA binding domain is selected from a Cas9 domain, a Cas12 domain, a transcription activator-like effector nuclease (TALEN domain), a zinc finger (ZF) domain, a transcription activator-like effector (TALE) domain, an Sso7d domain, and a methyl binding domain (MBD) domain.

25. The enzyme of claim 22, wherein the enzyme is no more than 300 amino acids in length.

26. A method for sequencing, comprising:
contacting a single-stranded DNA substrate comprising a genomic DNA fragment with a double-stranded DNA deaminase to produce a deamination product;
sequencing the deamination product, or amplifying the deamination product to produce amplification products and sequencing the amplification products, in each case, to produce sequence reads,
wherein the double-stranded DNA deaminase is an enzyme of claim 22.

27. A kit comprising:
(a) an enzyme of claim 22; and
(b) a reaction buffer.

28. The kit of claim 27, wherein the kit further comprises:
a TET methylcytosine dioxygenase and a DNA beta-glucosyltransferase; or
a TET methylcytosine dioxygenase and no DNA beta-glucosyltransferase

29. The kit of claim 27, wherein the kit is free of TET methylcytosine dioxygenase and DNA beta-glucosyltransferase.

30. A reaction mix comprising:
(a) a DNA substrate that comprises cytosines; and
(b) a double-stranded DNA deaminase having an amino acid sequence that is at least 80% identical to any of SEQ ID NOS: 21, 40, 47, 49, 50, 55, 58, 59, 62, 63, 65, 67, 70, 71, 76, 106, 107, 110, 112, 114, 117, 163 and 164.

31. The reaction mix of claim 30, wherein the DNA substrate comprises cytosines and at least one modified cytosine.

32. The reaction mix of claim 31, wherein the modified cytosine is a 5fC, 5caC, 5mC, 5hmC, N4mC or pyrrolo-C.

33. A method for base editing comprising:
contacting a fusion protein with a target sequence to produce an edited target sequence
comprising at least one deaminated cytosine or deaminated modified cytosine, wherein the fusion protein comprises a dsDNA deaminase fused to a DNA binding domain.

34. The method of claim 33, wherein the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain.

35. The method of claim 34, wherein the fusion protein further comprises a guide RNA complementary to at least a portion of the targeted sequence.

36. The method of claim 33 wherein the fusion protein comprises an enzyme at is at least 80% identical to any of SEQ ID NOS:1-152.


SEQUENCE LISTING (169 sequences)

SEQ ID NO: 1 - protein, 186 aa, unidentified
  REGION 1..186: note = MISC_FEATURE - LbDa02
  source 1..186: note = Lachnospiraceae bacterium
MGTHNNKTNTSRQNIKKERIGSMNDGNSESGSDALDKKIKQAEQAQKKASELSDKYGYGTAPKKTVASDGTIHTLSGWKPLDENSEFTRVSTDDVINKSNDIGHKLRNAGANDQGVPGRYNASHAEKQLSIISDKPIGISQPMCTDCQGYFKSLAIGEGKEYVTADPNYIRIFSPDGTVNEIERQR

SEQ ID NO: 2 - protein, 456 aa, Actinomyces viscosus
  REGION 1..456: note = MISC_FEATURE - AvDa02
MTGNELVAEVKDSETVWTGTRLIEDGFDLKEAFESKSWVAGGLATLATAADTASAVMDPLGEAMSAGVAWVIEHLGSLNEWLNELTGDSDAVAAAASTWSNIGTKLGSCADDLETVCTGRLAGQESLAVATFKSLQAGSASHLRMTGGVASAISGGLTVASVIVRMVHDLVRDAIADVIGKVASKVAIGVLTAGLAAPWAVASVIADVSSWVTRLSKEVADVVLSARNLKTLLDKASKLFDDVGEKFAALVAMLMGRGSKKADDAVEAAEDAVDAATDGAKGADGAVPPGKGGGGGGGGTPPKGGGGEGDAVKTSKEAYARAKDLRKALQEKVAYKDKTPASDHVRTLSGWSKTRRPPGYQDPNVDEVLEKCKEIGHEPKPHRRNDNGIEGQYNASHAEKQLSLTAKEPYIGVGKPCCSDCQAYFSKLAQHEGRDWYVTDPKKTWIFRADGSIEYK

SEQ ID NO: 3 - protein, 467 aa, Candidatus Scybalomonas excrementigallinarum
  REGION 1..467: note = MISC_FEATURE - CseDa01
MNRLLEEVYHENETEEKIQYEYDLCGNRLSKKQWKDKNILEEKSWYNQKNQLIKRQLEKGTVFYTYDKQGNTIQETGEEVDWIYSYNPFGQQIKSKNTNGYFQENFYDGEYLRAGVKKNESSSKFLYYQGELLSEITKKQEERKGNRLILGYGVVASEELGLQNHREEHEHHNFHAKSDRRTIGFYHLDEQNSTVFITGRNQEIQNSYTYDAFGNVRNQKETHHNRILYTGQQYDKESNQYYLRARYYNPTLGRFTQEDVYRGAGLNLYDYCKGNPVIWYDPSGYENCKIDKGNVSNKENGFEDEIIQAKIAQQTAFELAEKYGYNGAPPKRKTVASDGEINTLSGWKKLKDNTDFIRVSPEDIIDKSQEIGHNLRNAGANDQGIKGKYNASHAEKQLSLKTDKPIGISQPMCQDCQNYFRCLAISESKNFVTADTNMVRIFKPDGSVINYKSDDSFSIIKIKNKIK

SEQ ID NO: 4 - protein, 260 aa, unidentified
  REGION 1..260: note = Isolated from environmental sources.Organism unknown.
  REGION 1..260: note = MISC_FEATURE - MGYPDa06
ARFYNPVVARFTQEDAYRGDGLNLYAYCANSPAFYSDPTGNLSQCMKDAYLEARRAGATKEEAFAAAKAAYEQNHVDSDPRPTVDVPDDRSMTREQWNDAYKDARVIDHATEQIDRAIQNQQLAHDISAQYGYDSSPGKKTVATDGTVATLSGWRDLGPDSQFARVSPAAVAEKSAEIGHNLRNAGALDQGIPGQFNASHAEKQLSLIADGPIGISEPMCPDCQRYFNALATAERQQYVTADPNTVRIFNQDGSVTTINR

SEQ ID NO: 5 - protein, 164 aa, unidentified
  REGION 1..164: note = Isolated from environmental sources.Organism unknown.
  REGION 1..164: note = MISC_FEATURE - d38_MGYPDa829
DFHTYYVGTESVLVHNNGGNCMKTASALAQGGSKGGSTAINLPEYDGKTTHGVLVLDNGTQVQLVSGNGDPRYTNYRNNGHVEQKAAIYMRENNISNATVYHNNTNGTCGYCNTMTATFLPEGATLTVVPPKNAVANNSRAIAYVKTYTGTSNDPKMSSRYKGN

SEQ ID NO: 6 - protein, 274 aa, unidentified
  REGION 1..274: note = Isolated from environmental sources.Organism unknown.
  REGION 1..274: note = MISC_FEATURE - MGYPDa23
QEDNTTQEVITWLFEGFVPVAKIVDDQKYSIVSDYLGKPFQAYDSEGELVWAAEHNIYGKVKTLKGDKTFIPFRQLGQYEDIEIDLYYNRFRYYDCHTGTYISQDPIGLAGNNPNIYAYVFDSNTEVDVFGLEVIFDETLANMARQAHDVLREKDKTKIKFNHSTVAIAEVEINGEKQLFASGNGAVLSPAQRDKLKELGIPEENIFSGKRFKKILEDLEATKLANHAERVIIRNVPEGAIFTGKWGISWAGNQRNESCVNCLSFVEKVSNKHH

SEQ ID NO: 7 - protein, 337 aa, unidentified
  REGION 1..337: note = Isolated from environmental sources.Organism unknown.
  REGION 1..337: note = MISC_FEATURE - MGYPDa26
GGEGHPASKTIAARASQTGATGQNHSKPAANDAEALYWASLNQQAEQKAQALAIEGWGTGTDGPQQQTAQAKIAEVYYFHTDQVGLPEELSNAQGQLVWQATYKTWGNTQAEEWKLVEADGSKTHPLDAGDQPESEAKHQNLRFQGQYLDRDTGLHYNTFRFYDPDIGRFISPDPIGLEGGINLGSYSPNPIGWIDPWGWSCGELSSKARQVHNMAGGKVPDARSIRNSTVAIFEVKLPNGKTTLYAAGSSGKLNPRQRAELVKLGVPESNILHGKGVNLPKDGNPVNANHAEQIIIRGIPDGSSVTRKGISWGGKQTNAPCIHCKPSVDGIGGIYD

SEQ ID NO: 8 - protein, 275 aa, Flavobacterium sp.
  REGION 1..275: note = MISC_FEATURE - FlDa01
  source 1..275: note = Flavobacterium sp. Leaf82
MVYDKPEPIKNLITWVYEGGSFVPSAKIIGEHKFSIINDYIGRPIQVYNEVGDVVWETDYDIYGGLRNLKGDKSFIPFRQLGQYEDVETGLYYNRHRYYNPESGGYISQDPIGLLGGSASYKYVHDCNNCVDIFGLNPVIFSEELSKIAQEAHNVLLEPGKSPRGFNNSTVSVAKVDVNGVSTLYASGNGASLSPAQRTKLVELGVPEENIFSGKRFKEIIDGDTGTLTKLSNHAERVIERNIPKDASIKEWGISWASKQKNEMCNNCKTHFGCK

SEQ ID NO: 9 - protein, 1380 aa, Chitinophaga silvisoli
  REGION 1..1380: note = MISC_FEATURE - CsDa01
MLISNKHFVPVIGLDIHIVILFGFPIPLPHPYLGFVLDPMDYIPFLGASTKVNHVPRGVSDTSGIIIILFHFPMGGPWLLAPLIGHDSVNFYGAKNTLVEGRMLSPTGHMLMTCNDIGLPLSLKPGKKLIPMPGMYLPTSYSIPLSFGKPVIVGGPYVPDWAGVLMNLLMSFGFGALLKGIGKLGKKMMTKFNHALKGKLGSNKLSKFLCKHGFEPVDLVQGIVISEGLDFELSGPIPIVWERTWNSDSAHKGLLGHGNHLFYDMRVQEFAAENATVVLLRDGRSAVFDALPYSGDKEYNRHEKMLLTRTDADEFVLYDYEDKLFYTFKKIRETDEQFRLFAIHNKMNFLVTLHYNQKGHLLRITDSAGRQLHIKNDFEGRITSVTAHHRGQEMLLVSYEYNEEGDLTAIADALGQATRISYRNHLMVSKTDRTGQTFYWEYDNKKRCIHTWGDGGLLEGFIEYHPSEGYNLVTNSLGETTTYYYSPDFVISQIRDPMGNSTFINYTEDFEIYQEIDEEGTVAGYTYDDMGNMTSVVQPDGSSYSFLYNEAGEMILGMDAEGNSSTYTYYPDNGLLHTNTRPDGSIQIMRYNEQLLLASVEDTDDNITTMEYDEDFNLVKMVLPDGGEATWTYDVWGQCTSSSNPARQVQRFRYDALGRITDVETPDGNHISMEYDAYFQVLRAVDKHHDVKFSYTPLGNLRTREENGAKVNYIYDTECRLRTIINEKGESYQFVRNAKGESIRETGFDGISYGFVRDATGKVIKVERPGQKHSTYEYDYNGMLVRAEHSDGTWSSFAYDRNGLMIEATNENGTVSFKRDAMGNVIEERQNGHVVSTQYDENSRRMRVQTSLGAQVNLLRNRMGDITGMRANDSISRDPWTLQIQYDLAGMETERLLPGGISSSFTYDKAGMPASQTINSNKRTIRKTYYRWDANQRLREMVNALNNSSVKFAHDNFGNLAWAQYEDGHFDYRQPDKTGNLFRSVSQNDRKYGPGGQLQETPDARFEYDAEGFLIKKISSGLKVWEYEWTGRGTLQQVTRPDGKTVRFEYDALGRRTAKIYGNEITRWVWDGMNPVHEWTYPVDERPQIRIDDMGNIMQQEEPVPAETLITWVFEGDSCIPVAKIINGKQYSILSDYLGVPFAAYDGSGGECWSCELDIYGKVRKLAGSKGFIPFRYQGQYEDVETGLYYNRFRYYSPDEGVYISKDPIGLDGGSRLYSFVNDPMSLFDLLGLHPIFDDALAEKARQAHNVLKRNIPGKPEIGFNQSTVSIGEGTLPSGEKQLFASGNGAKLTPAQRAKLIELGVPPENIYSGVAYMKKPVNKSKVKSVRKAYNLENHAERVIIRNAPKGTKFKKWGISWAKYQKNEPCSNCKPHVQCAS

SEQ ID NO: 10 - protein, 125 aa, Simiaoa sunii
  REGION 1..125: note = MISC_FEATURE - LbsDa01
  source 1..125: note = Lachnospiraceae bacterium sunii NSJ-8
MSLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 11 - protein, 186 aa, unidentified
  REGION 1..186: note = Isolated from environmental sources.Organism unknown.
  REGION 1..186: note = MISC_FEATURE - MGYPDa20
QDPIGLLGGNNLYQFAPNAQGWVDWLGLSYGFNIKANRWTNFETGKFVQKPSTLSGQAQAVHNLAGGLKPSKISIEKSTVAIFEVQLRDGSKTLYAAGSSGVLNPRQRALLKQIGVPEKNILFGKGSVLPVTKVNSNINHAEQIILRNIPKDAKVTRKGISWSSKHKNAPCSKCQSSVDGAGGVYD

SEQ ID NO: 12 - protein, 215 aa, Clostridium roseum
  REGION 1..215: note = MISC_FEATURE - CrDa01
MGAGAEAIYKASKDGFKEAKNALRTVKDEKNGFVDGVADTNAPWKAIDDYRNQRQGLEVLPEDYEFIKGDGKNTVAFNDTCDKRYFGVNSTLRTDAEKGLAKKYFNKLVEEGYFPKGSIYPRGKAQFITHAEGYTLIKAYEENGIDIGKSVTIYVDRPTCGFCQNNLPKLQASMGIDVLTVINKNGDVFRCDLRKYTSMTVSDVNAEKLIKIKSK

SEQ ID NO: 13 - protein, 1560 aa, Pseudomonas entomophila
  REGION 1..1560: note = MISC_FEATURE - d22_PeDa01
  source 1..1560: note = Pseudomonas entomophila (strain L48)
MTDAKKREAQVAVAPLNTIDAKDVGRGAAAFDAWLQSVSGGYVTLERIKTVAGALPVVGNIMALVDALGDVVTLVKSKNRQLLDWVSLGINLIGVLPAPPTMAAARMSLRPTLFLVRQELRNSAKMLLGDSLIQVLIGHLNATIAGTIDDFVKQAQPKIAGILADAGALGTKMTSEIATGLEKVALSQLDAKGDMSKAGKQASAAADKLLNDPKAAFSNFLGAVHSAYKAAGKGLANSATSKLLPDQIKTKVLANTGQLRALGPEMARQLNKLADPGTQMSIGWMLTVLGGAVKGFRKRNKNGQPGVSKPGTTTQVEREKGKGELAVSGSQAKAQGTASPKRKPCPPEPKARTGHSISFSLGSEFILHTDFSLPGPFPIEWERTYHSRLAEYDQGTLGARWITEFTTRIDVVGKGLLFHDHDGRSHAFELPKVEQSLFNAIEDLLLVRSRENELVICRGFERKEYYLRVEERYYLQKITLASGAGCMLHYEHRHNGRPVLSDVITYQDDPRQIHRHLGTLIDEHGYVTGLWEMRDGQPLRQLCAYHYDDQGDLVAAQDEHGAAWHYQYQNHLVTRYTDRTGRGMNLQWDGSAFDAKAIREWADDGSYDTRLEWDENIRLTYVTDAHGQETWYYYDIQGHLYRVRYPDERSEWLFRDERKKLLRHVHPDGSEDRYDYDEQGSIIRHIRADHTQTHFAYDDRRHLIKIRDAEGGLWLRDYDQKGNLVETVDPLGNKTEFAYTPSGLVKAIKDANGNEKKLAYNAADQLIEYTDCSGKTSQWEYDDFGYLTQFIDAAGSKTVYEYKTGQLAKVIHPDNTEERFERDAEGRLLAHADALDRCTTWTYNAVGLLAERVDAAEHTLRYRWDKLGRLIGLENENESKANFLYDPIGRLLQEQGFDGLVTRYQYDPDTGRLASTQVGQRRIDITFDPVGRLASRVASLGDQRQEETFAYDGNGNLIQAINAASKLQWFHDEAGNLTREHQYYLGTEVPMVAVWQHEYDVLNNRVATVRPDGHKVSVLTYGSGHVLGMTLDQHELLAYERDDLHREVVRHQGNQLMQTQAWDLAGRLQEQLLGSHDGQSTLLKRQYQYDAVGQLTDIHDTRRGHLAYQYDPVGRLLQATSRLGVETFAFDPAGNLLDDKTQELNRPLESDPRRNKLMDNLLREYAGTHYQYDERGNLIHRLHNGEQARLSWDLFDRLTRFDDDKLTVVYSYDALGRRLHKHSTAHHQDDPRAGSGWNQMQRAKRQRELGCGYTLYGWDGDNLAWESSPPQDEGETGRTVHYLYEPGSFVPVAQALRKQPIRLLRQPDWSDREYDFDQDPLWQHEVKPQPVDAIAWYQCDHLGTPMELTDHHGEVAWTAQYKAWGEIKETRSEWAKQVGLNNPIRFQGQYHDHETALHYNRYRYYDSRAGRFISQDPISYAGGTNLFAYAPNPIWWIDPLGLNKFGSGKGVHTAVVNVHDSDGEHTVVSCILHSGNMTEEERALGFPRSSLATHTEARAVKQIPLQQGDVMVIDGQYPPCPSCKGKMNRASTETGAKIQYTWPENGETKTWTAGQKRRRR

SEQ ID NO: 14 - protein, 378 aa, unidentified
  REGION 1..378: note = Isolated from environmental sources.Organism unknown.
  REGION 1..378: note = MISC_FEATURE - MGYPDa16
MFGCGHAMRGKAGTASCGLMNFLKYGTAALSVGTGGYDIYQIGKENYQKYVVNGEKFSWGDAALDTLRITLDATSIAGGIKLATETIPYCFVAGTLVETEYGQKPIEEIQAGDKVLSENPETGEIAYKTVEETYINETDELIHVHVNDETISATPNHPFYVDKLGWTLAKNLRAGDILVLSNGEFVVVEWIQHEILENPIKVYNFEVQDFHTYFVGKSSVLVHNECTPEEAWKAIDEYRVKKDGLEPLGDSIPKRGDGKGTVAFVEVNGNKIFGINSSLLSKPEKELGKKMYEDMRKAGYFSTVKSYGSGHAQVFTHGESNALFRAYNIYGDNIGKNVTIYCDRGTCGICQTNLGYIREFLGLEKLTVINKNGKKFEF

SEQ ID NO: 15 - protein, 399 aa, Burkholderia cepacia
  REGION 1..399: note = MISC_FEATURE - BcDa02
MSSDHPGGYVEHNRLKMFEDKRFEYDVYGRLVRKLSGHGPAKELVLEYDDWNQLKAVVRKDRLGIGTTHFEYDAFGRRIRKFNGSYASTDFRWGGMRLVQETYHDRQGEEALTYLYEANSYVPLARIDQGKPAANDADARDAVYYFHNDVSGLPEELTDAGGELVWQARYKVWGNVVQEEWIAPVRHQPALAWGEVRAVIESPDHVPRPQNLRFQGQYLDRETGLHYNTFRFYDPDIGRFISPDPIGLNGGRNLYRYTPNPLVWIDPWGWACGELSAKAREVHSLARDDFALRNSTVAIVEAKVGDKTELFASASGGKVTPAQREALERLGVPKENIFWGAKAVNGYERIENHAERIILRNLPDGSEVTRWGISWGGLQRNASCIYCQPHVDEAGGIFD

SEQ ID NO: 16 - protein, 177 aa, unidentified
  REGION 1..177: note = Isolated from environmental sources.Organism unknown.
  REGION 1..177: note = MISC_FEATURE - MGYPDa01
YFDGETGLHYNRFRYYDPVVGRFVHQDPIGFGGGNNFYIYGPNSGSWYDPFGLAKRPPHKLKAKTKDENGNVKSERDYVSGGMTEEDKKLGYPLCSLVTHTERKALKEDDYSPTDTIEMHGEYAPCSHCKGAMNTAVDSGRVARILYYWKGKIWEAGAAARKLRNKKKRSAGMCSND

SEQ ID NO: 17 - protein, 410 aa, Pseudomonas fluorescens
  REGION 1..410: note = MISC_FEATURE - PfDa01
MSAAARLDDPIEHTGSLTGLLAGLAVGAIGAALIVGTGGLAAVAIVGAAAATGAGIGQLVGSLSFCSHEAGQIVSGSGNVHTNGKPAARAHLDKAKCDEHGSAPQIVAQGSSTVHINGQPAARVGDCTVCDGKISSGSGNVFIGGGTDTTDPINPEVPEWLERAVLVVGLASAFVLASPVVVVAGLVGAVAGGLAGNWAGGELFGQGSDGQKLMAFGGALLGGGLGAKGGKWFDARYEIKVQGVGSNLANVKIVPKGSEKVASIAESEAALGRANHAKTELPSSKEYKVKTVSSNDKKTLSGWGDKKPEGYEKVSAEHVKAKSEEIGHEVKSHPYDRDYKGQYYSSHAEKQMSIISPDHPIGVSKPMCNDCQGYFSKLAQYKNAEQVVADPKAVRIFKPDGTVETILRSE

SEQ ID NO: 18 - protein, 615 aa, Pectobacterium polaris
  REGION 1..615: note = MISC_FEATURE - PpDa03
MAGTETHYGYDEDGNCVTVRNGEGEIRHFLHDGRGLLIRETAPDDILHYRYDAAGRLTEVTSATSHIQLAYDKRDRVLEEHNSGSVIRRHYQDASHTVTRSLLWEGEEDSAALTSSFCYSATGELRQVQLPDGAELMLAHDAAGRESIRHSNGGFTQQREYDAMGWLTREMSGQQQDGRLHASQTREYLYDGAGNLAGVRHNRETWGYRLDATGRVLSVLSGGAGRAVETDEAYRYTRNGLPQDTTRLTEWQAGRLTQHDDTHYQYDKAGRLIRKQVIQPGYRPQVWHYRWDSRNQLRVVDTPTGERWFYRYDPFGRRTGKRCEQTQDDVRYLWDGDQIAEVRHYRNGERVARRHWVHNGWELLVQQRQNTDGRWETDFVTSSQNGEPQALYRPDGTLCWQAPKSTLWGQRPGSTEDPADPGLAFAGQYRDTESGLCYNRFRYYDPTGGCYVSPDPISILGGESNYGYVQNPLGWVDPFGLASCEHLNKMAKEVHDLAGGLKTPDARAIRNSTVSIVEATIDGKKVLYAAGSGGRLNPRQVEALERLGVARGNIFKGARVTNDFTKLENHAERIILRNLPDGAVVNRWGISWGGLQRGIPCDHCAPFVRDAGGFF

SEQ ID NO: 19 - protein, 817 aa, Lucifera butyrica
  REGION 1..817: note = MISC_FEATURE - LbDa01
MELSRITALEIVHSLNEHGKIYIKGIAPDTLEAKEIEQVADKAKIEIHLPGQPQPFFAGVCIRIELCYQAGLKIVEIEGRSWTYELDIAKKSRSYQQLGISYQELLNEVLKDYPAKAFLITLPDSKKYDRFAVQYWETDWDFLRRIAAREGTVLVPEYCDSSHRPRFWLGLPNLSAKSLTQTAEQASRSELGNYLKVALNADDSVKEKDFITYQAKGHEILRLGDLVDYGGKKLTVVHIVSKVDKGVLTSAYMLAGSEKMALANPENRNIQGVSLPGKVLDSRGINSQVHLDIDARQEKSAALWFPYASDLGNFLYCMPHNGESVNLYFKDGYEANAILVTNARKNGGQCPKTADYNNRYFTNYERKELFLSPDTVSFTVDESAAEKIAIKLNDADGITIESTKPIVFGAKQGLRLEAAKNIDIEGMLGVYGFSGPASIILKDNKTNFYGGNVLVDGANKSPCPPVEQPAEPKKKSILGKIASVGEAAWETFKKEPLIDKVQTVLDIASCCPVIGSAASVVNGAISIGRGNYGEAALNFACAVPGAAWAKAGAKLAEGVKLGATAAKEASVVAKTAINAAEDAARIAARGAKIAKTAAEEAGRVVLGKGKTMALKALEKIPKPKIVWEESLVLCADGMGHGTPFRFPRLLIEAGEGSGKAAENMAKKYAKEIEQSRAAYKRAAEVGEQYEEKFGTKTFASNGKKTMSGWDGKTTIAPTECTRVEPREVMDYSEEIGHKLKNSGSNDHGIPGQFQACHAEKQLSLLTDSPIGVNRKMCTDCQGYFQKHAAYTGEEKFVTDPEMTRIFHPNGEVSKILW

SEQ ID NO: 20 - protein, 208 aa, unidentified
  REGION 1..208: note = Isolated from environmental sources.Organism unknown.
  REGION 1..208: note = MISC_FEATURE - MGYPDa10
PGSVFGAGAHTIARTTARDMAATRTAMRQGGKKAAQATEHTAATTEAQRVAESQAAHTRATRVKQELPKTGRNRPKVVSSDRNNDTISGWSKDRPPGFLDPNVEEVLQVTDEMGHPRTPYFRDQGVPGQYFASHAERQMALNAEWPHIGVSKAMCADCQNWFRSLAQYQRRDWYVTDPNGTWIFRTDGSVVTSSELQVPTGQPITGTH

SEQ ID NO: 21 - protein, 310 aa, Actinomyces viscosus
  REGION 1..310: note = MISC_FEATURE - AvDa01
MAGAISGGLTLASLAVRMVHDLVRDAISDIIGKIASKVTIGVLTAGLAAPWVVQSVISDVASWVTRLTKEITDVVTSARNLKNLLDKATTLLDDVGEKFAGMAAMALGYGSKNTDNAAKGAKHADDAADAAKGADGAVPPSKGGGDGGGTPPGKGDKDGGGGGDDPAKKSQEAQQRAAEKAKEVNTKKAYKSKTVSSDHVKTLSGWSARRPEGFQDPNIDNVLTKCDEIGHTPEPHDFKDNGVPGQYHASHAEKQLSLTAKEPYIGVSKAMCPDCQGYFSKLAQHEGRDWYVTDPNNSWIFHADGTVTKQ

SEQ ID NO: 22 - protein, 229 aa, unidentified
  REGION 1..229: note = MISC_FEATURE - PbDa01
  source 1..229: note = Peptococcaceae bacterium
YQNDPNGMPLRLLDENGIIKWEGHYSAFGLVDRVSVEAVGQPLRLQGQYFDDESGLCYNRHRYYDAVVGCFISSDPIGLDGGLNPYRFAPNVLGWIDPWGLSCAVLSARLARIARIVHNLSGNPRAIRQSTVAIARVRINGKYQLFAAGSGGRLNPAQRAELVRLGVPEGNIFHGRGVTNGFSQIENHAERIVLRNIPEGSSVVHWGISWGGLQRNLPCVSCRPYVLGT

SEQ ID NO: 23 - protein, 1447 aa, Pectobacterium wasabiae
  REGION 1..1447: note = MISC_FEATURE - PwDa01
MREEILSRIARVGAMHAGNRPKPPPDLPEPGQGKPPTSPGKAIKHSSFWGALAGAAVGALIAAAAFTVAAAAVAGAVALTGITGGAGLALVVGVVKLAVGFGAVFALGDLIGGVTNRVSAMVDSASPSSGAVKDGSKTVFVEGNPVSRAEIDAVLCDKHSGPQLIAQGSATVFVEGYYAARVGDKTVCGATIKEGASTVFFGSGQESPLSVADEFSGWEKALILAVEFLMPPSRGLFRGLGKLFTKGPMAVLRGMKAGAAHALEGLRSAVQCAKNGFKESKGLARVTESIRGFLKDPVYIASGEVIEKRLDLELGQTVPLLFERTYRSGSPHSGLLGHGWHDSWSEVATVGRTEQGDIQVTLTLAQGYDIDFTFVVGATVVYCAEYPEFKLVKRHNGFHLWHRDSQTWRAFTAKQGDQLLLSAITDNHRNRIDFLRDPKGYLRKIRHSDGIELLLVWQGEFLRQVQRIDGGQKTVLAEYRQDEQGRLVEADAAHAYHLFYDYDSHHRLIRWHDNDQTWARYEYDPQGRCVYTTCADGYLTANFEYLADRVVMIDGLGQRHEYGFNDLYLMAWEKSPLGHITHYEYDDVGNLLREISPAGRVVEFAYLDNSGLVSTFTDGSGHQWHYDYDHYERLIGITDPLGRSWAWQYDSKGNPLSLTGPDASEVRFAWNRYGLLTEVSDEQGRTQASLFYDHRHRLMSATDAESRTRQLRYDQQDRLTSWTRPDGATYRLGYRRASWKLPEQLLRPDDKQEQRQYDKHNNLLNYTDGNGAVWAQTYGPFDLLTSRTDAEGRTWQYEYDKESQQLVCVTAPDGNRWQWWLDADARVIRERDMAGTETHYGYDEDGHCIAVRNGEGETRHFLYDGRGLLIKETAPDDTLHYRYDAAGRLIEVTSSTSHIQLEYDERDRVVQERNSGTAIRRHYQDTSHTVTRSLHWEGEEDSAALTSTFRYSATGELRQVQLPDGAELTLAHDSAGRESSRQSQAGFMQHREYDAMGWLTREMSGQAVDGRLQPTQTREYLYDGAGNLTGTRRNREAAGYQLDASGRVLSVLSGGAGRSVNTEEQYRYTRSGLPQDATRLTEWQAGRLTQQDNTHYQYDKAGRLIRKQVVQPGYRPQVWHYRWDSRNQLRVVDTPAGERWFYRYDPFGRRIGKRCDQTHEDIRYLWDGDQIAEVRHYRDDELVSRRHWIHNGWELLVQQRQNADGRWETDFMTSSHNGEPQAAFNLAGELRWQAPRTNLWGQRYTDNTENLDPGLAFAGQYRDAESSLCYNRFRYYDPNGGYYISPDPVSIFGGDVNYGYAHNPLGWVDPFGLASCEHLNRMAKEVHDLAGGLKTPDTRAIRNSTVSIVEATVDGKKVLYAAGSGGRLNPRQVETLERLGVSRENIFKGARVTNEFSKLENHAERIILRNLPEGAVVNRWGISWGGLQRGIPCDHCAPFVRDAGGFF

SEQ ID NO: 24 - protein, 555 aa, Bacillus aerophilus
  REGION 1..555: note = MISC_FEATURE - BaDa01
MDARAKHYQELREQMVDLKKALQGVANLGDDFTGKGADNIKSFYKELSGNVDMFISFIDKQIAFHDGVSGTLDDTSFGGDTFVEEHFLDNAVHMGIKNSKSIVKDQKKALKTIFEDIDDLISLEVFDSKTFDEKIADAEDERKKTVKDLIELDQNLKDEYALSETEEKATMALYAEMINATNDGKSISPMNFDKKAFQDSEIYKAKSDIEKQTSEYLKIKKDQEEAREIAKEQEALANRPWYEKALDYGGNIVNELTGVNDAKRAATGVDPITGEELTSGQRVAAGGMAAAGYIPIVGWAGRIFKGGKAIYKTSKATSAAVRAVDIYKTSQKSFDALKTSQKGLYGLTATNGFSEAITGRDMFGNKVSKEQQEASMNTALAILLPFGMKGPIKKTPGVIQKSNKQISESYDEIKRFREKAGLKPYAIDSGDTVAAVKVNDRTFFGVNSTITKESQQATRELRQKWLKEVQWVPPKKSSPKHLGHAQSLTHAESHSLIRAFEKEGQLPQKVTMFVDRKTCNICRGEMPALLKRLGVDELEVYSGGLEKPLVIKATK

SEQ ID NO: 25 - protein, 1478 aa, Pseudomonas panacis
  REGION 1..1478: note = MISC_FEATURE - PpDa04
MSDALWAARMGDALTHTSMMADILGGVLEVAANVAITALATAAVAAAIGVTVATAGIGGCILGAVVGIVVGMAMSKTGADKGLSKMCESFANALFPPVVEATIAVGSADTFVNSIPAARAAGSIPSHVAPAGTELELPPPEPDTAPQAEPGFLDMAEGFFSQLWRPTVATPAPGVVPALTDMVLCARHPPMPPQLLAEGSDKVTINGQPAVRSGDRSMCDATVVSSGLISPNVTIGGGTVVVQEIRSGKTPGVGLAIEVLMMLKGAKGKTLSDLPCMLLSGVTSYAVSQAMGAAANATMGSPNPVHAATGAKVLGGNTEMDFVLPGILPITWQRFYNSRDKRRNGMFGAGWSVSYEVCIEILPHPDGGEDLVYTDEQARRIHLGAMPLGTAMFSAGEGLSIRRHRNGQVLIESEDGLYRLFDSAPGDSAFLRLSQLGDRNDNRIYLDYDERGQLVRLRDTFDLIQIELTRERNRVCQIVRVYPDHRREPLALYSFDAAGDLAHVSDATGQTQRRFAYDSSQRMVEHQLPTGLRCFYEWALIDQMEWRVIRHWTDEGDEYRFEYDLQAGITRITDSLQRVSTRLWNSQHQIVSYTDNLGQTWLFEWNDERQLLSATDPKGGRYAFSYDDSGNLIGEHDPLGRSDSTVWLPHWALPRFQIDAAGNSWTYQYDSRGNCIRETDPLGYSTQYRYDAHGQIVEIIDAAGKSKRMRWNQFGQLIEYIDCSGYPTRFSYDERGFLQTITDALGERSHFSVDAKGRLLTSQSPDGQMDYFQRNANGQLVSHTNAAGHTTHYRRNRRGQVSQRTDAHGRTVEFGYDAFGRLQALTNENGERYLFTWDAGDRLEKQQDLDGSAKYYAYDALDNLASVESVPALYGTGLSVVPPTRAKPIVHRLERDAIGRLTAKVTDDGRTEYVYDVLNQLLAVTFTDHQGNSQALGFSYDALGQLLEEASPAGSLRHHYDELGNLTQTQLPDGRWLNRLFYGSGHLHQINLDGVIISDFERDRLHREVMRTQGQISTYLGYDRSGRLRSRQRRYTQQASLLPSAEQSNFEYDLANNLVVKRDQHAKGQRHQLLHYDATGRIIASQGDIQGQAEIFAYDAAANLLDGSQQGAGLVVHNKLITYQDKRFRYDGFGRVIEKRSSQRGLQSFRYDAEHRLIEVRNHGTAGDSVICMKYDPLGRRQEKCEWRNGFQTNKTTFVWDGLKLLEEQHNSQKSLYLYATSGYEPLARVDGRGHQQKIRYYHNDVNGSPLQLTEADGSPVWEATYKVWGNTLQEVQEPYYIEGQNLRYQGQYLDRETGLHYNTFRFYDPDVGRFTTRDPIGLAGGLNLYQYAPNPIGWIDPLGLSCSLSAKARKIHDVARDPFAKRNSTVAIAEVTINGKTTHYVAGSGGTLSPAQRKLAAEMGIPPENILSGTKVRSSNADPTQAKLENHAERVIIRNLPEGAKVEKWGVSWGGKQTNASCSNCQPHVDASGGIFD

SEQ ID NO: 26 - protein, 2582 aa, Streptomyces sp.
  REGION 1..2582: note = MISC_FEATURE - SaDa02
  source 1..2582: note = Streptomyces sp. A0958
MAVIVSLASALTWGGLSLEAVAVPPKPREAMAVELPDLPESTRTEQDESAEKHLTTAPEQATTPYAPQAVEQWSPGVGTADLTDVEPGRSVPVENVPTVSLGVPEEGDPAALAGEWTVDLKAPEDSQAAEVDGLLMEITPPATADPDAEVTVGVDYTSFADLYGPQAADRFGVVLLPNCVVDAPTEGECAPEPAAEEAADGATAQVQPLASEIELVHPKGTAKLRAAAEKEGETLRTRRVVSATVPVSELLGPDSGSKASGAMSAAGSAETGSRAVGVLDTGASAAGDFTATPLQSSGSWAAGSSSGAFTYGYQVQVPEAAGGLTPQVALSYSSQSVDGRTSATNNQASWIGDGWDYNAGSITRTYASCREDAKKAGSNNASHKTGDLCWGSDNATLTLGGATTELVWDADQKEWFTANGDGSRIQIIKDTSKDNTDADGEYWIVTTRDGTRYHFGLNHLPGWSKGDPVTHSVLTVPVFGNHAGEPCYKAGDWKGSDCEQAWRWNLDYVEDVHGNAMSLWWKKETNHYARNFNWKAPVSYDRDGYLLHIDYGQRKNTIFSAEAPGRVAFEVAPRCYAEGSLACNEANFTSKDPGKYRIWYDTPADLRCASGKMCWNAAPSFWSTKRLDRIETSAQRRTDTTARQAVDRYQLKQSFPSLRTGPNTALWLETITRTGFARNGDTDASEQLNPVRFEANADDMPNRVMRGDKDPRPGFSRLRIGRVINEYGGETVVTYKQPEGQCATGQGLPGKSDKAALKGNTRLCYPSFWHPDPEKEDIDWFHKYVVQEIEELPNVDGAYSTSTKYTYGTAGWKLAQQEFTKKSTRTYSQFAGFDRTTVLTGADDKTIGSKRTKAVTRFFRGMGDSVPVKDITGTHIAYDREPFAGRIAEELTYAEATDADTAWLTRSVTVPEATELASRTRGDGLDPLKAWRVTEPRQLAYSKDGKSTVHTSETKTGYDAEYGLPVRIETLKDKADPGLVGDVSCTELEYVHRTDKNIIGLTKQTLSSATPCATADFTDLKKLASGSRTAYDSGEYGAALTGSTRGLATQTWSLKADGSGFQSDGTVGFDSLGRVVKATDPDGRSSTTEYTMVHGQTFGVTETNSKGHASTQEVEPGRGTTTKSTDANGHATQTEFDPLGRIVQAWAPGRARSSAAVPDFAAEYHISKNDPEESDRFPPYVVTKSRGHRDRVETSVTIYDGLGRERQAQEEATGGGRLITDTLYNSSGEVWQTNNAYLADGKPNGRLFTPLADTAVPNATRYTYDGLGRVVKELPILGGSEMAARSTRYEYGADWSTVINPSGAASYRVRTDSMGRTTQVDTFTDAERTQYTSVKYEFDALGQLVKAYSAQNPKRSWSWKYDGRGRMVSATDPDSGTSTTAYDHRDRPVTTTDARGVTVWTEYDELSRPTKQRLDGPTGDLLAWSTYDTAAGGKGLPAASGRVTDGQEYTMSVDGYTADYQPRSTTLSLPESVATTWGLRKSYTSTYHYSDTGLLLDGTIPAAGAFDSEKLVVRYNEDGLPLSVSGKDWYGSEAQYSPYGQLLRSTLGAQPHRVWALAGFDDASGALTDQQVYREQNGDTSVVGGKLASHRSYSYDNAGNVTGIRERSTGIQERQCFTYDPIGQLTEAWTSADLDHCADGPVKEGGALNVTAGPDDSGYWQSYEYDLLGNRTKLTEKDLTGATAKDAVTTYTYGKADGSQPHTLTKVTKKYTTPAGAQVTAEAERLYELTGETKQVTSLENGDTQDLTWTWDGQVERLTGQGSGGKTSYVGPGGKCLDLNGGKPEENRPVNLYSCNGAASQKWSFQVAPGQSDPDVGTMTFHGGTWCVAPGSPSAGSDLRLHKCDGSADQKIKRNASGQLMHVGTGLCVAVEGGATADSTSTVLTTCSATSAAQKWEAQNETRYIYGPDGGRLLTVQGKQATLHLAETEVTTQAAGKLVSTQRSYGAPGGSVLRHQYGYQKASTLAAEVGDHQGSTYAEVAMTDGMPVRVRKQDPFGNERGAAASGTGPRTSAAFLGVSPDDASGYTQLGARMYDPAVGRFLSADPVLDIADPMQANGYTYAHNNPVTLSDPTGLAVSLTASETAAALAGAGLSLAQIAQAKSDSGRSLSSVVLSVAWSTLKDFIGFNDAMACFGGDMWSCGSLIIGAIPWTKLGKIPSVLKAVKRTINAIQAWRSAKKKAEAVLAAAKAAQHKALAAKKAAIEKAKKAAQAAKKKAAEKKQTTSNKAVNQTKKTGSPVQKQAQAKAAPKVSSASATHKSSGGGAKADTTKSGGSSGGSSRSKGGSSGDGGAGKAGESSGSCSVANSFVPGTKVVMADGSTKAIEDVKVGDKVLATDPETGRKTVETVTAEIKGEGLKHLVKVTVDTDGDRGDETASVTATDGHPFWVEALDAWIDATDLKAGEWLRTSAGTYVQITAVERWTAAGETVHNLTVRDTHTYYVLAGATPVLVHNTGGNGHMCDISVTSSDGSKSDISLESGDMTPEEASLGYPNNSAFTHTEHRFSRMAGASTGPKVSLPNDPFAGKFPLSAGDSVKMQGQLPPCSRCKGAMNRMVNELGVSVTYEWSGAKGAGSWTAGRRRR

SEQ ID NO: 27 - protein, 448 aa, Neisseria subflava
  REGION 1..448: note = MISC_FEATURE - NsDa01
  source 1..448: note = Neisseria subflava NJ9703
MAGGAVNRRYAYDKAGNLIQSADQRSGVLNYVYDKIGRIQEARNSQSGRSETFAFDPAHNILSDKAAEGKGNNLVSGNRLKEYNGIEYTYDALGNLIYRQLPNGENQYYQYDLENQLVRAEIKKPAGNTEIWTYAYDPFGRRLSKERQDKLAWTSTDPKRTHFVWDGTRLLQEYTYKGSYTYIYTDQDSYEPLAQIFDNAKDSKQYLAYFHNDQISIPREMTDIHGNLLWYGEYTAWGRLKKDERVYKNAHQPFRLQNQYFDEETGLHYNLMRYYEPEAGRFVNQDPIGLLGGINLYQFSPNVQTWIDPLGLNAKLSEMAQSVHNLAGGLKTPDARAIEKSTVAIFEVELKNGSKTLYAAGSSGRLNPRQRSLLEKLGVPKENILSGKKYTLPKDVVKNINHAEQIILRNIPDDAKVTRKGISWGSKQRNAPCSRCKPSVDGAGGVYD

SEQ ID NO: 28 - protein, 1613 aa, Escherichia coli
  REGION 1..1613: note = MISC_FEATURE - EcDa01
MSEGPGGPQGATAGGTLAMRMLSQQAMVASQMKRAANDKAIAQMLASKKSGPPAARLGDEIQHKSFLGALAGAVLGAIVTIAEGCLIMAACATGPYALVLVPALMYASYKASDYVEEKQNQLESWINSFCDTDGAINTGSENVKINGELAARAAVTLPPPPPPGAIPEVPQGEPSWGDIATDLLESAAEKAVPLAKAWGNAVITLTDSNAGFMDRVSAGASLLFPAGPVLMEFATMVGGRGEIKKEVDFPEAGEDTALCDKENKPPRIAQGSSNVFINNQPAARKGDKLECSAVIVGGSPDVFIGGEQVTYLDIQPEFPPWQRMILGGITIASYLLPPAGLLGKLKNLARLGKLGNLLGKSGKLLGAKLGALLGKTGKSLKSIANKVIRWVTDPVDPVTGAYCDERTDFTLGQTLPLSFTRFHSSVLPLHGLTGVGWSDSWSEYAWVREQGNRVDIISQGATLRFAFDGDSDTTVNPYHAQYILRRRDDYLELFDRDALSSRFFYDAFPGMRLRHPVTDDTSDDRLAHSPNDRMYMLGGMSDTASNRITFERDSQYRITGVSHTDGIRLKLTYHASGYLKAIHRTDNGIQTLATYEQDARGRLTEADARLDYHLFYEYDAADRIIRWSDNDQTWSRFTYDEQGRCVNVTGAEGYYNATLDYGDGCTTVTDGKGTHRYYYDPDGNILREEAPDGSTTTYEWDEFHHLLARHSPAGRVEKFEYNAALGQLSRYTAADGAEWLYRYDERGLLSNITDPAGQTWTQQCDERGLPVSLVSPQGEETRLAYTPQGLLSGIFRQDERRLGIEYDHHNRPETLTDVMGREHHTEYSGHDLPVKMRGPGGQSVRLQWQQHHKLSGIERAGTGAEGFRYDRHGNLLAYTDGNGVVWTMEYGPFDLPVARTDGEGHRWQYRYDKDTLQLTEVINPQGESYRYILDNCGRVTEERDWGGVVWRYRYDADGLCTARVNGLEETILYSRDAAGRLAEVITPEGKTQYAYDKSGRLTGIFSPDGTSQRTGYDERGRVNVTTQGRRAIEYHYPDEHTVIRCILPPEDERDRHPDESLLKTTYRYNAAGELTEVILPGDETLTFSRDEAGREVFRHSNRGFACEQGWNAASQLVTQRAGFFPEETTWGGLLPSLVREYRYDSAGNVSAVTSREDYGRETRREYRLDRNGQVTAVTASGTGLGYGEGDESYGYDSCGYLKAQSAGRHRISEETDQYAGGHRLKQAGNTQYDYDAAGRMVSRTKHRDGYRPETERFRWDSRDQLTGYCSAQGEQWEYRHDASGRRTEKRCDRKKIRFTYLWDGDSIAEIREYRDDKLYSVRHLVFNGFELISQQFSRVRQPHPSVAPQWVTRTNHAVSDPTGRPLMLFNSEGKTVWRPGQTSLWGLALSLPADTDYPDPRGELDPEADPGLLYAGQWQDAESGLCYNRFRYYEPESGMYLVSDPLGLQGGEQTYRYVHNPNESIDPLGLAPCPWQIIDRFRSLKAGLSRLGEHIPHQGDGFGTVAYIEINGRKIFGVNSSVLSDKAKDLGRYWVDRLKLGRGVSQVAYHAEAHSLMRAFEKFRGKLPNEITMYVDRYTCPNCQKYLPLLMKEMGIDKLIIYSKNGKVVLLPK

SEQ ID NO: 29 - protein, 151 aa, Hormoscilla sp.
  REGION 1..151: note = MISC_FEATURE - HgDa01
  source 1..151: note = Hormoscilla sp. GUM202
MDEINSEQVAAIISAQSARANLGSPPRNKTIAGVEGGPRTLSGWKQEPGYLQTTERVMSYCEEIGYQLRPAGPLDKGVPGRYNASHAEKQLSVAEPHKPIGVSSEMCLDCQEYFQALASYTGRYQIVADPKVTRIFHPDGAVSVLQTELSI

SEQ ID NO: 30 - protein, 319 aa, Aquitalea magnusonii
  REGION 1..319: note = MISC_FEATURE - AmDa01
MLNYPSSDSTEYRYDPLGRRIAKLSQGKTTLYGWDGDVLAFETQDDAAVHYLFEPGSFIPLAQVHTDAIQGVKVPAWNQYNPYDPERDPLLQAGPEPSAPRAVYYYHADHLGTPQALTDAQGQLALEMDYQAWGEAREVIAEAAASADIRNPFRFQGQYHDDESGLHYNRYRYYDPEIGRFISRDPIGLMGGINIHSYAPNPIEWVDPFGLERFGSGKGKHTATVEVINKNGNVKSCQTLQSGSMTPEEKELGFPRSTLATHTENRAMRQVDIASGDVVNISGEYPPCTSCKGAMNKKAKEVGCVVNYSWPEGSWSTKK

SEQ ID NO: 31 - protein, 275 aa, unidentified
  REGION 1..275: note = Isolated from environmental sources.Organism unknown.
  REGION 1..275: note = MISC_FEATURE - MGYPDa408
RAAGQPKLCEVYYFHTDQVGLPEELTNAQGQRIWQASYKTWGNTVREAWAYANIDGSPIFTQDQGQMPDPQAREQNLRFQGQYLDRDTGLHYNTFRYYDPDIGRFISPDPIGLNGGINLSSYAPNPVGWSDALGLAGNWSIGGQPITAPVLPPYDGATTHGVFVYLNSNGSIQKIPLQSGGGTAYPNYANAGHVEGKSALFMREHGIQEGIVFHNNIEGTCGFCVNMTETLLPEGAKLTVVPPEGANPVKRGATGEVKTFTGNSSDPKQPTKKGC

SEQ ID NO: 32 - protein, 2330 aa, Streptomyces zinciresistens
  REGION 1..2330: note = MISC_FEATURE - SzDa01
  source 1..2330: note = Streptomyces zinciresistens K42
MYVDTTLPKIRYLAHVLVTLTIVVMVALMVSLIPGVNVAQATEQKSDPPGLRNEKSIPGEVFTPKQRERPNGADRDWKASEASWPAGKRTAPIAAAPAGGRSASVGTSPVSVYQASGARTASGADAGLVDIEVLDRKAAEKAGVNGVLFTVTPTVPATAGRAGSATAVAPSGKARVTFDYAKFEGNFGANWGSRLELIDTATGKSVTRSNDSSARQLTGEINLPEARAGKAEPLALAAVAQAAGSGGDYSATSLSPSGSWMGGSSSGGFSWTYPVDAPAVPGGPEPEVALGYSSQAIDGRTASTNAQSSWIAEGWDYEPGFIERRYRSCADDQGTGANNPGQAGRTGDLCFGSDQMVMSLDGSSTELVKDDTSGTWRPADDDGSKLERKTGADNGARGGEYWVLTTTDGTQYHFGLNKLPGAAAQRTDSTLTVPVAGNQAGEPCYASAFADSFCDQGWRWMLDYVVDPRGDAMSLWWAKESNYYGGNNKPAGMKYDRGAYLKNIDYGQRADKLFTQSAAARIAFTVAERCIPGSGFTCAEADRTPANAKYWPDTPIDQQCNAGEQCKDRNSPTFWSTKRLTKITTQVNNGGLRDVDSFALDQSFPATGDGTSPALWLKSVERTGYGQDGTTATLPKVTFRGEQMDNRVDGLEGLEPFSRYRVNAVDTENGGTVGVTYSPQECRRGGTMPAGPDTNTLRCYPQNWTPKGALEPITDYFHKYLVTQVREEDNVTDAVARVTSYEYLGGAAWAYDDSENTEARRRTWSQYRGYERVRTRLGSGTDVRQLTENRYFRGMHGDKLPSGTRSVEIEDSEGGKITDYPQYQGQLREEITYASDGGAIAAATKLTPAAVKTAERERAGTTPLQAYMVSPVTSETREHVKDSTWRRSSETTTYDSDGLPTQIEETTGSGRKACTRIEYARNTAQHILDTESREVTTTGACGAPGGKVVDDTRSLYDGGAYKAAPTQGLVTEVQELNAAGDGYITVDRTEYDIHGREIRTFDVEGNETRTAFTPATGAVPTKQVDTDPLGQTETTEFDHRGLPTAELDANGKRVDMRYDPLGRLLKVWDIDRNQATQSASAEYDYTIRSDGPTVVTSKTLKGDGSYATSYEILDGLLRTRQTQDAAVGGEGRIVGDTFYDSAGRAYKSNDGYFNEQNAAPSLLSVGDNAVPSQTRIDFNGLGEETAEVTYSYGDEKLRSTTVKDGDAVTVIPPKGETVTTTYQDADGRDTKLREYTEADRSAWQDTVYSYDDLDQLTKVTEPGGGEYTFTYDARGRQTSATDPDGGTTRFEYDRGDNVTATVNALGKKLVTTFDKAGRATSLREDSADGPKRAEWTYDTLGKGLPVASVRYDKDGNAYRDEVTEYDANYRPRTTKTTIPAAAGKVAGEYTYTYSYTSTGNLAWVELPKVGPMAKERLVFRYNRDDLPISLSGAANYVTDITYSKYGEILRSEAGPAGRKLFSTYFYDDFTRRTDRVVHDRSVQPGRIDDTTYAYDEAGNVTAISRTPGEGMPDAQYGTDTQCFAYDQLRRMTSAWTAGEKCAPDGPSKETVGGPEPYWQSYEFDASGNRTKLVEHDPAGNVAEDITRTYSYGGDAGGPNRLREITSAGADGTTKTTFGYDKAGNTTTRQNGGTTQSLSWDIEGNLESVTEPRAGGGEKKTSYFYDASGERLLRTGSDGATTLYLGETELTVRADGTESVERSYAHPDGATTVRSSDGGREVLLADQQGSTQTSVDMTEAGMPVTRRLLTPFGEDRGAAPSTWPGQRGFVGGTRDADTGLTQLGARPYDPATGRFLSVDPMVDYQQPATINPYAYANNAPATFSDPDGLFFPVLVGIAARIAIQAAIRAAARRAAQIAARKAAEALRKRILAEARKRAIAAARKKAVAKAKREAARKAAAAKRAAAQRAAQRAAAKRAVARQAAARAKQRAQRAAAQRAQAARAAARKARPKPRPKPKSQPRPRTKPSQKAKPAQQRKASPKQSVGSRAREEIRSEARGQAEDAVAEQASCRTNSFVPGTLVVMADGTRKRIEDVKLGDKVLATDPKSGRTTAQPVVSTILGDGTKNLVKITIRTGEASPENIRDAARPGGETDGGVPVDTGLLQAQPGDGDGNGATTGTGTGTVVATDGHPFWVPALGKWVDAGELEPGQWLQTSSGSWLQITAVQAWTQNATVRNLTVADTNTYYALAGTTPVLVHNCGGAQSLADRASEIHAQAGSEIAMSKSTVAVVSAQTPKGVVNVVAGSGRGLNRAQKDMLSGVEFPARNIPGAHAEQNALLFINEMGWSPIAGGASRSVCSEVCAPLIRAGGGRITGRVYQNESGTKIRTFEW

SEQ ID NO: 33 - protein, 281 aa, Ruminococcus sp.
  REGION 1..281: note = MISC_FEATURE - RaDa01
  source 1..281: note = Ruminococcus sp. AF17-6
MNKGFNSAVNKAGVKPSCFVAGTLVMAVAGMVAIEKIKSGDKVISTDPETFETAEKRVLETYIREDSKLIHLVINGEEIITTETPPFYVKNQGFIKAGELIVGDELLDVNGNVLLVENFDVELTDEPTTVYNFQVEDFHTYHVGKCRLLVHNANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 34 - protein, 159 aa, unidentified
  REGION 1..159: note = Isolated from environmental sources.Organism unknown.
  REGION 1..159: note = MISC_FEATURE - MGYPDa624
DPLGLEHGNMPRKGAGVGNGGSKYTLNKDFQGKFTLKRPELPVYDGKTTSGVLVTDDFKQIRFNSGNGDPRYTNYANNGHVEQKASLYMQDNNISKATLYHNNTNGTCGWCNNMTETFLPEGATLKVIPPSNAVANNSKAVAIPKVYTGNSNVPKVRKK

SEQ ID NO: 35 - protein, 1399 aa, Enterobacter chengduensis
  REGION 1..1399: note = MISC_FEATURE - EcDa04
MFEAARVGDDIGHSGALAGMIAGTIVGGLIAAAGGILAGAMFIAGLGASCLGVGVLLVGASLAVGYYTGELATAARDGIADAGASSMTKKGVITSGSPNVFINGKPAAMATDSAVKCSDDGSQQMAEGSSRVSINGLPAARIGDRTTCDAKVMTGSDNVIIGGDPQQTLPIQPEVPEWLYKVSDLTLLFAGLIGGWGGAAGKVGALSKLLGKIPGINKLARIACRAGTLMTGVAAAGIIARPVDIVSGQKFLSGDDELDFVLPSRLPVRWQRYWRSGNPGDSVLGRGWSLFWESRLEPYQDGLAWRAPSGDYVAFPKVPKGMRTYCESEKNWLEHHSDGSWSVYDVSGERWHYAPLKDDAPSLLQRISEPCGNEILFEWNADNTLHALTDSAGQRVVCRYDRDRLVGAWLDDEICLVSYAYDEQRQLVCVTGRGGSVRRRFRWQDGLMSAHEDANGLLSEYRWQEIDGLPRVVGFRHSGGERLTFEYDFDNGTRRATREDGAQAHWLVDDDDNVARFTDYDGRQTTFVYRDGELCDVILPGGAMRRSAWDKYGRMTSETDPAGRRTEYHWYRLTDRITRTVYPDGTSSQAVYDLRGRLLSETDPAGNATAYRYPDDLENLPESITDAIGGVVRLVWNHQGLLTQRTDCSGSVTRFTYDRFGQLIASEDAEGNITRREWGRAGLLSAVIHPDGSRESLVWNERGQLTGWRDPLESEVSWAYNALGLPVSLTDRIGRVRQWRYDPRGNLLRLDNGNGAEYRFTYDAVGRPLSETRPDETVRHMEWDARGFLCALEENGRPAADGGIARRVQQFSYDDSGLLIGRTQRHAEYRYFRDLSGQIARIRRTPTAEGVALGIESDEIAYRHDAAGRVLSESGINGEVCYEWDALSNLTGLTLPGEQKLAWLHYGSGHVSAIRFGQQLVTEFTRDRLHREVRRTQGAREQFRQYDSLGRRTLQRSELNTDVALPEQTLLERLYRYTARGELSGVSDTLRGEVDYGYDAEGRLLKHYEARQGYSRAQFSYDAADNLAASDDPAPVTDNRLQHWQALFMKYDHWGNLVSRRNGLYEQHYAYDAENRLVSARGTGPEGRFKASYHYDALGRRTRKTVTTTHGTTETRFLWQGYRLLQEQQESRLCSTYIYDPNEAWGPLARVDHLHDQSSGEIYWFNTDLNGAPLEVTDERGAVRWSGQYGSFGEVRHQSEGFSRVVNRSAMARQPLRYAGQYADGETGLHYNLFRYYDPQVGRFIVQDPIGLNGGWNLYQYAPNPLGWIDPLGLSFGTGKGTHTAHATLFDSEGNVKAQGIWQSGNMTPEEKALGFPQSSLATHTEARITRELDQIASPGDKLVIDGQYPPCTSCRGKMNTFKLNTGADVVYNWPSDTGQINEWQASGKKSARRKNCSG

SEQ ID NO: 36 - protein, 310 aa, Burkholderia lata
  REGION 1..310: note = MISC_FEATURE - BlDa01
  source 1..310: note = Burkholderia lata (strain ATCC 17760 / DSM 23089 / LMG 22485 / NCIMB 9086 / R18194 / 383)
MWNGILLVQETYHDRWGEEALTYLYESNSYVPLARIDQGKAAANDANARDAVYYFHNDVSGLPEELTNAEGELIWQARYKVWGNAVQEEWIARAPQQPVPEWGELQVATATSVHMPRPQNLRFQGQYLDRETGLHYNTFRFYDPDIGRFINPDPIGLLGGTNLYRYAANPLVWIDPWGWACGELSGKAQEVHNLAGGGDARSIRNSTVSIVEAKVNGKPQLFAAGSGGRLSPAQRQALMKMGVPEENIFYGKKSIDGFDKLENHAERIILRSLPEGAEVSRWGISWGGLQRNASCPSCQPHVDQAGGIFD

SEQ ID NO: 37 - protein, 261 aa, unidentified
  REGION 1..261: note = Isolated from environmental sources.Organism unknown.
  REGION 1..261: note = MISC_FEATURE - d16_MGYPDa17
ITSQYYLRARFYNPVIGRFLNEDTYYGDGLNLYTYCHNNPVEYVDPSGNWCKKKFKQKVKEYLEKGYSKKDAKVMANYEQIRENYDVNSAEQYLRAVLDYENLSPEAWKSIDQFRESHGLNTLGNGLPKKGDGTVAFINANGEKIFGINSSLLSEDKKMLGKKYYESMKEAGYFDNVTSYGNGSGQVFTHAEGNSLMNVYDLYGKSIGKDITIFCDRTTCGICKNNLGYFKDYFGIDSLTVLNKNGDIVIINKGTYIKIKS

SEQ ID NO: 38 - protein, 215 aa, unidentified
  REGION 1..215: note = Isolated from environmental sources.Organism unknown.
  REGION 1..215: note = MISC_FEATURE - CgmDa01
EDYYRFLPEFKKVPITAREPEKPAEKPTLAPTEQSGYTEKGFTGRVQDKITELRMWDSLDSFRETRGLPTTGTEPTKPGDGKGTTAVIMVNGEPVYGLNSGFRNAEEQQFIRECYEELIGSVFEQKENTHYGKGQSYFLTHAEGYALLQVWKNNGDLSGQDITIYCDRDTCGYCRGGLKELGKTLGMNSITIISKSGEAIYYEFGVGMTRWQPLS

SEQ ID NO: 39 - protein, 2563 aa, unidentified
  REGION 1..2563: note = MISC_FEATURE - NoDa01
  source 1..2563: note = Nostocales sp.
MRYHHVRVTDPGVTGGTVPGVALYAKKITYTGQGKIEGRYSITFTRDRDRGEPRRPDVVIDARNGFKQVTADLLRRVDILLDDKPIRAYELNYRTGAFAKTLLQSVTQFGEDGSPFTTHTFDYYDDIRSADGQYQAFGPAAGWSVPGDSLGESVPDGHASALSATTSRSTGGHLYVGFNPAVVSKSGSAGAKVGFNAGTSEGLLSLTDVNGDSLPDKVFRTGAGVFYRPSLSGPGGAPKFGDTPIRLPGLPGISEESTRSTTSGAEAYFGVAAQLDHVSTTTRTDRYLTDVNGDGIADLVTGGSVLFGHLDAQGNPVYSANSNDTPVPIGGGTVNGTIVGDQTAEIERQVDAFPLLDSVRRWVAPYDGTVRIAGGAKLTSDPGDDRAADGVRVAVQQEDTELWAQRITARDHNTYVPSGVDSVAVHRGDRLYFRVQSVADGRFDQVNWDPDVTYTGVTPATDVNSLDVYRYLASRDFTLGGRPSTVTAPVTGTLHLSGDVSKVGPTTDDVAVVITRNGTDVFSRTLPAASATTVPIDLDIPVSAGDTLSWRLKVDSPIDAGTLHWVPSAHYTAAEGGVPVVDQQGNPTLVIDPPFDLDLYPVDTATAPQGTYTADRTGDLVVEPNLALAGDAHVVFTVKRRGALLAKRVIDVAGGQVPAPESLRVTVPVTAGDELFFDFSTLDPGLPAKVTGQSASVSTDGTTFTPVPSALHAAAEQGAFAAPYRGWAAIGYQGNRDRATAPIKQSDLVIDASYRDKIPGAPKESDVPGFDGRVSKPNAVVLAPRPALERWGSDDGNTWVGAGSAASSRWGTDRIDVAQDADIAGATGVSRVARIQQISTTFGASIPGVPIGAGASVARGRSSGQVDFLDLNGDRFPDVVGPAGIQYSDVTGGLGATRGSLGTGSVRESDSTAFTASANAGSPARTEPNGRGMSAPAADKPATTAQSGSELPSLGIGGSVGGGQSGAAFDLLDINGDTLPDRVYSDGTAALNLGYRFAAPEPWPGGPLADSNSRSSGVDLGFNTDFYGLAGGVSATSGQAKTEAGLQDVTGDGLPDRVFSRDGQPLSVAINTGSGFTAPTPLGGSQAGITTDTNAGLGAGAYFTFGFCLGILAGACVVFNPGADTSRGIGRSEVALRDVNGDGYADQVRSTRDDELVVAENRTGRTNLLRSVSRPLGARFELDYTRDGNTENLPQSRWVLSRTTLFDGHPGDGQDFQVSTFHYADGRFDRLERQFLGYGRVVSEQRDGDAVYRSTTSEYRTDSVYTRGLFTRTYTADGGGHLFTETLNSYVLRDVATGKVADPASTSATVFPLLAHTEQRFYEGGAAAGKTTATDNEYDQFGNLTRVLDTADTGTADDVETRMQYTAADAACRDRNIVGLPDVVDVVAAATKTTTRHRESTIDCATGEVRQVRAILADGSKAVTDMEYLGNGNLKAVVGPVNKAGQRYRLDYGYDPVVGVHIETIVDSYGYRTAITHNYKYGLPETTLDKNFQRVHTDYDRFGRVSFVAGPYEIPDNRVTVSFEYHPEAPVPYAVSRHIDTTAGGVRDDTIDTVQFTDGLGRVLQTKKDASVAPAPGVTPEAVMTVSGHTVYDFAGRAVEQYYPVTEPKGDKNYVFNPAVDSVAPTRLGYDILDRTTSTVIPDGSVSSTSFGFGADRSGTTRFETVSTDANGKQRRSYSDTRQLTTAVLEANPAGSQPVIWTSYDYDAMGQITSVVDDRGNTTKAAYDNFGRRTVVDSPDSGKVESGYDLADNIVSKVTPTLRAQRKAIQYDYEFTRLTAIRYPVFSGNNVTYTYGALGAPENGAGRITSIHDAAGTVTRGYGPLGELTRETRTLGKDVGTWEDRGQSFTTTTRYDTWNRVLETTYPDGEELTYGYDTGGQVTTATGVKNGNQYKYLARMDYDKFNQKVLQETGNGVRTTYAYNDTDRRLATLKSDQPDGREFQNLSYGYDKVGNVTTLTNDVAVPGAKELGGPSTQTYGYDDLYRLTSATGSYRNDNDKLNHYQLDLTYDTLNNTTGKTQKHDLTTTSGVTQPQYNTSYTQKYTYSALPHAPSKIGTDTQKYDAGGNLIAITAGRSTDAVVLAAADTSGGKRTQYVWDEENRLECAFDDESSTVDQDPASCDGRHEQPTVRFVYDDAGNRVLKDGEETHVSIGTTYSRVDGKAVKHYFVGDTRLLSKKASDSGYEHDQFYFHPDQLGSATYVTDERGKLASHQEYFAYGESWVEETRARKPVPYQYNGKEFDKETGFYFYGARYYNPRTDLWQSADPATGSYLDGTPNGGVTQPFNLSSYSYASDNPVRLADPDGLSTWNRVMGGLKLVGGFSEAATGAAFGTATSWTGVGAVAGAVVFVHGADTAATGLRQLWTGEDESSLTSLAIQHTTGLSKETVDRGEAVVALGASLGMSAATKAPQVLGATAGGGGQTTRAVGSAGLIGGGLSQPGALRAAAVEVHDLALLEKGGEIRHTMSTVALHEARLADGTTQIFAAGSSGRLSAAQIEKLVELGVPRANIVRGGLHAERNILAAVPEGAEQVRWGIAWGARNKPIPCDKCAPFLKGVLEGAE

SEQ ID NO: 40 - protein, 2563 aa, Amycolatopsis sp.; identical to SEQ ID NO: 39
  REGION 1..2563: note = MISC_FEATURE - AshDa01
  source 1..2563: note = Amycolatopsis sp. Hca4

SEQ ID NO: 41 - protein, 192 aa, unidentified
  REGION 1..192: note = Isolated from environmental sources.Organism unknown.
  REGION 1..192: note = MISC_FEATURE - MGYPDa18
MNYMPDKAATEAQAAAKANLYIALQKMFPTASKRAIDEVAKTILEKRAKIARFREIAGLQPYSEDSGDTVAMTEVGGHAIFGVNSSITKESHEATKELRQKWFKMVKWCPPKKKEPKHLGHVQSLTHAESHALIRAYERLGKLPKRIIMYVDRKTCNMCKGEMPALLKHLGVDELTIFSGDSLEPLVLKAVK

SEQ ID NO: 42 - protein, 397 aa, unidentified
  REGION 1..397: note = Isolated from environmental sources.Organism unknown.
  REGION 1..397: note = MISC_FEATURE - MGYPDa687
MVFEGFVPTAKLVNGKAYSIISDHLGTPILAVDSEGNEVWNRQLDIYGRVRKEYKHSSLGDDVFPFVPFLYQGQYYDFETGLCYNRFRYYSPETGTYISQDPIGLAGENPTLYGYVKDSNWWVDVFGLSENYHLGDYQISSPTLPAYDKNKTEGVLYYIDKKGEIQAKEFSSGGKTDFPNYANSNHVEGKSALFMRDNAVREAVVFHNNTDGTCSFCVAMTETLLPKGAKLTVVPPENAVAKKRGYINVPKTFIGNENEPKRNEKNAQINQVMYLNDFKQEYLINSLNELNNILTKRFFEDSNHFILTFEDYGFPQLSCFVKKSMSVMYYLDMQKSFISKNNNIYSEENEVFYEDEGLEHKVFLPKSSIIPINKFYESAQYFFKTCSKPMNIDWLEL

SEQ ID NO: 43 - protein, 866 aa, Pseudomonas putida
  REGION 1..866: note = MISC_FEATURE - PpDa02
  source 1..866: note = Pseudomonas putida ND6
MPDGNRLQFKYDEFSRLLEETDPLGRKTTYEYHHLTTLVTQVSYPDGSTWRARYDDKGNLLAEFDALGQMTEYLNSDDGLPHTIIDATYKSKYLWWNTLAQVERYQDCSGKSTYYRYDERQHLVAVTDALNQTTTLARKPDGEVLRISHPDGTTETFTYNVYGQVLSHTDGKGQTTRLMRTARGLPSSRQDAKGQRVRYEYDKAIRLTALVNENNATYSFAYDTSDRLSEEVRVDNLTRRFSYDVGGHLTRLDEIGYGENAERPERHTLFERDAIGRLVAKINRDASQTFAYDDGDRLLSIERQPTGIGKQLGVTEEKLEYTYDLLGRLTKEITPAGTLGYEYDPLSNLTTLTLPDGRKVNHLYYGSGHLHQLNLDGLVISDMERDDLHREVYRTQGKLTSCFGYDAMGRKAWQFASTLSADKLSQVHNTGVNTSLLVEHAYNPIHRRYQYDPAGELVRTLDKLRGEIKYEYEANGQLRSRDTGSLIGSEEFRYDPAANRLDFNARQFDKVKDNRIKQWRDQEYRYDPWGNLIEKRSGHSKLQHFSYDCENRLVRAETLVNGKLESQGEYRYDSLGRRIAKQAEINGEVEQKRFLWQGLRMLREESPAKSILYLYEPGSYAPLARVDQVEGEGQKVYYFHTDQIGTPLELTDSRGEIVWQATYRSWGSVEQLLVSEVEQNIRFQGQYFDYESGLHYNTFRCYDPEVGRFATQDPIGLTGGIHLYQYALNPSAYVDPLGLSFGSGKGTHNAIATLYDSSGNVKATGIWQSGNMTPDEAALGFPKSTLATHTEARITRELSPQAVPGDRLVIEGEYPPCNSCKGKMNAFKGSTGADVEYRWASGDGTTTEVWNAKSKRTQKISGSGCG

SEQ ID NO: 44 - protein, 373 aa, unidentified
  REGION 1..373: note = Isolated from environmental sources.Organism unknown.
  REGION 1..373: note = MISC_FEATURE - MGYPDa03
SGNRLKEYNGIEYTYDALGNLIYRQLPNGENQYYQYDLENQLVRAEIKKAAGNTEIWTYAYDPFGRRLSKERQDKLAWTSTEPKRTHFVWDGSRLLQEYTYKGSYTYIYTDQDSYEPLAQVFYDNQDEAQYLAYFHNDQIGIPREMTDIHGNLLWYGEYTAWGRLKKDERVYKNAHQPFRLQNQYYDEETGLHYNLMRYYEPEAGRFVNQDPIGLWGGANLYQFAPNAAMWLDPLGLAKFGSGKGTHNAIVSHFDSKGNLVGQPRSYQSGGMTPEEKALGFPRSTLATHTENRAMRDLTPDLKPGDTVRIKGEYPACPSCKGAMNRAHEKTGARIIYEAPGTEPWEAGKSKKRTRKAPRPAGKKRQTPPPGNC

SEQ ID NO: 45 - protein, 1415 aa, Lysobacter sp.
  REGION 1..1415: note = MISC_FEATURE - LsfDa01
  source 1..1415: note = Lysobacter sp. 5GHs7-4
MTTAAKHFDPQLGIDIHMYVFPPVPLPVPLPTPHIGIVLDPFDYLPFLGGTVHVNGIKRATAGTGGLNLHIPMGAYHPAFLPKLPTGPQTDDELFMGSMTVSADGDPFSKLAMPVLDCNVVGMVPPFRLRKPKKPKLSLTLPTAVNLAIPTNVNVGGPPTISLMAMAMKGLFKLLGPVFKRGGKAFKKLRQKVFGNMKPGFLKCKVLRAEPVDIRTGSVSVTHEDFVVPGRLPLAWTREYGSNNDHVGACGYGWETPADIRLELDADGSVLFHSGEGVAVFPQAPEAPGLAHAVREFVDGARLYAQGDEWRVRSKDGLRYAFARPAANLVALTTQTLPIQRIEDASDNAWRFERRGADLVRIVESGTVDPVSGQPLQGRFLQATSRQGRIEALELHDPATGRNHPLVRYEYNDAGDLVSAFDPLDAPRTFDYRQHRMVRHTDRLGLSFHYAYDEQWRVVHSWGDGGLYDYRFAYDDLLGDVEITDSLGHTSLVKFDEYRLPLCEIDPLGGVTVFEYDEAGRTTAVVDPAGLRTEFVYDERGNLLQLTRADGSTLQVQYDEEDRPVAVIDPLQASWQQRWDERGLLVEQSSPLGARSTCSYDPHGQIIAYTDALGAVTALHYDRHGQVQAIVDPLGTRSSFQHDALGRLLEQANAAQQRTGYQYDAKGRLLNVVQPGGTTVRAAYDAEDQLIRYTDEAGAVTQLAYVGIGQIARRLQPDGHLVQYEYDREEQLIAVINQRGERYELKRDALGRIVEEVDYWGQARHYRYDAVGRLTGTTDPLGQVIAYATDPLGRITAKTLPAAERPGQQIKEIFRYDPRGQLIELRNEHRQVKRTFDSEGRLVEEDQDGFVIANTFDALGNRIVRTASAGNRVACEFDLRGQLSRVTINDDAPILIERDTLGRALTERLSPQVRRQLRYDERGLLTAQAVLKDEAPLFDTSYAYDAVGNLTQRHDSAHGADLYTYDPMGRILAHTDPRGKLTQFLNDPAGDRLRTRVHAKRQQQVVGGEPQPDEWTREGDYTGQHYVFDRAGNLVRRGDANSAGNDDERALHLRWDANQRLLESTKDGATTRYGYDPLGRRVFKRNPTHTTWFYWDGDALLGEVSHDNDRIEVPALVNDGVVVDLFEARQRKRVFDGLHKRVREYVYYPSSFVPLALIDRQRDAAVNTSPAGTAAPEAATTTEVFHYHVDPNGCPTRLTDASGKVVWSVSYATWGEVTRVHVAEIDTPIRLQGQYSDVETGLHYNRHRLYMPELGDFASQDPLRFMAGENLYGIAPNTYAWIDPKGLSCQLSHIARQVHDLAGNHPKAKQMSTVAVVEAIVDGKKILYAAGSGARLSQMQRELLEEYGVPASNIFHGAAVRANDQLANHAERVILRNLPPGAKVTRWGISWAGNQKNASCPNCQPFVDAAGGVLD

SEQ ID NO: 46 - protein, 1467 aa, unidentified
  REGION 1..1467: note = Isolated from environmental sources.Organism unknown.
  REGION 1..1467: note = MISC_FEATURE - MGYPDa02
MADTGYFDSVSANLKTGVGAQVKGISEGVKANSEAGVSPSVNALDTGVKAAAAIGGLADGLSEAAMLPVLGAMGMKGMACLPISKQLDPVIGVDIHLVTIPPSPVVPMPHPYVGVLLRPQDFIAAAVSSFIPPPPTAEQTGDADSAKLAEVGHTVLTMAVGMLGATVKIGGFIPRAVASTPTRSIPHIPMGAGWAAPSAAIPKNNGHAFMGSLTVLADGMPFSGGGAHLHLDCNDVGIPSVHKVPGMFLPTGVINPIPPARQILTSPVPVPLNPMAALARKCTGAFGRFYKKKTRKLADRLHSKVNRTIKSESLKNMLHKAICTVTGHPVDVASGTFFTDEEDFWLDGPVPLSWERTWYSRSDYQGPLGNGWHHAYDMGVVADTEEGTLTLRMSDGIPVAFPLPTAEEPSFILSERKEARLEQDGGYCVWDMAEDLYYRFTRKEYDSVRLLESVTDCNGLGIRFDYTKEGLLRSITDSAGRRLRVEHDTRSGRILEICGPHPEDPEKEITLASYEYDADGNMTLQRNAAGDVMTYEYAGRLIVKETWRNGLAWYFEYDGTGVGSRCVHTWGDGGIYDHRLTFREGVTEVLDSHEKLTVYHHRGGLVWKKVDANGGEHLWRYDDSRRLLAQTDPLGNSTLYRYDRWGNCTDSSDPCGGSVSAVYPGKGNLRNRPVSVTTPDGGTWEFGYDRSGNLVSRTNPEGAVTRMTYRNGAVASVKDPYGVVTRLAYDRFHNLTEASDSRGNTSLYGYDLLGRCVSVTNPKGAVQKREYDPVGRVVRVLDFDGNDIRLSYDGIDNLTEYRDNVQHVEYGYSGMWKLTRRRDHRGVVNFRYDREERLRRVTNERLQSYEFALDAVGNVTAEKGFDGAVRHYLRDRGGRVVRETLPSGTEREYGYDACSRVTRVSYPTAGDPDQTYAYGLSGRLVQASRGESTVEFAYNSLGLPTRETADGNTILRTYDHTGRILTLDSTAGASLHYTRNGYGELEGFTATGGSDADGAGSWESAHRHDTLGFEVERILPGGIVRSFAYDDIGRLVDARTRKDSRTRHMRRYRWGVADRLLSVEDSRRGETRYSYTPTGQLERAEYPDGREQWRKSDQTGNLYPDPDMKLRRYLGGGRLEQDGEWHCEYDADGNLTERYLGTGRWLDGKKDRWRYRWNADGSLAKVVRPDKREVEFTYDALGRRLSKSFGTTVTRWVWNGNVPLHQWKQHREYSVMEDRWNTDTKHRDMTVWLFDEDSFVPVAMIKEGRSYSILTDQLGTPTEAYDTEGNEVWSRVLDMDGNVIEETGNKGMVPFLFQGQYYDRETGLAYNRFRYYSPKMGMYVSQDPIGLAGGILNLYGYVDDTNYFVDILGLWGGKKGQHTAIVSVYDKDGNLILTKVFTSGDMTPAEHSLGFPKSTLATHTEARAVAQISLSAGQRMEIVGKYPPCNSCKGKMNQAVEATGASITYYWTDEQSGERKEWKAGYR

SEQ ID NO: 47 - protein, 157 aa, unidentified
  REGION 1..157: note = Isolated from environmental sources.Organism unknown.
  REGION 1..157: note = MISC_FEATURE - PvmDa01
INEKRKNNTPWEIIDNYRVEKGMTKLDDIVPSPGDGLNTVSYIDVGDQTIYGVNSSLLTDSEKDLGRHVFKEMQDMGLFGNPNLHYGQGGSQVLTHAEGNAIMKVCSEYKSQLNGKIVLYCDRKICPFCNANMSALKQYYGLDEIVIINKNGSVHKY

SEQ ID NO: 48 - protein, 197 aa, unidentified
  REGION 1..197: note = Isolated from environmental sources.Organism unknown.
  REGION 1..197: note = MISC_FEATURE - MGYPDa917
GQYYDFETKLAYNRFRYYSPETGAYISQDPIRLLGNNPNLYAYVKDSNWWVDVFGLSENYNLGDYQISSPTLPAYDKNKTEGVLYYIDKKGEIQAKEFSSGGKTDFPNYANSNHVEGKSALFMRDNAVREAVVFHNNTDGTCSFCVAMTETLLPKGAKLTVVPPENAVAKKRGYIDVPKTFIGNENEPKRNEKKCSN

SEQ ID NO: 49 - protein, 336 aa, Acetivibrio cellulolyticus
  REGION 1..336: note = MISC_FEATURE - AcDa01
MYKITISDGTILKESIEHVSFSTETTNDYNSRHTNPRNSMIITGKIDTDEKTAGLYKWALLPGSNPECYKEITVEQYQKELLVRKVSFNKAFVVDYSENYSNSTGVGTFTLYIRQFFAKDIEVTSKESNNTVINKVEEPVKIIQKKIAMMENIPKVESAKKTTMSFTDRLAKQSEQDLSGLSDDKLKEIFAKEIEQSKEAYTRATDIIKTKKLKGCVASNGKITLSGFDGKTTKAPKDFTRVSLDTMFDYCKEIGHELPAKMPDFYDNGIKGSFFACHAEKQLSLLTDKPIGISKPMCPNCVEYFSKHAIHTKQVKITTDPKKTRIFFPNGVIREI

SEQ ID NO: 50 - protein, 346 aa, unidentified
  REGION 1..346: note = MISC_FEATURE - CbDa01
  source 1..346: note = Clostridiaceae bacterium
MEYKVTISDGTVLEKSIQHVSYIVYPPDDYDIRRNYPRNSMVISGIIDIGEGTVGLYKWALIPGTNKECYKDVTVEHFYAGQLVSKVKFSKAFVVDYSESYSNSEGVGYFTIYLRQFAGMDIEHSNEISQEQQKKYQEQVQTNIEQTSELEKAEPKISSAVSLDNVNNVNSKPVMRITDRLKNQKDPDTLLTKEEVKLRYAKEIEASKEAYKRAIDMAKKKKVKGCVASNGKITLSGFDGKTTKAPEGFARVPLDTMFDYCKKIGHELPPKVPAFYDNGREGSYFACHAEKQLSLLTDEPIGISRDMCPDCVEYFKKHAAYTNQVKITADPKKTRIFFPDGRVEEF

SEQ ID NO: 51 - protein, 153 aa, unidentified
  REGION 1..153: note = Isolated from environmental sources.Organism unknown.
  REGION 1..153: note = MISC_FEATURE - HmDa03
IRSFHTYFVGCDEWGFSLWAHNNSCGPEGGATSNAPVPRLGPRGVDPAHHNANIMVRDASGRLRHHERIVSGNMTPEEQALGFPRGMMASHTEARAVRNIPLQPGETMVITGQRPPCTNCRGAMNQAARDTGATIIYRWRENGVTRTWTAGGN

SEQ ID NO: 52 - protein, 1521 aa, unidentified
  REGION 1..1521: note = Isolated from environmental sources.Organism unknown.
  REGION 1..1521: note = MISC_FEATURE - WWTPDa05
MGGMAAARLGDKIGHVSVWARLARVGLRLAAGMVEALAVGALITLAIGGSVATMGCGAIIAGGLIAGFLGSVSGWSDFKEKKIKEMTDDIGSPEITGELGQPGSPDVRINQLSSMRAIIDAALCSKHSSNPPPQIAEGSDTVFFDTYPAARKDDKLTCSAKIVEGSDNVFIGRNKASYLDIADDTEWWEVALEVVAGLALARGSFPGRLGCMALGALAGMAGDALGRGFRSLIGHPVNPATGGKVLDGTDDTDFVLPGPLPIEWKRFYSSHDARSGLFGRGWGLPYTVELHIACKPGAPDTLTYIDHQGRQIDLPMVEPGHALFNSAEGFTLGRTPSGHFDLTTQDHLHHQFDRAPAVPGVHVLKLQRLADRNGNWLRFRYDEDGRLTAITDSLGRILRLRYQGSGRQVTAIDLDKPAPDEAPGRLVSYQYDANGQLAAVIDRLGESSRWFAYGDGLMVKQAWPSGLECHYQWEAPGAGGGPEGERRVLRQWTSDGESWDIRYEITADGGTTTSSDALGRRECWTWDARYNLTGYTNPLGHSWQLAWNAHRELLGVTLPDGAAWRYAYDANGLQVARTDPLGRTTRTRWHERWFEPRQISEADGGIWRFEYDERGNLTVQIAPDASQNLTAYDNRGLPILLIDARGGHNTLSWSVRAQLVSRTDCSARTTRYHYDGYGNLARIVDAIGQETRLTHDALSRLVTATLADGAHLAWHYDATGRLIGETDALARRTGYTLDARGRLTGRTDAAGRQVAFTYDIAQRLTRLTPENGQAYDFTYDAADRLIEERRPDATRLGYDYDANGELAAITQYPGLGDDAFGQIESEGDRPRHQPPAPRRTELVRDGAGRLTEKRSQSVDQGKDGKATEDGKAAAEVVRYAYDASDQLVEAIRLGQDGAPVHSVRFDYDALGRIVAETSTDHLHGTTHTLTHEHDALGNRTRTTLPALPGSPARKLHYLHYGSGHLHHVRLSLDEGEGLDAHSRWHGLADFERDGLHREVLRTQGALATRYQLDPLGRRLASRAHRGTGLGVLPDTQPLGNHLPGSGTETFGKTYRYDPTGELREAQHPLKGQARYDYDPTGRLEAVLRTGGAGPTGPVTRHPSPVTEHYAWDPAGNPIDPGQEQRRGYVRDNRVRVFEDKRYAWDGFGRLIDKRAGKHTRQRFEWDAEDRLIAVTTTRRPDTEHATTQTTRFEYDALGRRIARTDAFGRTEFIWEGMRLIEERRGAQASAYVYEPGSYVPLARIDAGGGAGEAGQGGAANDATGTANAGDDAPAGARVYYFHTDPSGLPEELSDPDGRIRWRAAYRAWGNTVQESWEATDLDGRPLALDASDITAPLEQNLRFQGQYLDRDTGLHYNTFRYYDPDIGRFISPDPIGVGGGANLYQYAPNPVSWIDPLGWDPHEATITVYSSDGQVRYQEPLRSGGATGTSWPEQIASHTESKGIADPRIQYGDRVVFTDATLPPCNGCRGNMNNAAAAKSLAVEYHWSDASGRKGTWSTDPTKAARAVGVRARRNAVKKSC

SEQ ID NO: 53 - protein, 1417 aa, Serratia sp.
  REGION 1..1417: note = MISC_FEATURE - d22_SjDa01
  source 1..1417: note = JUb9
MSEAARVGDAIGHSHALAGMIGGTIVGGLIAAAGAVAAGALFVAGLAASCVGVGVLLVGASLAVGYLTGELATKARDGIAEAGAGSLTPAGKIVTGSSNVRINGKPAAIATVSQVACDQDGPSMQMAQGSDKVFINGQPAARVGDKTNCDAQVMEGSPNVRIGGSTVTTLPIKPEVPDWLYKISDLTLLFAGLVGGVGGAASKLGALGKMLSKAPGINKLGRTACRFGTLMTATTAAGIIARPIDIISGQKFLSGDDELDFVLPSRLPVEWQRYWRSGNPAQSVLGRGWSLFWESSLKRYQDGLVWRAPSGDYVSFPMVPRGHKTYCEAEKCWLMHNSDGSWQVFDVSEQAWHYPALSDAPSRLQMLTDLTGNTISLFHDDHGRLTELVDSAGQRLACRYLTTANGLSRLSSVLLFTPDGELPLVHYGYDDEGQLVTVRNRAGEVTRRFGWQDGLMVSHQDQNGLLNEYRWQEIDGLPRVAAYHNSAGEQLALYYDFANGTRWAVRDDGTRALWQLDDDDNVAQFTDFDNRQSAFIYARGELCGVVLPGGAQRQSEWDRYGRLLSETDPLGRTTTYQYSRNSGRLFSVTYPDGSQAFQHWDSQGRPTQQIDALGNVTRYHYPDEEESLPASVIDALGGEVKLTWNAQGLLTRYTDCSGSVTAYAYDALGQLTHRTDAEGHLTRYHWDRAGRLQTLLHPDGSEERFDWSAQGQLARHQDPLGSETRWQYNLLGQPVSITDRIQRTRHYHYNCRGWLTRLENGNGGDYQFSHDAVGRLTAERRPDRTDRYYRYGPAGQLSELRETGPLDPQAPPAQRLHQFRYDEAGQLTWHANDSAEWHYHYDAAGRMNRLMRTPTAAGSKLGIEPDSVQLRYDKAGNLLSEQGVNGELQYQWDALSNLQALTLPQGDQLQWLHYGSGHASAIRFNQQLVSEFSRDRLHRETGRTQGALHQQRQYDVLGRRSGQSSAFGHISKPEEGVLWRTFRYTGRGELEGVSDALRGEIHYGYDAEGRLLQHREAQQGKPGHRLCYDLADNLIGEQRVSHDPDAELPPAPVVDNRLEYWQRMFYRYDGWGNLTSRRNGIYQQHYVYDADNRLIKAHGRGPQGDFAAQYHYDALGRRTRKTVTYKGKAPETTRFLWEGYRLLQAQRDNGTRRTWSYDPANAWTPLAALEQAGDGQQADIYWLHTDLNSAPLEVTDAEGNLRWSGNYDTFGRLQGQTVTGAERRKGALVDQPLRYAGQYQDDESGLHYNLFRYYEPEVGRFTTQDPIGLRGGLNLYQYAPNPQGWVDPLGLAFGSGKGTHNAKATLFDSNGNVKATGVWQSGNMTPDEAALGFPQSTLATHTEARITRELHPQAAPGDRLVIDGEYPPCNSCKGKMNAFKTATGAEVEYKWPADTGDGTKIWNAKSKGTRKIPGADC

SEQ ID NO: 54 - protein, 502 aa, unidentified
  REGION 1..502: note = Isolated from environmental sources.Organism unknown.
  REGION 1..502: note = MISC_FEATURE - MGYPDa09
MADTQYSQVSTFTWADGNDTIEYDYDDNGSLISKITYKRTGPETIEFEKGLRYDYNLQNRLVAIWDFGSDDVPYTANDILLTEYSYNTSGIRTQKIDYETTPAQLTTYLIDPANHTGYAQVLEETTYDDDGSDGWAPATRIYYTIGDDVLSQTTSTYSGGWPTGITKYLLYDGHGSTRQLIDSGQNIGANYNYDAYGTLLQDGSAESNPGITPQQDTSLLYAGEMFDTNSQMYYNRARWYNTLNGTFNRTDPYAGNTQDPQSLHKYAYVHNNPVNAVDPSGLSGEFSLASKIMTFAVIAMLFNANVATAPRSLGDGTSDETSNIIAVLFLCLALTALAAIFAPVFAKFFQGVKNAFIQGIAKITRKLVTNNRAVKIKQGLPSSLQDKAVAVAGRDKALSGWSGNKPSEFVKVDPEDVLRLSNKIGHQPKSAGAFDHGIPGRFYASHSEKQLAFLEHDVIEVSNTMCDDCIDFFAKLSQNTAKAKIVADSETVRIFLPNGEIF

SEQ ID NO: 55 - protein, 148 aa, unidentified
  REGION 1..148: note = Isolated from environmental sources.Organism unknown.
  REGION 1..148: note = MISC_FEATURE - MGYPDa05
KSGFGSADEAFDAIAEFRSRTTITDTQGGRGTVAVTEINGQRYFGVNSSNLSGADKNLARSWRDRLGLTRGQDQVVFHAEAHSLMRAYERTGENLPSNMTLYVDRNSCGTCQTYLPRMAQEMGIDNLNLSFTSGRTAAIRNGAFEWVN

SEQ ID NO: 56 - protein, 327 aa, Vibrio spartinae
  REGION 1..327: note = MISC_FEATURE - VsDa01
MPAAARLGDNCAGHGCFPATPIMAGSGDVSINGKPAARKGDTVLLHACPCPQFPHGMHGRSISAGSSNVSINGKPAARVGDAIDCGGSVAAGSGNVLIGDTPYKSPTHKCGEGAAMEQSPFLRIQPLAEAPPFDWASLPFVEEVYERSAKDKQKVVEKKSKKIRKGTNYNHKIQSPSDGYNVISSFRKKAGLQPYSSTSGDTVAFIEINGKKFFGVNSQITKTSQVSQKALREKWMKEVEWVPPKKNKPKHLGQVQSLTHGESHSLIRAYERLGNQLPKELSMYVDRKTCNMCRGEMPALLKRLGVDKLNIYSGGSTKPIVLESIKL

SEQ ID NO: 57 - protein, 188 aa, Bacillus aquiflavi
  REGION 1..188: note = MISC_FEATURE - BaDa02
TGYSSLPIVGMGTKPLGEIRPQMFSVKSKEGREMGNRKSINGYEGIGKFREKVGLKPYSIDSGDTVASITVNNKTYFGVNSTITKESQKASKALRQRWLREIEWVPPKRTAPKHLGHAQSLAHAEAHSLIRAFERQGSLPKTVTMYVDRKTCNICRGELPALLKRLGIDELEVFSGGTTKPIIIKATK

SEQ ID NO: 58 - protein, 195 aa, unidentified
  REGION 1..195: note = Isolated from environmental sources.Organism unknown.
  REGION 1..195: note = MISC_FEATURE - HmDa02
MFYVLGKGWTAAGELKPGDRIVGLDPRESVSVTALRLTGRQEKLYNLRVADYHTYFVGDAAWGFALWAHNEYNAPTSSPVPRLGPRGVDPAHHNANIMVRDASGRLRHHERIVSGNMTPEEQALGFPRNTLASHTEARAIRNIPLHRGETMIITGQRPPCPTCKGIMNQAARESGATIIYRWRENGVTRTWTAGN

SEQ ID NO: 59 - protein, 262 aa, Snodgrassella alvi
  REGION 1..262: note = MISC_FEATURE - SaDa03
MIQETGPNHPTSLYIYTDHNSYEPLARIDTDGNYEQHIRYFHNDLNGAPEELTDENGKILWECSFQLWGKRIHEIEHEPIEQNLRYQGQYLDRETGLHYNTFRYYDPDIGRFTQPDPIGLLGGFNLYQYAPNGLTWVDPWGLSFGSGKGTHTANATLFDANGNVKAAGTWQSGNMTPSEAALGFPKSTLATHTEARITIDLKDRASPGDRLIIDGQYPPCNSCKGKMNVFAKDTGATVNYNWGENSERKTWKAGKTKNMGCK

SEQ ID NO: 60 - protein, 1641 aa, unidentified
  REGION 1..1641: note = MISC_FEATURE - PdDa01
  source 1..1641: note = Prochloron didemni P2-Fiji
MIDPNSSETEYKYDKLYRLIEQIDGESGKTKYRYDKVGNVRFVTDPRDKVTEYQYDRVYRLQKEIDANDETREYGYDLAGNLTSIQDRRDNTTIFEYDDVNRQTSRINPYGARFKIDYDKVGNVIKETDELGRSTKYDYDELNRLEVVTNAEDGTVVYGYDKVGNVISVQNERGKITRYEYDEINRQIKITNALGFETIIDYTNDTDSNQLVVLVTEQVDADSSRSSEWRTDSRERLQTFTNADDTTTTYSYDGNNNLLTVVDELGRQTAYTYDELNREIRVTDAEGRTVETDYDANGNVVKVVDELGYTVEYGYDQLNRLTSELDNLNRSLTYSYDSEGNQTKVTDSNGGETKYKYDKLNRSLEITDAEGGKTTYTYDGVGNLKSVNDPNSNNGGNSNENGHTVTYSYDARDRLIKEIDALNQETTYNYDEVGNLTSVSDRSGNQTIYKYDEINRQIERIDADGNSFKTEYDKVGNVTKETDELGRITTYSYDELNRLLEVTYPAIDGVVSSSSSTYDAVGNLLTSTDELGRTTEYKYDKINRNTEIKTPLGFITKFDYLDDTDNNLLVLTVTDAVGRETVSRTDGRGRLIEFVNADVSNVTYSYDGNDNLLTVVDELGRATAYTYDELNRETRVTTPLGNTTTTEYDSNSNVVKITDHLNNATRYTYNELDLLATETDARGFVTTYTYDEEENLASVQDPLDNVTTYTYDNLNRLFQETAVGVGTRTYSYDEVSNLTQLLDRNGRIVQFEYDDWNRQTAELWLNGDRIVNTIAYDYDAASQLVEVSDAVAKYSYKYDDDSRLTKEIRDTFGFTDGVVLDYTHDGVGNVLTVTDTVGGIQLSTETYTYDAQDRVISISQTGNGVTPKTAVVTYDDAGQLEQLEVLNAFTTAQTFDLDGRPVTRTHTTAVGEVLAYGYEFDSLNRITAITSPNGESSFSYDSTDQLTSASSDFQEDENYDYDETGNRLGNEIGPHNRLLNDGVYSYEYDDEGNRISRTEIATGVVTDYEWDWRNRLVEVSSSNELGEVLSSSQYQYDAFDRRVAKLVDSNGDGIYELVEGFVYNGDSILLVLNGDEVNQRYFYGPGIDWVLAEETAGEGVEYALTNHLNSVEFILDSAGEVINEIVYDSFGGIVSETNPGVDVRYGFTGRDFDKETGLGYNRTRYYDFVTGTFLSVDRLGFEAGDVNLYRYVGNSPTIYVDPSGMFWEGFAEFIDENVNKPVGQFIADNPLVEDAVRIADSVAVGVSQTANLVGFDIREFREDIYGEGVNDNLNEAVVTASTVATLALGGYALSGLAAGSLGKVAATSINTGLFNTGFGLTTQLLFNGGDSRQLDYNSLRLDFAFGALGARLGFGVTARVGGGFATQFAVSTAFEGTFAGFEQVGRNLISGERLTQGVGENILSSILFQGFSEALSNPRQALQAGRRLLRGADNLADDFLGVVRGPQLATVDGVDSRLLRSVDEVPNVPKGGDNVFASIDDPKSRGELTKLIQQNGNVVDLSDRLIDVATSISNRSLHELAKDVHKLKGDKRAITQSAVAIVEARLVNGKKQLFASGSNATLSPRQRELLKQFGVPEENIFSGSKFRSSDKLKNHAERVILRHLPYRAKVLRWGISWASKHRNIPCPNCYPFVEKAGGIIE

SEQ ID NO: 61 - protein, 198 aa, Burkholderia contaminans
  REGION 1..198: note = MISC_FEATURE - BcDa01
MGEAREVIARASKAAGITPRSALRFQGQQFDDETGLHYNRYRYYDPSSGRFVSKDPIGLAGGLNRWQYAPNSVNWIDPLGLARFGSGKGTHTAQVTVRSAEGSQRHCGVFQSGNMTPEEKALGFPQNTLATHTEARAVSQVPLAPGDHMIIDGQYPPCTSCKGKMNARALQSGATIQYNWQEDGQQKTWMANPSKGCK

SEQ ID NO: 62 - protein, 294 aa, unidentified
  REGION 1..294: note = MISC_FEATURE - DaDa01
  source 1..294: note = Desmospora activa DSM 45169
MKGREISKPSDTEQKRYNYTLYFLMILLLSIVFILLLPPDIAQAHNCGDFRDCYSTLRAAVAATLGLSMFAVMLSIGLDFVPIVGQTKGMIEAISGRDLITGEELEDHVRLLGILGPAGRIAGGAASVGRGASHFSKAESGYIEIQRFRRILNMPRYSLTNGRTGTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 63 - protein, 2435 aa, unidentified
  REGION 1..2435: note = Isolated from environmental sources.Organism unknown.
  REGION 1..2435: note = MISC_FEATURE - MmgDa02
LTIDSVDGIGGIATSGDVFVSGVGLTVNSGINVGANTVRLQTSAGISQGFLGTITAGAVAVSNSGTAAVSLASSNSFGAFAATSSAAASISVTDSVSGGMTIGSVAAGGTLPAINGITTTNGTITIGSSTGVTVNQPILSGGAAVTIDADNQAINQSVNAGTAIVTLSPYNGNRAINLGAKPGGVLGLTNGDLSQITASVLRISNTASSGLTVSAAITAPLGWNALALLSSGPITETGSLAVANLRVQSSNSSITLIGANAVSTVAVQNFFANTGPVAVVSTLPLTTGTVDGFSGVSAQSADILLSGAGISVTQPIIAVNLRLLTSAGVSESGSGTISATGIGVRNTGSGSISLPLSNSFQTFAATGAGEIVANAGGQPLTVGTITADSTGTFTPLSGITTSNSDVELYTSMFGSVAATQPINVGTGTVRVTGDGGFTESGSGAITAAALGVYINNNTVTLDGANNFATFAAAMGTFGSVVSINDAGHDLTIGTVGADSAGLFASVSGVTVSFFPSMTVSAQSLSVTQPIVVGIGNPVRFVTTNGVTQSGSGTITGNLGIRNSGSGNVSLTLGNSLGTVAIAQTVVAGTVSINDTAAATIGTIAADAQSLFSATSGITTAGADILFTETSAIAINQPLNAGTGIVRLVTAGGISESGAGGVTASALGVINTFSGSVALPQANNVGVFATSNSVNGGAITFVNGTTALTIDSVSADASGAFAATSGITAIGSGGVTLSSGALSITQPISDATGTVRLVAAGNVSQSGMGIITAATLGVRHSTAGAVNLNLANVGPTQFAATSSLSGASITFTSAQAVTVATVSSNTQFSTTTGIITSGGGNVILSSGALTITQTINAGPGTVTINSAGGESELGGSITAATLAITNSGSLNVSLGVGNSVGSLSITNTVGNVSFTNAQSLAVTGISALNTSGTVSVTTTGAGHSLSTSGTVSAGTSSGSATLTSSGILTLGGDVAATTVTLSSVGGTSQSAGVITAAGSLGATNTTSGDIVLTQSNLIPSFHATNAVGNVSFMTGQSLTITGITALSTTGTVSVRTTGAGHTLTANSAISAGTTSGSVSLLSAATLTLTANVTGATVLLSSVGGMSQTNGAITAATSATATNATSGNIALAQANQAAAFTAANPVGNVTFLDNRTLVVTGITTLSTSGIITVTTTGAGHNLSTSGVISGGANSGSATLMSAGTVTLGANITAATMAINSVGGSSQSASAIVSGGSLDTTNTGSGDVVLTQTNLVVSFHATNTVGNVSFVNGQSLTVTGITAQSASGVATVTTTNAGNDLSTTGTITAGTTSGAVTLTSTGTLALGTITAATVTLNSVGGVNQTSGLITASGALSATNASSGDIRLTAINQIASLSATNSVGNVVLRDGRSLTITGITALVNTGTVSVTTTGAGSNLTNSGTISAGVTSGSATLTSAGTLTVSASLTAATVTLSSVGGMSQGGAITAATLTATNATSGDITLGQTNFVALLAATNPVGNVAFSDSQSLTVTGVTALSTTGTAILTVSGAGHNLATSGAISGGTTSGSVVFTSAGTLTLGANVSAATVASSSVGGTNQTGGVITAATSAGATNTTSGDITLTQANLAAAFSATNLVGNVAFSNGQSLAVTGISALGTTGTVSVTTIGAGSNLSTTGAISAGTTSGSATLASAGTSTLGASVTAATVTLSSIGGTSQTAGVLTATTAGTTNTSSGNITLNQANLVNSFSATNLIGNVAFTNGQTLSVTGITALSTTGTVSVTTTGVGSNLSTSGAISAGTTSGSVTLTSAGTLSLGANVTGATVTLSSVGGMTQTASLVSAASAGATNSGNGNITLSQPNSVASFSAMNTVGNVAFTDGQTLVIAGIIAQSTSGTVTITTTSAGSNLSTSGAISAGASSGSVTLTSAGTLALGADVTAATVTLSSAGGTSQTSGVITATTAGATNTTSGNITLNQANLVNSLSATNTVGNVAFTNGQALAVSGITALSTTGTVAVTTIGAGSNLSTGGAINAGLASGSVTLTSAGSLILAASISSATVTLNSATGASQTAGAISAATVGATNTTSGDITLNQPNLAGSFSATAFVGNVAFTNGQTLVVTGVTTLSTSGNVNLTTVGTGSILSISGLISANATSGSIAIASSDVLILAANVTAATVTLSSATGTNQTAGVITAAGISAANSTSGDIALGQTNSTAAFSATNLAGNVSFANGQLLAIGAAGITTSGGNIAIFAPILSINQPIAVGTGSVSLTTTAAGVTNTLAASITAASTTWTATAPSNLSVGGVSRLTDDEAFALIDKFRSKANGLEPLGGRIPVKGDGLGTVAIVETPGRKFFGVNSSLLSDASKDLGRDFFQVMKSKGLLEGASAYGNGAAQVLTHAEAIALMRAWKFNGGKLGSEVTLFVDRYTCANCRKYLVEVMEAMGIKKLTIVTKSGEQIVL

SEQ ID NO: 64 - protein, 183 aa, unidentified
  REGION 1..183: note = Isolated from environmental sources.Organism unknown.
  REGION 1..183: note = MISC_FEATURE - MGYPDa21
DLIEQLAERLGNHANLTDELIAAELQAMGVAATDDIVARVWSGLNASALRQSGALRAVALEVHNLAGSQIAINQSAIAVVEVTLADGSRQVFASGSGGRLTTEQVRRLIALGVPPENILFGAAFTNGFTQAENHAERILLRNLPQGAVVNRWGISWAGSHRPIPCDVCRPFVAQAGGILEGAQ

SEQ ID NO: 65 - protein, 369 aa, unidentified
  REGION 1..369: note = MISC_FEATURE - RhDa01
  source 1..369: note = Ruminiclostridium hungatei (Clostridium hungatei)
MKPLEGPVNSVGGMIEQVTDQATGVVDNAEQSAMSSMGNGSKLLSGFAGSSGSQNRDAGKAGESKGQEKNSGKSGQEGGKSQGGGDFLGLLKSGVHTRLMTFGLMNIKKLGAKVIAAGAAKVTGVIKKMLTPKVKFRLGAEEHELWVEKGKNGNVVMMASKKPGPIKRKIEEGEIPDNGEISNKRRKVEAEKDEQQVVQQNEAMASTIQAVTAGVGEAGKKEPWEAIDRFRKSNGLEPLGDRIPVRGDGLETVALMEVSGNKVFGVNSSLLSDELKNLGRDFFKVIKEKGLLGNAKHYGSGEAQVLTHAEAHALMKARKEAGGHLGDSVVLYVDRLTCPNCQKYLPEVRAAMGIKTLKVITKGGIELIL

SEQ ID NO: 66 - protein, 162 aa, Micromonospora sp.
  REGION 1..162: note = MISC_FEATURE - MsDa01
MTTGGGSDISRPPATRDTSATATEAPAQPELVPEGLTDAARDLPRRQAKDPTSGVALIGGERIPMRSGRDPDAAADLKPAYKLIATTTDHLEAKLAARMRRDHITQAAVVTNNPPCDYTPYGCEKILSRLLPAGARLAVYVRDDDGQVRHWRTYTGNGKAIA

SEQ ID NO: 67 - protein, 1305 aa, unidentified
  REGION 1..1305: note = Isolated from environmental sources.Organism unknown.
  REGION 1..1305: note = MISC_FEATURE - HgmDa01
MSAYEPLLKKPKAPPAPLRATVASKRPQPPIQTALEVSQPGDALELEADRVADHVLRSAAPAPRMNAGSDARIDREADKEEQDTEEEYAPNHNSLVVQAMLQRMCAKCDAEEDREEETPPGKLQRQALVVQRECGDCSEEEEEEQVQREAATPGPTAAAATPRFQAALSSARASGGEALPSAVRGFMEPRFGQDFRGVRVHRDAAASELTERVHAKAFTVGKDVFFRRGHFEPNSEQGKRLIAHELTHVVQQREGMRSVQRELLQREELEAPPAQSAQAAPAEQAATEEEALLANDEMYLEAHARGFKMAVNGTTYKSWKDPALEAFVLYFLRRYIGGSRDAALACLGDLRATGGIGFQSQLKPGQILNDKGIYGIEIDGALVQSSDAWVKQQRPGLRPVKPKLGIQKIPDIVSEGGGAADSAEEAVERFSPEGRILFPLGDGKIPTFVGGAEIKVAFQFTGAGAALGFNDFPHRINRVDFDWSISTETEGGKKEVDSGPLLEGGANQYSFDVEPGGYLVSVHLKSEHFQSGTELELKRPFEVIDEQARLGEAFDERFVGEGEAQPFELDQEGNLRLKPGAPEARSLKDQILELEFQIGQLEASRDGGGLPADEAAGYIEVFRERIEALKKLEQGSTSSSQYRIHGVFQSREDSSVREIVAVLHGLETSKEGQSSYAFTLNDFTLSPGKAVQHVGAAPAPAGDDRNAAIASAEGAALEALKERWRANNDYPDGHVRFVVPLKHGSGVKELTFDTHTLRKDARTALTVAAIGAGAVSIVLSGGTTAPVVLLVLEGVSLAAGAALAADEIARAIETNTLTKKQLVLNMLTLVPIAGSLARAGGITVRGMQLIQVSAGVAEGVLITAETQESLRAVMAEFAVQIAKAKTPEQRAQIERQRDQVIARLLGAAAVSGGISLVSTALGARGAIDDVRGPKPRAAVEGAEPHVRAGEVEGPTARPVEAEPAAPRRPAPDVETPEARRRGPVADPANGKAATAEATVLTERHLTLAGETHTLSVVRKSDGRHLLTLCSPKPCGELANEIEDLLAQLATDHPARRELGRLHEAARKAQADLDTGQLALDAASIEANRIATALDTLSTKHPEVGKILATGGGRAETRQPAEASREPQAESLEPVGKTEGSGGSDRESLTPEGAWREIDRFRTKAKGFEALGDRVPQRGDGLGTVALVEGGGTRHVFGVNSSSLSESSKDLGRKVFSDMKSKGFFPDTKHYGQGAAQVLTHAETHALMRAWENSGRRLGKSVTLFVDRFTCANCRIALPDVMRYMGIEELTIVTKSGVVIPLRVGG

SEQ ID NO: 68 - protein, 303 aa, Xanthomonas campestris
  REGION 1..303: note = MISC_FEATURE - XcDa01
  source 1..303: note = Xanthomonas campestris pv. campestris
MLKRIMRRAALLLALAWPAAGLAETVEYIHTDALGSPVAITDAAGNVIERTVYEPYGGMVNKAAVDGPGFTGHVADSATGLSYMQQRYYDPQVGRLLSVDPVMAHGNPLGAFNRYWYANDNPYRFTDPDGRQTIPSENKLGSSDPAVINDFHQAQGEHAAITLNVALDLFLSQFGDGPGGAAAGVGGVVAKSAEAVSALTRAREIQGALKPITQSKVTTAVTETAEGVRVVTSSEGRLRPAQRAALQVGEVAGKGAARTHAEVNGVNAARDMGLTPTSVSPSRTACPGCQEAMKKLEIPIKDK

SEQ ID NO: 69 - protein, 759 aa, Actinomyces sp.
  REGION 1..759: note = MISC_FEATURE - AoDa01
  source 1..759: note = oral taxon 448 str. F0400
MWERVSAAYKRWLGRHEALARVLYRLSRISQRAALIALVVALVVVPRLGIALVPMVFMLGCLAVLTVAARTRTVTWRSVTLMMSASVPWALVVALVTQAVGSACGLTPLDDGMKIALAAFVEEPGKLMPLLVVALVAPGRVRRFALADWALLGLAAGAGYTIAEDGVRRLNAPGLLQSILGENRVGYSLNPWTAGSFSTSGSGLTDLLTGKHSSAIMTVGHQVSTMTLAMGIGLGIALWRARRPLRAAGRLLSWVPPVGALAMAVADHAGYNATVSSSSSWVDSGEGIPAWLRLMWLTGGRGHLQVRVSVGLFLVCLLVDAYRRLGAGPSGRTVFEAPRVPMPGLAGAPPPVRVVVQAVVALVAFTWSDAAVVLGAYADPAMDRAARMANGRAMGVQVRGVRADAMAATTPGSEPGARWRFRLVALVVGVAGAAACGWYGTLVARGIGHSVIADGDGVFFAGLLDGLAAWWESLGLAGQILVTALGVLVLTASGLSLALALGAVGVGTWAFSHGHGLASFLRSPRAAARGYVMTVTPGQLALDLLDFALTFIPGSALGLGVRTSAESLAASRAMREGIEAQEQAVRQAADAFHNELVLESRDAYQRAVRAKESLPKIERYNTKTVSADRVQSLSGWRLGRRPGGFQDPNVDEVLRVTDDMGYPREPKGCRDHGEPGRYYASHAERQEALTAETPAVGVSKELCEDCPDWFRHFAQHRGRTWYVTDPDGTWIFGADGSVTMPSGQQIAFGELFPGKYLNH

SEQ ID NO: 70 - protein, 257 aa, unidentified
  REGION 1..257: note = Isolated from environmental sources.Organism unknown.
  REGION 1..257: note = MISC_FEATURE - HmDa01
MYFAAKDAEGAYKLGQHIREAGWTEFSEETWEGIKKPFEEGDTFHASAETGQALFMMYGMARPFVPGSPTEAAAADATAATRSEVLSESATTRTPPIANEPPGVSGESAPKGFSDVPTDNYNHGPFEVPEEGNFDTGGTRTPRLGPRGVDPNHHNANVTVRDADGNIIHHERIVSGNMTPEEKALPFPKNTLASHTEARAMRNVPLEEGQTMTITGENPPCPSCKGAMNQRMRETGGTVKYQWREGGNTQKWPPDKK

SEQ ID NO: 71 - protein, 274 aa, unidentified
  REGION 1..274: note = Isolated from environmental sources.Organism unknown.
  REGION 1..274: note = MISC_FEATURE - HgmDa02
NLMTLGLINLKKLGMQILAAGAAKISGAIRKMLTPKVKFKLGSETHELWVEQDKTRNSVMMASTKGTEVEENSKASRILAESGELKDKLEKTKKKQNESQANANLQDLAQGIQAAGQGESKAANNGPWEEIDRFRSKTKGLEPLGDRIPERSDGLGTVAYVELNGTKVFGVNSSVLSDESKDLGRKFLKSMQAKGFLNEVKSYGQGHSQVLTHAEGHALMRVWKKTGGNLGSNVVIYVDRLTCGICQENLPKVKEFMNIKTLQVICKNGTKFTF

SEQ ID NO: 72 - protein, 817 aa, unidentified
  REGION 1..817: note = Isolated from environmental sources.Organism unknown.
  REGION 1..817: note = MISC_FEATURE - MGYPDa13
MVTFLWLWVFARCWWCEKVLVGLRSARGAFGVVSPFVWGRPAGSLQFPPEAWQRVLWAWQGWLSRHEGVARGLFRLSRVSQRITVVLLVVAVLVVPQLAAAMVPAIWMLACLGVMVLLSRTRTISWRAVSVMFSVSVPWALAVAKATELVAASGGMSTSDDGTSIALAAFVEEPGKLVPLAVVTLVAPGRARRLAAADWALLGYAAGAGFTAAEDGARRLAPQGMLASLLGGDKGLDYSLNAWTAGSFRLWNSDGLLGRFMASGGPSPLAVGHHVSTMTVAMAVGLGIVLWRTGRPLGRVVAWVLPAVALVQVVVDHAAYNASVASLTSVSWLDSDDPLVYWLGAVWQVSGRGNNAIAYSVVLFGLCLLADARRRLRTGALGVTAGEAPRVPSMTAIGGPAFVRAPIEAVVALVVLSYSDLVVIARGYADRRMTRSQRMIEGRLTAAQVMETRRDAMAATTPGVEPSARRVFALITLVVWALTGLVCLWCGVVVAQAIGSSLLFGDSDSGFFAGLLDELARWWESLGPMGQLLVTALGVMLLMSAGSTFALAMGAVGVLTWAAAHGHGLASFIHNPAAATSSYLSNVTAGQLAWDLLDFALTFIPGSVFGAGAHTIARTTARDMAATRTALRQGGKKTPQATEQATARTHAQRVAESQAAHEKSVNAANEVHKQRRYRTKTVSSEHPAESDHALSGWSEERRPTGFQRPNVDEVLRVTDEMGYPRTTHPYDQGTPGLYHASHAERQKALTAEWPHLGVSKPMCEDCNGWFRKFAHYQGRDWYVTDPNGVWIFRKDGIVNAPDGRIFNPNEVVPPTYF

SEQ ID NO: 73 - protein, 488 aa, unidentified
  REGION 1..488: note = Isolated from environmental sources.Organism unknown.
  REGION 1..488: note = MISC_FEATURE - MGYPDa11
MGNELVAEVKDSETVWSGSRLIEDGFDLKEAFESKSWVAGGLGTVATALDTAAAVMDPLGEALSAGVGWIIEHLGSLNEWLNELTGDSDAVAAAASTWTNIATKLSSCASELDKVCTGRLAGQESLAVATFKTLQAGSASHLRMTGQLAGAISGGLTVASMIVRMVHDMVRDAISDVIGKLASKVAIGVLTAGLAAPWAVSTAVTEVSSWVTRLSKEVADTVLSAKNLKDLLYTARALFDDVATSFAHIPSKIGDGLASIPTKVSEAASRGMDNLAKGADRRIENVLDVVDQAMPQTRAVTPDGQVLTLADQWRMMRQTDTPRYSGGGSGPRGGNGAKSVPVKKSQEAYDRAKELKKNVSSEKRVFLKKTTSSDHVETLSGWSMKGRPENFQEPNITEVLAKQEEIGHDVRRAGALDHGVDGQYYASHAERQLSINAEEPYIGVSKVCCKNCQQYFSKLAQHEGRDWYVTDPKGTLVFGADGSVVRQK

SEQ ID NO: 74 - protein, 1885 aa, Propionibacterium acidifaciens
  REGION 1..1885: note = MISC_FEATURE - d36_PaDa02
MVDLGAYEEPVAFDDGVADALRSAASALSGTLSGQAASRSSWAATASTDFEGHYADVFDANARAACDDCSNIASALDALAADVQTMKDAAASERDRRRQAKEWADRQKDEWAPKSWIDDHLGLDKPPAGPPETPVVDAQAPTVATWSEPAQGQAGGVSSARPDDLRTYSSNVTGANDTVTTQKGTLDGALSDFADRCSWCSIDTSGITTALAAFGANNTNETRWVDTVAAAFEAAGGSGAISAVSDAALDASLQAAGVTQSRQPVDVTAPTIQGDPQTSGYADDPVNTTTGNFIEPETDLAFSGGCASLGFDRVYNSLSAGVGAFGPGWASTADQRLLVTEDGAVWVQPSGRHVVFPRLGNGWDRAHNDTYWLHTTTDTTGPTPGDAPTTGAAGGAGVFVVSDNAGGRWVFDRAGRPVSVSRGPGTRVDHRWDGDRLVGLTHERGRAVTIEWNDHHTRITALTANDGRRVDYGYDPAGRLTEAASAGGTRTYGWNEAGLIATVTDPDGVVEAANTYNEHGQVTSQRSRFGRLSHYTYLPGGVTQVADEDGGRANTWIHDQTGRLVGMVDADGNRQSIGWDQWGNRVQITGRDGRTTVCRYDARGRLITRQEASGARTDYEWDEADRVVQVTVTDTTSSSHGNTSSAGGSGPSVTSYEYEGAGRNPSTVTDPEGGVTRLTWDQNLLTEATDPAGVRVRLGYDGHGDLVSTTNAAGDTARLVRDGAGRVVAAITPLGHRTEYRYDEAGRLASRQDPDGALWRYEHTTGGRLSAVVDPDGGRTVTEYGPGGVEEATTDPLGRRLEQEWDDLGNLAGVRLPDGREWSYVHDGLSRLTETVDPAGGLWRREYDVNGMVAATVDPTGVRRGLAWAADGSVTVSDASGTARVGVDGLGRPVSVSVSSAPAPGEAVPMGMSLEETVGTGAPAPGGAGPDGPDARVVVRDLCGRPVEALDADGGLTRLMRDAAGRLVEEISPAGRSTRYEWDRCGRLSAVIGPDGARTTMAYDAASRLIAQDGPGGRVRVAYDRCGRLSTVTAPGRGKTTWGYDRAGRVRSVRSPAWGLVRFGYDPAGQLTAVTNALGGVTRYDYDECGRLVQVTDPLGHVTRRTYTAADRVETLVDPLGRTTQAGYDAAGRQLWQTDDTGERLAFGWDEAGRLERVATGGEGLPGQTCCALTRPGRRVLRVTGPGGARDELVFDRLGRLARHARGGRTVGEWSWDPDGACTAFTGPDGQRVRYAYDDAGALVRVEGTAFGPVTVRRDTAGRLTGMDGPGLTQRWDRDETGHVIAYRRTKNGVTTSSRVSRDESGRVTAVDGPDGTVRYGYDPAGQLARIEGPDGRRESFTWDKAGHLTRRSVERPGARPETTLYSYDPAGQLASTDGPDGRTLYTWDAAGRRTGQDGPDGHWSYSWAPSGHLTAVTRRTPHDARTWRISRDGLGLPRRIDDTDLAWDLSGPVPALTRFGTHTVTGLPRALAIDGTLTSTGWRPARPTSADDPWAPPPPVVETDGARLGVGGAVGLGGLEILGARVHDPTTFSFLSPDPLDQPPLAPWATNPYSYAANNPLAFTDPTGLRPLTDTDFEAYKHDHGGLGGWIADHKDYLIGGAMVIAGGVLMATGVGGPLGGMLIGAGADTIIQRATTGQVDYGQVAVSGLLGAAGGGAASALLKGGGRLATELGATGLRTAITTGAASGTASGAGGSGYGYLTGPGPHTVSGFLTSTATGAVEGGLLGGASGAAGHGLSTTGKNVLGHFEPTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 75 - protein, 275 aa, Bdellovibrio bacteriovorus
  REGION 1..275: note = MISC_FEATURE - BbDa01
  source 1..275: note = HD100
MLAEKRAEHIAFLLASEGGEVAFVEDKGTVYFARFPVGVAAPSSAVVKLLQGLFDRFVDHSFFILRQRIYTTAGLTEMCRGMVKVVAKRITENIRPRDHGELAGLQFVEIGDASQILLPVAYLSQENQKSIQEVAGWLGSHAAVIPARQLELASGLARWVPRGSVLHDYDRDIAAFLLNDQGQLLSYGVNSNSKNKTLHAEVNLVQRLHRETGRPIPAGAVLYSTHKPCKMCAGMIYHWCEDPSQLKVYYSVEEKGGLSRQTVLDQHGLNHHISK

SEQ ID NO: 76 - protein, 1876 aa, Planctomycetes bacterium
  REGION 1..1876: note = MISC_FEATURE - PbDa02
MSHATSPSGLVTRFTYRDPTTDPDPGDPDNRLRGPSLIESREGSGPAVWTIDHQRSSSPVGFVVTVSDSLGRSAIHSTPARAAINKGRYTSSWTTVAGDWSRKVDLFSDRQSVARRIPFNNFSFASTYRFFDEGRTHIRVLGFEEGGSRAHLALDGFWTTQVEPLQGPTQNYAIETKQGGKVEGLIRNVSWEVAPGSLATVGITRNEDGEVEEVMNPVGVTTTTERYLSANKQGLPSAQIVAPGGTTYLPLGATPLREEFDYDLMGNVTSHRDTAGLATITAYTALDQIREVRLPSGLVQTNVYDQAGRLQSTSTNQGGRVTYVYNEFDQVIRVDQEGVGSTHYQRDEMGHLTQVTTPRGEEVRYEYDNRWLVSATEHHAAEEVAGPLELQRRITYQRIARGNVLHETRTEGSAGANSYTTSYGYTAVGLVRSVVRPPMTLGESFRPQTTYHRGSNWLVNRVEQDVREDREDKLASYVRDDADRVIAVRNGTAEATNPHRSQSEVLLDLAGNPNTFLSGDFLYDPSAPVGGTVLSDRPRSTVTRDGLARVKEVRNGSGCVIAELTYDSAGRVVVERRPNSNEQLEVVSRNVYDPLTGRLQSSEGYDYPGAGKAKREYYYDAEGRVKKTFVRDGGGNLITVNEVWYDAIGRVVRRQSRDKDTNPTEQRESRYEFHPQTGDVTRETQVVNGNSYTWVYTYGVGGRLETTTDPLGKVSRLVYDAEGRLQKRIKKDGVEITYEYDGWDRVKTVTGPTETITYEYDARNRVISEANALVKREFSYEDDLGRLESVKVTFLQVGLQVPKTFTYRYNAAEHRYGGLTEFVDPNGLETKYSYNRELDVERIEQRQGSAGPFSEVGSFAYDCSQRMTEWRTRGGSWERDYDATGRVLRQHGAQPNHSISYSHDAFGRRKTATYAHLQGLKFAWAYDGIGRMFDEQISYGDGAPAYRETLTFDEAGNRSSRILNGQTTLYDYNEFNQLVRSRERVRTVANLTLSATTGPPASPGTDVLGPSYAPGLAVDGTIPTTSGANQAFVSGTADVEHLLEVSVTSGAVDLTGLEVHLPTDRNLPDKLHLEYFDGSAWVPLPVQSATGADVVMSGSDQDGLRARGARVFASVWPPINTSQVRVRQAQGEGPAQPTTHDNALAVNEVRLFTASSEVEVETSYVYDDNGNMKGVTRGAYERTYEYDQNDRLISIDDVGGAVPVNMRMAYCPSGKLTRQEVSGSVEHLLYQGDELYARYDASDALTKRYILGPGLDRKIGVVPYSGGVAENTRYFFPTPNGSVHQVTEWDPLRNGGLGAAVLANTELTSAWGVPILQDRIPELLAGGGPPVVSIFAPDFGYTGRRKLGNSGLMDYRNRIYDPLLGRFLTSDPIGTKAGLNTYAYVNGDPVNLTDPFGLDPYDVHPRPEEWYNQPGLYNKFQALRGEIGALWNRHVNPWGDAGAEVLEETLDEGEKLPGAMRTLGEAMQADPGGTTRIMLSQLPGVARNMVMQPIDQTAEAHYYHLLGDAVGERKAVIKSTTGFMALFGVAQGTRSLALRTTGVLRAIREVCFVAGTLVVTQEGRVPIEDVSVGDLVLSRDPETGEIGFKRVTRLFSGKTTEIAEVRLFAPDLTDDSASVVVLTCTPTHLFRTRGRGWVEAKSLKPGDQLVTETGSLAVVAESPSIRQVEALSRFNLEVEDWHCYFVAPNESSASILVHNDCWDLINYYRRVHPNLPDLPSPDYVPGSQKIIPGSGVADKTLGTVAFVKIGERVFFGVNPTAMSDAMKNLHRLFFDTVLKPGGYSGTRSARTGKALTAGQYGGMANLFHAEATALLRAWKHNAIPKGGVLRMVVDRHTCRLCMKPLGEIVHALGLKGLEIVQKNGKVVRIKPIAPGQ

SEQ ID NO: 77 - protein, 417 aa, Pseudomonas syringae
  REGION 1..417: note = MISC_FEATURE - PsDa01
  source 1..417: note = pv. theae
MTSAARVGDPIEHSEALNGLLLGLAVGAAAVVVAAAVGVSIVASGGLAAVAVVGAVVSLSAGVGELIGSLSYCTNTAGHISKGSSNVFINGRPAARAHLDTAECDKHGQVPQVIAEGSSTVYINGQPAARVGDRTICDGKISAGSDNVYIGGETEQTDDINPEVPGWLHGVVLGIGFGSAVVLTSWATAIVGLIGGAVGGYGGGVIGGRRYGEGSDEQKVMAFGGAFIGGSLAGKGQQWFDSRYEFKIQGFGSNLGNLKIIPKGSPRASNVAESDAALGRASQAKSELPKTRAMRPKTVSSNDVKTLSGWSNKKPAGYEKVSSEQIKAKSEEIGHELKPHPYDREYEGQFHASHAEKQMSIISPNHPLGVSKPMCIDCKGYFSKLAQYSNKEQVVADPDATHIFKIDGSVEAILRSE

SEQ ID NO: 78 - protein, 2046 aa, Actinoplanes durhamensis
  REGION 1..2046: note = MISC_FEATURE - AdDa01
MLVSSRFRLLCHALIAAMIAAYLVSFPFTPASAAPVATAGAGPVNRPLPATAAANHGLPAGESAPPVPPAVATDGSKPRVPTAAEKAKRKLSAALAPTTKPIARGGFLLGDTSLVVYWDAEVNTGDPTSWGRWFATVTDTETGAEQRSIELGQADLSRCASPALLCRSFGAADGWVLDPARTYTVTVTEIAADGTEQTSPPSDAAHPRKVDDVPTVAAGQVVGCGCSTVLGPTVTAQALRGATVNTATGQYLRVERDFTMPSYGIPFNHARYYSSGNTNVGMFGPGWSSSYDIRIVAADNGAVRVRAEDGSEAVYLARADGSFDAPAGIRATLSRNGTGWKLVPPDQRVLAFDADGRLLSIKNARGLGITLAYATAGPLSTVTDASGRVVRFEFRTDLRLITKIILPDGRSTQFDYQDGRLLKTQDPRGYVTSYSYDAGGRLAEITDARGAKLIRNTYDANGRVTQQLDPEGGKTTFDWKPTEQIAVTVDPDGVTVSDGYKNNVLLYSQNGNSDTVNHRYTGKLNKNLVIDPKGNQEETKFDDAGNPISRTAPDPFLFTEVNTFDDRSNVTAVKDGRGNTWDYRYNQFNEMISQKDPKQGTGYEYTYDDKGQVSTRKDPRGKITEYEYDAYGDRTADVAPTGRRTEMFYDGTGRMTSVVDPRGTVAGASKDTYRTRFTYDQMNQVTETWQPGKGQPSRTTYDELGRVVVSTDPLSNSSRYTYDLASRLVEVKDPIGNVTATTWSAGGRRTSETDGEGNRTSWTYDNNGRVSTETSPRGNVTGADKALYTSVFHYDFNGNLIRTDRPYGTDGRRVEVDTAFDSLDRPTEQGDQFKNGTKVGYDNNGNVVSMTNERGEKLENFYDEANRRTGSSGGSGAAAVEYDDAGNVIKQTSPTGGVSTTSYDDDGRVVAVVEPRGNATGADPADFTTRYGYDLAGNPATETDALGNVAAAGYDPNNRVTSSTDANGNVTKYTYDAADRLASVIGPDATGGQALTYGYNADGQVIKRTDPLGHTASLEYDRAGRTTVSTDGIGRRREYVYDADSNLVEEVTARIVELGRPDPNRPARTIFYAYDNLGRLISKQLGSGGTAYTYGYDAKNRLVSAADPGGVLSYDYDVTDRLTTVTRGGEVFKYDYDSTDRIIGRTYPDGTKIGADYDDGDRITSLTATRGGTSANYGFHYDAADNLTGIDYPSATGITESRSYDRAGRMTRIAATKGDTTLSAFDLTLDKVGNPTKIIKATGEPGQPTITEAAAYTYDKANRITAECYGAQTCAGATAARTDYTYDLVGNRTTMKKVATGENTTTSYAYDAADQLTTETVSGTRSSSRAFTYDLEGNQTQAGTDRFTYALDHTMTSASVNGTTTNYAYDAQGNRVSGTSGTGSSAITQTWSWDVNASRPLLAQERQTGAAARDYLYDASGLPLGLIAADGTHSYLRDWLGGVSGVVSPAGAAEWEYAYDAFGVDRGTSKKADDAPDNPYQYAGTYQDTSQGDRYAMGVRSYDPSTGRFASADPATQPATDQAVSTYSYTNARPTVLTDPSGADPDGMADTYADYYQAMACANYPPACAATTDANTAAAGEGEQQVENPDWQNSKKLVDEAGSFVSKIGDEIVNLILDLVGFNDAKACITEGDILACINTALQAVPWGKMFKAAKVAIKAIGVGRRLVDAYSRLKSAKNALAAIPRYIKKLTQTAEEAADSKKYADEAAKAAKGAKDAGGKAKATSQKAADNAKKAKKKESTTGESCEVAKGGTTRLASNSFAAGTPVLMADGSRKPIEKVQAGDTVMATEPSAALTAPSQVIATVAGTGEKSLVDLTLVGAGAGGGGPPAVITATTGHKFYSLDRDQWVPAGELKLGETLRDNRGGKITLTGLSEHTAETTVYNLTVDDEHTYYVVAGDNPVLVHNCGAGADAESLAEAARYRADELHKALDPIARSKRTTAVISAYDHEGQIVDVVSASGRGLSRAQKELLGAGEYIAKSTGKRSHAEMNALAFIESKGWTPIAGGASRNVCGKCSAGLRASGARLFGGTFPGRGSRMRLFRWF

SEQ ID NO: 79 - protein, 2407 aa, Kitasatospora setae
  REGION 1..2407: note = MISC_FEATURE - KsDa01
  source 1..2407: note = strain ATCC 33774 / DSM 43861 / JCM 3304 / KCC A-0304 / NBRC 14216 / KM-6054
MAVTGSLVGAPPAFAEKPPKVWVPPTTPLQEVKSVSGRSDNVTGTAAVGGVPREWKRAAVAPAKAGSATVDVTDVSAEQEAARRLGGGAAPAAGGKAKVKAGDLPVFLTDLGAANDRDRAAGVPSVKVDLLDEGRAKAAGSTGPVVRLEGAGGAAKGGHKLQVSLDVKALTGNAMWSDRARLVLLPECALTTPDAEQCRTSTPVPSTVDPLSGLLTADVQLPEAPAVPAGTATQAGFVQQAGFVQQASAASSGAMVLAAQAAPSGSSGSFAATAIAPSAAWSAGSGSGNFTYSYTFDMPTAVGGAAPSVVLGYDSSSVDGRTASTNSQSGWVGDGWDYSAGSISRSYKACLKSGIDMSGDECWAGQALQLNLAGHSGTVVKDDTTGVLRLQGDDGTLVTPLTGLANGAWNGEGFKVTTTDGTQYYFGANHLPGGDNTDPASNSVNTVPVYHPHAGDPCYSSSTGLASWCQMGWQWNLDYIVDLHGNLIKYQYAKESNSYARGGGQNNGTGTRTAYDRYSNVSSIQYGLRLSDQIASKGAGNPAAKIDFTTAERCFASGSITCSDAQRVVANQSSWPDTPLDQACTASGTCTNYGPSFWTSKRLTGIQTTVYVGGAARVVDSWALNQVFSDPGDGNAPTLWLSSIQRTGKNGQADIALPAVSFTAMQMANRVDGLVPAAPQFNRPRMKEITTETGGRITVNYKPAECSRVNNTMPASEAGNTMACMPVKWVPPGSQAGTKPIDDWFHKYLVSSIVESSLVTSSIVAKETQYAYGGGAAWHRNDSEFADDDTRTYDNFRGYQSVTTTTGSGSDGPRTKSVSTFLRGMGGQVTDSWGSTITDAEELAGFVRETQNYESDAAGAKVIGGELTTPWLSKITATHTQSGNLPKVTARYINTANVKNRSLLSNGQWRTTERSATYDEDHAGRLVTMDVLGDTTLSGLPQRQCTAVTYASGSNPALVEFPSRMLTLAGACAQTPSATNTISDALTYYDGQALGVIGDKGDQTETRVLESYGTGNTPNYKVTAKAQFDAYGRQTSTTDPNRTDTAHPNGATTSTAYTPATGELPSKVEVTNPLGWKSTTLVDPARGLPTKATDENGRTTLETYDALGRLTAVWLPGRDPNDLDGNDTPNRKFTYALNGSTSPSVVTSESIREDRTYAKSLQIYDGLGRVRQSQSIPVQGQVGRLISESFYDSHGWQIKSTPSYYNADSGPIATVYLPEDSEVPTQTWNEYDGLGRITASKFISYGQEQWRSTASYPGTERTDATPPPGGYATTTLTDATGAAVEVRQYKAGTPTGPYDATTYGYDVAGKPTWVQDSSTPAHRWTYTYDLLGQRTGSTDPDTGTSATVYDKAGHAVTTTDARGKSTSITYDLLGRQVASYDGTSTTDASKKLIEWTYDSLALGKPAATTTYADVPNGTGKVAYVGAVTGYDIGYRPTGMSMTIPSVTGEEALAKTYTWSTTYTPVLGLVKNTKLPAVGGLAAETVGNSYDVDGNFQTSSGLDYYVQDMQYDAFGRPTRTTVGKAGKQVVSTQQYDPGTGNVVQSTLDKQTATTTHVDFTNYTYNKVGSMTSAKTIQDGTSTDLQCFTYDYLGRLSSAWTDKGTTTTAPAPSVQGIGNCTNATEPTALTASARIGGPAPYWQTYSYDLTGNRTKLVQHDTTGAVVNDKQTDQTFAPGPNTATTDPTTGGGTGGPHALLTSKTSSNGTSKVTTSTYDATGNTTSLTSTSGTKTLTWNNQGKLAKVHDTAAPANDTSYVYDADGNQLVRRVGGKTTINLGTDELTLDTATGKTTATRYYAAPGGLTITRTADAAGATTLYYQASDPHGTAGVQMDAANLGVTRRPTDPFGNARGTQPGNGAWAGDKGFVGGTIEGTGFTNLGARQYDPATARFLSVDPLFDNSDPQSWNGYAYANNNPVDGSDPNGTCRVLDDGYCHGAPFIPVNHNSGDDGSGTDTSTSTSATGKAAHDASQEALRQKQAADEALAKAKQAREELISKIVDVVGDLIGFNDARDCFTKGDVMGCINTALNFVPWAKVFKAVKIGIKAFKLWREGEKAYTAIRSAERVAKEAQQAFSVARKTEKEVAEAEQQAAKAAEADAAKTAKEADAAKADSAGTSESSASCPAGNSFPAGTLVQLADGTTKPIDQLEPGDTVLATDPQTGETRAEPVTATITGHNDTEFTQLTLQGTESGDSLTVTSTAHHPYWDETSHQWVDALDVKAGHQLRTADGTLITVTALRTYSTQPSSAHNLTVANLHTYYVLAGATPVLVHNCGGELLDRARELYGTRADEASTVAVARVRSTSNPDKVETWVATERTGLPDEWKGGNAPLRGERYISGQGHAEATIMNRLGSDWEIVGMASSTRMCPTCFAQATGPGVGLTPSPIGKGAGVSRTGNTPWRVVLGGGG

SEQ ID NO: 80 - protein, 141 aa, unidentified
  REGION 1..141: note = Isolated from environmental sources.Organism unknown.
  REGION 1..141: note = MISC_FEATURE - VRDa06
MEISEKKQKRLDSVFIEIAKQIGTLSYCTRAKVGAVLVKAGNLISFGYNGTPTGMDNGCEENNVTKEEVIHAEMNSILKAAKSGNSVDGSTLYLSLSPCQNCCKLIIQSGIKRVVYLEAYRDLKPVEFLKQFIKVEKYEVQ

SEQ ID NO: 81 - protein, 1167 aa, Sphingomonas sp.
  REGION 1..1167: note = MISC_FEATURE - ScDa03
  source 1..1167: note = CF311
MASSPPPILFTPVAARDLAGWAAAGQLYALVDGCVSPDIPAMAEQAGQGRAACLYLGRAAINYREQAPYLFRVDPAQAEALATRFDGEPWGVFLVSGAGFEATRRHLRRFLKVRSPEGAGWLFRFYDPRLLPAFLRSSTPDELNNFFGPVQAYLTVDARGEGMMAYRAPETVRAAPRPAVGARHRISEANVAAFRRRALGDRIAASFEGTPQKARREPQSDDVLVTNPDGGVMRLGFGASGMLDRVTSPLGRQWRVASNEKGQLKGLKLPSGLALGLDYDAAGDITHVSRGGETRFRAGYDEQHRLTRIDFADGSFSGIRYRDDGIEATRDTRGKYIAAQRDRVGRVERYDYAGDTLVAMVDGNGNATRFDYGAGERPEALVQPDGGREAYAFDPAGRLQQITRADGHVLDVTQDEAGRVTRLATPDGEVSTFTYDDRGQLVAAKNGDATLAWTYDEGGWLIEERQGDQRVSYVYDEAGTLTGLTYPTGETIRYTRDADLRLSSITDWAGRRHVLDYAPEESGWRLTAPDGLTTDSFQNPIGLTTGVRVELAGQPVFEQGFAWDGDDRLAEQRDSRLGAMRFGYDAEGQLLAVERSNGAPEAFAYDGAGNCISGPTGPAQFDALNRLIGQGSERFDYDGRGNCIARSGGAQAWRYAYDGFDRMVRAEDDRGRVIRFGYDPLGRRLWKEVTQGGEQRITRYVWAGEQLIREIEQVHGQAGEAWQGVGPERTRDYCYWPQTYKPLFLREGGAIHQYHCDPLGVPMRLTDSAGNVVWEAERSGFGAMTALLGGVDQPLRLPGQYHDAETGLHYNRFRYYDPALGRYLSRDPIGVAGGLNAYAYAGNDPVNRADPTGLWWKAALAIVAAVAVAAVVVLTAPISGPLLIVAAGAAAGAAGAMVNEALNQKEFCLSCILLAGLKGAVVGAIASVPFLLLPEIAGLGAFIAAGASSGFIGYAADWGIDNLTGKPRPWSWKDAAISTGLGGAFGAAGRYISGGRTGTGRGVVPEETAPVAPRAANAADVAESQAALGRANTAKAGLTKKEYAVKTTSSNDAKTLSGWKQPRPEGFDGATAQQVQNKASQIGHELEPHPFDKDFPGQYNASHAEKQMSVLSPDHPIGVSKPMCADCQGYFAKLAQHNGADQVVTDPNGTWIFKSDGSVVKPGDPLP

SEQ ID NO: 82 - protein, 535 aa, unidentified
  REGION 1..535: note = Isolated from environmental sources.Organism unknown.
  REGION 1..535: note = MISC_FEATURE - WWTPDa04
VNWRVLTPADQFELHGYFQTNGNTDIDEIAEGFNLTGTGLLGVSVVPTPQTKAIGGIGAGLCFATSSGLNLYQRTSQGFNTRHDLLENGMDLAMIALSLSGAPRIAGNYLAKVHTGKLQVFGEGLESLFLHGKKVVVLAKLEVGAGRSLLCVCGIQAIQSVGSIVGRDDLSTDEKFHLLVETLGLFALQGLLMVVSRRTDKYVKYPTRSAESPPPPKAMGKTDHSPLENGKPLEGESRSLKHEGEKPPDADGRGKVGEGEKTSDSENGAHPETEKQPEGESRALSEEERSSPNESKPKSMESETNLDEIPGQTGAERHETIVSRGKRGKGEPKEDARKPEEPPKPEPVVEPEPPKAWPRETDAPPPPFEKGKPAERSWNQWFQNERMPNVPNVKLGELKVVAYAEINGKQFVDYNQNARLKPLADEQTAIYEIIKRRASKLGKEISGFPNWKHLNLHAEGGLLYQLYRAGLTRGAKIVITVEGRKVCGFCLKDLIELAKALEIKELTVFEKNSFNELPPETLFWKEGENTWTDLN

SEQ ID NO: 83 - protein, 846 aa, Catenulispora acidiphila
  REGION 1..846: note = MISC_FEATURE - CaDa01
  source 1..846: note = strain DSM 44928 / JCM 14897 / NBRC 102108 / NRRL B-24433 / ID139908
MATFAIGGRLRRSVAAVATAALGLAAFVAPTTAQAQAAAGPGSAAQHPASALGRSGFAMADPAPPPALPAPLDSSGPAQVQALEQQYQTDAQNDQQKYDQLAAEKGTLEQRAGDVQNRESSLETQATNLEDQSNALNSQAETLNSEIDAHNAEPHTFELPDEEAEYAAYNEEKANLDEQKANLQNQIDALTAQSKKLQSDQAQADADQTQLETDVQTHNDAVSALEGDVGKLEAERQQILTQIDSLLQDYAGAEPGGEGAPLAAEGGDESEPAAAAAPSPGARALPSGGGDQSAPPRSTYAPVQNAPSGSGSGQAQAQAAPAPAPTQTPVTVTLAPSTVSGLPASEAENLQPSETFDGLIPEANGDYAAEEIQPPAGESVPPAQKAFDNVVNKGGKASTRIGGRPATIDKIVPEAAAPAENQGGDTPRPAKAAAPPARSSWVPAVNQPNPANGPPVSIDALKSLLDQQGLGSDADQFDLEYSPTVLGQDGEPAYAVAPTDAAGNPELGAEGKPILRFSNLGLQNPEVAQDAFENEGLDVEPAQDDSPCPHSFSGATRVLMADGSTKAIAEVGVGDLVENAEPGGRAEVHRVDQVHTTTTDAAFVDVVVASAAPGGGGTLTGTANHPYYDATAGGFVDAGALRAGDRLQSAGGGQATVSGVHARFGPLVTYDLTIDGLHTYFVVAGSAPVLVHNCDGDLLDIAKDSATRASGYQKLAGSDWEVKNKTTSVIRARFPSGDPKNPWVYKNVVSSSGSGLSPAQISAIEANGDVAVTDNLEGFTHAEYNGLRYIDSMGGQPIAGGASRSVCTTICGPFIRGTDGNISGPVYQLEHGTKIRTFYWPGSTPG

SEQ ID NO: 84 - protein, 3065 aa, Streptomyces pristinaespiralis
  REGION 1..3065: note = MISC_FEATURE - SpDa01
  source 1..3065: note = strain ATCC 25486 / DSM 40338 / CBS 914.69 / JCM 4507 / NBRC 13074 / NRRL 2958 / 5647
MAPFLPVAPNVVLLDMLRLIFVTTRSRKASVERGETMFKRRTRRALVISCVVAVAATGVPQPAFAAPSGDGGTGIVRGLKNFFGVDDDKPAAEPPELPDTAVPSNEQIPKGRAAPAPKRVKELTDRRTPDARYWQLSDGRVQSEVSAVPTHYEAGKAWQAIDPTVKPTDRKGFTLANTTNVARTYFGERPGELVRFELDEGHWAAIGVKGVTGSLKPQADGSTVTYKNALGAGTDLTYTVGNGTVKEGIVLDAAPRTAPSYEFTLTTGGLQAVRRAGGSIALYSESSGPTRPALVIPAPFMTDAKKDGESPYEAAGTDRVEQKLSGKGTSYTLTLRPDAKWLSAPERRYPVVVDPTISIAPTSSQSQDVMISSDGPASNYDGNWRLSVGNTGSGSSRALLRFPLGAVPAGTKLDSADLKLYFDQTHTTGDSEVRLEAHRATAAWDETTATWNSASGITGELSGTSVLVDDRDSGTTAAKGSWPASGNTAFTQYAVNQDYLYNKDSVAGDTYTWQPNLPEDGDYQVEAHYVPASDRAANAPYTVTYSGGSKTYAVDQRAGTGGVWKTLGTHPFKAGTAGKIVLGDGPASTSTAVLADAVRFTKGGVVTKRPGESNTWHSFPVTRTVQSWIDGTHTNHGFVVKAGDESAGGPKGGPRYEAAEYAYKGEVANFPKLVLTWGRPGATLEAPQVIHATGAELKWSSYQDQDPISDRDDIVEYQVHRSVFQTFTPSPQTLIAPVPEGTTSYTDTTATPTPADSVDAFGNVFYYMVAVKTKDGQVVPAPTRIVKLPKAGRTTLVLQGDAADTTLSTTQPATGHDALEDSGIGRPWLSVGNNSSTYGTTRALLKFPALSQVPSASRVIDAQLDLWGTTTTTGSDGAVYEVHALNKAFAEADASWNKANATTAWTTPGGDMDPAVADTVGTVTNDPARQSWNVTSLAQSWVSDPASDKGLAVRLKDESSTGPQERTIFLSSEAEEPQLRPQLVVTYLAKSTDSTYFSPYTPARMTPGDRYTVDVTVTNTTTSAWKAADQALSYTWSLPDGTDATTGGNQLFTALPEDLLPGQSATVKAALQAPINSDSGNKRLDYVLKWDLRNKTAGTWLSATESIPPLAQNVRVEDPTSNQLGLEKFYSYAGKNTGAGSSMMTNLYAGNTVWQYNAFSNPGRGLTTFARFAYNSQDTSDTVLGHGWSAQAAMPLRLGTALDFHPNPNPTEVTLPDGDGTEHTFRKQEDGTWKAPAGVHYLLRQGEGVDCTPNKDGDPKAWSLTRPDRSQFFFDCDGYLTSITDKNGNTQTYTYEERKSNNKPVKFLRYITDPAGRQSLTVEYYTKADTSSPKIIDHVRSMTDISGRKVTFEYSDKGLMTKLTDGAGSSQPKVFGFVYDAEQGNKNAKLVKVTDPRGNATSLDYYYPSEGDDPKFHWNTQTITDRLGGTTGYVYRDPDATAGSFIETKVTDAENHTTTQSLDGYGRPNTITNAKSETTKLAWDADNNVTRLEEANGAFSTWVYDQKTGYPLEQKDAEANKNGTAAQTFTYSTGLNGHIADLFRKKSPEGRTWQFGYDTRGNVTSVTDPKGVASATEGDYTSRSEYDTYGQLIKAIDANGHATANSDFQPVGYPGTITDAGGHTSAFRYDVRGNVTTVTNAKGAEVTQTYDAFGRPLEKKEPKDQAAGDFIVTPAPVYDANDNITKVTAPNGAVSTAGYDAADQVTESTVPGDLATDAPRRTTTTYDKVGNVLTTTSPKGNETATVGDHTTTTTYDAIYQPVAVVDANGGRATSEYDNVGNIVKETDPRTNATPDPDDYSTKYEYDLNHRVTRTTDALGWTTSGRYDKDGLAIGRTDAEGNESLATYDERGALVESRVPVSKDAGGTIKYHTTRYEYDPVGNKTRTITPRGVATTDDTDDFASETVYDELNRVKEERSPFDKDTPGYTTPDTTVYAYDEVGNLESVSAPPSDGQTVRNTTRYTYYDNGWARTSTDPWDITTSYDYNQLGQQTKNTITSAGGSQQRTLSWDFYRSGNERAFFDNGVPVGRQAVLVDSSDFHNTATQGTWTRGQAEQQYGHDIASHPAGTGTAQFVWQLNIPQNGTYEVFVRHPKVSGAATDATFAVTHDGGTTSRSVDQTKDPGTWISLGKHAFVEDGPQKVTLTDKANGTVLADAVKLVRDNTGESDTEQKDFTYAYDANANKVEVKDNSPGAKIDTYRIAYDGINRVSKVEELAAGAVKNTSALTYDENGNPLTSTHDLTWSKIEYDARGLVAKVTNADSPTAGNQQITTLTYTGRGQLLRQTKPNGNTLDLTYFLDGSVKQSVEKTSGGTVVSQHDLEYSPNGHRSKDVLKLMNADDSTAYINNTYTFDYDPQDRVTKVGKAGDSPSTETYSYDANSNITEQSVAGVTTTHRYDRNRLLSSTTAGVASTYNYDPLGRLDTTSTGGQSLEKYYYDGFDRTVKTRSGTGAAATTTNYVFDPFDRTVSQSTSGTDPKTTAFTYLGLDSTLLREEVDGKADKSYQYLASGKRATQIKHKDDGAKEYSQYTTSPRGDVEAITKEDGKTRSTYGYTAYGSDDESQMTGEDKPDAQNPDKESYNAFRYNSSRWDKASGTYDMGFRNYDPGLNRFLTRDSYGGALADMSLATDPFTGNRYAFAGGNPISFVELDGHLFGLSLSDIGHAALDVVGLIPVVGEVADVANGIWYAAEGNYVDAALSMSSAIPLVGYGASAVKAGRYAKKGMDALDSANDARKTTDRAGDAADAGNDVRKADGDAPSTPAKTDAKTCKVNSFVPGTQVVLADGSSKAIEDLREGDNVLATDVESGETRGRQVTDTRSHEGDKHLVTLTVDVDGTEGHATGEITSTAGHHYWLPDSGRWAKAEQLEPGMWLRTSSGTWVQITAVDHDHAKKRVHNLTVAADHDYYVVAGGRSLLVHNECGPDYDQAASDKAGALHENLKVPGKKDGGYAYKNDTTAVVMARDQAGNIRTIVASNRRYVPKAIKSKLDESAGEVFANGKGHAEMTALQYMDSQGWEFLGGAANRNVCAYCENAVRGRGLKLSGPTFPGFVQTLAGRFQFFGERMFRR

SEQ ID NO: 85 - protein, 182 aa, unidentified
  REGION 1..182: note = Isolated from environmental sources.Organism unknown.
  REGION 1..182: note = MISC_FEATURE - MGYPDa14
RSTLFPYTTLFRSARFNPELARKSKEAYERAKAAKDRLAKRYKDKTASSDNAKTLSGWGKRRPNHFQKPNIREVFEMQEKIGHPRLPHFRDNGVKGRYLASHAERQEAVTAVEPAIGVSKRLCEDCHDFFIRLAQHEGREWYVTDPGGTSVFRPDGTRLGTDGEVTGLGQGYRYSERGARRK

SEQ ID NO: 86 - protein, 242 aa, Actinosynnema mirum
  REGION 1..242: note = MISC_FEATURE - AmDa03
MASIGEVADRLALVVELVGRCRDALAAARVLVREARAGLAEALEGAAGLEGDAARALDAFAGVADGVRDELGPLLDGIAARLEAVRAALLGEGGGAGDDEPPAVPWDRVERLRRELPPPVVPNTGQKTHGRWIGPDGQARPIVSGRDDKSVLVNPLLRGKGAPGPTRRDSDVEMKLAAHMAARGIRHATVVINNTPCRGPLRCDTLVPILLPEGSTLTVHGINENGTRTRIRYTGGARPWWS

SEQ ID NO: 87 - protein, 158 aa, XP12 phage
  REGION 1..158: note = MISC_FEATURE - xp12da
MEVPTKRSQEQWDGFFYRMAVLAAAMSKDPDRKVGAVLVTPDRRQVSPGYNGFPPEVPDLPSLLADRAFKLANMRHAEDNCLRQSPFPTAGCTVYVTSFPCLPCACKLRDAGVRRLVAPRPDLGHHRWGKSWATSIAVLTLAGIHITYLEEYNQCDCC

SEQ ID NO: 88 - protein, 172 aa, Bacillus virus G
  REGION 1..172: note = MISC_FEATURE - gp317
MVNKNMEIGIPKLEEYYMMMAFISGSRANCLNRAVGCVLVTPDNGSIISTGYNGVPKGLPHCTTCRRREEGFGPGEGLHRSRATHAEANVIIQAARYGRKVEGTIMYVTDMPCSDCCKLIINSGIKKIYYCNEYPGSEAAEILTASNIEVEQLEKSKIMRHLKLYVDAIGMM

SEQ ID NO: 89 - protein, 1624 aa, Acidobacteria bacterium
  REGION 1..1624: note = MISC_FEATURE - AbcDa01
MAAGASENSGVQFKCDCGGFSSGECEECRKKREEESHSDHQVQKKCATCEEEDEQVQHKQAAGGQAASVHGTARAGVARASQPLPHLDRIQSSFGRHDVSSARAEIGGPAAHANASMGALAYTAGDRIGFRREPDVKLAAHEAAHVVQQRSGAKLAGGVGRPGDAYERQADAVADAVDRGESAEPILDRTISSNPSGSEPVVQHALAINAFHLTEPPIAMTRPTIGSAPEPIGIGGRSEGATPAQGASQVAQSAIAQADKGAAPAPASQPAPPVGGTEADTSTGGSFRAYCYNADYPVPDDDSDEEPKDPDTTKAKEEVHPDLPRPDETDICPIEVAVNAQAPLSSTSAAGESKAISAASTVPSAKPGSPPSGEAAQKTAPLGEEEIQSGTEAAANAPSPLEDAIAQAEAGRDNAVASFADSDALTGTLSPRITALAAPVQFLSDSSTGGDLEQNRTAEARVSDFFGNASQRLFEGLTLATDVVPDQFGATAEVIKSNIGEAIGHQKDVLSANVETARAQALSTADAARAQVHADHNATVAAINEQADSAIDALISSHDGSIGAIDEIESASLDEVNATYSESYDAHVALGPEYADLAEARGQQYAEFYQGCKKYNPDGTEWKDGFFAGYLTNRRAQAEVNAANETVKGYRKSLTETANDQAAQAMKGRKKDRCGIIATGRRTRTAVDDRLTTLMDALEAGREESIGQANATRHTMLASITSGLSSALAALDLLEHDGKQSLDDTGYLQQLAVEQAAYASATAVQEAVAQAVENVESALAGLRATLGESPAPALEDLDRILAQAAVGLNGGIDLLLACVDASITSATARLEASGSAAFAALDSDAKTSSDRINSQSETFSSQMDSLAAGAASTFGTMRDRHAEQAQQTSDAGAEGFTEAEIGFQNACDKTLTAIDATLTQSEQHLAQDFQKQVGGLDDDTKTGIPFQARLAASKEQPAWKSVVAIILIIAIIIVVALVIGPAVIGAVGAAAAALGASAGVATAVGAIVGGAIVGALSSGAMTLVQNWASGQRLTKGLGHAMLIGALTGAAGGAIGLGVNAGLNAALSGGRLAMSAGAQFAVRAAVNVTSDSLLNIGQQMALTGHVDWGEFAQGVAMSLVLHGSRRVQAFQARVTAVGARGVAGGVSRLGGGVEPSAASARASEFAAKMEQHAATAQKESKEPLDWTRPTRAPEPAAAPPPPESTGKATPSEATPPAPAETKAAAPPPEAATPPAATPEAAVTPEPVESAATKVTGEHPSPAAVHEAEATTPSGPAAEPDVVPEIGPEPQSATKVGVDDHEMYVRRGSKGVEVGMCSGVCGPLKARIDEMQAALGEPDSKLHGQLEDLRTKATDLEQRIDRGELTKNEIEREITDLAARLRLAAEEHPLLGRSLEPDVPATAGPLQAEALFDEPLTESEKGLTKGQLRQKHILEVQAERQHAARGASTEPEPVGRIPETPKAPLDLPEYQESIEAARRALANLPEAPGVKTTAGVEGSSPTRSGYKPTKQFPMAEEATERAVAEGHEAGRAPEPHEFDRSATAGSAGKVRGSHAEKQAAVLSPDDPVGVSKPMCSDCQAWFRQRAIDRGVPQVVADPDGTRVFMPDGTVHFQQHPEGFPGPRGNR

SEQ ID NO: 90 - protein, 2423 aa, Wolbachia endosymbiont
  REGION 1..2423: note = MISC_FEATURE - WcDa01
  source 1..2423: note = of Cadra cautella
MEHNKDTSQKSTIEAKKLELPKGGGAIKGISEAFEANSFTGTGSFSIPIPLPTCRNFSPSLSVDYSSGGGNGVFGVGFNVSIASIIRKTSKKIPTYDDEDIFVLTGADDLVAINQTSNSEYDITYYKPRVEGLFAKIERLISKNDNRVYWQVTSKDGSIAIYGKNEQAQIYDPKSNRRIAEWLLEELSDVYGNKISYYYRSSKVEQGSGRVIDRITYGHNKELKYNHFEVLFDYGENDFTSPIDKKYNEERKAIIRPDPFTSHITGFAVRTELLCRAIRIYHRFDAEPILVNGTYFDYDERPTITYLRAVKQVGYYEREGKRFTKTTPILVFSHTSDDSTYISTVSSVTLSDGSKPSKELLQILKEQDFAFSEQNAEISEENSNWKIKDSEKTLVIKEKGNFLDLYNSNNKNSFKPVKIQGFAGYLNDRRFQSVDLYQEGIEGILYNDGNTILYFRPNGDGEFAQAKLLERFPSLLRKTTYSLTSLEGNGELQLYVNESGFQGYHDFDSNSKWSEFKPLESQVINLSSEIQEMIDVTGDGRTDIVVFEGESVRVYPSKGKKGYKKAIYSKAPKDLPNQRASSESTLVRFADMFGDGKSHLVKIENGKVECWPNLGYGKFGEKVTFKNAPYFRDGFNAKRLYFADIDGSGTTDLVYANHNSLDIYYNNSGNSFSKPVTLKLPVDYNHIGRIEFADILGNGTNCLLISYLDQSLKLQHLYYDFYNGVKPHLLKQIDNNMGNITRLHYAASTKFYLEDRKENRPWVTSLPFPVQVIEKIETIDNIVGSKLTNKYRYHHGFYDYTEKEFRGFGFVETWDTEDFSSSDQTEREHYVAPIYTKTWYHTGAYKKAEDLVESYRKEFYQGDREALKITPQVFDENIISIEDKRQAYRAIQGQMLRQEVYGLDENPDLYQHPYSVTESSACVKLLQSSTNSSHKVFKYGIFFVNLQETINYHYERNCCDPRIQHDFVLEVDEYGNVIKSASITYPRRIEPNNYSEQQKLHATLERNFYVNNTKEFYLLGALYKSQAFEIGGLRVAVSGYTNINDLRTHVTQSLQNTIKFEENLNYTSPQVRLLSESHNYFWNKEQTKNLALGLVTQQALLHHTEAMAFSDSQVKEVFAEKIASDQMLKDLGYKKTSDFWWVPSPTQYYYDSDKYYLPEKVIDVFGSETAVIYDQYNLLPIKTIVKATESSNYETTVEYDYQRLTPVKLTDHNDNVSEVILNPLGVVVATSLYGSEKGEKKGDKPLLEYQVRKNPNFEDVLGLADRANGPEYYLQNATSFFYYDLDAWRKNGQPVHVINLQRETHVSEGSFTRIRQIVTHIDGFGRNLETKMLTDSESEKWLASGRVVYNNKGTEVKKYEPFYSNSPFYDLEESVRNQGVSDTLYYDPLLRNVHIDTAKGFFSKVEFDSWSTKNFDLNDTVKDSTYYQEFTERWNSATEEEKIALKDEKDALSKAEKHYNTPTIEHLDSLGRKFLQIEILSNNEQSNVELKSHVKLDIQGNELESVDSRFYEQNQGKSENEKIKNFVHIYSMAGLLKSISIDAGEAWNLANVAGSSVYNWDGKGSLTYTEYDKLQRPIKVKVTDQDLNLNGNIVEKFFYSDDIQDKATNRYGTLVAHYDQAGLTEAKENDFKGQTLKSSYILRKNYKREANWQNVESITTTYADLEREIFNSSVKYNAIGEIIEKTDSQNNTHSPIYDIAGRVKQIKLKKQEEAIKTYVSDISYNPKGQRTSITYGNGVVTSYSYDSKTFRLIKLESVKEQKKLQSINYTYDPVGNITTMNDESYKTIFPNNKNIDTKSDYHYDSLYRLTEATGKEHPALSGKEEIIAPIPHLNNQNAISNYTEYYDFDYGSNITKIRHNGGNNSTKEFYISNKSNRSLPIIDRRSVNESNIDESHDERGNLLKLHGSQNLHWNYRDNIAYVDIITRPSGKNDSEYYVYDSGGQRVRKVTETYQNNETSSTRVEKIYFGDIEVTRTYQESGLSKEKYTVHVMDDKSRVALHHYWTKGTSDETRESQSRYQLSNHLDSVALELNDNAEIMTYEEYLPFGGTALIAGKTVREVTEKEYRYSGKERDESTGLYYYGARYYAPWLARWINPDPAGTVDGLNLYQFVGNNPINYVDSIGETKKRDRYNVKFNQQSKTSIAVSRRKVGANFSLFEEVHESGGKGEKKLHAETSYITGQRSEGDLKDNIDKEIWKLDRSPCQNCTREIIEYVKDRLEAQPNFKLSLGMENIYTKAEGIEKGKGSMEQARRINATSLAAMKVIFPVALNLKYARMYENVENKENTKSGNPHPLVKQRQEFANDNLAQGHAENMKLVIDPLKTLAGKIEEIANGQPGDNIESIRGAIESMVKSDNILKNLLQGKEGVMIDTWKSQGMTTVGSVKSTMKAILEGADNLIKYSVPANDNQASSSNKKRKRAPGT

SEQ ID NO: 91 - protein, 1545 aa, Xenorhabdus innexi
  REGION 1..1545: note = MISC_FEATURE - XinDa01
MGLIDDIVTRSARVGAIHASQRGQGPKGPAAAREGDPIQHSSFWAALAGAVTGALISAAIFGAAALLVAGTGGAAAVVLTAALGTAGTMALGDLILGASSAVTDMINSFCSPDGMLTSGSPNVLVEGKPAARATVDTAVCSKHPSPVPPLIAQGSETVFINGQPAARVDDKLVCGAAIKGGAGTVFIGSGQHAYLDVQEEFSLLERALLVAVEYLVPPTRGGLKLAGKLLTQQGRKVVTRAALKEAKAMARDAKSILAGAKQGAKKAQQWAKNAAKNPKALTQKAMAGAKAGAKKAAGNVKAGTQKVLKGAKTGATNAGKKIKDNYNDRVGCAKKAFHETKGAKRYWEATKRFFKGDPIDVTTGALFDQRTDVELGQTIALTFLRSWSPGESGLLGENWTDSFSGCAIVIGDRVEIRTTEGASLYFILMATDLNSVNPEHPQFTLSRANTGYVLHEKNNPVRQHFSVEVSDNKECQTWKLSSLSDRNGNAIDFHYSEQNQLIKVTHSDGPELVLLYRQDGLLKEIRRSDNDLNEVMARYDYHDNGWLAEADSTQQFHLFYEYNEQGLISHWSDGDQTAVDYIYDEQGRCTDSVGSGGYYPVHFEYEPGITRSMTPQGYTTIWHYNEEQLVTQVETPCGHITHYEYDEWGNLRRQILPEGQIITLDYLADTGLVTAFTDATGATWQYRYDDLDRLSSMTDPLGRTWKQQYDDNGNAECFIAPDGSKTTLIRNEFGLVTAAEDDEGHKRTWEYDEHKRLTKLFDEENRSLKLGYDSHDRMQRLTSGSGALWLWEYDRHHRVSLSDRPNNSIERFRHDRHGNLTDWTDARGVKWHIEYGPFDLPMARIDGEGNRWQYRYDLDSLQLLEVVNPQGESYSYILDADGRVITETDYAGTQWHYAYDGNGNCIEKRDALGNITRYEYDAAQRMTAMHTPEGTTTYRYDILGRLLEVIAPESAPLTFEYDDKDRIIKETQTHGKIERDYPDNATVERTLRNIDGQCWQVKAEANKVGELRLLSINGEHTLDLERDEDGHEWHRQSDKGFILRQEHSLMGQLTAQRAGRNTEFFASHEVADIPQPTLAGLDREYRYDAALNLVAANDERQWLRYVVNGNGQVTSVSEGERLREHYQYDASGYPARRFDGVNEVDGERLYQKGHRLRQLGQHLFEYDDAGRMTAMQLWQDGHRPQLTKFRWNSQNQLIGVQTPGGQQWDYRYDAFGRRTEKVCERAGSRTTYLWDGDVPAEIREYRHNRLYSIRHLVFDGWQLLAQQVQFFSLNPENRQELIAGEIQTQYAVCAPTGEPLALFDAAGHRVWRQPPHSLYGLRLGRLGENAELNPGLQFAGQWLDDESGLVYNRFRYYSPVASCYLTPDPIGLAGGEKPYAYVGNPAQAVDPLGLAGCEDPWKIVDRFRRSKNKMEPLGDRIPGAIDKDGLHTVAFFEMNGRRVFGVNSGTLYKKDKALGKQWNEKIDYLTKEEKGTSAFHAEGHALMRAHKKFGGVMPKEITMYVDRVTCNHCERFLPALMKEMGIEKLKLFSKNGTSSVLHAAR

SEQ ID NO: 92 - protein, 373 aa, Xenorhabdus japonica
  REGION 1..373: note = MISC_FEATURE - XjaDa01
MTALQLWQAGHRPQLTRFRWNSQNQLIGVQTPGGQQWEYRYDAFGRRTEKACERAGLRTTYLWDGDVPAEIREYQHNRLHCIRHLVFDGWQLLAQQVQFFSLNPENRHELLAGEVQMQYAVCAPTGEPLALFDAAGHRVWRQPPQSLYGLRLGVLGENAELNPGLQFAGQWLDEESGLVYNRFRYYSPVAGQYLTPDPIGLMGGENPYAYVHNPSTWVDPMGLAGCPWEQIDRFRSRTRLPVLGNRVPAPNDGLQTVAFVSIGNRKFFGMNRQAIPGLEYAAKRKEWFRKIYKSTGVEPSTISKAAADAEGLALVRAYERITKSGGKMPKEVTMFVDRPTCDFCKNYLPLLRESMGIEKLVVYNKGVSNPLIF

SEQ ID NO: 93 - protein, 445 aa, Hungateiclostridium clariflavum
  REGION 1..445: note = MISC_FEATURE - HcDa01
MMHGKNSLKMATESIGLIKAGKANMLDNIANITKTQKGYEFVTTDGIIFKMIDDDIPAAAKSVVKSMSDSTDELMTCLKREPEALFELVEETKELDNTILYRLRDDTEIKIFKSDLTPEQIICSTIGCFTGDTLVTTKEGLKRIDEVKTGDYVLSKDVKSGEIGYKKVNYVYIKNTNKLVQLIVGTEEINTTSSHLFFTDSGWWKSAKNIKVGDRILTDDGELKEVTATRVVELEEAIRIYNLNVDEFHTYFVGTSGLLVHNDCTAEMIGAGREAIANARLKGITDSNILSKLGTVAAENVPLARKSLSKASELSSMLGYGSKAIASDGTTNTLSGWAEITQGAEYFNRAATPQDVIAKSNEIGHTLKTDIFDNGIPGQYNASHAEKQLSLLSNKALGVSRPMCSDCVEYFKKLAKFSNTEKVVSDSNITRIFMPDGITIIEIPH

SEQ ID NO: 94 - protein, 3637 aa, Streptomyces sp.
  REGION 1..3637: note = MISC_FEATURE - SsDa01
  source 1..3637: note = SID1328
MFTTDQALAEGLELGRVKLPSASLSSWASWFTDPHWGKLPHQQSGTAAGKRHHVSAAATRAHRGAGHKPGHGRGELGAYKAHKSSAKAGLSASFRGFNPRTSKRVSARSTQKSTYYHNADGSFSRKVSPFPLNYKDPKGEWQQIDTAVARGPDGRWDEKSNAVDVGFAPKAADDDLASFGKDGKSVGFALEGAAKVPGVAKGSTVAYPEVLPDTDLELTSTSVGLRQALVLHSADAANSWVFPLHLKGLTAELNKSGGVDLRDAKGKGVASIPSAYAFDSKIDKVSGERATTHAVSYELVTSHGETALRMTLNPAWLHAKARQFPVTVDPDTSVWDYVHSTYTESGYPVADRSQEQTLKIGSYDSGSHSAISFVQDWRDSFDGSGVSLVSANLHLYDIWASTCTPETFNVAQVSAAWDPTDAMTYPGPAHGASIGSVTPTVANACANTTGDQSGGDWVTVPLDTAAMQKWASGTTADHGLAIYAATTDALHWKQFGSMAYPGDEPYIVYDYTGDVAPMVEATFPANNSSISTLTPELQAFADFQQNPVSQKYDFKVYSASGTLVADSGLVTTPNWTVPAGKLTWNTSYTWTAQAYDGTKYSTVSPSSALTTEVPQPFLTSELSQNSSDHGYDATIGNYTTSDTDASISTVGPSLDVERDYNSRDFRTSGAFGAGWSSIFDAKVTEQKSAAGALTGVVVTYPDGSEVGFGKNSDGTFSAPSGRFAMLRAQTSPAGYTLTDKDDTVYTFTQPLGTGVSGISSVKDANNRSVDLTWTSGHVTRMKSNVSGRSLNLTWSTPSGATAAHVATVVTDPVDPKDSSSAQTWTYGYTNDQLTKVCSPLSTAKCTTYGYTGGSQFRTEVENAGASELWQFNEAGGTTAASSVGDSEGTDNATYSGVTLGQAGPLAGSTATAAKFNGTSSYAELPPSLVSSSNQQTMSMWFKTTTAPGVLFSYSTLPLSYTMAQGQYTPGIYVGTDGKLNAEFWNSTGVSPIVSPSAVNDGAWHEVVLAGAGSLQTLYLDGAKVGTRSGMISYSNGYHSALQNHTYIGAGFLGGTWPDEPHYSTTSDQGFPTYFNGTIADVALYPDALTADQVSRIHTAATKASPLLTTVTRPSGKAFAGITYDTDAARVTQVTDENGGVWKLNPATITGSSNTYRGAVLGGAPNQYYRFGESAGASTALDEVRGGTGAYSSVTLGSAGPFSDHTTGAFNGTSSKVTLPSGTIPGTGPESVDMWFKTATANGVLVSASKDPLGTTTPTAYTPILYVGSSGKLYGEFYFSGGSPKPLGTTAAVTDNKWHHVVLSTSATSQSLYLDGTLVGTQSGTAAMGGQPYTYVGAGYLGGNWPDESHSSSTDNTGYASYFKGSIAEAAVYRSQLTGDDVDAQYDAALNSTGLLPVKTVTVTTPTNATLSYQYDVTHGNRLLSETDALGNTTSFGYDTSGFEHTVTDPNGSMTVTGHDVRGNIVSSTSCQDQAANKCNTEYYTYYPDDTTAQLTTADPRNDQLLTERDGRSASASDNTYLTTYTYDSAGNQTAITGPAVPGFPNGRTTQTVYSDGTATYPAADGGVVPAGLPVKTVSPGGAVNQVAYNKNGDVASTTDGIGLVTSYTYDGVGQVSTQNVVSDTYPAGLTTTYTYDAAGQVTKEDDPQITDRVSGATHRASTTTVYDEDGNTTSQTVSDASGGDRPRTQTWTYDAYDHVATEADANVAAGASYGNTTTYSYDTSGNKTKEVTSAGNETRYTYDAEGNLLTQGLMYTGDPTDPQPATLLTESSRAYDPSGRLASITDAMGNVTAYTYTDDGLTATVTKKSGDGKSTYVLESDTYDAAGNLLQSTSDNGETVTKYTVDAASRTTSTELDPTGVDRVSTVSYTPDDQVASETDRDASGYDRTTTNTYDDMGNLLSETLYGDASGHPAGWWKLDQGSGTSVTDSSGTGNTGQASNVTWTDSSAGLTGTSTAQGVATNGPVLDTTASYTVSAWVKMSSLPTHNATIVAQNGTTNSAFMLQYNYAHAGSPLWSLNDTSKDAAGPTFPAAYSTAVPAVGAWTQVVGVYDASNGSMKLFVNGSQSGTAGTNTTPWAATGPLTIGRAKYAGADSDALPGGVSNVQVYQRALSATEVSSLYGKGRTGGTVGSSTAQTTKYTYDKRGLPSSMTDAENHTTDYSYDEAGNLALTTAPAVQAETDGTNPVSVRPVTTSGFNTFGEAVEERDPNLLVTSTAYDAEGNKVSETLPSYTPAGAAQPNAGTTVWTYDSEGNQTSVTTPGGKTTSNLYDQLGDVVQTTAPDGTRTHAVYDTAGDQLSATDPTGAQTQATYDFMGRPLTQTTLERYPSTRTLTSTNSYAVTTGNPYGAHVASTTSPGGVSSTYAYNRAGDITSVTDGAGNTTRYAYDFEGNPSKTTLADGTYTTTDYNASGDPTATHSYDAGGTQLWSTSQSYDGVGNMTAATDANGHTSQFTYDAAGTITQETQPVDASTSITTTFGYDQAGNRTRFTDGRGNSWRHTYTPWGQQEKVIEPTTAKFTSAADSTTIFAYDADGQATNVTLPGGTTTSMTYDGNGQLKTMSGSGADAVTATRSFSYDTDGRVLSADTAEAGTAGAADHQSATHDAFTYDDRGDVLTTSGSAGSSGFAYNNDGAMTSRTDASGTTSYGYDTAGRLSSLNDAATGTQLTYSYDQLNQLKSVKYGATGQTRTFGYNTAHELTTDTLVQGASTLAGFTYGYDKNGNLTSKTTTGVSGASSNTYGYDWANRLTSWNNGSTSTTYAYDASGNRIRKGSDVYTYDQRDELTSDGVHTYDYSARGAMTQESSASGTLAYKTDAFGDQIVAGDQAYNLDAMGRNITDRDTSAGGLTRTFAYSGADNTIASDGDFTYTHDTGGGVVGVKPVGGGSGALALTDQHDDVVGTFTSGATALAGSASYDPLGKVVSPTDTVTGHLGFQSGWTEPGTGDVGTASRWYNPDTGQFLNKDSMSLDAVPNSVAANPFAYVDDNPLAGTDESGNCSWYDVVCGAKKAAHHVAHAVKHVAHKVYHAAKHVARKVVHAVKHVAHKVAHHVRDVYHATVRVVRRVYHYAARHVRRAYHQIVHAVHSAYHAVRKAVHRVVHAVKKAAKKVAHAVKKAAHAVAHAARTAYHATVKAAKTATKFVKNHAAAITSVVVSTAIFAGCEAFTAGIGSIGCAAVAGAAGALVEQGFKCADNGGDDCSVGAFAGSAVEGAVTGAAGGALGELGGKILAKAAPKALEVAGGLFGKGATEAGESATADATDEAAAAAKSEASGTKSQSHSDEAPAEEGPITCETHSFTGSTEVLMADGTTKAIDHVKVGDTIANSVPGAKGREAHKVTAVIVTRTDHDFVDLAIKKTGGKRAVKAGAKSLAKKAARKAAFGLAASAAVLAALSAGHSHGDTHPATTPVAAVSKASSIQAAEPKPAHLTTTFHHPFYDETQSAFVEARDLNPGDILQTPTGTAEVTGVRLYHANTTTYDLTIGTLHTYYVEAGSTPVLVHNISLPCPSELEAGLEKAKNASSSFERPGGMSGHAKLSDGTSHDLSSGGDGRNLRSDWEAPPGTTDENFHHLENQTAALMRQSGSHEAYLYLHKAEGAAYGACKYCVSAMREMLPQGSKLMVIWRNEEGAIRNRVFIGGAD

SEQ ID NO: 95 - protein, 152 aa, unidentified
  REGION 1..152: note = Isolated from environmental sources.Organism unknown.
  REGION 1..152: note = MISC_FEATURE - AncDa04
MAYAKEIEQSKEAYKRAVEVGEQYGEKFGTKTVASNGKKTLSGWDGKTTKAPEGFTRVSPEEVMDYSKEIGHELKNSGSYDHGIEGQFQACHAEKQLSLLTDEPIGVSRKMCPDCQGYFQKHAAYTGQEKIVTDPEKTRIFYPNGEVEEIPW

SEQ ID NO: 96 - protein, 164 aa, unidentified; identical to SEQ ID NO: 5
  REGION 1..164: note = Isolated from environmental sources.Organism unknown.
  REGION 1..164: note = MISC_FEATURE - MGYPDa829

SEQ ID NO: 97 - protein, 150 aa, synthetic construct
  REGION 1..150: note = Synthetic construct
  REGION 1..150: note = MISC_FEATURE - chimera_10
MYAKEIEASKEAYKRAIDMAEKYGYNGAPPKRKTVASNGKITLSGWKKLKDNTDFIRVSPEDIIDKSQEIGHNLRNAGANDQGIKGKYNASHAEKQLSLLTDEPIGISQPMCPDCVEYFKKHAAYTNQVKITADPKKTRIFFPDGRVEEF

SEQ ID NO: 98 - protein, 150 aa, synthetic construct
  REGION 1..150: note = Synthetic construct
  REGION 1..150: note = MISC_FEATURE - chimera_10
MYAKEIEASKEAYKRAIDMAEKYGYNGAPPKRKTVASNGKITLSGWKKLKDNTDFIRVSPEDIIDKSQEIGHNLRNAGANDQGIKGKYNASHAEKQLSLLTDEPIGISRDMCPDCVEYFKKHAAYTNQVKITADPKKTRIFFPDGRVEEF

SEQ ID NO: 99 - protein, 1560 aa, Pseudomonas entomophila; identical to SEQ ID NO: 13
  REGION 1..1560: note = MISC_FEATURE - d22_Cd4_PeDa01
  source 1..1560: note = strain L48

SEQ ID NO: 100 - protein, 154 aa, synthetic construct
  REGION 1..154: note = Synthetic construct
  REGION 1..154: note = MISC_FEATURE - chimera_05
MYAKEIEASKEAYKRAIDMAEKYGYNGAPPKRKTVASNGKITLSGFDGKTTKAPEGFARVPLDTMFDYCKKIGHELPPKVPAFYDNGREGSYFACHAEKQLSLLTDEPIGISRDMCPDCVEYFKKHAAYTNQVKITADPKKTRIFFPDGRVEEF

SEQ ID NO: 101 - protein, 154 aa, synthetic construct
  REGION 1..154: note = Synthetic construct
  REGION 1..154: note = MISC_FEATURE - chimera_07
MYAKEIEASKEAYKRAIDMAEKYGYNGAPPKRKTVASNGKITLSGFDGKTTKAPEGFARVPLDTMFDYCKKIGHELPPKVPAFYDNGREGSYFACHAEKQLSLLTDEPIGISQPMCPDCVEYFKKHAAYTNQVKITADPKKTRIFFPDGRVEEF

SEQ ID NO: 102 - protein, 150 aa, synthetic construct
  REGION 1..150: note = Synthetic construct
  REGION 1..150: note = MISC_FEATURE - chimera_20
MIDPLGLAPCPWQIIDRFRSLKAGLSRLGEHIPHQGDGFGTVAYIEINGRKIFGVNSSVLSDKAKDLGRYWVDRLKLGRGVSQVAYHAEAHSLMRAFEKFRGKLPNEITMYVDRYTCPNCQKYLPLLMKEMGIDKLIIYTKGGKVVLLPK

SEQ ID NO: 103 - protein, 261 aa, unidentified; identical to SEQ ID NO: 37
  REGION 1..261: note = Isolated from environmental sources.Organism unknown.
  REGION 1..261: note = MISC_FEATURE - MGYPDa17

SEQ ID NO: 104 - protein, 150 aa, synthetic construct
  REGION 1..150: note = Synthetic construct
  REGION 1..150: note = MISC_FEATURE - chimera_19
MIDPLGLAPCPWQIIDRFRSLKAGLSRLGEHIPHQGDGFGTVAYIEINGRKIFGVNSSVLSDKAKDLGRYWVDRLKLGRGVSQVAYHAEAHSLMRAFEKFRGKLPNEITMYVDRLTCPNCQKYLPLLMKEMGIDKLIIYSKNGKVVLLPK

SEQ ID NO: 105 - protein, 137 aa, unidentified
  REGION 1..137: note = Isolated from environmental sources.Organism unknown.
  REGION 1..137: note = MISC_FEATURE - AncDa05
MACGELSEMAQQVHNLAGGKPDARAIRNSTVAIFEVKLPNGKKTLYAAGSSGRLNPRQRALLVKLGVPEENILSGKKFTLPKDGVTKNANHAEQIILRNIPDGAKVTRKGISWGGKQRNAPCSHCKPSVDGAGGIYD

SEQ ID NO: 106 - protein, 142 aa, Pseudomonas entomophila
  REGION 1..142: note = MISC_FEATURE - PeDa01
  source 1..142: note = strain L48
MYAGGTNLFAYAPNPIWWIDPLGLNKFGSGKGVHTAVVNVHDSDGEHTVVSCILHSGNMTEEERALGFPRSSLATHTEARAVKQIPLQQGDVMVIDGQYPPCPSCKGKMNRASTETGAKIQYTWPENGETKTWTAGQKRRRR

SEQ ID NO: 107 - protein, 148 aa, unidentified
  REGION 1..148: note = Isolated from environmental sources.Organism unknown.
  REGION 1..148: note = MISC_FEATURE - AncDa03
MYAKEIEQSKEAYKRAIDIAKKKKVKGCVASNGKITLSGFDGKTTKAPEGFTRVSLDTMFDYCKEIGHELPPKMPAFYDNGIEGSFFACHAEKQLSLLTDEPIGISRDMCPDCVEYFKKHAAYTNQVKITTDPKKTRIFFPNGRVEEI

SEQ ID NO: 108 - protein, 144 aa, synthetic construct
  REGION 1..144: note = Synthetic construct
  REGION 1..144: note = MISC_FEATURE - chimera_08
MYAKEIEASKEAYKRAIDMAKKKKVKGCVASNGKITLSGWKKLKDNTDFIRVSPEDIIDKSQEIGHNLRNAGANDQGIKGKYNASHAEKQLSLLTDEPIGISRDMCPDCVEYFKKHAAYTNQVKITADPKKTRIFFPDGRVEEF

SEQ ID NO: 109 - protein, 192 aa, unidentified; identical to SEQ ID NO: 41
  REGION 1..192: note = Isolated from environmental sources.Organism unknown.
  REGION 1..192: note = MISC_FEATURE - MGYPDa18_extN

SEQ ID NO: 110 - protein, 150 aa, synthetic construct
  REGION 1..150: note = Synthetic construct
  REGION 1..150: note = MISC_FEATURE - chimera_18
MIDPLGLAPCPWQIIDRFRSLKAGLSRLGEHIPVRGDGLETVAYIEINGRKIFGVNSSVLSDKAKDLGRYWVDRLKLGRGVSQVAYHAEAHSLMRAFEKFRGKLPNEITMYVDRYTCPNCQKYLPLLMKEMGIDKLIIYSKNGKVVLLPK

SEQ ID NO: 111 - protein, 195 aa, unidentified; identical to SEQ ID NO: 58
  REGION 1..195: note = Isolated from environmental sources.Organism unknown.
  REGION 1..195: note = MISC_FEATURE - d22_HmDa02

SEQ ID NO: 112 - protein, 159 aa, unidentified
  REGION 1..159: note = Isolated from environmental sources.Organism unknown.
  REGION 1..159: note = MISC_FEATURE - AncDa06
MNLGTPEEPWEAIDRFRKQKNGLEPLGDRIPVRGDGLGTVAFIEVNGRKIFGVNSSLLSDEEKNLGREYFEKMKEEGYFKNAKSYGRGEAQVLTHAEAHALMRAYEKFGGNLGKDVTMYVDRPTCGFCQKYLPELRKAMGIDKLTVISKNGETLILNKG

SEQ ID NO: 113 - protein, 197 aa, unidentified; identical to SEQ ID NO: 48
  REGION 1..197: note = Isolated from environmental sources.Organism unknown.
  REGION 1..197: note = MISC_FEATURE - d41_MGYPDa917

SEQ ID NO: 114 - protein, 369 aa, Clostridium hungatei; identical to SEQ ID NO: 65
  REGION 1..369: note = MISC_FEATURE - RhDa01_extN10

SEQ ID NO: 115 - protein, 445 aa, Hungateiclostridium clariflavum; identical to SEQ ID NO: 93
  REGION 1..445: note = MISC_FEATURE - d21_HcDa01

SEQ ID NO: 116 - protein, 148 aa, synthetic construct
  REGION 1..148: note = Synthetic construct
  REGION 1..148: note = MISC_FEATURE - chimera_06
MYAKEIEASKEAYKRAIDMAKKKKVKGCVASNGKITLSGFDGKTTKAPEGFARVPLDTMFDYCKKIGHELPPKVPAFYDNGREGSYFACHAEKQLSLLTDEPIGISQPMCPDCVEYFKKHAAYTNQVKITADPKKTRIFFPDGRVEEF

SEQ ID NO: 117 - protein, 150 aa, synthetic construct
  REGION 1..150: note = Synthetic construct
  REGION 1..150: note = MISC_FEATURE - chimera_17
MIDPLGLAPCPWQIIDRFRSLKAGLSRLGEHIPVRGDGLETVAYIEINGRKIFGVNSSVLSDKAKDLGRYWVDRLKLGRGVSQVAYHAEAHSLMRAFEKFRGKLPNEITMYVDRLTCPNCQKYLPLLMKEMGIDKLIIYTKGGKVVLLPK

SEQ ID NO: 118 - protein, 164 aa, unidentified; identical to SEQ ID NO: 5
  REGION 1..164: note = Isolated from environmental sources.Organism unknown.
  REGION 1..164: note = MISC_FEATURE - d38_Cd11_MGYPDa829

SEQ ID NO: 119 - protein, 149 aa, synthetic construct
  REGION 1..149: note = Synthetic construct
  REGION 1..149: note = MISC_FEATURE - chimera_01
MLPCPSELEAGLEKAKNASSSFERPGGMSGHAKLSDGTSHDLSSGGDGRNLRSDWEAPPGTTDENFHHLENQTAALMRQSGSHEAYLYLHKAEGAAYGACKYCVSAMREMLPQGSKLMVIWRNEEGAIRNRVFIGTSNDPKMSSRYKGN

SEQ ID NO: 120 - protein, 133 aa, unidentified
  REGION 1..133: note = Isolated from environmental sources.Organism unknown.
  REGION 1..133: note = MISC_FEATURE - AncDa07
MACGELSEMAREVHNLAGGKPDARAIRNSTVAIVEAKINGKKQLYAAGSGGRLNPRQREALVKLGVPEENIFHGKKVTNGFTKLENHAERIILRNLPEGAKVTRWGISWGGLQRNAPCPHCQPFVDGAGGIFD

SEQ ID NO: 121 - protein, 129 aa, unidentified
  REGION 1..129: note = Isolated from environmental sources.Organism unknown.
MSGALSEMAREVHNLAGDKRAINQSTVAIVEAKINGKKQLFASGSGGRLSPAQRELLVELGVPEENIFSGKKFRNGFTKLENHAERVILRNLPEGAKVTRWGISWAGKQRNAPCPNCQPFVEGAGGIFD

SEQ ID NO: 122 - protein, 158 aa, unidentified
  REGION 1..158: note = Isolated from environmental sources.Organism unknown.
  REGION 1..158: note = MISC_FEATURE - AncDa09
MIGLEGGNLYRTAPGVANWGSAYGLNKDFQGNYNLTKPVLPKYDGKTTHGVLVTPDGKQIPFTSGNGDPSYPNYKNSGHVEGKAALYMRENGISEATVFHNNPNGTCGFCNNMTPTLLPEGAKLTVVPPSNAVANNSRAIAVPKTYTGNSNDPKISPR

SEQ ID NO: 123 - protein, 153 aa, unidentified
  REGION 1..153: note = Isolated from environmental sources.Organism unknown.
  REGION 1..153: note = MISC_FEATURE - AncDa10
MGFEKEIEQAKQAQKRAFELAEKYGYNGAPKRKTVASDGKINTLSGWKKLKDNTDFIRVSPEDIIDKSQEIGHNLRNAGANDQGIKGKYNASHAEKQLSLITDKPIGISQPMCPDCQNYFKSLAISESKEYVTADPNMVRIFKPDGSVTEFKR

SEQ ID NO: 124 - protein, 151 aa, unidentified
  REGION 1..151: note = Isolated from environmental sources.Organism unknown.
  REGION 1..151: note = MISC_FEATURE - AncDa11
MGEADPVKKSQEAYQRAKELKKELNKKKAYKSKTVSSDHVKTLSGWSKTRRPEGFQDPNIDEVLEKCDEIGHEPKPHGAKDNGVPGQYHASHAEKQLSLTAKEPYIGVSKPMCPDCQGYFSKLAQHEGRDWYVTDPNNTWIFRADGSVTKQ

SEQ ID NO: 125 - protein, 140 aa, unidentified
  REGION 1..140: note = Isolated from environmental sources.Organism unknown.
  REGION 1..140: note = MISC_FEATURE - AncDa12
MLAGGLNLYQYAPNPVSWVDPLGLAFGSGKGTHTATVTVFDSNGNVKHSGIFQSGNMTPEEKALGFPRSTLATHTEARAVRQIDLSPGDRMVIDGQYPPCPSCKGKMNRAAKETGATIQYNWPEDGETKTWTAGKKKRRR

SEQ ID NO: 126 - protein, 1543 aa, Burkholderia pseudomallei
  REGION 1..1543: note = MISC_FEATURE - BpDa02
  source 1..1543: note = 1710b
MALPAVKHLDPVVGIDVHSVLVTPGTPPVFLPHPYVGFMLDRREYIDAALGVIGSIVFTFTPLGKEAEAGLKWAKKQAKAELMSDPLIAEGVKLGKEAAPVAGDIAGAVGAGVGMGSMMGRPIFVNGFLRATAGTHSFHVPGLHFPLGESFAPPPEDFEPSNDGESYMGSRTVLANNDPMSFMALPAMSCWSVGLEPPPHNGAHTQRTALSMPSSVMLPIPVGRPVLVGGPPVMNMAALASSLFKAFRGSDWAKALADKLHLKPGFLRCNVLRAEPVDAITGEVVVQQHDFTVPGRLPLVWNRYYTSHDTYVGAVGIGWQTPADIRLELTRHEGAVGVVAYFPDHATAFDAIPDAAGWPARVYDWQHGQALYRQDDRLVLRTRAGFEYEFALPAHWQHALERLAEDATLTLPVERMADLNGNAWVFERGLDSSLTRLVEWKAEEATGRMIACAASASSRAGDHVSLLTALTLIDAGGRAHPLVSYQHDRNVDLVAAIDAMGQPHRYAYTDGHRMISHTSARGISFYYSHRRHDDGVWRVDHAWGDNGLFDYRFIYDPARHETRITDSLGHTTLLQSDERGMPVARIDPLGGVTSYRYDAQGRTNAETDPAGRTTAWEYDTFANLLMRTLPDGTALRTEYNTGHKPVCVAVPGGGQWRYTWDERGNLLMQTTPSQASVRYAYDQYGQLIAHTGPRGTVTRFDYDRSGLLAVRTDALGHRTQYTHDALGNLVHVINALGQTSRYEYDNIGNLTRAIEPGGREVHCVYDADGNLVRYRDATGNVTQLVYTALGHISKRQTPDGNVVEYRYDTEEQLVGVVNGRGEIYEIKRDELGRIVEETDYWGQPRHYRYGAAGELLDITDPLGQTIEYRCDRLGRVVEKRMPDPLHPDGVRIDRFVYDQHGDLVLAENPSSRVEFRYDADGRVIAERQGDNFTIASTYDASGNRTERKTRLVADSDVVEHTVRYAYDALDAVIAIQIDDAAPIVLERDAVGQVCVEQLSPELRRELSYEAGGQLAKQTLLGGTGLLFASEYAYDANDELVEKRDSRTGVERFQYDPVGRIVAHTDPTGRLRSYVYDPAGDLLKTHIHERRTAGATEMTQTGTWVREGEFEGHYHAYDRAGNLIRRQDVGQDLTLRWDAAGQLAEAVAVRPAIAGAGGGQVRIGTQYEYDAFRRRVGKLVLTRAAGSAELVLSRISRFFWDGNTLVGQCTRGGGEGGGTAIPDAGDRMPAAQFIPVRDNRDDAPVSGFEHAYEWVYYPGMFRPLAVVHCDLAATRASVPTTTNLLPLIGAVYFFQSDLNGAPVQMHAPGGRVVWEARYDPIGRSEQLGLQLVEQPVRLQGQYFDAETGLNYNRHRYFDPNSGIFISQDPIRLSGGLNLYQYAPETNNWIDPLGCSGHRRRHEKMPPEGAPLTTGNLFRHAQDEGIPPIFSRKDDEFYHRLIEIYAGTGVLNAYIRGIASHVEPKAGLILNEDGNGWKIGSLYINYPDGPCPGCRRLMPFILNDGSILYVTFPTLGLDGYSYGHFHGGVSGFFREGTPCNLRHPE

SEQ ID NO: 127 - protein, 2427 aa, Kitasatospora setae
  REGION 1..2427: note = MISC_FEATURE - KsDa02
  source 1..2427: note = KM-6054
MAAVPSAEALAAKRARDTIWTPPNTPLGSQTKSVDGENLVPGRLPGPLEPEPADWTPGGPASVPAPGSADVTLGFDSAEAAAARKATGGAAPASDGAALRAGSLPVVIGAAKDAKSGAHRIRVELVDQAKSRAAHLDSPLIALTDTEPDTPPSGRTTKVSLDLKGIGAQTWADRARLVALPACALETPDRPECQQQTPVQSSVDLRSGLLTAEVILPAATEGTAPPTKSSLGSGTASGVVQAGLTTAAPAKAAPTVLAATAGASGSGGSFSATSLSPSAAWGAGSNVGNFTYSYPIQTPPSLGGTAPSVGLGYDSSAVDGKTSAQNSQSSWLGEGWGYEAGFIERGYKSCNTAGIANSSDMCWGGQNATLSLAGHSGTLVRDDTTGVWHLQSDDGTKIEQLTGAPNGLQNGEHWRITTTDGTQFYFGRNHLPGGDGTDPASNSAFKEPVYSPKSGDPCYNSSTATGSWCTMGWRWNLDYAVDVHGNLITYTYAQETNYYSRGAGQNSGSGTLTDYTRAGYLTQIAYGQRLSEQVTAKGAAKAAALITFTAAERCVPSGSITCTEAQRTTANASYWPDTPLDQVCASTGTCTRAGPTFFTTKRLASLTTQVLVSGAYRTVDTWTLTHSFKDPGDGNAKSLWLDSIQRTGTNGQTAVTMPPVTFTAVMKPNRVDGDLTLKDGTKVTVTPFNRPRLQQVTTETGGQINVVYTTSSDAAHPACSRLAGTMPAAADGNTLACAPVKWYLPGSSSPDPVDDWFNKYLISAVTEQDAISGTTLIKATNYTYNGDAAWHRNDAEFTDAKTRTWDGFRGYQSVTSTTGSAYPGEAPRTQQTATYLRGMDGDVKADGSTRSVQVANPLGGPALTDSPWLAGSSFATQTYDQAGGTVISANGSVAGGQQVTATHAQSGGMPALVARYPASQVTTTSKSKLSDGTWRTNTTVSTSDPAHANRPLSSDDKGDGTPGAELCSTNGYATGTNPMMLNILAERTVTKGACGTPVTSANTVSSARTLYDGKPYGQAGDLAESTSALTLDHYDTGGNPVYVHTAASTFDAYGRLTSVSEANGATYDAAGNQLTAPNLTPATTRTAYTPATGAIATTVTQTTPTGWTTTLTQDPGRAEALVSTDANGRATTQQYDGLGRLTAAWSPERATNLTPSQKFSYAVNGTTGPSVVTSQWLKEAGGYAYKNELYDGLGRLRQVQRTSDTYSGRLITDTVYDSHGWPVKTASPYYEKTTAPNSTVYLPQDSQVPAQTWVTFDGIGRTTRSAFVSYGQQQWATTTAYPGADRTDVTPPNGKYPTSTFTDGRNQVSALWQYRTATPTGNPADATVTTYTYDAANRPATRKDAAGNTWSYGYDLRGRQTTVTDPDTGTTTTAYDVNSRAVSTTDGKGNTLVVSYDLIGRKTGLYQGSIAPANQLAGWTYDTLPGGKGKPTSSTRYVGGAGGSAYTQAVTGYDAGYRPTGTSVTIPASEGKLAGTYTTGLTYNPVLGTLKQTDLPAIGAAPAESVMYTYNISGVLQKSYSDTYYVYDVQYDAFGRPVRTTTGDAGTQVVSTQLDKTDYTYNQAGDVTSVTDVQNGTATDAQCFTYDHLGRLTQAWTDTAGSTSTTSGTWTDTSGTVHNSGSSQSVPALGACANANGPASTGSPAKLSVGGPSPYWQSYGYDSTGNRTTLVQHDTTGNTTKDTTTTQTFGPAGSVNTATGAPNTGGGTGGPHALLTSSTTGPTGTQVTSYQYDQLGNTTAVTETSGTTTLAWNGEDKLASVTKTGQAQATSYLYDADGNQLIRRNPGKTTLNLGSDEVTLDTAANSLTDTRYYSAPGGISIARTTGPTGASALAYQASDPHGTANVQINVDAAQTTTRRPTDPFGNPRGTQPAPNTWAGDKGFVGGTKDDTTGLTNLGAREYQPTTGRFLNPDPLLDAGNPQQWNGYAYSDNDPVNSSDPSGLITNALADGDTYVARPAAFCVTMSCVEQTSGPGFWEDKRVGDAVFAAVVQATTQSNGNGSSQTKKEKGIWGQAWDWTKKNGGAILGALVEGAVFSTCFIGAGFAAPATGGITVIAGAAACGAVAGEAGALTTNILTPDADHSVDGITNDMVVGEITGAAVSAASEGASSLAKPAVRKLLGMEAEEGLEAAGRAATGPCNSFPAGVTVLLADGTTKPIEQIAQGDQVTATDPQTGTTQAEPVTDTIIGHDDTEFTDLTLTNDADPRAPPSEITSTTHHPYWNATTSRWTDAGDLKPGDHVRTPDGTELTVNTVYSYTTQPRTARNLTVADLHTYYVLAGNTPVLVHNTGPGCGEPGFVSDAANSLSGRRITTGQIFDASGNPIGPEITSGGGSLADRAQSYLADSPNIRNLPAKARYASADHVEAQYAVWMRENGVTDASVVINQNYVCGLPLGCQAAVPAILPRGSTMTVWYPGSGSPIVLRGVG

SEQ ID NO: 128 - protein, 3904 aa, Providencia alcalifaciens
  REGION 1..3904: note = MISC_FEATURE - PaDa01
MNKLFYRLIFNAARQMLMVVSDITRSHRASPAGSGENRVENVAKSRVHWSVKLIVTSLWLTLGMVSFSASSSTIVADGNALGNQQPTVVNTQNGLPQVNIQAPNREGVSRNQYSQFDVDKNGAILNNSRTNTNTQLGGMIQGNYWLAKGEAKIILNEVNSRDPSQLNGFIEVAGKKADVIIANPAGITCNGCGFINADKALLSAGKTLIENGKIKGFEVDKGSINIVGQGYNGNGTNYTALIARSVNINAKLHAKDLAITTGKNTVAADGKTILKIDSTSTDDKSEFALDVAVLGGMYANTIKMRGTENGVGVRNAGHIGAEAGDITLSAEGKIGNAGVITASQHIALDSQQSINNTGTVLAQNDILLNAKQTIINADKGQIVAGRDAVLRAEKINSDRTALLAAGVDSKGKLTNTGSLAVKGDKTVVLQGDILAKDELMATGSDLDLSHSNIQAKNMKLAASTADIRTQEAHLRATNNATLIAKRGIDNQKGEIVANQLTLSAPEFIDNQHGKLVQIENSKNTLNTKAFNNQQGEINLAGDTSITTQQLNNQSGQLFIREGKVDIQSQNLDNRQGTILAPGKQGLILKADTLDGQKGEILTRGTLGLTAKSVNLDQGTTQAEQITLQANSLSLRQGNMLQTGDKAMTLNVVNGLDNTQGSVASKGDLNVQAGTVDNTDGKLLTSKNHRLDLMSTGELNNTRGVVQTDKTLMINVDKLINQQGKISSLSGASLLTANHLAGEKGTIFAQTALRIESADINLNQGFTQANQVSIFANNFSHQGATLLQTGEGKTELHIQNQFDNQKGEISSNGQIDIVANGLNNQDGKIIAAKLGHLTLTIQQVLNNTQGTLLGNQGIQLAAERLINQSGKILASFGDNQLALKQLDGEKGEILSKGKLALTGEELNLNDAVTQADNIQIEGQNLSHQRGKMLQTGSEKGQIKLAHTLDNQSGNISSQGTLRVNVDKLSNQQGVMVAAKVGALILNAKQHVDNTQGTLFAQQDFTLNTQSFTNIDGKVISKQGDIRLATEDLQGQRGEIIAQGDLSLSGKDIDLTAANTQAQHIQLTANRLTHQNGTMTQLSERQGTIEVSQQINNNAGDISGNGSWLIKTHALDNQQGKIFSARMGKLDLLIQQTLDNTGGTLTARQGVFVETQSLINRTGKVIASMGDVTFNSQLLESDDGEILAANTLNIEGRTLSLNQAVTQADNIVITANSLEHQGGKLLQTGDKAGKITVQGALNNQVGEIGSNGDLSLTANRLDNRDGQIITAKKSHLKVDLIHELLNQSGALIGEYGLNITASDIQNQQGKLVARHGDAKLDVAKGINNQAGLIAAEQLLQMSNQALKNQLGYIQANTIDINTNNQLLDNTQGSLLAKQQLTLNSGKLENQKGTIQSGSDMLIDTHGQQLNNTQSGDNKGIYSQGGLTLTTGGLNNEQGRLVAKNTLALNNQELNNQQGLIGSQSANKMQTQQLDNSQGTIKGQSVNLDTQGQRLTNLANKAEHGIFASQNLAMTVGELINRHGHIQANQITLNTQQNKIDNFQGEVLASETLQANSGEIDNQQGRMQASQQIAINTNGQLLNNNNTQQSDGILSGGTLTLTTGKLDNQQGQIQSAGAQILNSQLLDNQKGSVYSGSSLSIDTQKNSVLNQEGVLVSQAQLTLSARNLNNLQGTVQGKQGVNVHAETIDNQKGILLSQASAEVTGSSLNNREGTLQSQGKTNLTIAQHIENQQGKIQSESELVISGQNLDNTNGTVQGLKNIALTVVQQLQNQQGWIKANETVDITALSIDNQNTSQTAKGIEGQNIALHTTYLNNQTGIVRASQTVETTIAQKLNNEQGMISSENQITVTDGTNGKVLAISNQQGTIVSNGSANISANQLTGNGKLIAQKTLTLNLKQTFENVGRLQAGEYLTANFAQGVTNRGLISSLGELVLTTSTIINQVPGEISGQSTRIQASGQISNTGLIDGSLTHLVANSLDNRGTGRIYGDFLAIHGNDLANDKQNDKAAVIAGRKHVNLAVENLLNRDHALIYSDGDLVIGNTLDDQLQVTGHAKSVKNYSANIEAVGNLTLKTALLENKDIHLQLTDDAMEVSREHFDWYDFGNGRRYKIQPRSGNQTRYAINDDGTLNRNVGIHYEESNRWRMFVHGDWTKDFYEYVYDRIIYETQVIKRDAALITSGQHLTIDGQQLNNENSRVVAGKNLILTGYDLNNAEAQGVLRIVENGNTIYRYKGGGTWETYTSTSKYQGVNSEEDLALHLLEVTENAGGINKTQLDSVKVNQLDGQAESVSDANSQENQGVNVTEQALKEGKNTPLDLKPGQQMEVTQLPSISAKVDVQDDKSVDSSLHVDDTNIGKEADIQGQVDAGQGKKITVEAEPQGKDNLDTVIRTVGPNTQLPDNSLFNLTPSSDSQFLIETDPRFTNYKKWLSSIDIVTHEQLHKRLGDGYYEQRLVRNQLIETTGQRLLGNDKNDEEQYRALLTNGVAFGHQFNLTPGIALSPEKMANLTTDIVWMVNKEVTLPDGRIEMVSVPQVYVRARQGDLNGNGALLAGRNISANMTGKFLNSGEISSRELTDLHAENIENSGRIQGKDVQLDALKDVKNIGGEIRGLDNVSLSVGRDILSETAQRGDGQSQWLDRPASIYVTGDNGQLTLKAVHDISFIATDVGNLGVEGKTTIIAGRDISLETRDVSSAFDYTHNSSNYYRGANSTEVGIQIQTQGDLTLSAGQDLSARAANVTSDGELAINVGRDINVTSGIESSDYAKHTKNTEKGFLSSTTTETHDEGHERIAISSILSGDRVNINAGRDVNVQGSNVLGTNGVSIDAGNQLTVSTSDETLYATHMNKTTKSGLMGTGGLGFTVGTHSQKVTTESDSNQKKGSVIGSTAGDVTLTAGGSANIHGSDVIAAKDIDMTGSDVTITAAENSRTDITTVESKSSGLTVSLGGAAGSMLDGMAQTAKSAKQEDDSQLAVLKGMKAGLQGVQAQQAGELAGLKEGGSTADAFGVNVSYGSSSSKSTTKTEQHTASGSSLSAGDNINLTATGKKDGSQGNLTVQCSQLDADKNVTLTAKNDINLTSATNTQTVDGKNESKGGSIGAGISTGGWNVNASVNKGSGFEKGDSQFYTDTKVTAGKQLTLNSGKDTTLIGAHASGETVKANVGGDLTLSSQQVTDKYDSKQQSGSVSGSIGSGLNTSASVSANKTEMHSDYQSVDKQTGINAGKGGFDITVGKHTQLDGAVISSTADANKNTLDTDILGFWDIKNKADYQVDSQSGGFSTGGASVGDQFVTNAAGSLLTNVNNKGKDSNTTHSAVSEGEIIIRDKANQNQDINNLSRDTDNAHEKLNTIFDKEQEQKRIEKTQLVGELGKQITDIAVTNATINATKEVNANHPTLTGKAREEAIQAEINQSGWGVGGDNRRIVEAGTALVQGLANGDVSKAVANASAPYIANTIAQHIGEENKAGRIAAHGIANVALALAKGENSGAQSLGAMTAEAVGMLSTELYGKDVSQLSEDEKATVSAFATLAAGIAGGLVGGDTSSAVNAAEAGKTTVENNFLSATKNEKLMKALDDQKEGKNLLQASQNIVYLTNEDRASNVLLEQYRNDQLNESQQQDLANQLNQYGYELQTVYGFSETEAKAAIQNLVNGGAFVAAYADAKAYNEALSYLKMYGVQSGQAAVGADALVALPGVPGSIIRNTLIAGGSYQTGTGVGQVIEGNYTEGGINIGLGSLAVFGGIAGNKVVEKPTGSIVSPETTIVQESSKILDKKTHTSIPKVEAELIDKETGKIFKDTNQGNRPDYFLGDKSRPTLINDRIEAKVEKNPSKYLPNGNMASAHAEVGTIQQAFEDGITVGRDMNMKVTKEAVCGYCRGDIAAMADKAGLKSLTVYEESTGKTLYWNPGMKSLKEKK

SEQ ID NO: 129 - protein, 1885 aa, Propionibacterium acidifaciens; identical to SEQ ID NO: 74
  REGION 1..1885: note = MISC_FEATURE - PaDa02

SEQ ID NO: 130 - protein, 602 aa, Bacillus subtilis
  REGION 1..602: note = MISC_FEATURE - BsDa02
  source 1..602: note = subsp. subtilis str. 168
MSKVFESKSLIEEAKSRKKQYETLEEQLNTLKKAFQGVADLGDNFKGNGADNIKDFFQGQAEIVDSWLTLVSAQIAFLNGISGDIKDQELNDSYVETSFLDHELPNGDLKASEIVSAHKEEIDSILSGISDIIDLDMYTLDDYADKMGDAQKIRRDTITAVDKLDESLTTEYQNLESLDNAVLTKYSVLMQATSNGKSASPMYYDKKAFHSNEVYKSVIEVENQGTTYIDAKTQQAEARRLQEKAEEEANKPWYEKTWDGVCNFTGEVTGYYDYKRATEGVDPVTGEKLSTAERVTAGAMAAAGFIPVVGWAGRAFKGGKAIYKTGKAAIAAEHALDAYKTGKSLDILKMTEMGAYGLVASNGFSEAVTGRDMFGNKVSEEKRKQGALEAITIIGGAGLAHYFDRLYQKNAPYVNKVSNESLISNIAKTTEEKQTRLQYLRNKHGVLSKEDLHHRINLRAEVLNELSRIKSSGLTKKQRGPAVAGVLDKKTGNYYFGINNIDGKPPKVLHPLIHDRIVNMPTELKEGYIKTSGAGSHAEVNALNEALLQRPDADLKDLMVYVVSARKINKKMPEGVPMPRCPHCEYITQNTNYIPEALKYGK

SEQ ID NO: 131 - protein, 164 aa, Escherichia coli
  REGION 1..164: note = MISC_FEATURE - EcDa03
MIRLGLQGGEQTYRYVPNPCGYVDPSGLAMTSKIVNKLPSAKDLATKRASELLAKATSNTQRSKLPRAVSAVVDKRTGKIYYGESGWPHPTEIHPTLQNNMPSASKERWAIENCAEFKAVNEALKANAKISDLEVHTVLVKTGEAFPMCKNCQISIFGAVVTSG

SEQ ID NO: 132 - protein, 2106 aa, unidentified
  REGION 1..2106: note = Acinetobacter multispecies
  REGION 1..2106: note = MISC_FEATURE - AsDa01
MNKNSYRIIYSKARQMFVAVAENVRSQTKASGQSEASAQSNIDNTESQAFHQLWQVKALVASISLWMPLAPVYAGIVADSAANAANRAVIGAGKNSAGTVVPVVNIQTPKNGISHNIYKQFDVLAEGAVLNNSRQGATTKTVGNVAANPFLATGEARVILNEVNSSAASRFEGNLEVAGQMADVIIANPSGINIKGGGFINANKAIFTTGKPQLNADGSIKQFTIDQGKITVSANPNSKFGLGGNNNDANYVDLYARALELSAELRAKNDIQVIAGANNVSADLQDVTAKTGTGTAPTLAVDVKALGGMYANNIYLMGTEKGLGVTNAGTIQAVNNLVITSAGKIEHSGTISSTSKTQGLVNIQTTGTGAAADINSSGSINSNSMLNIDSGNNLNVNAKEIIINVGSLASSPLIVNAKGNINLAADTRIMDDSQGGDVYIDAANINLAAGSELKSNRGTATIQVQKDLVAAKGAKLIAAKDLNILSNGKLSLTENHIQASLGSINLQANSANTQNLIDLQGGTIYAGKDLNLYSSGDLNLKNLGFSLENAATRVKNIKAYSGRDLVWNNADKALPLITGLVQLDAANNLTVTAKEISNKDSIQLHANQIALNSALTSQKNIDVSSEIADLVLSQALKAQGDINLTALAGGVTANSLKATSSAGKISILANKNINLNSTQTSKAMPSADKDELTTDQTVISGLKGVTLGSIGDGTVNLQSVQVNANQGDILVSSNNGINLKANNDVVVTANKGQVKTVNNVLKGQTVSIENSKSDIKVQNTDLGSTVGKLAVNSRAGMSTIIDSVLTSKGNTELYAKDLLTLQGVNATSDQHLAVSSGRTVYSNAEYTPATKWIADKVTNLTSKGVTSVTATGNQVLQNTNLTGGAVLLEAGGFILGQTGLNLNAVGSDLLKNHTKLNSLNGDLTIQTNSNLTIDPKVYSLKAVGDIELVSKTGTLTLKGYGGTAGNGSEQVVKLDTANGGINLEGAKVELQGSNLKAAKDISIVSTNGDLVVDGVRNTITNQHTDEYKAKIIKLDEEIKILQKSSDYEAIIAFFMKEALKVYARISSGELIEGSPEMDRELQKALQKTTKEFPEYVSKYGPLRDQKDFYSEMIKLFNVGYVNGEVHSGSKIISNTGNIQLVSNKGLSISGSSMDAKDGKVNLEAAGALDGEGYNIQGKYNNPTENSVKKGVIKGSIIIDANQDSYEQGSPSDNDYRWFSPVSPTTINGTKGVSIKVIGKQSTDNLILQGVAITSENDVNIEAYKNIIFDVAINNSFNKSQKTETKSKWYGKKKTITTTQTDEKSGGLSVDIDAKNINIKSQAKNTPAMSGQNRTSIDMYSSQLTAIDGKINIQAGGDLNFLTADDVSLNTTDISKKSSFLGVKLNKSHTTNTRNVKSELPAELNADYIGTKSGFDTRLKGTVFNYLEGAEIQAGGTITLESASTTVTETLKKESNSVVWQSMQDKGSITETAQLPSFNGPVLPTFIAAGGLSVQVPISEKDVNKVELRNEILKLANQPGNAYLKELVNRKDVDWQTVLLTQKDWDYKSQGLTGAGAAIIVIIVTIVTMGSGTAAAAGAAGGAAASGTTVGLGASMLGAAGVTTTAAGAIVPSALGAMANAAITSLAAQASVGLINNGGDIGKTLKDLGSKDSIKNLAASVVTAGLMSQVGSALNLKPDSTLLPDRLANNFASSVGSTLVQTAIKGGDLGDNLQVALLAGLAGALQGELAQNLKGLEDVNYILHKIAHAAVGCAAAAATKQSCEAGAIGAGVGEIIAQEMLHGRNPTFVSAEEKAKIEAYSKLIAGTISAYAGYDVNTAANSATIAIQNNAFKDKALLKLEATRKYLDDKSKAALDGLINAYKKGDIEAAKKFKNQLDDSVTTWANSSSSYLNIETKSALGEIVIVVSDLVIPTNYVEILPVGKLSKVAKILKIGEDGTKSAGRLAEELAELQKVDIKFGKTLPGAKAPITVTAESNIGGKHMFDTNQTARPEVNRTNTPTLAAGNAKIDPSNPNLTMKNAHAEIALIQRAYDAGLTKGETMQVLVRGKEVCDHCGQVMKTMYERSGLAKLIIHDTTSGSTTTYYKVIDAKTNIATTKIVVSPIFGH

SEQ ID NO: 133 - protein, 484 aa, Nisseria gonorrhoeae
  REGION 1..484: note = MISC_FEATURE - NgDa02
MKPLRRLIKLLAACAVAAAALIQPALAADLAQDPFITDNTQRQHYEPGGKYHLFGDPRGSVSDRTGKINVIQDYTHQMGNLLIQQAAIQGNLGYTVRFSGHGHEEHAPFDNHAADSASEEKGNVDDGFTVYRLNWEGHEHHPADAYDGPKGGNYPKPTGARDEYTYHVNGTARSIKLNPTDTRSIRQRISDNYNNLGSNFSDRADEANRKMFEHNAKLDRWGNSMEFINGVAAGALNPFISAGEALGIGDILYGTRYAIDKAAMRNIAPLPAEGKFAAIGGLGSVAGFEKNTREAVDRWIQENPNAAETVEAVFNVAAAAKVAKLAKAAKPGKAAVSGDFSISYKNFSTVKPKVIAKGTINGKTFRDVNQSAKIGSPDSPTLIAQRVNAKIQADGKPRPNATVANSHAEIGVIQQAYNAGETKGASMTMTVSGKDVCGYCKGDIAAAAQASGLKSLTVNATDNVTGKNKTYYWTPGMKSIKERK

SEQ ID NO: 134 - protein, 786 aa, Escherichia coli
  REGION 1..786: note = MISC_FEATURE - EcDa02
NTSSGGSVGVGVGVGSGGWGISVSASANQGKGSEKGNGTTHTETTVDAGKQLAIISGRDTTLTGAQAGGETVKVDAGRHLTLTSEQDSDRYDSKQQNASAGGSFTFGSMSGSASVNLSRDKMHSNYDSVQEQTGIFAGRGGFDVTTGQHTQLNGAVIASTATADKNRLDTGTLGFSDIENRADYKVEHQSVGISTGGSIGGQFAGNMANNLLVGANHEGHADSTTQSAVSAGNITIRDTKSQKQDVADLNRDAAHANQTLSPIFDREKEHQRLQQAQLIGEIGNQVADIARTEGQIAGEKAKRDPAALNQARAELEAAGKPFTEQDVAQRAYNNGMAASGFGTGGKYRQAIQAATAAVQGLGGGNLSAALAGGAAPYIAEIIKQTSPDGAGRVAAHAVVNAALSLAQGKNALAGAAGAATGEVVGMIATQMYGKPVSELSETEKQTVSTLATVAAGLAGGLVGDSGASAVAGAQSGKTTVENNYLIVFEKTELEIAKQTLKNSKNPAEREAAQKKYNALREKDIASDKEVIDACANGNAGSSACAGARLKVFASKEGYEDGPYNSKYNQQYADAYGQIVNLLDITSVDAQNQQQVKDAMINYFMVTKGVDRQTAESYTETKQGLEIIAASVTPILGSVAAKQLSKIVDANLKVVARGNVDGARFSDTNQGVRPSQLADFNKPTLINDVVQAKIDKRPDKNYPNGNMGTAHAEVGVIQQAFDKGMTQGREMAMSVGGKEVCNYCLSDVRIMAEKAGLKSLTIYEEATGNVLFWQQGMKKIENRGPAK

SEQ ID NO: 135 - protein, 480 aa, Segniliparus rotundus
  REGION 1..480: note = MISC_FEATURE - SrDa01
  source 1..480: note = strain ATCC BAA-972 / CDC 1076 / CIP 108378 / DSM 44985 / JCM 13578
MSRPSQLRSTIDSATSSAKGQRPAEDAGVAGVRAATRDPFAEASSLLVHLVINRVPPIKHVLDKVTLDEGAAMALAQQWSDEFPAALREQRDALVSTRESISQNWEGEGASKTYQDRQRELEDLADSLAAACEGAGFLTSAVNELMVGVREQIIEWIAQLVSMLMRRLISVVATFWIPIVGEINEAAFIFEGVARVVETIERITSLITRVEGILGEVSSVAQVLGGSTQQVQQLGSALGEVPQGLGASPLRALGRVGSVRQSGLTGSHGAVIRSVVPRRSKSPGGSAHAQSAGTSTRGLGSQTGATPSASSGTHPQGGAVNASTRSLSPDSGTRGPVTRSQTAAERAQSSNANAQAEQARLDARARDLHSRLVNARTGEPDTRAQNSRTSVAMSTREGRDVLAGGGRDLNPSQRQAAGPNDVVATQPGAHAEVTAINGARAAGLTPSQIAVSRPICDNCQDAIQQSGGTVHPDRMGATWP

SEQ ID NO: 136 - protein, 588 aa, Neisseria sp.
  REGION 1..588: note = MISC_FEATURE - NgDa01
  source 1..588: note = GT4A_CT1
MPNKEENARNGLPETDWRDAQYDMLYLPVTETVRYHYDFNGNRTATVLPDGRQINYLYYGSGHLHQISLDDEVITDIERDKLHREIYRTQGKLASRYELDPLGRLKRQIATLNDLTETGKGKTKVAAGYGQTAVKRSYGYDRTGNLTHSTDQRTGTTKFEYDKLGRITQAGNELFAFDPAHNILSDDLNTITDNRLKTYNGTTYYYDELGNLIHRELADGEVQNYFYDLHDQLVKAEIFKKDGTKETWSYTYDALGRRIGKGRLKTSQKVSETSFPHDLSGNDLENQTRFVWDGSHLLQEIHPDGRYTYIYADQNSYEPLAQVRDWTTVEDENRQQTRYFHCDQIGIPREMTDKDGNLLWFGNYTGWGRLKEETKVTDSAYQPFRLQNQYADRETGLHYNFFRYYEPDAGRFVNQDPIGLWGGDNLYRFSSNIQIWIDPLGLACIPNPNRKNDEDLARRIDKLSDNLTEKNRDGFTTLALARVTLADGTSQIWIAQAGSSLSSKPSRRQQSLAGADEIIQNLHPAGRSRKDGNHLNDAERQLIREAKKRGAKIKSLGATKPMCGRCEKGARRAGILRRIITPIKSRHC

SEQ ID NO: 137 - protein, 347 aa, Amycolatopsis mediterranei
  REGION 1..347: note = MISC_FEATURE - AmDa02
MGMLFQRIHGEGSTVQRQSFQPSTGAPPTLEPPAVPLEGMPPEVFELPPLGSSAARAAERAAVDAAASQAERVVAERAVEEVVVQVGERIVSRRLLAGLTAVAIADEWNPVGWFLDGVIVGLLIYDAVEGYQTIRTSAQTRPAQSPPSAEAPGATSGVRIVDPVPVRPVVAPQAGPSAPVHAPGTGGGAPVTEPATDATPLEAARRADPELDRLMDIAITLQLEVGAVPDGSLVTAVSRGGPRTQSRRDRMSSPMIQSIRELMSQAGLLNPRDGTLPFHAEKKMVVQWGGPFAVNQNICPNCWQFLRWFAGTMGRRIVVADPAGVHVITPNGGFLTGPLVVRIPASR

SEQ ID NO: 138 - protein, 144 aa, Triticum urartu
  REGION 1..144: note = MISC_FEATURE - TuDa01
MKEHEIYKPPGSSIVELNGVVYEFLSGDRSHPQSDRIYAMLDEICKTLSLKGYEPLTKEVTFDVDEEDKEVCISQHSEKLALALGLISTTRGAVIRIVKNLRICEDCHSVMKIVSEVYDRVIVVRDRNRFHHFKNGSCSCLDYW

SEQ ID NO: 139 - protein, 236 aa, Ostreococcus lucimarinus
  REGION 1..236: note = MISC_FEATURE - OlDa01
  source 1..236: note = CCE9901
MRGESDGRNYGDAWMRRRVFTTAKALTTFERALVKVCATKATTVRVDRDDDDDDDDDDDAVRSTIDVGEFEDVTDWARRAVERGAEESRARCRAALPALEAMPTERSFFLAASDALEASVDDEGEGRSRRGRDRKVFAALHSPTRGGVYAVAANTNGGNAVLHAEMNLLFPADDARARRLIEPDTTLLVTLQCCRMCAARAVELGVRRAAYLREDPGPLASRTALAALARESRFDL

SEQ ID NO: 140 - protein, 1340 aa, Nostoc sp.
  REGION 1..1340: note = MISC_FEATURE - NpDa01
  source 1..1340: note = PCC 7120
MKTAETKTTSHQHQGNSAKPFFDANHEQKFFGAGIVQTKLFFQPQYTSSELGIQRQCRDCGIEQQGQPLPQIQRQPIFESENVETQSHLQRQATTSDTAPVTYLQPQEEATPELEETSGDLASPVIQSKGDGSIPPGDGDNSFRSDTETPFFQAKLTVGEPGDRYEKEAEAIAHQVVSQPQPSQQPTTHPAVTQLQRANNEQPGSPIPTNLESRLQQAQSSGTPLDTQTRLNMEAGFGADFSQVRVHTGQDAASLARSLGAVAFTTGNHIFFNTNQYQPASATGRNLLAHELTHTLQQDAAIQRQPAVSSTSHTVQMLPESIIGQINDYARYIPGYTLFTVVIGFNPLTGNAVERNASNLLEGLMGLVPFGTFIFDKLREMGILQSAFTWVEGELGRLDLSVGRIERTLEAAWEDIHLVAGFDDNLAVLRQNFQQLYQDVEAFARSLINQIIQLIKDAAINLAEGLLANNQAWALIKKILKHDPLRNQPVAATTVEILEDFLRLIGKEQELEQMQERGTLEQTAAWLDTQVGTFISLLGELGSLFTSAWDAIQPENLPNLSTNLESIINQTIGFLQRVWDFAITVAAKVLELIKQALLGWLSSFAIDIPGYHLLTVIMGKDIFTEQSVPRTVINIIRGFMSLLPNGEQKFQQMQETGVIPRAAQRIEALMGELGISWPFVQELFLNIWNTLTIEDMVTPIDTFIRVLEQFREPLNRLLTFVIEVVKIVLELLLEIMNFPSDIIGNIMNNALQALDDIQQDPVGFLLNLLRAVKTGFTNFFDNILQHLLSGVTDWLFGQVRKAGIEPPTEITLESILNLVLQVLGVSMDRIWSKLGDRIGQENVARIRGAIDRLSGIWNFVRDVQQRGIAAIWEYIQTQISNLWNMVLEQVRSWIVSRIIDRVIAKIVSMLDPTGIMAVVNSFIAFFNAVQSAIEYFREILEIVDSFVGMVAEIASGSIDQAAQFLENLLAQSLPVAIGFLANQVGLGNIGDKLQEIIAGIQGLIDQALDWLIDRAVNLGQGLLNAIGLGEDNLEETTGEPDTPESQALKQEVALAVQQAFAEEHTGAETESIIARIRSQFQAQGLDDLFLEPNSNGTGFILYAKASPKYPLGIFQKEANMTQAESEQMLHSAIRLTLVDSIGFNRQNIPLVESTEKNIGRGGEAWQPENLTTEIRAVTWNTSGANRRNNITHAERQFMNWITNPSQENVLLKLKKLEIQNINESPCGACSNQLAHLIREIREKQTSAGQVYLEEAHLYWTKRHLGESPTSASDITILRASGWTLHAPQNQLPIQGIEDSNLQELPGVLEYEPGFNPNDRAVVIHPPASSSSRSGGVRTRV

SEQ ID NO: 141 - protein, 388 aa, Orientia tsutsugamushi
  REGION 1..388: note = MISC_FEATURE - OTT-1508
MFAIHKKEQLKTALNEAAQYQEKYYQGSRIDGESPISLAGPRIKNLIRDSEQQKKLDYLAKVIAVYCKKKCVAVAYSEDNDEYYISYNTVAHGSDCSKAEYACVNMEVREIVKSFYSDIKHGLNSSNLRKILVDSILNDKNTEKSLMECVLKLVHGYSYIQTGDSSYDFSGIQPLFINSKNTKNTNIKLDGFSRLGEKYKELRKISNDLEKKLAEELDEEEKKELISDHNLIHKVKSELSQFHTVRFNLDELLPYFERNEGIENKLYVIKNNSSINEQQHAEMVVLEYVHNKSKVGELSGNVYIGMSKLSCFTCQKVIEYYNNSESNVKLENHGTHGKVYNNWAFPAGILDVVSVDDLIKDLSNSDDCSSISAFPFGKDSASSDSDGY

SEQ ID NO: 142 - protein, 3144 aa, Burholderia pseudomallei
  REGION 1..3144: note = MISC_FEATURE - BsDa01
MTSKKAFAVSMNKNQYRLVFSRVRGMLVAVEETAHATGKVSKGEAPRSVANRALSALESFALRHAAFAVLIAAGVTPMWARAQVVGAGANAPSVIQTQNGLQQVNITKPSGAGVSLNTYSQFDVPKQGVIVNNSPTLTNTQQAGYINGNPNLGPNGSAKIIINQVNSNNPSQLKGFVEIAGQRAEMIISNPSGLVVDGGGFINTSRAILTTGTPSLNADGSPGGFSVTRGLITVQGAGLTATNVDQVDLISRAVQANAAIYASNLNVVAGANQVNHETLQATRIQGDGPAPAVAIDVGQLGGMYSNRIFLVGTEGGVGVRNAGTIAADAMGLTLTTDGRLVQAGKISSVGNVAVSAAGGIENSGTTYGQQSVSLNTGADVANTGTLAAQHNVGVTAGSLNATGLLGAGVNSDGTVTQAGDLQLTTAGQLNATGKNVAGGNVSATGAGVSLAGSTTAANGSLSLSATAGDVNLTNATTSAQGAVTANAAGTVINDHGNLTSGGSTTLTGGNVSNQSGNVSSQGPLSVTAAGQIANQAGVLVSESTIALRGGTVANNQGTIQSAGHATVDGVTIDNTAGRITSLNTDGMVLTATGQLTNVAGTTANGAQGGVIGGNGDVTVQGGNIANHATITSNTNLHVSGQSVDNSGGALQAAQGVTVDAGTHLANGGGSIVGQTAALTGTTLDNSSGTVQADQVSLTATNVVNHGGTITQTGNGAMAVNVTGTLDNSRGGTLQTNSADLTLAPAALVNDGGTITHAGTGTLTLGNGSGSVSNVGGTIASNGHISAQSGSLNNASGSINAQSGLTAAVSGVLNNTNGKLLSNTDLGIASGTLTNDGGQIGASTNATIHTGSMTNRGGTVVAPNLSVTADSTLDNSGGTLEANQLALTAPNLTNHGGTITQFGSSVMGVNVSGTLDNSAGGVIQTNSTDLTLAPAQLNNAGGTITHAGTGTLTIEPGNGASALNNAGGTIVTKGQAVVDASSWDNSGGILAAQGSITGAIAGDVKNSQGLVRAGTSLSLTNGGALVNQGGHIQAGQQTAGDTSTLSIQSASVNNADGSIADLGAGKMTVQGGSQITNSHAGGVSGMGAITGNGDVTVSATSISNTQGGQLSGASLHVQGTTLDNSGGQIGNVTNSSGNVDVTTSGTVTNTNGQISSTHDLTVTAPTLQGGGTYSAAHDANVNLQGDFSVTPDYQFNVGHELAFTLPGTFNNSGNVQSVNNLNVNAGNIVNSGALSAGGLLHTQSNDLTNTGAIVGGSVSLNATGTVANVGPTALIGASDSNGTLEILANDIENRDDTTATDSMATTAIFGMGKVVLAGGKDASGKYTNAALINNSSALIQSGGDMELHADKITNTRRVMTTSTGSVDPATLAPFGVPIKGQTGQVGVKDPTSIGGVYTDPPHGGQWNSTYQYTTYYADSATATTVTSISPAAQIVSGGSINASTVANLQNYWSSIAAVGNVQMPKSYDANGWAATGQQAPSVTVSYSGQYHYNNYDNTEHDWQLPFGNAPFVTSRPGGYTQAAPASVKQYSLPSYDSTLGSNGTISGTGVSINNTAGNASIPSLGLLPGQAVPGLTIGGLSGSASGTKSGASAVHGGVTTIDPVIASATALNVLNNLTIPQGGLFKPNPSPNASYVIETNPAFTNQKSFISSDYFFGQIGVDLTHIPKRLGDGFYEQQLIRNQVTSLTGRAVLGPYTDLQSMYKSLMAAGASLEKSLNLPLGASLSAEQVSLLTSNVVMMETRVVDGQSVLVPVVYLAKANQQNINGPLITATDIDLKDAQNFTNSGTVKADNTLSIQGKQIDNAFGALQSGGLMSLTTTGNVDLTSAKVQAGSLNLNAGGDLILDTAVKTDKRVSRDGATSITTTLGPTAQLDVTGNAAIKTGGNFQQNAGNLSVGGNLGMNVGGDWILGAVQTGEHKIVQRANGVSNTDINNAVGSSVKVGGQSSIGVGGDVTARGAQIDLGQGGTIAAKGTVTLGAASATSTVNSNSSGSDSHGSYAETLHTSDQALTGTMLKGGDTITLASGKDITISGSTINLDKGNADLLAKGDVNVGAATETHTFESHETHSHSNVVSGVKVASGTDQTATYSKGSTISADGITVESGRDINVTGSNIVGTNDVSLDAARNVNITTSQDTVQSSSYYDKKESGLLTNGGLSVTFGSRSMGQTDQSKQVTNNASVVGASSGNVSISAGKDATITSSTVVAGQNLDVTGQNVAVNSAYDTYNDAQSQHFSQSGLSVGVNGGVVGLAQSMASTVRQGVQSGDSRLAAVQGVAAAEQAYQSRDGLKNAATALSSGKVSEAANGVQVQLSIGSSHSSSNETTSITQAKNSSLIGNGNVHVTATGTPDANGNAQPGTGDITMTGANVLGKNVSLNANNAITLQSAQSTEQDTSSNSSSGWNAGVGIGVGKQTGISIFANGTNSHGQGNGSAVTQTNTTIAAGNTLTMKSGGDTTLTGAQVSGDKVKADVGGNLTMTSVQDTSNYASNQHSAGASGSFTFGYGGGADVSIGHTGIDANYASVIQQTGIVAGKGGFDVNVANHTQLNGAQIASAAPAESNTLTTGSLGFKDVQNSMSYSASSEGFSTSSGPSFAHTGDSASGVTKAAVSPAAITVKSDQQNGTDSTAGLSRDTANANQTVKNTFNLQQTQNDLAFAQAFGKAATFAVAEAATQLENSSPQMKALFGEGGAGRDALHAAVAALGAALSGGNIGGAVAGSLAGDALQSLAQPIIDQAVSQLPLDAQSAARKALNEVVATAGGAAGGALAGGGSSGTLAGAGAAANNELYNRQLHESEAQKLQQLQKNQSPQEQYRLAAAECSLVHCADNIPDSDPNKAVLQKMQNDGAQFTYEQGVLKKAGAFDGYGKLDSLSDAYDRNQVSNRLVGAVQGVGSTAAGIGAATGGCYTLVACIAGAAVAGVSFDYAKAGFTQLVNGNPTPTYGELALQSLGMSPSGAALTYAGLGLGAAVGSVAANNAAAQAAAKGVPQSVESIQAGIKYDLMQQVADLRASLTGTPRTMGNMGVAQISIPGVQSKMAASSQIPDPTAAQRALGFVGEVNETFPSASVWTGGDTPYLLNRKVDSEAKILNNIAAQLGDNTSASGTINLFTERPPCESCSNTIIKFQEKYPNIKINVMDSNGVIRPSKR

SEQ ID NO: 143 - protein, 1050 aa, Paenibacillus polymyxa
  REGION 1..1050: note = MISC_FEATURE - PpDa01
MSVLSDGIGYESLRFYGPFQPLHIEQLQITRTINDHTILHISGMLSEEQGAACIGQNMEQEPIVIRQLDDQGQSLRRLFHGIVTQMSVNCTRGVYTFELKAASHSYQMDIKLKKRSYQDIHRTYDDLVTALVRKYQYGDAIDTVTNYAKLDTFVLQYEETDWAFLKRLASRFGSILVPEVTAASPKVFFGMPEGKQHKVERDVFYRVRKTFHELDAEKPGEQRAGSYVTYMIESLQYYALGDLITLPIGQGKELVVVRAVTRLEDGLLRTRYDLQAEQNIRYARYENDQATGISLTGTVLKVQQDFVQLQLDIDPKQDPAKACWFPVATRYVAEEHSGWFDMPEIGEQVELYLPTHREQDAYVTDSLRQQRHTNGQPNVKVWQHVQGSGVEMSEHELTLSTSGEFSITLHEGNGITIDSPGNVQIQGGHVKLVAGQELSLEAGTALYLKGGASSMVLDGETDTKAPVIYQEGTVKAPVFVADLPPVPEPPLMSIKAFEAAQSAAKDSSSSHASTPKAKVTSPAELKQANAMMGTVSKLLGSIPVMGKVAGVVVGALSGPAAGVILRATAAIPVRSKGTPTVGGSSKGSEVHPLKYLAGLAMQGLISIHEHEKAKQAYYNKWILGKVYTSARHIAHSGSTLELVQNLLKESNAMVHAYQQVPEEVRKRWRANYDSRMAQQQPKQQVEKPKSWWDRYLGYTGSSAVIKSQIAMEQAHASEKAANSLVELYNGIEQANNVRYQNAGNSIGEFLDYSSFGIPKGLYQAYMERARNQGNSGSDAINFATFGITEAIRGAVTPEDPLSPDHLSNIISVAGFLEGVGSFLKPKTILNSPVKEPVHPGPKIINEKTESPQLSTPKPNKDTRIEGTGNVADEVRPIDPKTGEIIYHDWIPARPIDLETEQYLIDKVVEARSGLSNKYKDKGNFAYVKAEIEGLDQSEFFAHSKISDPNKKIPGTEKFSLDPENPSFPATKAALDEGKGDIILRDMDTEYKILNDIDKKLGDNREAKGTITMFTEKDTCGSCNLVISKFKEKYSGITIELIHNNGKLIEP

SEQ ID NO: 144 - protein, 2615 aa, Salinispora arenicola
  REGION 1..2615: note = MISC_FEATURE - SaDa01
MRGTGLLFRRPSGRRIPTESGRWAGPTRRAFVLALAATLAVTSLPAAAFAVPAPGMSREQVDLPDLPESERVGRDEGAETDLTTAPEVPIDPYTPQAVDAWQQDSGVVDLTGLEAGDSRPVDDLPIALGVPEEGDPADLAGTWTVDLAAPEASQDAGVAGLIMKLTPPVAADPAAEVALSVDYTPFADLYGPQAADRFGVVLLPDCVYDAPDSGDCATDGGGSGGGAQARTAAPTDAADAPTGPAAAQAVPSEVGLVPADEAPTNSSAAEDEPTRRVVTGTVPVASLLGVQSAAARSGAASSAAASSAASMSTGSSVVGVLDTGASTAGDFTATPLLSSGSWAAGASSGAFTYSYQVHVPETAGGLMPKVALSYSSQSVDGRTSATNNQASWIGDGWDYNAGAITRSYANCRQDSKKPGANNSTHRTADLCWGSRNATLSLGGMTTELVWDEDEQRWFTANGDGSTVELRTDTGRANGDADGEYWVVTTRDGTRYHFGLHRLPGWSDHGSGADDPVTNSVLTVPVYGNHAGEPCYKAGNWAGSFCTQAWRWGLDYVEDIHGNAMSLWWGKETNYYARNFNFKAPVRYDRGGYLSRIDYGQRRDTLFTADPLARVGFTVAERCFDEGELTCSEENFTSSDPAKYRIWYDTPADLRCTAGKKCWNAGPSFFTRKRLDKITTSAQRRTNDTTLQAVDEYQLTQSFPILLTGPNTALWLESITRTGFARNGTTDAKVTLNPVRFEHNAEDMPNRVKRDHRPGFSRLRVARVINEYGGETVISYKAPTGDCATGTGLPGKDDTAALKANTRLCYPSYWHPDPEAEEIDWFHKYVVDSIEELPAIDGSSVATVTRYTYGPPAWRLAEREFTKKSTRTYSQFAGFAQVAVLTGADEPSIGSARTKTVTRYFRGLGDTVSVPDITGAEIAKDRKPFAGRIAEELTYVSAEDADEEWLTRSVTYPAAQLLASRERDDGLSPLEAWRVTEPRQVAHARSSGTGDDTRTERVVETRTTFESTHGLPTHVESLGDTGKTGDESCAFLEYHHRLDKNLIGLSKQVRSSPTTCAAATFDDLTTLSSASRVAYDGQDYGAALGDGTRGLATGTWSLKGDGSGFQPDGTTGFDAIGRVVTRTDVDGETSTITYTPATGQAFTVSEENALGHRQTREVEPGRAVSLKTTDVNGRVSEAKYDPLGRLVEAWAAGRTPSTAAVPDFRAEYATPAGKPPYITTFARGHEDQIETSVTLYDGLGRERQIQEEATGGGRLITDTLYNSSGEVWQTRNAYHTDGSPIGQLFTPLADTAVPNATRYTYDGLGRVTRELPVLDGVETPARATSYTYGDDHSTVVNPAGAASYRIFSDAMGRTTRLDTFTDSGHTEFTSIRYEYDARGHLVQATNSVDSTHPWTWTYDQRGRLVTAVDPDSGTTRMTYDRFDRQESVTNGRDITVWNGYDKLHRPTAQRLDTSTGTMLASYTYDSAPGGKGLPATATRYTDGLAYTQAIGGYTDDYQPTSTTLTLPQSIADTWGLRTTYQYDYTYTDTGLPESATLPAVGNLPAEKVLTRYTKDGLPLSVSGRDWYGAETVYSPYGQVLRSTLGAQPYRVWTTASYDNASGALTDQQVHREQVGDQSIVAANLVSHRSYGYDAAGNVTSIRERSLGIEERQCFRYDPIGQLTTAWTAADQGSCAADPAGGAGAVTAGTDGSGYWQEYEYDLLGNRTKLVEKDLTGDTAKDATTSYDYGRADGGQPRTLTKVTKDYVTPDGAEVTAVAERLYELTGETKTVTSVENGDQQVLSWTYDGKVERITGAGGRGKTAYVGLADKCIDLSRAVPGNPLQLFPCNGTIAQKWTFAPVPGQANANLGTLSVYDDWCVQPAGNTTGSAFATQECSGSTAQHLERLSTGQLKHPASGLCLAVKDEATDDRTPLVLVTCDGDSAAQQWEAQNETRHLYGPGGSRLLTIQDQQATLQLGESVLTVQRGGTLVNTQRSYPAPGGVVMRYAHLATSSTGLVALAGDHQGSPYAEVGLHNEMPVRVRKQDPFGNQRGAAPIGVNMQTHTGFLGAQRDDASGYTPLGARLYDPVVGRFLSADPVLDIADPMQSNGYAYAHNNPVTYSDPTGLSVSLTASEKAAALAGAGLSAAQVAQAQATMGRSLMSVILDSAWYMLKEFIGINDAMNCFGGDMWACGSLIVGAIPWTKVAKIPGVLKAVNRTISAIQAWRAAKRAAEAVLRAAKAAETAALNAKKLAIEKAKKAAQAAKKKAAAKAQTTSNKAVNAAKKTGNQVQKNAQAKSNPKGSSAASSGAGKSGKSGGGSGKSGGGAGKGAPKGGGDSGASARGNGGSSGASCKTNSFVPGTRVLMADGSTKPVEEVQPGDKVLATDPETGQTVAETVTAAIKGDGVKKLVKVTIDTDGDRGSETAEVTATDGHPFWVPELGEWIDATDLRSGQWLRTGSGTYVQITAIDRWDVSQATVHNLTVANTHTYYVVAADTPVLVHNCGLADAVAEHRANANGGLGVSAKKNIAAMEATIDGAAPRTTIATSGVHVNPGEVGMPATRLFTPPNPSRAFDSEVFLFEDLAQGLRPKSIGTINLYSELTVCPSCGGVIDQFRARFPGIRINIRTGED

SEQ ID NO: 145 - protein, 2034 aa, Chitinophaga pinensis
  REGION 1..2034: note = MISC_FEATURE - CpDa01
MPRIFRYLLCLLTLLTGFYHADAQQYPVTASTQIVPPYSVYLPDYAVPGSDKLRVILVQNDLTVPSYDIRLQVTVEQNGSVIMRSSPNFIPKPLTLSPGIPTIIGGIDLADYLNPANIQYSGGFSREVYEKTRSLPEGAYRISFTAYDYRRPQVQVSNTGANVFFFRKNDPPLLNLPICNSRVEKLDPQFLTFNWSSRNSPSPLPGSGTEYVFSLYEVRPAGSNPDYIVRSAKPIYTLVTEMNTIVYGPGEPQLRDSMQYVWTVQARDKSGRDMFSNNGLSLSCTFNYLGNNPFTQRNIPKPVLGGRSTGQRSVRFSWPLADAGYQVETYRLQYRAAKTGSTEFDWQTQESAGDTAFTANGLEPGRAYEGRLQWKIEGIYGPLSDVVTLKTDSAKTFVCGDPALMTAPANKEPLASAHVGSIFRVGNYDVILTEVSGSAGKFTGKGRVITLGFGIGLQMEFKDVTVNTDMVVTGGEMHAVTEGIDKFIQDKVDEQRGGNDVGKVKTGDIVPDITTKLKIFSPANIKVNVDAGTITLKDSETGKEEVINYKDKGKTLPLVLEDADGNLYNIDKGGKVTSAGKRDTSLTKEVLASLKTLNLSKGTVTFSASGDNAYAFDEWKDSYSGKSVLENKYESLADGKYRVSAKAIVPGVQEELVAILKGAGADITSSKLKFVTGKGIVLESKDLGDAKYSVKVTGGPGGDAQEVYAAYPDGKGGYISMGKLLVASYTPLQKTVVLIPIGEAVVNKEVLLDSLQHIYGKLGINYTVEVDESFKNNKDWDSNHDNVLQDRGSAFLSNNFTGEEKILKKLYKKRTKLSDDAVYLFVVDEVALADGDLLGKMPRNSQFGFIFTKDAAREAVVRTVLHEIGHGDYTLEHTFGEGIGLEKRTTDNLMDYSGGYRLLKYQWDILHDPGSVWGIFEDDADSENYFGVNISKIFLNKDKKTVSFLTPSATVLAVPWEKLGKVEFQYGAVYKSAATDQLNFGPDLTVGAVRSFELKDTGNAVQRYLYYDNTGTYKSESTGTAFQQEAAEDMVDGFIYPLQCDGKMRMYKFTRNTIPLYKAGDPIRKFVDFAETFRPFAVNTIMNDPLSFITLSQELPADVNNRCPYCIATTTVSMMKNHCAGAEYLWLDKIAQVRQVYPEYFTRFTQPEVVVEESVTGGRREKPGNWEVPLSFDMPLSTTHYSDSRPERATDYPWGKLLADSAEIKTAYTGDKVRFYKKFYTEFVNYITVTAIATDNKFWDTLTRRASVYEVFPHVRDEPYIHLQTVAVNKRGIALEIITKLYENSTTDFPHEDEVYLKLLTSFKGVADQTALLKYIEDSILFSNIYNPFNRFLMPAKMQIATMMTYSNMITNTGHCNYLIAEKAEQIDHPDIVAQLEGDMLQFKNAGIDFPEGNKILIDEKPYNYNDIVSVQVAGSFTIKINGVDSTFAKGTILNIPAIQVGLMSEINTADVAAKTAWLAFDVGTMVIGIGQAKILFTAGNYVRKSIVALDLLGSTAGIGLQLLDEDAISPQLRTSLQIASFVCSLPSMALAIPRIERKVSELDAVLNTKYGKSGLSAKQLREKAALERIRQELSAAAHLPEVAEDAAEISRRAVTISKEEDINKVLSWLNTAEDADPNIYLAVHAVGGDFKVMHGGKEVIMDHRSLANWILANEGLFPANKQLVLLSCGDIETAQHLSRKLKRSVVANDGPVRVYSNGVIEADNSFRYIDEAGYIDNSTSVVVGKQAVPPASDKDVVTLGLKAVKATTVSELVVEFKRIFGIGSELALRLSKNADFVKAYQENARILQVIANALPEVEVTVKGKKFATYREALLTGCADDPKLFTVMNSAIEEAIEGVKNAEDVKPFVEYAATRRKMTEYMNKNGDFNKDYFANAVLNSAVFRYAALDAKGWGKIDEARKAFTIHPNSNVLVVERRMKNGKPIQEVSHSGGRTADRTSESIEGTIKKVNEDNKHFDQFTVGGPRIQDSESKYFEDLLEKLEKKELRAEDIEELILYTERPVCPSCLNVIRDFKKRFNIEITVYEGKYKR

SEQ ID NO: 146 - protein, 1367 aa, Sorangium cellulosum
  REGION 1..1367: note = MISC_FEATURE - ScDa01
MLASSFFDLVLGLDIHFEMVPTPAPLPLPIPNPFTGIIFDPMGLAAGLVLGNLMAAVSGAPPTGPVVYWGFVPATNTGTEAIHIPGHILFPPGVAWAPFPKTPKPVIHPGEVPAPALPIKPENDAICVFGSKTVTVMGSNAVRFGDILLSCSEPIRLPSSVVLAIPKGAPILIGGPPSLDLMSAVMASLRTRFVSDSLHALVSRLRPSRFRNFLHRAVCFFTGHPVDVASGKVLTEAVDAELGGPMPFRVERIYSSACASRRGPLGHGWSLSLDQAIWTERGKVVLLAEDGREIEFDTFDHPGHTMLPGEAVYHPIERLSLRCLGGSRWEVEARDGTVREFAPVPGRPEARAALQRIRSRCRNHEIVLAYDARGRLEWVRDSAGRLVWLEHDDEDRLVALNLPLPRGRGFAAHRQYRYDDQGDLVAVSDALGHEWRFEYVTHLLVRETDRNGLSFYFAYDGLGEDAFCVRTWGDGGIYDHRLQYDKRGRVTVVTDSLGNTTRYYMNVVGLVTKIVDPLGGTTEYQVDPTTLQRTREVDPAGVAVERRYDDRGNLIELADGVGSKVRLEYDRGGRPVRAVDALGGEWRWEYDENGSLVRRVNPLGEATYLTWDRGLLVALALPDRRKIHVEYDEHKEIREIRSGGSGKSTYERDERGRLIAARDPRGAVVTTAYDAEGRTLERRSRSGAAERHRYDPEGHLVEIDSDARRIKLEYGHFHRLVRRSEGGGEISFHYDSEGHLLAARNEYGEQYLFTYDASGNVATETRFSGGTWRYRRDAAGRVGAAVDPAGSTSTMTYDAAGRLARVDHGDGTFLEFRYRADGELEWAQNEHSETSCHRDAIGRILAEVQPAGRVASTYLPGGARHRIESSLGARCTIVHDAGLGARELHLGGGASPEAVIAFTRDPMGAELSRLLPGGVELQWDRDIAGRPLARRTTRVDSGAARLDARAYEWRGEDQIAAIVDAARGARTFEHDRAGRLLSERSPRGDVARPLRRFSADELAEERASARFGPGSRVIDHAGVRYTYDAAGRLQTKERDGRRWSFHWNGHGLLRRVELPDGRAALFEYDAFARRTAKRIVRCGSSGEEVVDSETRFLWDGHTLLHEISIKHGLTTWYWEPGLFAPVARERGGQVWSVIPDHLGAPTTLFDTSGVQVWAAETDTLGRTAVEQGAPEDCPWRWPGQYEDPETGLYYNRFRYYDPDAGNYVSPDPLGLLAGTAEYAYAPDSLVWFDPLGLIVLQQVPYNDHPLFGAVSEFIQGKSRSDLRGRNVAAVLLDDGTVIVRASEGGGNHAERVLMGLSEVDPAKVVAVYTERSPCTGRINCHDLLDSSLGADVPVYYTHEMIRGQEGKTAQQIEADRNQFCRGG

SEQ ID NO: 147 - protein, 1539 aa, Burkholderia pseudomallei
  REGION 1..1539: note = MISC_FEATURE - BpDa01
MALPAVKHMDPVVGIDVHSVIVTPGLPPVFLPHPHVGFMLDLREYVEAAKGVIGSIAMTIAEDAVQDYLKDHPDDGKKLTDALNSLDNQKKQAERDPWVAQALKLDREVSSIKGDVTNAVGAGVGMGGAAGRPIFVNGLMRATAGTHSFHVPGLHFPLGETFAPPDPLPSDDAESYMGSRTVLANNDPMSFLALPAMSCWAEGIEPPPHNGAHTTRTHVSMPSSVMLPIPVGRPVLVGGPPVMNMAAAAKGMFKAFQGSKWAKHLADKLHLKPGFLRCAVLKAEPVDVTTGEVVVQQNDFTVSGRLPLAWDRYYASQDRHPGAVGFGWQTPADIRIELMRNEGGIGAVAHFPDHATAFNAVPADDGWPARTYDWQHGHALYGDDGRMVLRTREGIEYGFVLPSRWSDAVAALDGDDSRLTLPIDRMADLNGNAWVFERDVYGGLVRLVEWKRDGRTGRVVECGTSSGLHAGLLTSLTLIDAEGNAHPLVSYEHDRERNLAAAIDAMAHPHHFEYAAGHRMVSHTSARGVSFYYSYQQGDDGVWRVDHAWGDNGLFDYRFVYDRARMETRVTNSLGHTSITQMNERGVPVAEIDSLGGVTGYRYDTQGRASAVIDTAGRTTAWEYDAYGSLVAQTLPDGSVVRAEYDADGRPVCITMPTARQLRYEWDEHGHLRSRTTSSRAVSRYVYDAYGQLVSYSGSRGAITRFEYDRDGNLAAVTDALGNRTRYVRDARGRVVWMADPRGQVGLYEYDGNGNLTRAVLPGGKEIRCEFDDDGNLVHYRDAAGQSTTLAYSPIGLVERRIAPSGGIVDCRYDTEGQLVGVVNERGEQYALKRDPLGRIVSESDYWGQTWHYRYGGSGELLCSTDPLGRVVEYQYDRCGRIVEKLARASADDAFVQVHRFAYDQCGNLILAENDDSRVEFCHDADGRVIEEKQGDDFTINNVFDAAGNRIERRTRLLSEGALIEHVVHYEYDVLNAVVSIRIDGSAAVVIERDDLGQAVSEGLGPALRRAFSYEAGGQLAVQTLFASTGTMFASEYAYDPNGEVIEKRDERGRMERFEYDSVGRVVSHLGARGIVRRLAYDLAGDLLRTRVSGYDAAALADAEKAANWIRDGEYEGCYCAFDRAGNMVHRRDAEQNLALCWDPAGQLRETIALRPATADVASARKRTRTQYEYDALRRRTRKLVQIESEGEPDSVFSYVSCFFWDGDALVGERTTGGWVGRAGRAEEDERDATSLAPPSPAGSKNGNWLTLQPDHVCEWFYYPRTFHPLGVVHCYAGEKRELARATVGSPSKADATYFYQSDPNGAPVRMLDVEGNVAWEASYDANGGIEQFGIQAMPQPLRLQGQYFDAETGMSYNRHRYYDARIGQFVSEDPIRLSGGENLYRYCVNSISWADPLGLDRVPLFDPNNRLSFNAIWAYTGGLPTPSDVTVVRPATSWGRNSGVLKWQDHYYVFASAGKKSHSEDAIIDFIKQHDIKPSEIQGLYSILSPCAEEKKSCLEKTKGEGLEFDWSLFHQTDNVGSEVRKEIGALIKNMKGV

SEQ ID NO: 148 - protein, 1545 aa, Streptomyces coelicolor
  REGION 1..1545: note = MISC_FEATURE - ScDa02
MRLRRPRRPSDGPPGRSGARRLRVASAPLIPLTVIGAVVAGLIGPEPPSDGPLPGTSAVADSYDGFDADAAEQLRQDQCLMAGALRLGGPEMGGIAQTALNQTPEQLHVAADQEYWNDTPLALAYVKDRELIDQESATLDPRVQAWQDTLSGLSTPGGFKSVTDFSWPPGSGGGADFFDQVGYSTWLGEQWWKEEARFYEDPSAKADEATVKAVTDLGTPLYGDAVYDPSMSSEEWALAVEQEHAFDELLTGDLFSGMREDDARMFLSVGGFPSSAPEPGSLAFRLAVEDLKTRFAACTWTNPVDPGKVLGQEVATASVEWQREVAEQAIPRNQIIDANRDATKALATGAKALGEMLGQSWTADHLARWQDYWSPGGAGWIGDSPMIVHIHAASDKCLEVAGNSKADGAVVQMYTCNGGANQKWRVDGDSLVNVNSGKCLDVNRDGTADGTRIQQWTCKQNGAQGWEFTPNRTTQLRNTGSGKCLDLHTYANSQDAWLWQCNGTDPQKFDIVASGHNGTDALDYPTSGQFTQATKGITNARAKAAEQLGVIRRQAQIAGMAAADSEESLNTAYGIADSMGAPRGRALLVGQQKDQVTKASSAALNAMIKAGETALSATKASAGDSASIAARAVTQAAGTQAAFRTAAARAAKEQAKAAADAAAVQASNAKAARDTAKAKLTETQNAEADAKAAAATAHAKRLAAEKEEATAKAEKETAAQKQAEAAEHKTKAQQRATEAKDAKERAETADTTATEKRKGAEAARDKAKALRDDAWDAEQKANAARAKADAKEAYAQASASEDNAQEARKAANDADAAADDAETAAVAARSEADKATKAAADADAAATRAEAAAKRSRADSDAAQAAKLTADAAVRTATSAAADAIKASKTAATAARTAVELADDAEQHAADAKKEADAAKAEAVTALAGANESTGYAYTTAQAAVDAGNSAAQVAAPANDAIQLGSPYVTTDSAAGLAVLTGQSSKTIAEQQQAVAEAHAQNAEESAAQAQSVANAASGDSKAAYTLAAEALGYAADARNSAKEALGYSAEAASYATQAAQSLTRTIAYDTQATKDAAAADSAAGRAEGHAEDARDSADAAALDAEAARSAADTAEQAAEDARDAADHAATEAAAAEEAAKDAQKYAESAQQAAEQAEKEANAEQIDKGTVVDQTGAPIGDVFYVVDHIEKIGEPEVVKQSDGCEGWIDELFYNGDCTLTTKLRYKEVFDLYLCTAEDLDASQYMCPSGATLYLGEYTSDELSTEVTHTITIAEYQKGVDPVDILFGSWIKCAQKIAPGGASGSWGGCAWAGVDVASLFAGKIIRPIADAVKAVDAAARTGIGFADAYKALRTLGLPTDAAFRIGVKGLDGFVKTCTRTRGKLTARASTAVGGCPVGLVAYNSEEMSHWAYRYRTESEYFEGDHNVAVAKVPGWNDPRTGDFIIANSKFSGHSETEILGKLEAKGFTPGQITALYTERQPCPACASVLTGSLKEGTPVTWSVPYHPDYAKESRSLLDSYVRQANGQQRARPTTTQRLTEGNEAHD

SEQ ID NO: 149 - protein, 265 aa, Streptomyces sviceus
  REGION 1..265: note = MISC_FEATURE - SsDa02
MAMNGRDMNDSDIRRIGDLTLPLSVGPYFVTAGSDPVPLQEFAESVGRTVVLEECREWARFGSDRGFEICADQKGVVRAVLLDWTEESRFVNATAEAFAESLTALDQALAVILGTEVPQVAAAAYAELEQRLRTLDPGAFEGREHWWPLVLDDIRDTASAEWFTAFEILNDQGEKQIVTQAGDIGVHPEERLWARLRAAGVQPEQVLRVHTELEACFLPGHYCSLWLGQVFPEAQLTHNFPYGETAESRAEGIRQLREAAAQQPQ

SEQ ID NO: 150 - protein, 287 aa, Kitasatospora cheerisanensis
  REGION 1..287: note = MISC_FEATURE - KcDa01
MTTELTQRLHDRFGPAGLYRPDPAALSGTAAPARTAALLAAAGLPLQVGPYFAANPGQPLRLADWARTAGLSADGRPEAGWLRLGSDRGAELCVDGDGRVRAVYLAFRGPSVPVDDSLDAFLDNLCTLDENLRALAAAEQPDEVFRLFSAAESRMRGTDLRAFESDEQWWPRVLEDVRHTRGVPGYAAFKIRGRDGEPQILTADGSLALHPEEALRERLSAAGLDPDEVVEVYTELQACTMPGHYCALWMARAFPNAEFTHSHDYGDTAAERDAGIRELAAALAAGE

SEQ ID NO: 151 - protein, 965 aa, Photorhabdus luminescens
  REGION 1..965: note = MISC_FEATURE - PlDa01
  source 1..965: note = subsp. laumondii TTO1
MNNFDPKIYQHTPTITVHDNRGLDVREIHYHRATAEKNSDIRITRHQYDVLGHLHQSIDPRLYDAWQKDSSVKPNFLWQYDLTGSVLRTESADAGNTVTLHDIENRLVLTVTATGVKQTRQYEDNSLPGRLLSVTEQEPDERASHITERLIWAGNTPAEKEQNLAGQYVHHYDTAGATQLNHLSLTGAALSQSRQLLVDGQEADWFGDDKKIWDKKLSNDVYTTENKTDATGTLLTQTDAKGNIQHLAYNVAGQLKGSWLTVKGQKEQIIIKSLTYSAAGQKLREEHGNGIITEYTYEPETQRLMGITTRRSTDSKALQDLRYEYDPVGNIISLRNDAEATRFWRNQKMVPENIYTYDSLYQLISATGREMANIGQQNQNLPSTALPSDNNTYTNYTRTYAYDRGGNLTQIRHSSPASQNNYTTDITVSNRSNRAVLSTLTTDPTKVDALFQAGGHQETLLPGQNLNWNARGELQQMTPVKRDNLTDRDSDIEWYRYGNDGMRLLKVSEQQMQNILQQRRVVYLSGLELRTTQNGDITKEELQIITVGEAGRAQVRVLHWESGKPENINNNQVRYSYGNLIGSSQLELDSDGQIISQEEYYPFGGTALWAARNQTEASYKTIRYSGKERDATGLYYYGYRYYQPWVGRWLSADPAGTIDGLNLYRMVRNNPISYHDNNGLHPNSNNNGLERNHPLYYLDLAGVYYNVNDKEAILSTLKRSFAATSWGNKVSDKTQTILSASAEYGNLKHKKQMEYNNKKLNETSPDGKFKYAKTKLQDQAAHAGIAFNLHPISDNEKEGWTFNYKDEFVNLKDKEKSLGKGKLFPGVELLPDNPEHYKVQNVSEFISGIESAYKNNGVELHPFTSSLIKKHILKNEEVLPMMAGIAGLHAEVQAMNFRFIKGDLDSKVEIGSRSKRSYEIDMLDSYIFTERLTTKEKGKEFTACFNCTGILQWPTRVMTGTMRRQ

SEQ ID NO: 152 - protein, 500 aa, Blastopirellula marina
  REGION 1..500: note = MISC_FEATURE - BmDa01
MRQVVTKTFTSGDLASNEQVTYDYDATGIRISSLHEVDSNLDDVYESSTRTEYLIDPANFTGYQQVTRETTYDANGNVVKVIAYGFGHDEITQTTTTYDENGAVTSEETLVFGHDGHGSVRVLYDMAGALQQLFTFTAHGQMLAIHNALAAFVSSAESAALTTLLYSGEQFDSRIGQQYLRARYYDPNSGRFNRLDPFAGNSQDPQSFHKYLYTHGNPVAGVDPTGMFTSGQIGVAMAINASISGGLTAAYGAAKRWSLERILIASSISAATAALMTLGFFAIGAGLAFATTEAIAITATGLIFAPTVLGFAIANFKTAMAKDDPVDKVFATIDLSLAVFGSIRLGIDTRNIAFRQRAHAGALAERLAAIEEGYNKSAAGVMNQGSASAPGRSTRDGGRQQPVDPEIQNFLDGLDAISRSKYHGSCVEPRSGSSLRIGGMSLKNAWSAVVDIANGRPYPACKSCRALLKNFGINDGFEPDLAPLYLLIDSMFYDTADGDR

SEQ ID NO: 153 - protein, 2060 aa, Taylorella equigenitalis
  REGION 1..2060: note = MISC_FEATURE - BadTF3
MNRIVNLKTIFGDSLRFLSLKGKDELSNLFEYRVQAVAEDKPIDQTQLLGTDVSIEIDTEKSLRYINGMISSVRKVSEKIEDARKYFIYEFTVVPHLWYSTQTNNSRIFQEMTAVEVVKKVLEDYDVYIENKLFESYRKWNYCVQYNETDFSFVSRLLEHEGIYYWFRHDDKKHYLVLTDSKDTHPKLPVNGTAYFYHEDKVAYAHDQHVFDWQSGGQITSSSYSTQDYNFENPRGDLSGESNVGSKYSAGRELDIYEPFGGYISADESEHYAKVHLESLQALQDYAEARTRLRDLASGYKIELKQAPYAADDGEYLVVKTEYDISVQSYVSGDDVNYNLQTSALLIPADVQYRAPRIHKEPKMGGPQTAVVTGPAGEEIWTDKYGRIKVQFHWDRDGKRDENSSCWVRVSSPWAGSGFGGVQIPRVGEEVVVDFVDGQPDRPVVIGRVYNNENMPPVNLPDDATQSGFFTRSKNGDASNANRMMFEDSPGNELLSFVAERDMNTHVKDKQTQTIDGNVTSSISSFRSHTTHSTSDVTLKSGREAKYESDHERLIKTQLTEKIGGNLKEDYLDGVDETIKGTFTSTVSGVATHTMLGYHLNSETSDEEEVTGDVVQSVGVDEIFNINTKSKLNTGSLSWKTPKLGIESKSSSTKIDSASNVDVSAIFGYTMQTEASITNKAPLKFEFELLSSKNKITHVNDVSTSISLTGVKGSVGAQTLGLNVIYLSGVVQNNSKQYFSVGASGISGQLIGKENKDGSLATDLVGLRFNRGLKVWFSGPDSGGGSGKGGSGKGKSDKKEGGGDDKGGKDKNKKSDEPSADCKECKGLSSSKILLAPVVGMAVDGIVDIGMDLASGAVSQATSPHSIDFSLGEERFYHTDFALPGIKKISWTRMYRSNNYAFDQQSVLSPLGPRWMTPYMQFIYLHEETPVFVNFEGRLVRCPQQLPINKEIYDRNEELFWSQVDPDTLQIRYKNDNKLIFKKIGSVFRLHLCTDAQNNILKLDYDDESRLSNLENDLYNLYFRYDDQNRFDEVYYYETDLETGNKVEVVLAKYSYDADGNLITATDRYALSNKYKYQSNHLITRYEDKTARGVNLSWEIDGSHGRCVHESLDDGSEALYIRYERKELSTYVTDALGQTTKYVFNEDNYLMQIHYCDGTKRINERDKYNNITQVRYQDGTTSKFVYDAFDNLVEVLQPDNTSIRYDYDLDNNLITLTDPMGYEWHRTYNNLKLVESEIDPLGHVTKYQYNTKGQVIRIQDAKGGNKHILYHPTGEVASFRDCSGKTTKWDYDLLGRVVKMTEPCGTTEVYTYDYYSNICKVERSGTNPVYLEHDAEGRLLKFIDGLDRETRYEYNPAGRIETRIDALNNEVGYRYDVLGRLSKLINENNDKYLFEYDPLSRLVYERGFDGKEKRYQVNKLTGRLDSVTFVDQTLSFQYDIMGRMTKTSSADEVREYDYDLNGRLTLAKNPTSENQFELDALGNVLREIHSYDIFDHHVTKLWRFEYDELSNLIKTVRPDGKEVDYLRYGSGHLHGMLLDKERIVDYERDDNHREVKRHWAQDMLQTTAYDAAGRLANQHTQSGKYVKKSILNRDYQYDGANQLINIKDSRKGELSYRYDPIGRLINAKGPLGEETFSFDPAHNILSKEHSALSTYQHQVENTLPSGLSKVMGNLLRHISGSHFDYDQRGNLIKKESAKGIQTFDWDGFNQMKSSTFRGIGLSAGSRLDTQYRYDVFGRRIGKRVVDVRTKEVLEQTLYDWDGLTIASEDRIGEQLAVQAQIRKEAHSEIRKLRSEVQYLYEDGEFVPLVQYTNTAAVGFGYGVAGGGSKGVAPAIYHYVNDQIGTPQILMNAEQEVVWEAQAKAWGDTIISAPVSDDGVINNHRFQGQYFDAESELHYNTFRYYDPEIGRYISLDPIGLLGGLNNYQYAPNPVEWVDPLGWKFSNGKRRPPHKATVTVTDKNGVVKHKSNLVSGNMTEAEKKLGFPNNSLATHTENRATRLIDLNQGDTMLIEGQYRPCPRCKGAMRVKAEESGAKVIYTWPEDGDLKKREWEGTPCDKK

SEQ ID NO: 154 - protein, 199 aa, Gorilla gorilla
  REGION 1..199: note = MISC_FEATURE - APOBEC3A
MEASPASGPRHLMDPHIFTSNFNNVIGRHKTYLCYEVERLDNGTWVKMDQHRGFLHNQAKNLLCGFYGRHAELRFLDLVPSLQLDPAQIYRVTWFISWSPCFSWGCAGQVRAFLQENTHVRLHIFAARIYDYDPLYKEALQMLRDAGAQVSIMTYDEFKHCWDTFVDHQGCPFQPWDGLEEHSQALSGRLRAILQNQGN

SEQ ID NO: 155 - protein, 1427 aa, Burkholderia cenocepacia
  REGION 1..1427: note = MISC_FEATURE - DddA
  source 1..1427: note = strain H111
MYEAARVTDPIDHTSALAGFLVGAVLGIALIAAVAFATFTCGFGVALLAGMMAGIGAQALLSIGESIGKMFSSQSGNIITGSPDVYVNSLSAAYATLSGVACSKHNPIPLVAQGSTNIFINGRPAARKDDKITCGATIGDGSHDTFFHGGTQTYLPVDDEVPPWLRTATDWAFTLAGLVGGLGGLLKASGGLSRAVLPCAAKFIGGYVLGEAFGRYVAGPAINKAIGGLFGNPIDVTTGRKILLAESETDYVIPSPLPVAIKRFYSSGIDYAGTLGRGWVLPWEIRLHARDGRLWYTDAQGRESGFPMLRAGQAAFSEADQRYLTRTPDGRYILHDLGERYYDFGQYDPESGRIAWVRRVEDQAGQWYQFERDSRGRVTEILTCGGLRAVLDYETVFGRLGTVTLVHEDERRLAVTYGYDENGQLASVTDANGAVVRQFAYTNGLMTSHMNALGFTSSYVWSKIEGEPRVVETHTSEGENWTFEYDVAGRQTRVRHADGRTAHWRFDAQSQIVEYTDLDGAFYRIKYDAVGMPVMLMLPGDRTVMFEYDDAGRIIAETDPLGRTTRTRYDGNSLRPVEVVGPDGGAWRVEYDQQGRVVSNQDSLGRENRYEYPKALTALPSAHFDALGGRKTLEWNSLGKLVGYTDCSGKTTRTSFDAFGRICSRENALGQRITYDVRPTGEPRRVTYPDGSSETFEYDAAGTLVRYIGLGGRVQELLRNARGQLIEAVDPAGRRVQYRYDVEGRLRELQQDHARYTFTYSAGGRLLTETRPDGILRRFEYGEAGELLGLDIVGAPDPHATGNRSVRTIRFERDRMGVLKVQRTPTEVTRYQHDKGDRLVKVERVPTPSGIALGIVPDAVEFEYDKGGRLVAEHGSNGSVIYTLDELDNVVSLGLPHDQTLQMLRYGSGHVHQIRFGDQVVADFERDDLHREVSRTQGRLTQRSGYDPLGRKVWQSAGIDPEMLGRGSGQLWRNYGYDGAGDLIETSDSLRGSTRFSYDPAGRLISRANPLDRKFEEFAWDAAGNLLDDAQRKSRGYVEGNRLLMWQDLRFEYDPFGNLATKRRGANQTQRFTYDGQDRLITVHTQDVRGVVETRFAYDPLGRRIAKTDTAFDLRGMKLRAETKRFVWEGLRLVQEVRETGVSSYVYSPDAPYSPVARADTVMAEALAATVIDSAKRAARIFHFHTDPVGAPQEVTDEAGEVAWAGQYAAWGKVEATNRGVTAARTDQPLRFAGQYADDSTGLHYNTFRFYDPDVGRFINQDPIGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 156 - protein, 410 aa, Pseudomonas syringae
  REGION 1..410: note = MISC_FEATURE - ssdA
MSAAARVNDPIEHTGSLTGLLAGLAIGAIGAALVVGTGGLAAVAIVGASAATGAGVGQLIGSLSCCNHQTGQIVSGSSNVYINGEPAARAHADQAKCDEHSSRPQVIAQGSSNVYINGHPAARVGDRTACDAKIVVGSSNVFIGGGTETTDPINPEVPELLERGILLVGLASAFVLASPVIVIAGLVGGIAGGTVGSMGGAQLFGEGTDGQKLMAFGGALLGGGLGAKGGKWFDTRYDIKVQGVGSNLGNLKITPKGAAKVSNIAESEAALGRASQARADLPQSKELKVKTVSSNDKKTLSGWGNKKPEGYERISAEQVKAKSEEIGHEVKSHPYDRDYKGQYFSSHAEKQMSIASPNHPLGVSKPMCTDCQGYFSQLAKYSKVEQTVADPKAIRIFKTDGSVETIMRSE

SEQ ID NO: 157 - DNA, 45 bp, synthetic construct
  misc_feature 1..45: note = Synthetic construct
  misc_feature 20: note = 5caC
  source 1..45: mol_type = other DNA
acacccatcacatttacaccgggaaagagttgaatgtagagttgg

SEQ ID NO: 158 - DNA, 45 bp, synthetic construct; identical to SEQ ID NO: 157
  misc_feature 1..45: note = Synthetic construct
  misc_feature 20: note = 5fC
  source 1..45: mol_type = other DNA

SEQ ID NO: 159 - protein, 223 aa, synthetic construct
MATVKFKYKGEEKEVDISKIKKVWRVGKMISFTYDEGGGKTGRGAVSEKDAPKELLQMLEKQKKVTAGVGEAGKKEPWEAIDRFRKSNGLEPLGDRIPVRGDGLETVALMEVSGNKVFGVNSSLLSDELKNLGRDFFKVIKEKGLLGNAKHYGSGEAQVLTHAEAHALMKARKEAGGHLGDSVVLYVDRLTCPNCQKYLPEVRAAMGIKTLKVITKGGIELIL

SEQ ID NO: 160 - protein, 229 aa, synthetic construct
MATVKFKYKGEEKEVDISKIKKVWRVGKMISFTYDEGGGKTGRGAVSEKDAPKELLQMLEKQKKGGGVTSVTAGVGEAGKKEPWEAIDRFRKSNGLEPLGDRIPVRGDGLETVALMEVSGNKVFGVNSSLLSDELKNLGRDFFKVIKEKGLLGNAKHYGSGEAQVLTHAEAHALMKARKEAGGHLGDSVVLYVDRLTCPNCQKYLPEVRAAMGIKTLKVITKGGIELIL

SEQ ID NO: 161 - protein, 207 aa, synthetic construct
MATVKFKYKGEEKEVDISKIKKVWRVGKMISFTYDEGGGKTGRGAVSEKDAPKELLQMLEKQKKGGGVTSKPSTLSGQAQAVHNLAGGLKPSKISIEKSTVAIFEVQLRDGSKTLYAAGSSGVLNPRQRALLKQIGVPEKNILFGKGSVLPVTKVNSNINHAEQIILRNIPKDAKVTRKGISWSSKHKNAPCSKCQSSVDGAGGVYD

SEQ ID NO: 162 - protein, 210 aa, synthetic construct
MATVKFKYKGEEKEVDISKIKKVWRVGKMISFTYDEGGGKTGRGAVSEKDAPKELLQMLEKQKKVTAGVGEAGKPSTLSGQAQAVHNLAGGLKPSKISIEKSTVAIFEVQLRDGSKTLYAAGSSGVLNPRQRALLKQIGVPEKNILFGKGSVLPVTKVNSNINHAEQIILRNIPKDAKVTRKGISWSSKHKNAPCSKCQSSVDGAGGVYD

SEQ ID NO: 163 - protein, 217 aa, synthetic construct
MATVKFKYKGEEKEVDISKIKKVWRVGKMISFTYDEGGGKTGRGAVSEKDAPKELLQMLEKQKKGGGVTSFAKEIEQSKEAYTRATDIIKTKKLKGCVASNGKITLSGFDGKTTKAPKDFTRVSLDTMFDYCKEIGHELPAKMPDFYDNGIKGSFFACHAEKQLSLLTDKPIGISKPMCPNCVEYFSKHAIHTKQVKITTDPKKTRIFFPNGVIREI

SEQ ID NO: 164 - protein, 223 aa, synthetic construct
MATVKFKYKGEEKEVDISKIKKVWRVGKMISFTYDEGGGKTGRGAVSEKDAPKELLQMLEKQKKLSGLSDDKLKEIFAKEIEQSKEAYTRATDIIKTKKLKGCVASNGKITLSGFDGKTTKAPKDFTRVSLDTMFDYCKEIGHELPAKMPDFYDNGIKGSFFACHAEKQLSLLTDKPIGISKPMCPNCVEYFSKHAIHTKQVKITTDPKKTRIFFPNGVIREI

SEQ ID NO: 165 - DNA, 33 bp, synthetic construct
  source 1..33: mol_type = other DNA
acactctttccctacacgacgctcttccgatct

SEQ ID NO: 166 - DNA, 32 bp, synthetic construct
  source 1..32: mol_type = other DNA
gatcggaagagcacacgtctgaactccagtca

SEQ ID NO: 167 - DNA, 40 bp, synthetic construct
  source 1..40: mol_type = other DNA
aaatttaattataaaacgatcgacgacgaaataataaaaa

SEQ ID NO: 168 - DNA, 20 bp, synthetic construct
  source 1..20: mol_type = other DNA
ggatttaagtgtaaatgtac

SEQ ID NO: 169 - DNA, 21 bp, synthetic construct
  source 1..21: mol_type = other DNA
  misc_feature 5: note = 5mC
  misc_feature 10: note = 5mC
ctgtcggaccgcagtctacga